SEQUENCE LISTING (79 sequences)

SEQ ID NO: 1 - protein, 37 aa, Homo sapiens
  REGION 1..37: note = Description of sequence: native human OXM
HSQGTFTSDYSKYLDSRRAQDFVQWLMNTKRNRNNIA

SEQ ID NO: 2 - protein, 39 aa, synthetic construct
  REGION 1..39: note = Description of sequence: OXM analog
  SITE 2: note = d-Serine
  MOD_RES 10: note = (1-naphthyl)-L-alanine
  MOD_RES 29: note = Aib
  MOD_RES 38: note = X may be Cysteine-polyethylene glycol
  MOD_RES 38: note = X may be Cysteine-polyethylene glycol 20
  VARIANT 39: note = X may be Cysteine-polyethylene glycol
  MOD_RES 39: note = X may be Cysteine-polyethylene glycol 20
  VARIANT 38: note = Position may be absent
  VARIANT 39: note = Position may be absent
  VARIANT 38: note = Xaa is Cysteine, Cysteine-polyethylene glycol, Cysteine-polyethylene glycol 20, Lysine, Glycine, or Valine
  VARIANT 39: note = Xaa is Cysteine, Cysteine-polyethylene glycol, Cysteine-polyethylene glycol 20, Lysine, Glycine, or Valine
HSQGTFTSDASKYLDEKAAQEFVQWLLNXARNRNNIAXX

SEQ ID NO: 3 - protein, 39 aa, synthetic construct
  REGION 1..39: note = Description of sequence: OXM analog
  MOD_RES 2: note = Aib
  MOD_RES 29: note = Aib
  MOD_RES 38: note = X may be Cysteine-polyethylene glycol
  MOD_RES 38: note = X may be Cysteine-polyethylene glycol 20
  MOD_RES 39: note = X may be Cysteine-polyethylene glycol
  MOD_RES 39: note = X may be Cysteine-polyethylene glycol 20
  VARIANT 38: note = Position may be absent
  VARIANT 39: note = Position may be absent
  VARIANT 38: note = Xaa is Cysteine, Cysteine-polyethylene glycol, Cysteine-polyethylene glycol 20, Lysine, Glycine, or Valine
  VARIANT 39: note = Xaa is Cysteine, Cysteine-polyethylene glycol, Cysteine-polyethylene glycol 20, Lysine, Glycine, or Valine
HXQGTFTSDYSKYLDSKKAQEFVQWLLNXGRNRNNIAXX

SEQ ID NO: 4 - protein, 39 aa, synthetic construct; identical to SEQ ID NO: 3
  REGION 1..39: note = Description of sequence: OXM analog
  MOD_RES 1: note = Histidine may be modified to 3-(1H-imidazol-5-yl)propanoic acid
  VARIANT 1: note = This position is either Histidine or 3-(1H-imidazol-5-yl)propanoic acid
  MOD_RES 2: note = Aib
  MOD_RES 29: note = Aib
  MOD_RES 39: note = X may be Cysteine-polyethylene glycol
  MOD_RES 39: note = X may be Cysteine-polyethylene glycol 20
  VARIANT 39: note = Xaa is Cysteine, Cysteine-polyethylene glycol, Cysteine-polyethylene glycol 20, Lysine, Glycine, or Valine
  VARIANT 1: note = Terminal is either hydrogen or NH2
  VARIANT 38: note = Position may be absent
  VARIANT 39: note = Position may be absent
  VARIANT 39: note = Amino acid COOH may be replace with NH2 or OH
  MOD_RES 39: note = Amino acid COOH group may be replaced with NH2
  MOD_RES 39: note = Amino acid COOH group may be replaced with OH
  VARIANT 38: note = Xaa is Cysteine, Cysteine-polyethylene glycol, Cysteine-polyethylene glycol 20, Lysine, Glycine, or Valine

SEQ ID NO: 5 - protein, 38 aa, synthetic construct
  REGION 1..38: note = Description of sequence: OXM analog
  MOD_RES 2: note = Aib
  MOD_RES 29: note = Aib
HXQGTFTSDYSKYLDSKKAQEFVQWLLNXGRNRNNIAC

SEQ ID NO: 6 - protein, 38 aa, synthetic construct; identical to SEQ ID NO: 5
  REGION 1..38: note = Description of sequence: OXM analog
  MOD_RES 2: note = Aib
  MOD_RES 29: note = Aib
  MOD_RES <1: note = C-terminal is H

SEQ ID NO: 7 - protein, 38 aa, synthetic construct; identical to SEQ ID NO: 5
  REGION 1..38: note = Description of sequence: OXM analog
  MOD_RES 2: note = Aib
  MOD_RES 29: note = Aib

SEQ ID NO: 8 - protein, 39 aa, synthetic construct
  REGION 1..39: note = Description of sequence: OXM analog
  MOD_RES 2: note = Aib
  MOD_RES 29: note = Aib
HXQGTFTSDYSKYLDSKKAQEFVQWLLNXGRNRNNIAKG

SEQ ID NO: 9 - protein, 39 aa, synthetic construct; identical to SEQ ID NO: 8
  REGION 1..39: note = Description of sequence: OXM analog
  MOD_RES 2: note = Aib
  MOD_RES 29: note = Aib

SEQ ID NO: 10 - protein, 39 aa, synthetic construct; identical to SEQ ID NO: 8
  REGION 1..39: note = Description of sequence: OXM analog
  MOD_RES 2: note = Aib
  MOD_RES 29: note = Aib

SEQ ID NO: 11 - protein, 39 aa, synthetic construct
  REGION 1..39: note = Description of sequence: OXM analog
  MOD_RES 2: note = Aib
  MOD_RES 29: note = Aib
HXQGTFTSDYSKYLDSKKAQEFVQWLLNXGRNRNNIACC

SEQ ID NO: 12 - protein, 39 aa, synthetic construct; identical to SEQ ID NO: 11
  REGION 1..39: note = Description of sequence: OXM analog
  MOD_RES 2: note = Aib
  MOD_RES 29: note = Aib

SEQ ID NO: 13 - protein, 39 aa, synthetic construct; identical to SEQ ID NO: 11
  REGION 1..39: note = Description of sequence: OXM analog
  MOD_RES 2: note = Aib
  MOD_RES 29: note = Aib

SEQ ID NO: 14 - protein, 38 aa, synthetic construct
  REGION 1..38: note = Description of sequence: OXM analog
  MOD_RES 2: note = Aib
  MOD_RES 29: note = Aib
HXQGTFTSDYSKYLDSKKAQEFVQWLLNXGRNRNNIAK

SEQ ID NO: 15 - protein, 39 aa, synthetic construct
  REGION 1..39: note = Description of sequence: OXM analog
  MOD_RES 2: note = Aib
  MOD_RES 29: note = Aib
HXQGTFTSDYSKYLDSKKAQEFVQWLLNXGRNRNNIAKC

SEQ ID NO: 16 - protein, 39 aa, synthetic construct
  REGION 1..39: note = Description of sequence: OXM analog
  MOD_RES 2: note = Aib
  MOD_RES 29: note = Aib
HXQGTFTSDYSKYLDSKKAQEFVQWLLNXGRNRNNIACK

SEQ ID NO: 17 - protein, 7 aa, synthetic construct
  REGION 1..7: note = Description of sequence: OXM analog
  MOD_RES 4: note = modified with octadecanedioic acid
  VARIANT 7: note = glutamic acid modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 7: note = modified with side chain aminoethylethanolamine-Cystine-Glycine-OH
  MOD_RES 7: note = modified with side chain aminoethylethanolamine-ami noethylethanolamine-Cystine-Glycine-OH
  MOD_RES 7: note = modified with side chain aminoethylethanolamine-ami noethylethanolamine-aminoethylethanolamine-Cystine-Glycine -OH
EYEKETE

SEQ ID NO: 18 - protein, 5 aa, synthetic construct
  REGION 1..5: note = Description of sequence: OXM analog
  MOD_RES 3: note = modified with octadecanedioic acid
  VARIANT 5: note = tyrosine modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 5: note = modified with side chain aminoethylethanolamine-Cystine-Glycine-OH
  MOD_RES 5: note = modified with side chain aminoethylethanolamine-ami noethylethanolamine-Cystine-Glycine-OH
  MOD_RES 5: note = modified with side chain aminoethylethanolamine-ami noethylethanolamine-aminoethylethanolamine-Cystine-Glycine -OH
YEKEY

SEQ ID NO: 19 - protein, 7 aa, synthetic construct
  REGION 1..7: note = Description of sequence: OXM analog
  MOD_RES 4: note = modified with octadecanedioic acid
  VARIANT 7: note = arginine modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 7: note = modified with side chain aminoethylethanolamine-Cystine-Glycine-OH
  MOD_RES 7: note = modified with side chain aminoethylethanolamine-ami noethylethanolamine-Cystine-Glycine-OH
  MOD_RES 7: note = modified with side chain aminoethylethanolamine-ami noethylethanolamine-aminoethylethanolamine-Cystine-Glycine -OH
RTRKRYR

SEQ ID NO: 20 - protein, 7 aa, synthetic construct
  REGION 1..7: note = Description of sequence: OXM analog
  MOD_RES 1: note = modified with octadecanedioic acid
  MOD_RES 4: note = modified with octadecanedioic acid
  VARIANT 7: note = modified with side chain aminoethylethanolamine-Cystine-Glycine-OH
  MOD_RES 7: note = modified with side chain aminoethylethanolamine-Cystine-Glycine-OH
  MOD_RES 7: note = modified with side chain aminoethylethanolamine-ami noethylethanolamine-Cystine-Glycine-OH
  MOD_RES 7: note = modified with side chain aminoethylethanolamine-ami noethylethanolamine-aminoethylethanolamine-Cystine-Glycine -OH
EYEKEYE

SEQ ID NO: 21 - protein, 7 aa, synthetic construct
  REGION 1..7: note = Description of sequence: OXM analog
  MOD_RES 4: note = modified with octadecanedioic acid
  MOD_RES 7: note = modified with side chain aminoethylethanolamine-Cystine-Glycine-OH
EYEKEYE

SEQ ID NO: 22 - protein, 5 aa, synthetic construct
  REGION 1..5: note = Description of sequence: OXM analog
  MOD_RES 3: note = modified with eicosanedioic acid
  VARIANT 5: note = tyrosine modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 5: note = modified with side chain aminoethylethanolamine-Cystine-Glycine-OH
  MOD_RES 5: note = modified with side chain aminoethylethanolamine-ami noethylethanolamine-Cystine-Glycine-OH
  MOD_RES 5: note = modified with side chain aminoethylethanolamine-ami noethylethanolamine-aminoethylethanolamine-Cystine-Glycine -OH
YEKEY

SEQ ID NO: 23 - protein, 7 aa, synthetic construct
  REGION 1..7: note = Description of sequence: OXM analog
  MOD_RES 4: note = modified with octadecanedioic acid
  MOD_RES 7: note = modified with side chain aminoethylethanolamine-Cystine-Glycine-OH
RYRKRYR

SEQ ID NO: 24 - protein, 7 aa, synthetic construct
  REGION 1..7: note = Description of sequence: OXM analog
  MOD_RES 4: note = modified with octadecanedioic acid
  MOD_RES 7: note = modified with side chain aminoethylethanolamine-Cystine-Glycine-OH
EYEKEYE

SEQ ID NO: 25 - protein, 7 aa, synthetic construct
  REGION 1..7: note = Description of sequence: OXM analog
  MOD_RES 4: note = modified with eicosanedioic acid
  VARIANT 7: note = glutamic acid modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 7: note = modified with side chain aminoethylethanolamine-Cystine-Glycine-OH
  MOD_RES 7: note = modified with side chain aminoethylethanolamine-ami noethylethanolamine-Cystine-Glycine-OH
  MOD_RES 7: note = modified with side chain aminoethylethanolamine-ami noethylethanolamine-aminoethylethanolamine-Cystine-Glycine -OH
EYEKEYE

SEQ ID NO: 26 - protein, 5 aa, synthetic construct
  REGION 1..5: note = Description of sequence: OXM analog
  MOD_RES 3: note = modified with eicosanedioic acid
  VARIANT 5: note = tyrosine modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 5: note = modified with side chain aminoethylethanolamine-Cystine-Glycine-OH
  MOD_RES 5: note = modified with side chain aminoethylethanolamine-ami noethylethanolamine-Cystine-Glycine-OH
  MOD_RES 5: note = modified with side chain aminoethylethanolamine-ami noethylethanolamine-aminoethylethanolamine-Cystine-Glycine -OH
YEKEY

SEQ ID NO: 27 - protein, 7 aa, synthetic construct
  REGION 1..7: note = Description of sequence: OXM analog
  MOD_RES 4: note = modified with eicosanedioic acid
  VARIANT 7: note = arginine modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 7: note = modified with side chain aminoethylethanolamine-Cystine-Glycine-OH
  MOD_RES 7: note = modified with side chain aminoethylethanolamine-ami noethylethanolamine-Cystine-Glycine-OH
  MOD_RES 7: note = modified with side chain aminoethylethanolamine-ami noethylethanolamine-aminoethylethanolamine-Cystine-Glycine -OH
RYRKRYR

SEQ ID NO: 28 - protein, 7 aa, synthetic construct
  REGION 1..7: note = Description of sequence: OXM analog
  MOD_RES 1: note = modified with eicosanedioic acid
  MOD_RES 4: note = modified with eicosanedioic acid
  VARIANT 7: note = glutamic acid modfied with aminoethylethanolamine-Cystine-Glycine-OH, aminoethylethan olamine-aminoethylethanolamine-Cystine-Glycine-OH, or aminoethylethanolamine-aminoethylethanolamine-aminoethylet hanolamine-Cystine-Glycine-OH
  MOD_RES 7: note = modified with side chain aminoethylethanolamine-Cystine-Glycine-OH
  MOD_RES 7: note = modified with side chain aminoethylethanolamine-ami noethylethanolamine-Cystine-Glycine-OH
  MOD_RES 7: note = modified with side chain aminoethylethanolamine-ami noethylethanolamine-aminoethylethanolamine-Cystine-Glycine -OH
EYEKEYE

SEQ ID NO: 29 - protein, 7 aa, synthetic construct
  REGION 1..7: note = Description of sequence: OXM analog
  MOD_RES 4: note = modified with eicosanedioic acid
  MOD_RES 7: note = modified with side chain aminoethylethanolamine-Cystine-Glycine-OH
EYEKEYE

SEQ ID NO: 30 - protein, 4 aa, synthetic construct
  REGION 1..4: note = Description of sequence: spacer
  MOD_RES 1: note = modified at NH2 terminal with aminoethylethanolamin e-aminoethylethanolamine-aminoethylethanolamine-glutamic acid-glutamic acid-glutamic acid-gamma-glutamic acid
KGGG

SEQ ID NO: 31 - protein, 7 aa, synthetic construct
  REGION 1..7: note = Description of sequence: OXM analog
  MOD_RES 4: note = modified with eicosanedioic acid
  MOD_RES 7: note = modified with side chain aminoethylethanolamine-Cystine-Glycine-OH
RYRKRYR

SEQ ID NO: 32 - protein, 7 aa, synthetic construct
  REGION 1..7: note = Description of sequence: OXM analog
  MOD_RES 4: note = modified with eicosanedioic acid
  MOD_RES 7: note = modified with side chain aminoethylethanolamine-Cystine-Glycine-OH
EYEKEYE

SEQ ID NO: 33 - protein, 4 aa, synthetic construct
  REGION 1..4: note = Description of sequence: spacer
  SITE 1: note = gamma-glutamic acid
  MOD_RES 4: note = modified with side chain aminoethylethanolamine-ami noethylethanolamine-aminoethylethanolamine-Cystine-Glycine -Glycine-Glycine-OH
EEEE

SEQ ID NO: 34 - protein, 4 aa, synthetic construct
  REGION 1..4: note = Description of sequence: spacer
  MOD_RES 1: note = modified at NH2 terminal with aminoethylethanolamin e-aminoethylethanolamine-aminoethylethanolamine-glutamic acid-glutamic acid-glutamic acid-gamma-glutamic acid
CGGG

SEQ ID NO: 35 - protein, 4 aa, synthetic construct
  REGION 1..4: note = Description of sequence: spacer
  SITE 1: note = gamma-glutamic acid
  MOD_RES 4: note = modified with side chain aminoethylethanolamine-ami noethylethanolamine-aminoethylethanolamine-lysine-acetyl bromide-glycine-glycine-glycine
EEEE

SEQ ID NO: 36 - protein, 4 aa, synthetic construct
  REGION 1..4: note = Description of sequence: spacer
  MOD_RES 1: note = modified acetyl bromide side chain
  MOD_RES 1: note = modified at NH2 terminal with aminoethylethanolamin e-aminoethylethanolamine-aminoethylethanolamine-glutamic acid-glutamic acid-glutamic acid-gamma-glutamic acid
KGGG

SEQ ID NO: 37 - protein, 4 aa, synthetic construct
  REGION 1..4: note = Description of sequence: spacer
  SITE 1: note = gamma-glutamic acid
  MOD_RES 4: note = modified with side chain aminoethylethanolamine-ami noethylethanolamine-aminoethylethanolamine-lysine-glycine- glycine-glycine
EEEE

SEQ ID NO: 38 - protein, 4 aa, synthetic construct
  REGION 1..4: note = Description of sequence: spacer
  MOD_RES 1: note = modified at NH2 terminal with aminoethylethanolamin e-aminoethylethanolamine-aminoethylethanolamine-glutamic acid-glutamic acid-glutamic acid-gamma-glutamic acid
KGGG

SEQ ID NO: 39 - protein, 38 aa, synthetic construct; identical to SEQ ID NO: 5
  REGION 1..38: note = Description of sequence: modified OXM
  MOD_RES 2: note = Aib
  MOD_RES 28: note = Aib
  MOD_RES 38: note = modified with eicosanedioic-gamma-glutamic acid-ami noethylethanolamine-aminoethylethanolamine-lysine(Ac)-glyc ine

SEQ ID NO: 40 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-lysine( Ac)-glycine

SEQ ID NO: 41 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-aminoethylethan olamine-lysine(Ac)-glycine

SEQ ID NO: 42 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-aminoet hylethanolamine-lysine(Ac)-glycine

SEQ ID NO: 43 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with eicosanedioic-gamma-glutamic acid-aminoethylethanolamine-aminoethylethanolamine-glycine

SEQ ID NO: 44 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-glycine

SEQ ID NO: 45 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-gamma-glutamic acid

SEQ ID NO: 46 - protein, 38 aa, synthetic construct; identical to SEQ ID NO: 5
  REGION 1..38: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-gamma-g lutamic acid

SEQ ID NO: 47 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-aminoethylethan olamine-lysine(Ac)-glycine

SEQ ID NO: 48 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 39: note = modified with eicosanedioic-gamma-glutamic acid-ami noethylethanolamine-aminoethylethanolamine-aminoethylethan olamine-lysine(Ac)-glycine

SEQ ID NO: 49 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-lysine(Ac)-glyc ine
  MOD_RES 39: note = modified with eicosanedioic-gamma-glutamic acid-ami noethylethanolamine-aminoethylethanolamine-lysine(Ac)-glyc ine

SEQ ID NO: 50 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-aminoethylethan olamine-lysine(Ac)-glycine
  MOD_RES 39: note = modified with eicosanedioic-gamma-glutamic acid-ami noethylethanolamine-aminoethylethanolamine-aminoethylethan olamine-lysine(Ac)-glycine

SEQ ID NO: 51 - protein, 38 aa, synthetic construct; identical to SEQ ID NO: 14
  REGION 1..38: note = Description of sequence: modified OXM
  MOD_RES 2: note = Aib
  MOD_RES 29: note = Aib
  MOD_RES 38: note = residue is bound to side chain eicosanedioic-gamme- glutamate-aminoethylethanolamine-aminoethylethanolamine-NH 2

SEQ ID NO: 52 - protein, 38 aa, synthetic construct; identical to SEQ ID NO: 14
  REGION 1..38: note = Description of sequence: modified OXM
  MOD_RES 2: note = Aib
  MOD_RES 29: note = Aib
  MOD_RES 38: note = residue is bound to side chain octadecanedioic-gamm e-glutamate-aminoethylethanolamine-aminoethylethanolamine- NH2

SEQ ID NO: 53 - protein, 38 aa, synthetic construct; identical to SEQ ID NO: 14
  REGION 1..38: note = Description of sequence: modified OXM
  MOD_RES 2: note = Aib
  MOD_RES 29: note = Aib
  MOD_RES 38: note = residue is bound to side chain eicosanedioic-gamme- glutamate-gamme-glutamate-aminoethylethanolamine-aminoethy lethanolamine-NH2

SEQ ID NO: 54 - protein, 38 aa, synthetic construct; identical to SEQ ID NO: 14
  REGION 1..38: note = Description of sequence: modified OXM
  MOD_RES 2: note = Aib
  MOD_RES 29: note = Aib
  MOD_RES 38: note = residue is bound to side chain eicosanedioic-gamme- glutamate-gamme-glutamate-aminoethylethanolamine-aminoethy lethanolamine-OH

SEQ ID NO: 55 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-lysine( acetyl)-glycine
  MOD_RES 39: note = modified with octadecanedioic-gamma-glutamic acid-aminoethylethanolamine-aminoethylethanolamine-lysine( acetyl)-glycine

SEQ ID NO: 56 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-aminoet hylethanolamine-lysine(acetyl)-glycine
  MOD_RES 39: note = modified with octadecanedioic-gamma-glutamic acid-aminoethylethanolamine-aminoethylethanolamine-aminoet hylethanolamine-lysine(acetyl)-glycine

SEQ ID NO: 57 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-aminoet hylethanolamine-lysine(acetyl)-glycine
  MOD_RES 39: note = modified with octadecanedioic-gamma-glutamic acid-aminoethylethanolamine-aminoethylethanolamine-lysine( acetyl)-glycine

SEQ ID NO: 58 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-lysine( acetyl)-glycine
  MOD_RES 39: note = modified with octadecanedioic-gamma-glutamic acid-aminoethylethanolamine-aminoethylethanolamine-aminoet hylethanolamine-lysine(acetyl)-glycine

SEQ ID NO: 59 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-aminoethylethan olamine-lysine(acetyl)-glycine
  MOD_RES 39: note = modified with eicosanedioic-gamma-glutamic acid-ami noethylethanolamine-aminoethylethanolamine-lysine(acetyl)- glycine

SEQ ID NO: 60 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-lysine(acetyl)- glycine
  MOD_RES 39: note = modified with eicosanedioic-gamma-glutamic acid-ami noethylethanolamine-aminoethylethanolamine-aminoethylethan olamine-lysine(acetyl)-glycine

SEQ ID NO: 61 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-lysine(acetyl)- glycine
  MOD_RES 39: note = modified with octadecanedioic-gamma-glutamic acid-aminoethylethanolamine-aminoethylethanolamine-lysine( acetyl)-glycine

SEQ ID NO: 62 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-lysine( acetyl)-glycine
  MOD_RES 39: note = modified with eicosanedioic-gamma-glutamic acid-ami noethylethanolamine-aminoethylethanolamine-lysine(acetyl)- glycine

SEQ ID NO: 63 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-aminoethylethan olamine-lysine(acetyl)-glycine
  MOD_RES 39: note = modified with octadecanedioic-gamma-glutamic acid-aminoethylethanolamine-aminoethylethanolamine-aminoet hylethanolamine-lysine(acetyl)-glycine

SEQ ID NO: 64 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-aminoet hylethanolamine-lysine(acetyl)-glycine
  MOD_RES 39: note = modified with eicosanedioic-gamma-glutamic acid-ami noethylethanolamine-aminoethylethanolamine-aminoethylethan olamine-lysine(acetyl)-glycine

SEQ ID NO: 65 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-aminoethylethan olamine-lysine(acetyl)-glycine
  MOD_RES 39: note = modified with octadecanedioic-gamma-glutamic acid-aminoethylethanolamine-aminoethylethanolamine-lysine( acetyl)-glycine

SEQ ID NO: 66 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-aminoet hylethanolamine-lysine(acetyl)-glycine
  MOD_RES 39: note = modified with eicosanedioic-gamma-glutamic acid-ami noethylethanolamine-aminoethylethanolamine-lysine(acetyl)- glycine

SEQ ID NO: 67 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with eicosanedioic-gamma-glutamic acid-ami noethylethanolamine-aminoethylethanolamine-lysine(acetyl)- glycine
  MOD_RES 39: note = modified with octadecanedioic-gamma-glutamic acid-aminoethylethanolamine-aminoethylethanolamine-aminoet hylethanolamine-lysine(acetyl)-glycine

SEQ ID NO: 68 - protein, 39 aa, synthetic construct; identical to SEQ ID NO: 11
  REGION 1..39: note = Description of sequence: modified OXM
  MOD_RES 2: note = Aib
  MOD_RES 29: note = Aib
  MOD_RES 38: note = modified with octadecanedioic-gamma-glutamic acid-aminoethylethanolamine-aminoethylethanolamine-lysine( acetyl)-glycine
  MOD_RES 39: note = modified with eicosanedioic-gamma-glutamic acid-ami noethylethanolamine-aminoethylethanolamine-aminoethylethan olamine-lysine(acetyl)-glycine

SEQ ID NO: 69 - protein, 38 aa, synthetic construct; identical to SEQ ID NO: 5
  REGION 1..38: note = Description of sequence: modified OXM
  MOD_RES 2: note = Aib
  MOD_RES 29: note = Aib

SEQ ID NO: 70 - protein, 39 aa, synthetic construct; identical to SEQ ID NO: 8
  REGION 1..39: note = Description of sequence: modified OXM
  MOD_RES 2: note = Aib
  MOD_RES 29: note = Aib

SEQ ID NO: 71 - protein, 39 aa, synthetic construct; identical to SEQ ID NO: 11
  REGION 1..39: note = Description of sequence: modified OXM
  MOD_RES 2: note = Aib
  MOD_RES 29: note = Aib

SEQ ID NO: 72 - protein, 38 aa, synthetic construct; identical to SEQ ID NO: 5
  REGION 1..38: note = Description of sequence: 0
  MOD_RES 2: note = Aib
  MOD_RES 29: note = Aib
  MOD_RES 37: note = modified with eicosanedioic-gamma-glutamic acid-ami noethylethanolamine-aminoethylethanolamine-lysine(acetyl bromide)-glycine-OH

SEQ ID NO: 73 - protein, 38 aa, synthetic construct; identical to SEQ ID NO: 5
  REGION 1..38: note = Description of sequence: 0
  MOD_RES 2: note = Aib
  MOD_RES 29: note = Aib

SEQ ID NO: 74 - protein, 38 aa, synthetic construct; identical to SEQ ID NO: 5
  REGION 1..38: note = Description of sequence: 0
  MOD_RES 2: note = Aib
  MOD_RES 29: note = Aib
  MOD_RES 37: note = modified with eicosanedioic-gamma-glutamic acid-ami noethylethanolamine-aminoethylethanolamine-lysine(acetyl)- glycine-OH

SEQ ID NO: 75 - protein, 38 aa, synthetic construct
  REGION 1..38: note = Description of sequence: modified OXM
  MOD_RES 38: note = Side chain serine-histidine
HBQGTFTSDYSKYLDSKKSQEFVQMLLNBGRNRNNIAC

SEQ ID NO: 76 - protein, 38 aa, synthetic construct; identical to SEQ ID NO: 75
  REGION 1..38: note = Description of sequence: modified OXM
  MOD_RES 38: note = modified with eicosanedioic-gamma-glutamic acid-ami noethylethanolamine-aminoethylethanolamine-L-lysine

SEQ ID NO: 77 - protein, 39 aa, synthetic construct; identical to SEQ ID NO: 8
  REGION 1..39: note = Description of sequence: modified OXM
  MOD_RES 1: note = 1-triphenylmethyl-L-histidine
  MOD_RES 2: note = Aib
  MOD_RES 3: note = 1-triphenylmethyl-L-glutamic acid
  MOD_RES 5: note = pseudoproline-threonine
  MOD_RES 7: note = tert-Butyl-threonine
  MOD_RES 8: note = tert-Butyl-L-serine
  MOD_RES 9: note = L-aspartic acid 4-tert-butyl ester
  MOD_RES 10: note = tert-Butyl-L-tyrosine
  MOD_RES 11: note = tert-Butyl-L-serine
  MOD_RES 12: note = tert-Butyloxycarbonyl-L-lysine
  MOD_RES 13: note = tert-Butyl-L-tyrosine
  MOD_RES 15: note = L-aspartic acid 4-tert-butyl ester
  MOD_RES 16: note = tert-Butyl-L-serine
  MOD_RES 17: note = tert-Butyloxycarbonyl-L-lysine
  MOD_RES 18: note = tert-Butyl-L-tyrosine
  MOD_RES 20: note = 1-triphenylmethyl-L-glutamic acid
  MOD_RES 21: note = L-aspartic acid 4-tert-butyl ester
  MOD_RES 24: note = 1-triphenylmethyl-L-glutamic acid
  MOD_RES 25: note = tert-Butyloxycarbonyl-L-tryptophan
  MOD_RES 28: note = L-aspartic acid 4-tert-butyl ester
  MOD_RES 29: note = Aib
  MOD_RES 31: note = 22467-pentamethyl-3H-1-benzofuran-5-yl-L-arginine
  MOD_RES 32: note = 1-triphenylmethyl-L-asparagine
  MOD_RES 33: note = 22467-pentamethyl-3H-1-benzofuran-5-yl-L-arginine
  MOD_RES 34: note = 1-triphenylmethyl-L-asparagine
  MOD_RES 35: note = 1-triphenylmethyl-L-asparagine
  MOD_RES 38: note = 1-(2-hydroxy-44-dimethyl-6-oxocyclohexen-1-yl)-3-me thylbutylidene-L-lysine

SEQ ID NO: 78 - protein, 39 aa, synthetic construct; identical to SEQ ID NO: 8
  REGION 1..39: note = Description of sequence: modified OXM
  MOD_RES 1: note = 1-triphenylmethyl-L-histidine
  MOD_RES 2: note = Aib
  MOD_RES 3: note = 1-triphenylmethyl-L-glutamic acid
  MOD_RES 5: note = pseudoproline-threonine
  MOD_RES 7: note = tert-Butyl-threonine
  MOD_RES 8: note = tert-Butyl-L-serine
  MOD_RES 9: note = L-aspartic acid 4-tert-butyl ester
  MOD_RES 10: note = tert-Butyl-L-tyrosine
  MOD_RES 11: note = tert-Butyl-L-serine
  MOD_RES 12: note = tert-Butyloxycarbonyl-L-lysine
  MOD_RES 13: note = tert-Butyl-L-tyrosine
  MOD_RES 15: note = L-aspartic acid 4-tert-butyl ester
  MOD_RES 16: note = tert-Butyl-L-serine
  MOD_RES 17: note = tert-Butyloxycarbonyl-L-lysine
  MOD_RES 18: note = tert-Butyl-L-tyrosine
  MOD_RES 20: note = 1-triphenylmethyl-L-glutamic acid
  MOD_RES 21: note = L-aspartic acid 4-tert-butyl ester
  MOD_RES 24: note = 1-triphenylmethyl-L-glutamic acid
  MOD_RES 25: note = tert-Butyloxycarbonyl-L-tryptophan
  MOD_RES 28: note = L-aspartic acid 4-tert-butyl ester
  MOD_RES 29: note = Aib
  MOD_RES 31: note = 22467-pentamethyl-3H-1-benzofuran-5-yl-L-arginine
  MOD_RES 32: note = 1-triphenylmethyl-L-asparagine
  MOD_RES 33: note = 22467-pentamethyl-3H-1-benzofuran-5-yl-L-arginine
  MOD_RES 34: note = 1-triphenylmethyl-L-asparagine
  MOD_RES 35: note = 1-triphenylmethyl-L-asparagine
  MOD_RES 38: note = modified with eicosanedioic-gamma-glutamic acid-ami noethylethanolamine-aminoethylethanolamine-L-lysine

SEQ ID NO: 79 - protein, 40 aa, synthetic construct
  REGION 1..40: note = Description of sequence: modified OXM
  MOD_RES 1: note = 1-triphenylmethyl-L-histidine
  MOD_RES 2: note = Aib
  MOD_RES 29: note = Aib
  MOD_RES 39: note = modified with eicosanedioic-gamma-glutamic acid-ami noethylethanolamine-aminoethylethanolamine-L-lysine
HXQGTFTSDYSKYLDSKKAQEFVQWLLNXBGRNRNNIAKG

(19) United States
(12) Patent Application Publication
(10) Pub. No.: US 2025/0326814 A1
(43) Pub. Date: Oct. 23, 2025

(54) MODIFIED OXYNTOMODULIN AND METHODS OF USE THEREOF

(71) Applicant: OPKO BIOLOGICS LTD., Kiryat Gat (IL)

(72) Inventors: Laura MOSCHCOVICH, Givat Shmuel (IL); Miri GERZON-ZAKAR, Kidron (IL); Amit RIVKIN, Nes Ziona (IL); Antonio F. CRUZ, Miami, FL (US)

(73) Assignee: OPKO BIOLOGICS LTD., Kiryat Gat (IL)

(21) Appl. No.: 19/046,153

(22) Filed: Feb. 5, 2025

Related U.S. Application Data
(60) Provisional application No. 63/550,351, filed on Feb. 6, 2024.
(60) Provisional application No. 63/719,865, filed on Nov. 13, 2024.

(51) Int. Cl. C07K 14/575; A61K 38/00; A61P 3/00

(52) U.S. Cl. CPC C07K 14/575; A61P 3/00; A61K 38/00; C07K 2319/70

(57) ABSTRACT

Described herein are new modified oxyntomodulin (OXM) complexes. The application further provides new methods of treating diseases or conditions, such as metabolic syndrome, diabetes, obesity, and cardiovascular disease, for example, using the new modified OXM complexes.

### CROSS-REFERENCE TO RELATED APPLICATIONS

This application claims the benefit of priority of United States Provisional Application Nos. 63/550,351 filed Feb. 6, 2024 and 63/719,865 filed Nov. 13, 2024, which are hereby incorporated by reference in their entirety.

### SEQUENCE LISTING

A Sequence Listing conforming to the rules of WIPO Standard ST.26 is hereby incorporated by reference. Said Sequence Listing has been filed as an electronic document encoded as XML in UTF-8 text. The electronic document, created on Mar. 12, 2025, is entitled “P-629858-US_ST26.xml”, and is 229,555 bytes in size.

### FIELD OF THE INVENTION

Described herein are new modified oxyntomodulin (OXM) complexes. The application further provides new methods of treating diseases or conditions, such as metabolic syndrome, diabetes, obesity, and cardiovascular disease, for example, using the new modified OXM complexes.

### BACKGROUND

### Metabolic Diseases

Metabolic syndrome (MetS) is a group of metabolic abnormalities that includes hypertension, central obesity, insulin resistance, and atherogenic dyslipidemia. Metabolic diseases include cancer, diabetes, bone metabolism disorders, fatty liver, obesity, and cardiovascular disease. MetS is strongly associated with an increased risk of developing atherosclerotic cardiovascular disease (CVD). (Grundy, Scott M., et al. “Clinical management of metabolic syndrome: report of the American Heart Association/National Heart, Lung, and Blood Institute/American Diabetes Association conference on scientific issues related to management.” Circulation 109.4 (2004): 551-556) The pathogenesis of MetS involves both genetic and acquired factors that play a role in the final pathway of inflammation that leads to CVD. Typically, metabolic diseases are caused by abnormalities in the metabolism of glucose, fat, proteins, and others. MetS has become increasingly relevant in recent times due to the exponential increase in obesity worldwide.

Obesity is a chronic disease characterized by the abnormal or excessive accumulation of body fat, affecting more than 1 billion people worldwide. Factors such as a sedentary lifestyle, overnutrition, socioeconomic status, and other environmental and genetic conditions can cause obesity. Many molecules and signaling pathways are involved in the pathogenesis of obesity, such as nuclear factor (NF)-κB, Toll-like receptors (TLRs), adhesion molecules, G protein-coupled receptors (GPCRs), programmed cell death 1 (PD-1)/programmed death-ligand 1 (PD-L1), and sirtuin 1 (SIRT1).

Obesity is commonly associated with many other metabolic disorders, including type 2 diabetes (T2D), non-alcoholic fatty liver disease (NAFLD), cardiovascular diseases (CVDs), chronic kidney diseases (CKDs), and cancers. (Kyrou, Ioannis, et al. “Clinical problems caused by obesity.” (2015)) Additionally, obesity has been shown to be positively associated with the severity and mortality of the coronavirus disease 2019 (COVID-19) in patients. (Singh, Romil, et al. “Association of obesity with COVID-19 severity and mortality: An updated systemic review, meta-analysis, and meta-regression.” Frontiers in endocrinology 13 (2022): 780872) Adipose tissues secrete many inflammatory cytokines such as tumor necrosis factor α (TNF-α) and interleukin 6 (IL-6), which are a group of major contributing factors to metabolic disorders. Obesity also causes other complications, such as dysfunction of vascular epithelial cells and lipid accumulation in organs except for adipose tissues.

NAFLD is the hepatic manifestation of metabolic syndrome, and is a spectrum of hepatic conditions encompassing steatosis, non-alcoholic steatohepatitis (NASH), fibrosis, cirrhosis and ultimately hepatocellular carcinoma. NAFLD and NASH are considered the primary fatty liver diseases as they account for the greatest proportion of individuals with elevated hepatic lipids. The severity of NAFLD/NASH is based on the presence of lipid, inflammatory cell infiltrate, hepatocyte ballooning, and the degree of fibrosis. Although not all individuals with steatosis progress to NASH, a substantial portion does.

Diabetes is a common type of metabolic disease characterized by hyperglycemia. Several major types of diabetes are caused by complex interactions between genetic and environmental factors. The factors leading to hyperglycemia include the decrease of insulin secretion, the decrease of glucose utilization and the increase of glucose output, and the dominance of these factors varies according to the etiology of diabetes. Metabolic abnormalities related to diabetes lead to secondary pathophysiological changes in multiple systems throughout the body. Long-term abnormal blood glucose levels can lead to serious complications, including cardiovascular disease, chronic renal failure, retinal injury, nerve injury, microvascular injury and obesity and the like. Diabetes is classified based on different pathological processes leading to hyperglycemia, and can be divided into two main types: type 1 diabetes and type 2 diabetes. In the development of the disease, type 1 and type 2 diabetes are preceded by a phase of abnormal glucose homeostasis. Type 1 diabetes is the result of complete or almost complete insulin deficiency. Type 2 diabetes is a group of heterogeneous diseases, manifested by varying degrees of insulin resistance, decreased insulin secretion, and increased glucose production.

Currently, drugs used in the treatment of diabetes include insulin, insulin secretagogue, metformin, insulin sensitizers, α-glucosidase inhibitor, dipeptidyl peptidase-IV inhibitor (liptins), sodium-glucose cotransport protein (SGLT2) inhibitor, and glucagon-like peptide-1 (GLP-1) receptor agonist and the like. These drugs have good therapeutic effects, but there are still safety issues in long-term treatment, for example, biguanides can easily cause lactic acidosis; sulfonylureas can cause symptoms of hypoglycemia; insulin sensitizers can cause edema, heart failure, and weight gain; α-glucosidase inhibitors can cause abdominal pain, bloating, diarrhea and other symptoms; sodium-glucose cotransporter protein (SGLT2) inhibitors increase the risk of urinary and reproductive system infections and the like.

According to World Health Organization (WHO), diabetes is a chronic, metabolic disease characterized by elevated levels of blood glucose (or blood sugar), which leads over time to serious damage to the heart, blood vessels, eyes, kidneys and nerves. Diabetes occurs either when the pancreas does not produce enough insulin or when the body cannot effectively use the insulin it produces. Insulin is a hormone that regulates blood sugar. Hyperglycemia or raised blood sugar is a common effect of uncontrolled diabetes and over time leads to serious damage to many of the body's systems, especially the nerves and blood vessels.

Insulin regulates the metabolism of carbohydrates, fats and protein by promoting the absorption of glucose from the blood into fat, liver and skeletal muscle cells. Pancreatic beta cells (P cells) are sensitive to glucose concentrations in the blood. In non-diabetics, when glucose concentrations in the blood are high, the pancreatic beta cells secrete insulin into the blood; when glucose levels are low, secretion of insulin is inhibited. Pancreatic alpha cells secrete glucagon, another peptide hormone, into the blood to raise the concentration of glucose in the blood in the opposite manner, i.e., increased secretion when blood glucose is low, and decreased secretion when glucose concentrations are high. The secretion of insulin and glucagon into the blood in response to the blood glucose concentration is the primary mechanism responsible for keeping the glucose levels in the extracellular fluids within narrow limits.

The WHO reports the trend of increasing the number of diabetes and premature mortality from diabetes. For example, between 2000 and 2016, there was a 5% increase in premature mortality from diabetes. According to the WHO, the number of people with diabetes has risen from 108 million in 1980 to 422 million in 2014. In 2014, 8.5% of adults aged 18 years and older had diabetes. In 2012 diabetes was the direct cause of 1.5 million deaths and high blood glucose was the cause of another 2.2 million deaths. Diabetes is a major cause of blindness, kidney failure, heart attacks, stroke and lower limb amputation. Adults with diabetes have a 2-3-fold increased risk of heart attacks and strokes. Combined with reduced blood flow, neuropathy (nerve damage) in the feet increases the chance of foot ulcers, infection and eventual need for limb amputation. Diabetes is among the leading causes of kidney failure.

### Cardiovascular Disease

The increasing prevalence of cardiovascular (CV) disease and CV disease mortality in various human populations, including overweight and/or obese individuals, is a world health crisis of epidemic proportions that is a major contributor to patient morbidity and mortality as well as a major economic burden. For instance, obesity (e.g., persons having a body mass index (BMI, BMI kg/m2) of greater than 25 (overweight) or 30 (obese)) is a rapidly increasing problem worldwide and currently more than 65% of adults in the U.S. are overweight. And more than 80% of patients with heart failure with preserved ejection fraction (HFpEF) are overweight or obese (Kitzman, et al. Effect of caloric restriction or aerobic exercise training on peak oxygen consumption and quality of life in obese older patients with heart failure with preserved ejection fraction: A randomized clinical trial. JAMA. 2016; 315(1):36-46.) Weight loss of 7% has been shown to increase exercise tolerance and improve other measures of diastolic heart function in a study of 100 older patients (67+/−5 years) with obesity and clinically stable HFpEF (Id.; https://www.rethinkobesity.com/disease-progression/comorbidities-of-obesity.html). A majority of persons considered overweight or obese have limited options for available US Food and Drug Administration (FDA)-approved pharmacologic drugs for inducing weight loss, therapy has largely been based on lifestyle interventions directed at achieving weight loss. However, it is difficult to attain and maintain long-term weight loss with lifestyle changes alone.

### Oxyntomodulin (OXM)

Oxyntomodulin (OXM) is a 37 amino acid peptide hormone that is released with an additional hormone (glucagon-like peptide 1 (GLP-1)) from the L-cells of the small intestine. OXM reduces body weight in obese patients as a result of enhanced satiety and increased energy expenditure. (Wynne et al, Curr Opin Investig Drugs 2010; 11:1151-1157) The satiety-inducing effects of OXM are believed to be mediated through the activation of the GLP-1R antagonist exendin. (Baggio et al, Gastroenterol 2004: 127: 546-558; Sowden at al. Am J Physiol. Reg, Integr comp. Physiol. 2007; 292: R962-R970) Other effects of OXM such as further improvement of β-cell function and increased energy expenditure are attributed to the glucagon receptor pathway (Kosinski et al, Obesity 2012; 20:1566-1571).

Oxyntomodulin (OXM) improves glucose tolerance and stimulates insulin secretion in mice (Maida et al, Endocrinol 2008; 149:5670-5678). The oxyntomodulin peptide analog, for example, PEGylated derivatives, are found useful in treating type 2 diabetes and related disorders. These PEGylated derivatives are long acting analogs which bind to and activate both the glucagon-like peptide-1 receptor (GLP-1R) and the glucagon receptor (GcgR).

### Albumin (a “Chaperone” Protein Mediating the Increase of a Drugs Half-Life)

Human serum albumin (“HSA” or “albumin”) is the most abundant protein in plasma (˜60% of all proteins, ˜40 [g/L]). It is a highly soluble and stable (pH, structural, and temp), non-glycosylated, negatively charged and thus avoids filtration in the renal glomeruli and considered to be highly hydrophilic. Albumin contains 17 disulfide bridges that contribute to its structural stability, thermal stability, and a single odd cysteine residue in position 34. Overall, the Cys³⁴ residue makes up ˜80% of the free thiols in plasma. HSA is synthesized as a 585-residue single chain globular protein lacking prosthetic groups and glycosylation.

Albumin possess an extremely long half-life (˜19 days). A central contributor to the latter is its ability to bind the FcRn receptor, be rescued, and be recycled into the blood stream (Larsen, Maja Thim, et al. “Albumin-based drug delivery: harnessing nature to cure disease.” Molecular and cellular therapies 4.1 (2016): 1-12). Almost every body fluid contains some amount of HSA. In addition, HSA occurs within cells like ovarian cells, brain cells, peripheral nerve cells, lymphocytes, macrophages, and other cells. Tumor cells often take up HSA to a greater extent than non-tumorous cells of the same type. For example, albumin makes up 19% of the soluble protein of breast cancer cells.

All of the above characteristics attribute to the albumin long half-life, allowing it to take part in numerous important physiological activities. Due to its versatile and multiple binding domains it serves as a transporter and stabilizer to a variety of molecules such as: fatty acids, aromatics, ions, and peptides. The latter characteristics sparkled the imagination of scientist for designing Albumin-binding drugs that display an increased half-life.

There are several key attributes gained when binding an Albumin to a drug, specifically to a relatively small drug (aromatic and aliphatic compounds, peptides, and small proteins) with a short half-life. The attributes are as follows: drug protection from enzymatical degradation; drug physical stability; reduce renal clearance of the drug (size matter); the recycling mechanism of the Albumin bounded drug via the FcRn receptor; and molecules capable of binding to the neonatal Fc receptor (“FcRn”), such as IgG molecules and albumin, can be rescued from lysosomal degradation and can be recycled into the blood stream.

The Neonatal FC receptor (“FcRN”) was first found to the responsible for transporting antibodies of IgG class from the mother to the fetus. Since the early discovery, it was found that the FcRN receptor is broadly expressed in many other tissues, and despite the unrelated structure, can also bind Albumin (Zorzi, Alessandro, Sara Linciano, and Alessandro Angelini. “Non-covalent albumin-binding ligands for extending the circulating half-life of small biotherapeutics.” MedChemComm 10.7 (2019): 1068-1081).

Several families of Albumin probes (or Albumin binders in other words) have already been shown to successfully prolong the half-life of drugs. For each of these families, one can find examples of market available products displaying improved drug longevity, reduced frequent of injection, and increased safety. Among such binders are aromatic compounds, peptides, and nano structures, with fatty acids being one of the most promising albumin binding moieties. Moreover, the ability of serum albumin to bind long fatty acids with a high affinity inspired the use of posttranslational acylation as a safe and natural platform for prolonging the half-life of peptides and small proteins.

There are seven different fatty acids binding domains spread around three Albumin binding (and sub binding) sites. An illustration of the Albumin binding sites with respect to the Fatty acids, bi-valent ions, and known drugs is presented in FIG. 1. It was established that the DIII and DI loop are highly important for the FcRn binding, and that there are several fatty acids binding sites located at these regions as well. Therefore, upon designing an albumin-binding drug, must take into consideration the potential alternation of the Albumin conformation, and thus hampering its FcRn binding.

Long chain fatty acids (LCFAs, i.e., carboxylic acids having a non-branched aliphatic chain having 16-20 carbon atoms in its backbone) are essential for many cellular functions. LCFAs serve as an important energy resource and are also critical components of lipids, hormones, and proteins. LCFAs are known to be bound and transported by HSAs within the human body.

Fatty acids (FA) can be conjugated to therapeutic proteins to form longer-acting derivatives. This principle for prolongation of protein or peptide half-life is based on the fact that FA can bind to human serum albumin (HSA; also referred to as albumin binding probes). The association of a FA with human serum albumin in the blood stream can lead to a substantial prolongation of the half-life of the therapeutic protein as it will recycle together with albumin through the neonatal Fc receptor. FA and derivatives thereof (e.g., corresponding methyl esters) have shown similar albumin-binding properties (Spector A A, J Lipid Res 1975; 16: 165-79).

Formation of conjugates between LCFAs and many small molecules is known to enhance the serum stability and delivery of the small molecules by a mechanism facilitated by binding of the small molecule-LCFA conjugate with HSA.

Formation of conjugates between LCFAs and many small molecules is known to enhance the serum stability and delivery of the small molecules by a mechanism facilitated by binding of the small molecule-LCFA conjugate with HSA.

### Unmet Need for a Long Acting Oxyntomodulin

Existing GLP-1 receptor agonist therapies have the potential for harmful side effects, that may discourage the continued use of the treatments. It is well documents that side effects from both oral and subcutaneous administration of semaglutides include gastrointestinal disturbances, such as nausea, vomiting and diarrhea. When compared with placebo, subcutaneous semaglutide for 30 weeks induced nausea in 11.4 to 20% of the semaglutide-treated patients (placebo 3.3-8%), vomiting in 4 to 11.5% (placebo 2-3%) and diarrhea in 4.5 to 11.3% (placebo 1.5-6%). (Smits, Mark M., and Daniël H. Van Raalte. “Safety of semaglutide.” Frontiers in endocrinology 12 (2021): 645563) Where generally older patients with comorbid conditions were treated for 104 weeks, the incidence of GI disturbances was somewhat higher. Higher doses of semaglutide formulations are often associated with more frequent GI adverse effects. GI complaints are the main adverse-event related cause of drug discontinuation.

There remains a need for an easily-administered prevention and/or treatment for cardiometabolic and associated diseases.

The modified OXM and modified OXM analogs newly disclosed in the present application have a similar or potentially higher effect on weight loss compared to the GLP-1 agonists currently available. In addition, it is believed that certain embodiments of the OXM acylated variants recited herein will have higher longevity over the weekly approved GLP-1 agonists.

The present application discloses the benefits of albumin binding to produce new OXM prodrug complexes with improved pharmacokinetic and therapeutic properties. This application further presents new methods for treating a variety of metabolic diseases using these modified OXM complexes.

### SUMMARY

In one aspect, disclosed herein is a modified oxyntomodulin (“OXM”) having the structure of formula (II):

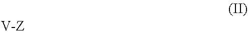

wherein:
V represents a binder complex; and
Z represents native OXM, an OXM analog, or an active fragment thereof.

In another aspect, the native OXM comprises the amino acid sequence of SEQ ID NO: 1.

In another aspect, the OXM analog comprises any of one of SEQ ID NOs: 2 to 16.

In a related aspect, the V of formula II is:
Eicosanedioic-gGlu-(AEEA)ₙ-Lys(AcBr)-Gly-OH;
Octadecanedioic-gGlu-(AEEA)ₙ-Lys(AcBr)-Gly-OH;
Eicosanedioic-gGlu-Glu-(AEEA)ₙ-Lys(AcBr)-Gly-OH;
Octadecanedioic-gGlu-Glu-(AEEA)ₙ-Lys(AcBr)-Gly-OH;
Eicosanedioic-gGlu-(AEEA)ₙ;
Octadecanedioic-gGlu-(AEEA)ₙ;
Hexadecanedioic-gGlu-AEEA-Cys-Gly-OH;
Octadecanedioic-gGlu-AEEA-Cys-Gly-OH;
Hexadecanedioic-gGlu-(AEEA)₂-Cys-Gly-OH;
Octadecanedioic-gGlu-(AEEA)₂-Cys-Gly-OH.
H-Glu-Tyr-Glu-Lys(Octadecanedioic)-Glu-Tyr-Glu-AEEA-Cys-Gly-OH (SEQ ID NO: 21);
H-Tyr-Glu-Lys(Octadecanedioic)-Glu-Tyr-AEEA-Cys-Gly-OH (SEQ ID NO: 22);
H-Glu-Lys(Octadecanedioic)-Glu-AEEA-Cys-Gly-OH;
H-Arg-Tyr-Arg-Lys(Octadecanedioic)-Arg-Tyr-Arg-AEEA-Cys-Gly-OH (SEQ ID NO: 23);
Octadecanedioic-Glu-Tyr-Glu-Lys(Octadecanedioic)-Glu-Tyr-Glu-AEEA-Cys-Gly-OH (SEQ ID NO: 24);
H-Glu-Tyr-Glu-Lys(Eicosanedioic)-Glu-Tyr-Glu-AEEA-Cys-Gly-OH (SEQ ID NO: 29);
H-Tyr-Glu-Lys(Eicosanedioic)-Glu-Tyr-AEEA-Cys-Gly-OH (SEQ ID NO: 30);
H-Glu-Lys(Eicosanedioic)-Glu-AEEA-Cys-Gly-OH;
H-Arg-Tyr-Arg-Lys(Eicosanedioic)-Arg-Tyr-Arg-AEEA-Cys-Gly-OH (SEQ ID NO: 31); or
Eicosanedioic-Glu-Tyr-Glu-Lys(Eicosanedioic)-Glu-Tyr-Glu-AEEA-Cys-Gly-OH (SEQ ID NO: 32),
   wherein n is 1, 2 or 3.

In another aspect, the V of formula (II) is conjugated to any lysine, cysteine, or glycine residue present in said native OXM, an OXM analog, or an active fragment thereof.

In one aspect, the V of formula (II) is conjugated at one or more positions between amino acid position numbers: 17 to 37 of SEQ ID NO: 1; 18 to 39 of SEQ ID NO: 5 or 14; 17 to 38 of SEQ ID NO: 6 or SEQ ID NO: 7; 18 to 40 of SEQ ID NO: 4, SEQ ID NO: 8, SEQ ID NO: 11, SEQ ID NO: 15, or SEQ ID NO: 16; or 17 to 39 of SEQ ID NOs: 2, 3, 9, 10, 12, or 13.

In one aspect, the V of formula (II) is conjugated at one or more amino acid position numbers between:
22 to 37, 25 to 37, 28 to 37, 30 to 37, 32 to 37, or 36 to 37 of SEQ ID NO: 1;
23 to 39, 26 to 39, 29 to 39, 31 to 39, 33 to 39, or 37 to 39 of SEQ ID NO: 5 or SEQ ID NO: 14;
22 to 38, 25 to 38, 28 to 38, 30 to 38, 32 to 38, or 36 to 38 of SEQ ID NO: 6 or SEQ ID NO: 7;
23 to 40, 26 to 40, 29 to 40, 31 to 40, 33 to 40, or 37 to 40 of SEQ ID NO: 4, SEQ ID NO: 8, SEQ ID NO: 11, SEQ ID NO: 15, or SEQ ID NO: 16; or
22 to 39, 25 to 39, 28 to 39, 30 to 39, 32 to 39, or 36 to 39 of SEQ ID NO: 2, 3, 9, 10, 12, or 13.

In one aspect, the V of formula (II) is conjugated at the C38, C39, K38, G39, of said OXM analog in SEQ ID NO: 2 or SEQ ID NO: 3, or any combination thereof.

In one aspect, the V of formula (II) is conjugated at the amino acid position 38 or position 39 of said OXM analog in SEQ ID NO: 2 or SEQ ID NO: 3, or any combination thereof.

In one aspect, the V of formula (II) is conjugated at the amino acid position 39 or position 40 of said OXM analog in SEQ ID NO: 4, or any combination thereof.

In one aspect, the said V of Formula II comprises the structure of Formula III:

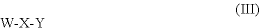

wherein:
W represents a binder;
X represents a spacer; and
Y represents an optional linker.

In one aspect, the Y of formula (III) is present and conjugated to any lysine, cysteine, or glycine residue present in said native OXM, an OXM analog, or an active fragment thereof.

In one aspect, the Y of formula (III) is not present, and X is conjugated to any lysine, cysteine, or glycine residue present in said native OXM, an OXM analog, or an active fragment thereof.

In one aspect, the Y or X of formula (III) is conjugated at the C38, C39, K38, G39, of said OXM analog, or any combination thereof.

In one aspect, W is a fatty acid. In a related aspect, W is octadecanedioic acid (C18 diacid) or is eicosanedioic acid (C20 diacid). In a related aspect, W is octadecanedioic acid (C18 diacid) and is represented by Formula IV:

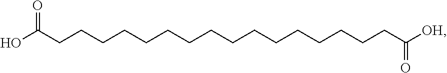 [SMILES: O=C(O)CCCCCCCCCCCCCCCCC(=O)O]

or is represented by Formula IV-A when linked to an amino group to form an amide bond:

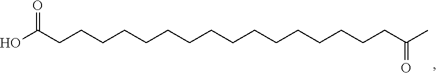 [SMILES: CC(=O)CCCCCCCCCCCCCCCCC(=O)O]

In one aspect, W is eicosanedioic acid (C20 diacid) and is represented by Formula V:

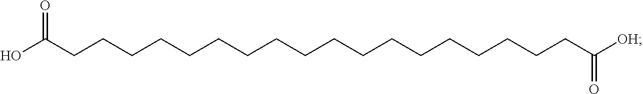 [SMILES: O=C(O)CCCCCCCCCCCCCCCCCCC(=O)O]

or is represented by Formula IV-A when linked to an amine to form an amide bond:

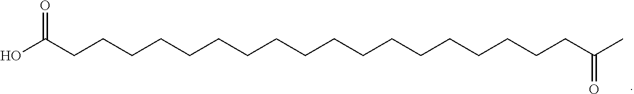 [SMILES: CC(=O)CCCCCCCCCCCCCCCCCCC(=O)O]

In one aspect, X is gGlu-Gluₙ-(AEEA)ₘ-Cys-Glyₚ(SEQ ID NOs: 33 and 34), gGlu-Gluₙ-(AEEA)ₘ-Lys(AcBr)-Glyₚ (SEQ ID NOs: 35 and 36), or gGlu-Gluₙ-(AEEA)ₘ-Lys-Glyₚ (SEQ ID NOs: 37 and 38) and n is 1, 2, or 3; m is 1, 2, or 3; and p is 1, 2, or 3.

In one aspect, X is gGlu-Glu-(AEEA)ₘ-Cys-Gly, m is 1, 2, or 3, and is represented by Formula VI:

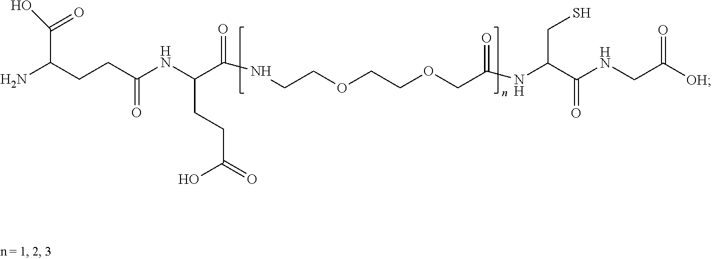

or is represented by Formula VI-E when linked to an adjoining moiety at the S bond;

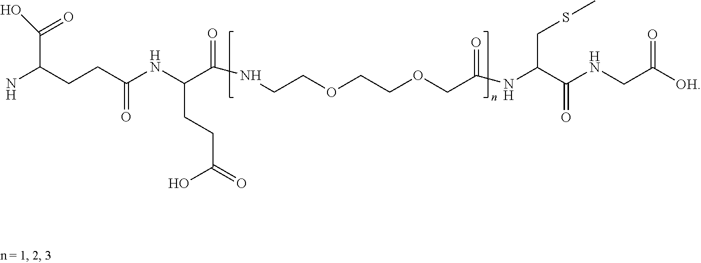

In one aspect, X is gGlu-Glu-(AEEA)ₘ-Lys(AcBr)-Gly, m is 1, 2, or 3, and is represented by Formula VII:

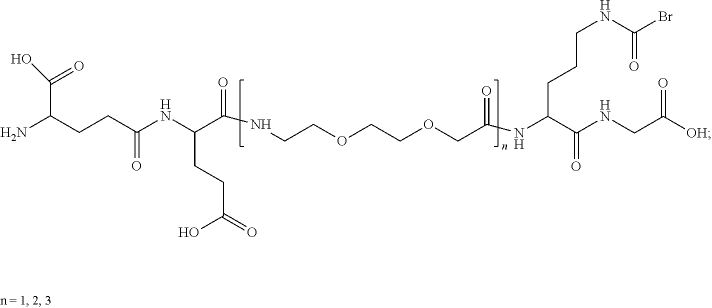

or is represented by Formula VII-C when linked to an adjoining moiety at the acyl carbon:

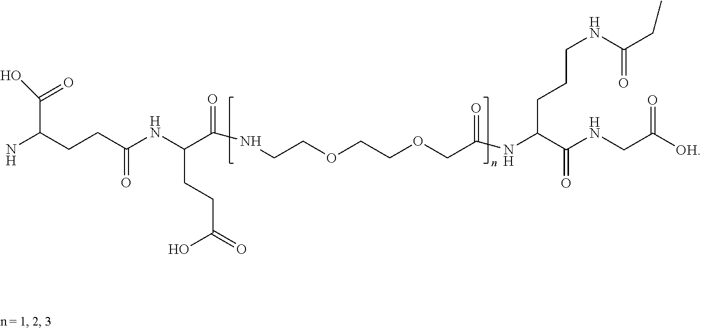

In one aspect, X is gGlu-Glu-(AEEA)ₘ-Lys-Gly, m is 1, 2, or 3, and is represented by Formula VIII:

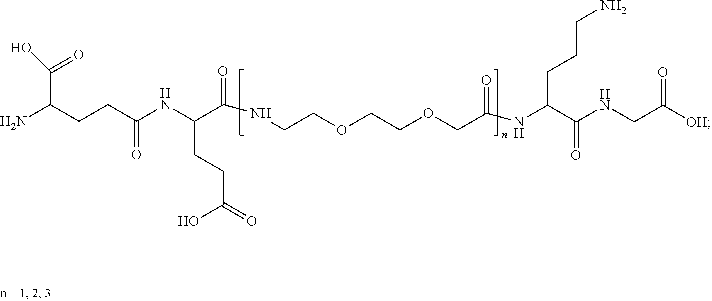

or is represented by Formula VIII-D when linked through the lysine amino group:

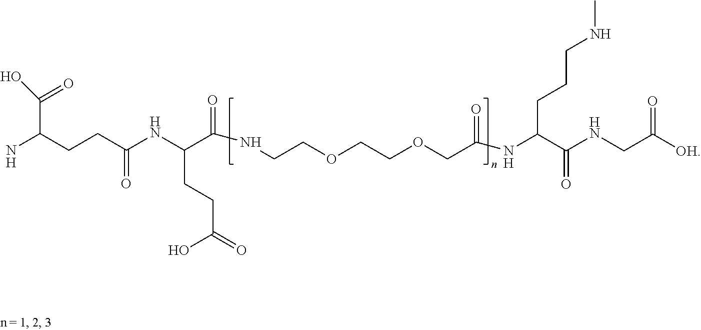

In one aspect, Y is Chloropropane-2-one-Fmoc-Mal. In a related aspect, Chloropropane-2-one-Fmoc-Mal is represented by Formula IX:

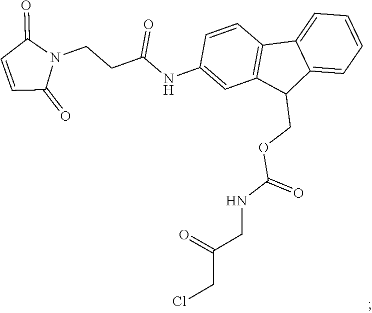 [SMILES: O=C(CCl)CNC(=O)OCC1c2ccccc2-c2ccc(NC(=O)CCN3C(=O)C=CC3=O)cc21]

or is represented by Formula IX-C when attached to an adjoining atom at the acyl carbon:

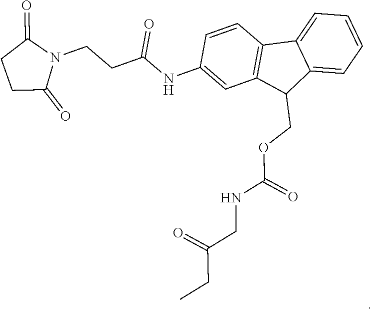 [SMILES: CCC(=O)CNC(=O)OCC1c2ccccc2-c2ccc(NC(=O)CCN3C(=O)CCC3=O)cc21]

In one aspect, Y is Mal-NRFmoc-NHS. In a related aspect, Mal-NRFmoc-NHS is represented by Formula XIII:

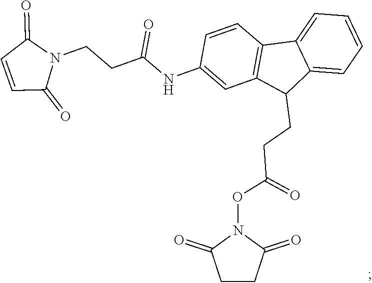 [SMILES: O=C(CCN1C(=O)C=CC1=O)Nc1ccc2c(c1)C(CCC(=O)ON1C(=O)CCC1=O)c1ccccc1-2]

or is represented by Formula XIII-C when linked at the oxygen to an adjoining atom:

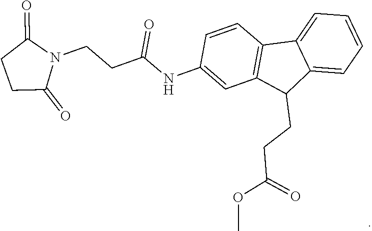 [SMILES: COC(=O)CCC1c2ccccc2-c2ccc(NC(=O)CCN3C(=O)CCC3=O)cc21]

In one aspect, the bond between the Z of Formula II and the linker is a stable covalent bond. In a related aspect, the bond between the Z of Formula II and the linker is a reversible covalent bond.

In one aspect, the modified OXM comprises the following formula:

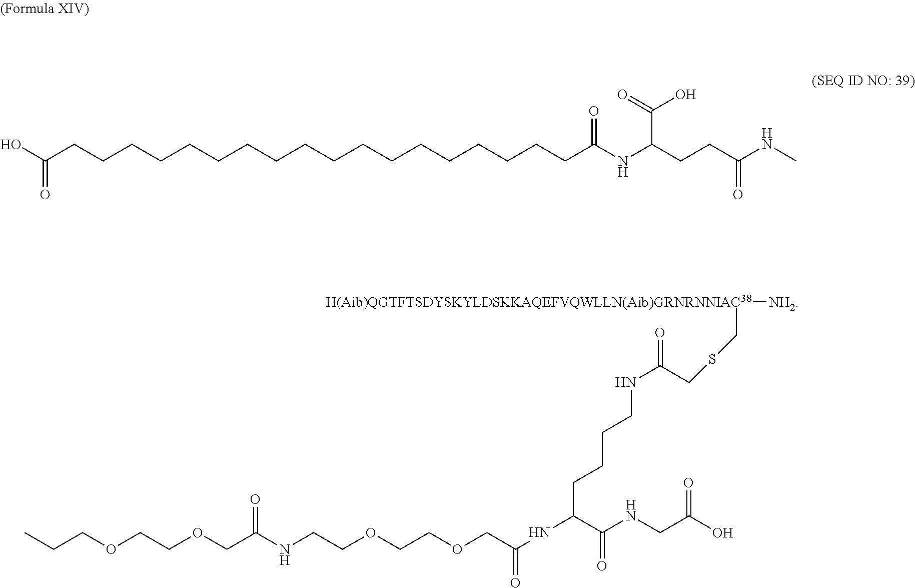

In one aspect, the modified OXM comprises H(Aib)QGTFTSDYSKYLDSKKAQEFVQWLLN(Aib)GRNRNNIAC³⁸(Eicosanedioic-gGlu-(AEEA)₂-Lys(Ac)-Gly)-NH₂ (SEQ ID NO: 39).

In one aspect, the modified OXM comprises the following formula:

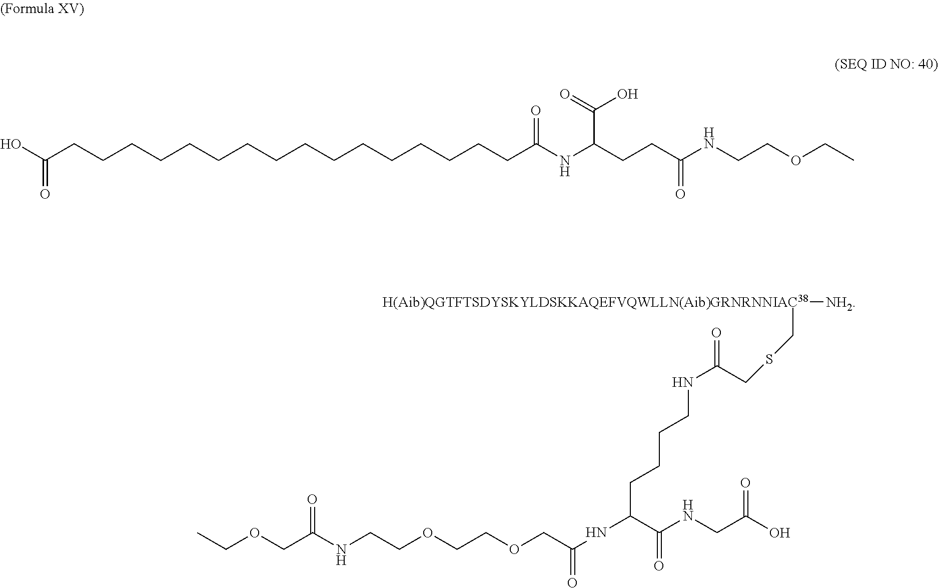

In one aspect, the modified OXM comprises H(Aib)QGTFTSDYSKYLDSKKAQEFVQWLLN(Aib)GRNRNNIAC³⁸(Octadecanedioic-gGlu-(AEEA)₂-Lys(Ac)-Gly)-NH₂ (SEQ ID NO: 40).

In one aspect, the modified OXM comprises the following formula:

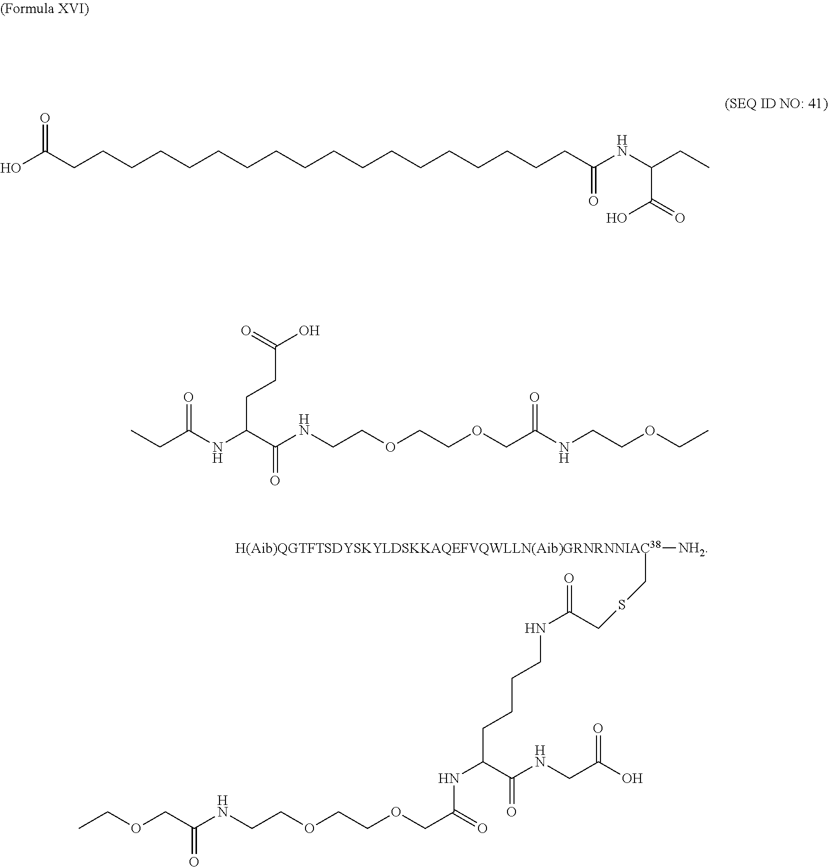

In one aspect, the modified OXM comprises H(Aib)QGTFTSDYSKYLDSKKAQEFVQWLLN(Aib)GRNRNNIAC³⁸(Eicosanedioic-gGlu-Glu-(AEEA)₃-Lys(Ac)-Gly)-NH₂ (SEQ ID NO: 41).

In one aspect, the modified OXM comprises the following formula:

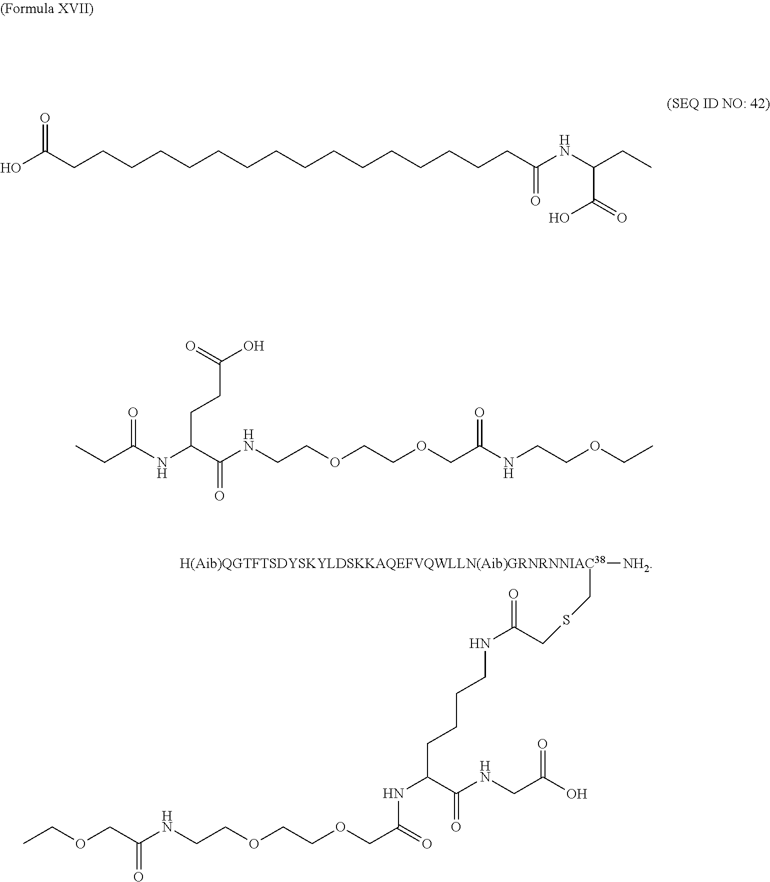

In one aspect, the modified OXM comprises H(Aib)QGTFTSDYSKYLDSKKAQEFVQWLLN(Aib)GRNRNNIAC³⁸(Octadecanedioic-gGlu-Glu-(AEEA)₃-Lys(Ac)-Gly)-NH₂ (SEQ ID NO: 42).

In one aspect, the modified OXM comprises the following formula:

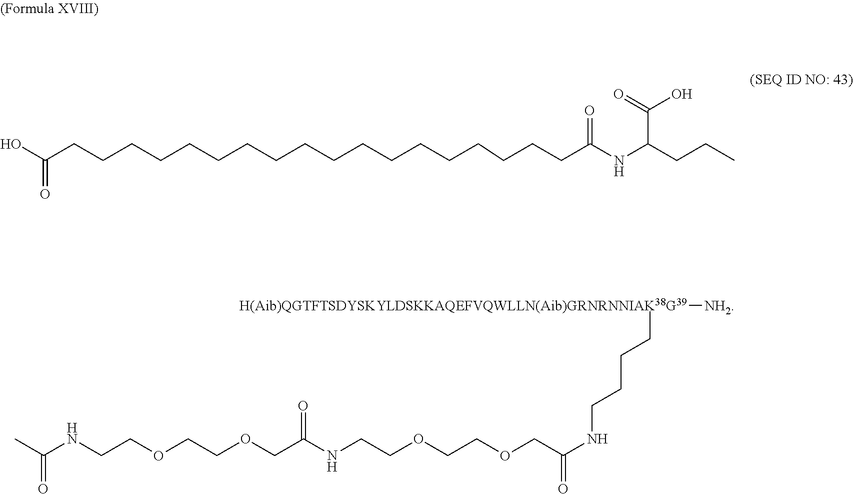

In one aspect, the modified OXM comprises H(Aib)QGTFTSDYSKYLDSKKAQEFVQWLLN(Aib)GRNRNNIAK³⁸(Eicosanedioic-gGlu-(AEEA)₂)G³⁹-NH₂ (SEQ ID NO: 43).

In one aspect, the modified OXM comprises the following formula:

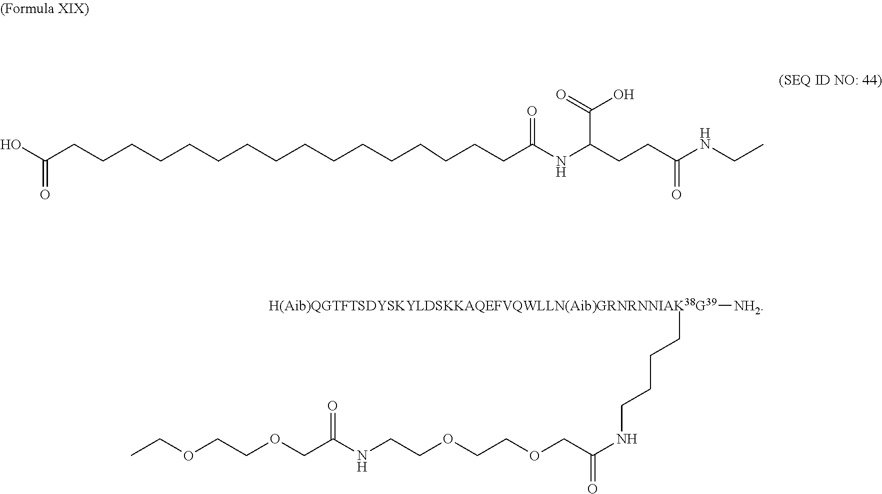

In one aspect, the modified OXM comprises H(Aib)QGTFTSDYSKYLDSKKAQEFVQWLLN(Aib)GRNRNNIAK³⁸(Octadecanedioic-gGlu-(AEEA)₂)G³⁹-NH₂ (SEQ ID NO: 44).

In one aspect, the modified OXM comprises the following formula:

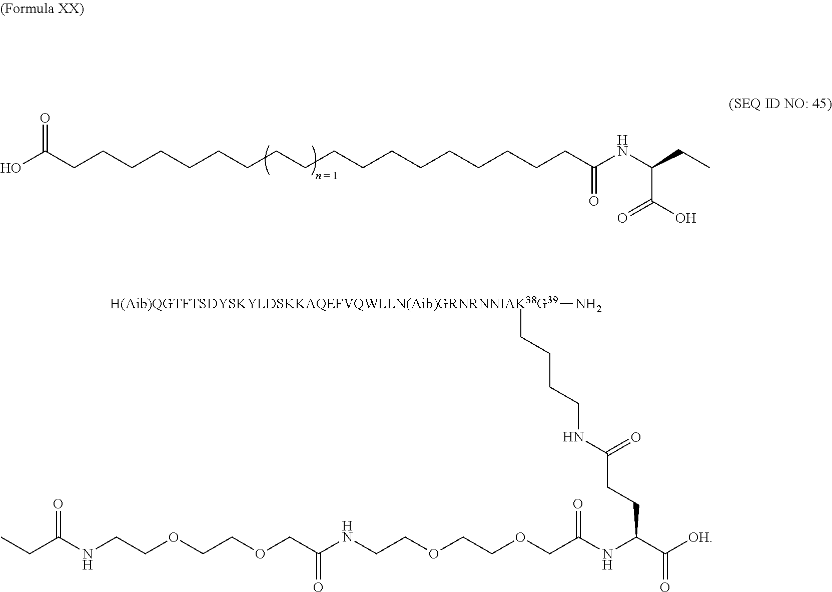

In one aspect, the modified OXM comprises H(Aib)QGTFTSDYSKYLDSKKAQEFVQWLLN(Aib)GRNRNNIAK³⁸(Eicosanedioic-gGlu-(AEEA)₂-gGlu)G³⁹-NH₂ (SEQ ID NO: 45).

In one aspect, the modified OXM comprises the following formula:

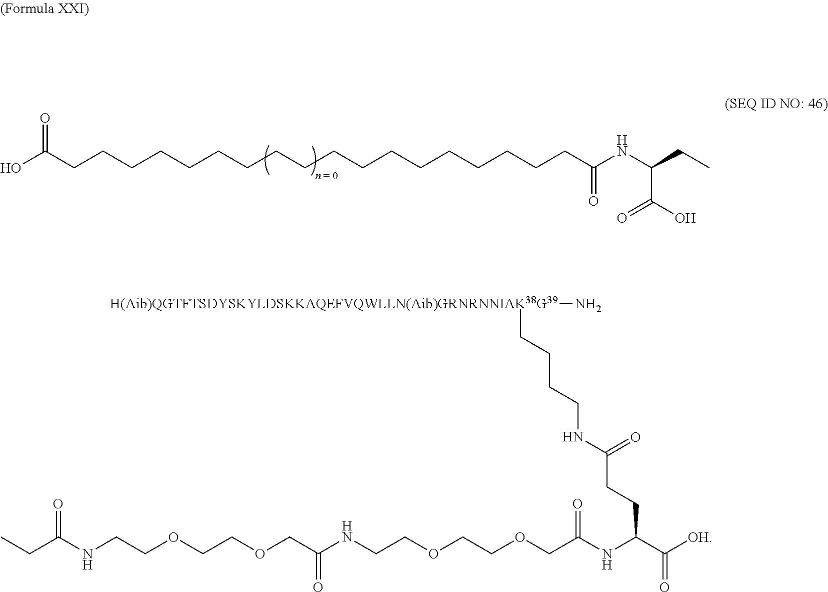

In one aspect, the modified OXM comprises H(Aib)QGTFTSDYSKYLDSKKAQEFVQWLLN(Aib)GRNRNNIAK³⁸(Octadecanedioic-gGlu-(AEEA)₂-gGlu)G³⁹-NH₂(SEQ ID NO: 46).

In one aspect, the modified OXM comprises the following formula:

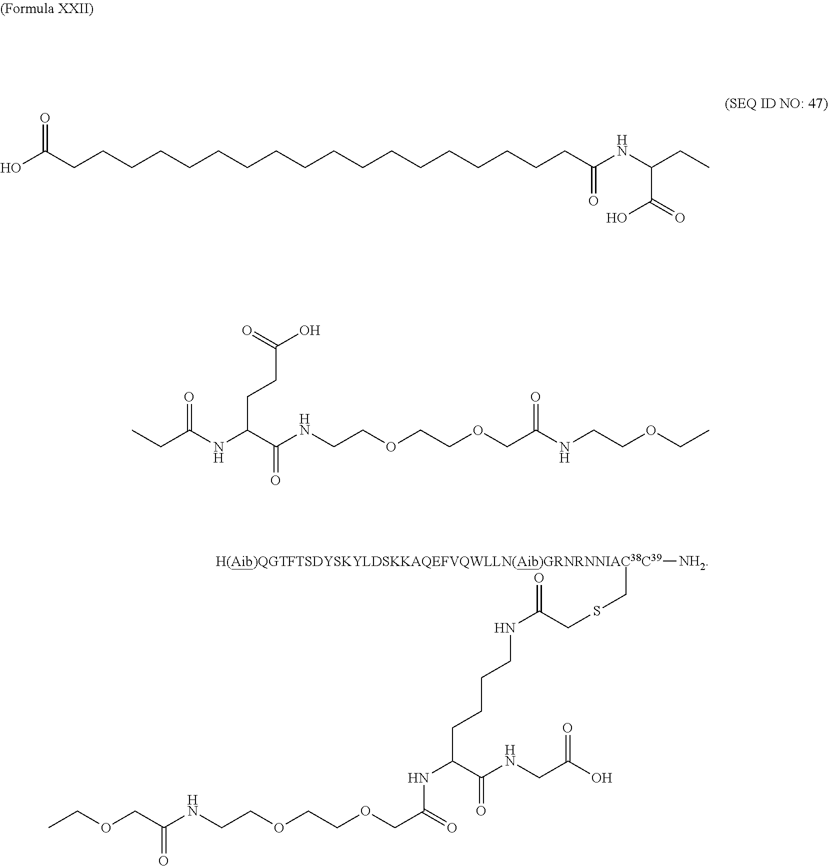

In one aspect, the modified OXM comprises Eicosanedioic-gGlu-Glu-AEEA-AEEA-AEEA-Lys(Ac-Cys³⁸mut.OXM (C³⁸C³⁹))-Gly-OH (SEQ ID NO: 47).

In one aspect, the modified OXM comprises the following formula:

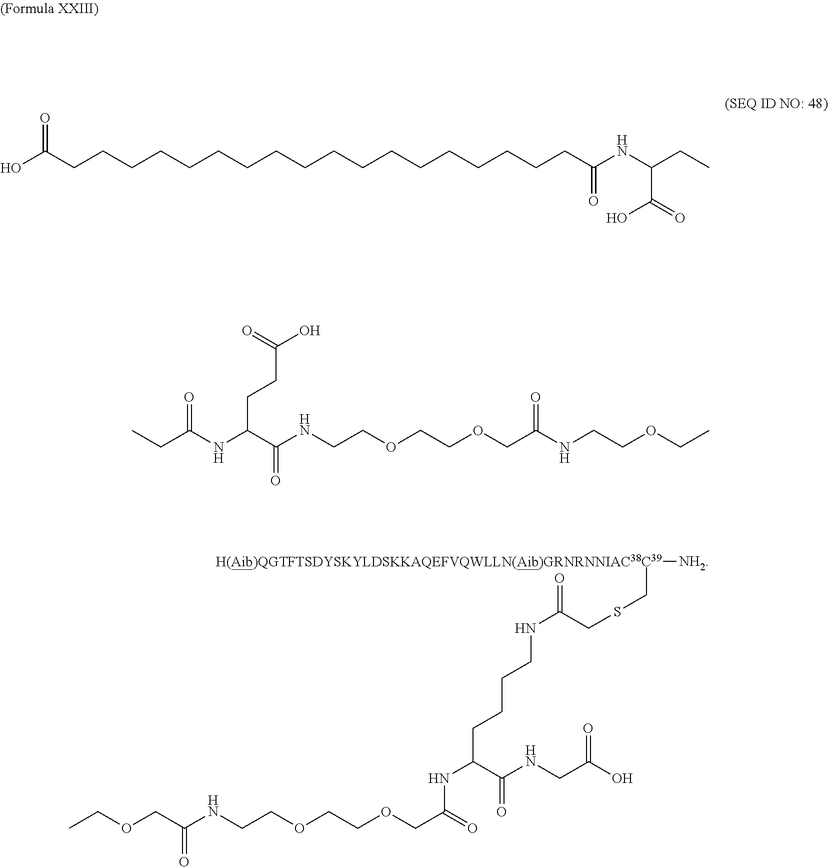

In one aspect, the modified OXM comprises Eicosanedioic-gGlu-Glu-AEEA-AEEA-AEEA-Lys(Ac-Cys³⁹mut.OXM (C³⁸C³⁹))-Gly-OH (SEQ ID NO: 48).

In one aspect, the modified OXM comprises the following formula:

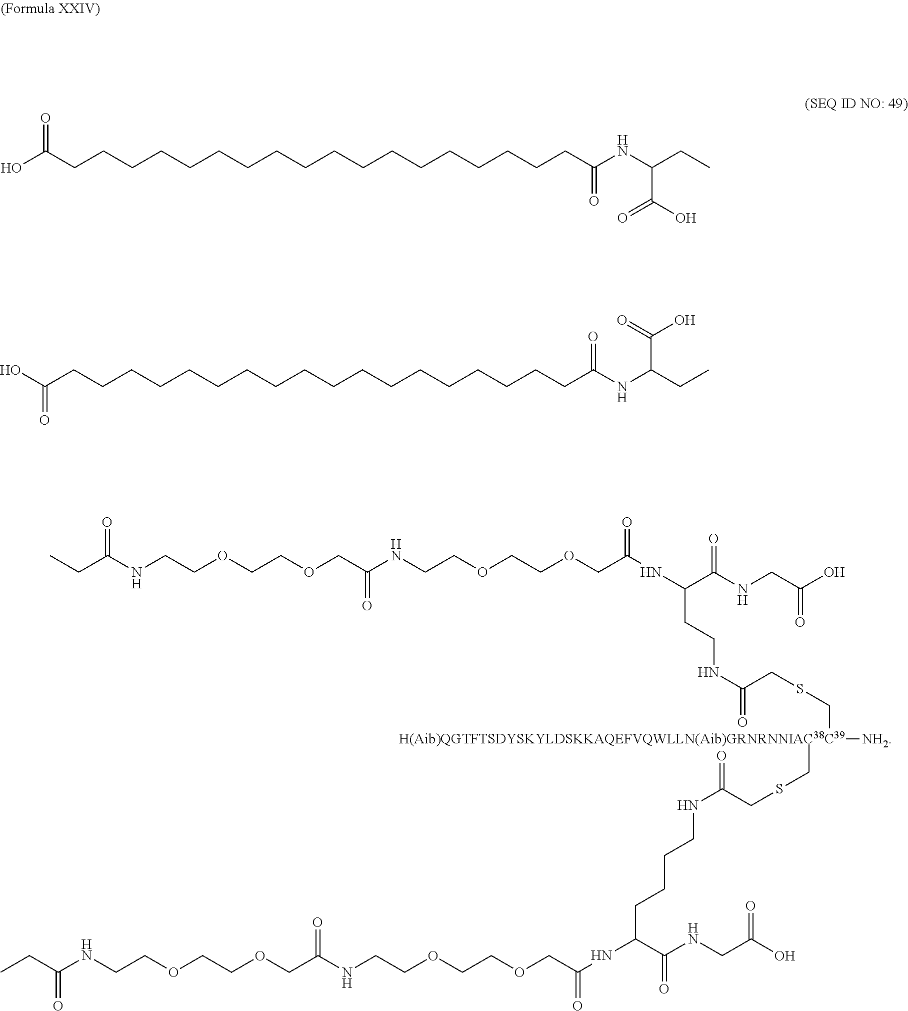

In one aspect, the modified OXM comprises H(Aib)QGTFTSDYSKYLDSKKAQEFVQWLLN(Aib)GRNRNNIAC³⁸(Eicosanedioic-gGlu-(AEEA)₂-Lys(Ac)-Gly)C³⁹(Eicosanedioic-gGlu-(AEEA)₂-Lys(Ac)-Gly)-NH₂ (SEQ ID NO: 49)

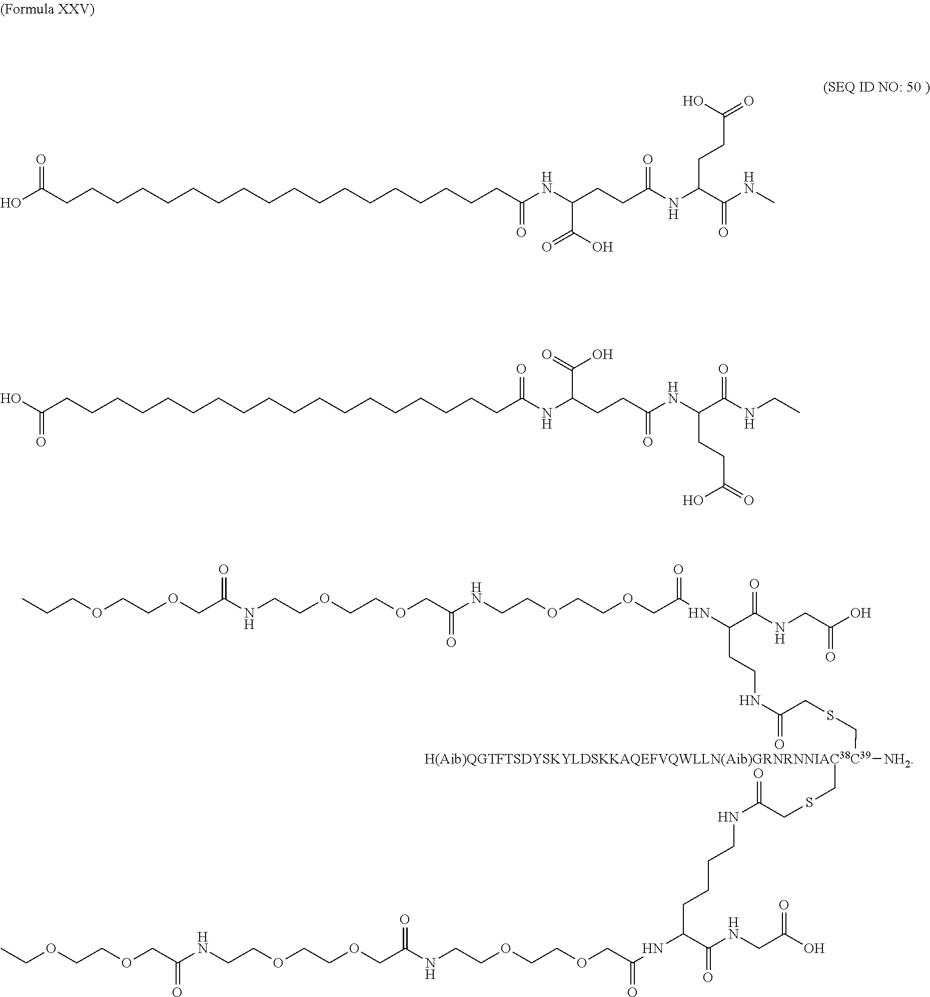

In one aspect, the modified OXM comprises H(Aib)QGTFTSDYSKYLDSKKAQEFVQWLLN(Aib)GRNRNNIAC³⁸(Eicosanedioic-gGlu-Glu-(AEEA)₃-Lys(Ac)-Gly)C³⁹(Eicosanedioic-gGlu-Glu-(AEEA)₃-Lys(Ac)-Gly)-NH₂ (SEQ ID NO: 50).

In one aspect, the binder complex V of Formula II is an albumin binding group.

In one aspect, the W of Formula III is an albumin binding group.

In one aspect, the albumin binding group increases the binding affinity of the native OXM, OXM analog, or active fragment thereof, to human serum albumin.

In one aspect, disclosed herein is a composition comprising a mixture of two polypeptides, a first polypeptide comprising any of modified OXM disclosed herein and a second polypeptide comprising any of modified OXM disclosed. In a related aspect, the first polypeptide is present in the mixture between 30% to 60%. In a related aspect, the polypeptides in the mixture are in the form of a pharmaceutically acceptable salt.

In one aspect, any of the modified OXM disclosed herein are in the form of a pharmaceutically acceptable salt.

In one aspect, any of the modified OXM disclosed herein further comprise a pharmaceutically acceptable salt thereof.

In one aspect, disclosed herein is a pharmaceutical composition any of the modified OXM compound disclosed herein or a pharmaceutically acceptable salt, stereoisomer, solvate, or hydrate thereof, and a pharmaceutically acceptable excipient.

In one aspect, disclosed is a method of treating cardiometabolic and associated diseases comprising administering to a subject in need of such treatment a therapeutically effective amount of a compound of any one the modified OXM disclosed a pharmaceutically acceptable salt, or the pharmaceutical composition of any of the modified OXM. In a related aspect, the disease is TID, T2DM, pre-diabetes, idiopathic TID, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome Crohn's disease, colitis, irritable bowel syndrome, prevention or treatment of Polycystic Ovary Syndrome and treatment of addiction.

In one aspect, disclosed is a method of reducing the risk of a major adverse cardiovascular event (MACE), comprising administering any of the modified OXM, or the pharmaceutical composition of the modified OXM in a therapeutically effective amount to a subject in need thereof, wherein the subject has type 2 diabetes and cardiovascular disease. In a related aspect, MACE is selected from the group consisting of CV death, non-fatal (myocardial infarction) MI, non-fatal stroke, revascularisation, hospitalisation for heart failure, and hospitalisation for unstable angina pectoris. In a related aspect, the cardiovascular disease is selected from the group consisting of clinical evidence of cardiovascular disease and subclinical evidence of cardiovascular disease; wherein the clinical evidence of cardiovascular disease is selected from the group consisting of prior myocardial infarction; prior stroke or transient ischaemic attack; prior coronary, carotid, or peripheral arterial revascularization; >50% stenosis on angiography or imaging of coronary, carotid, or lower extremity arteries; history of symptomatic coronary heart disease; asymptomatic cardiac ischemia; heart failure; and chronic renal impairment by estimated glomerular filtration rate <60 mL/min/1.73 m² per MDRD; and wherein the subclinical evidence of cardiovascular disease is selected from the group consisting of persistent microalbuminuria or proteinuria; hypertension and left ventricular hypertrophy by ECG or imaging; left ventricular systolic or diastolic dysfunction; and ankle/brachial index <0.9. In a related aspect, the subject has a BMI of no more than 30 kg/m2.

In one aspect, the modified OXM is administered for at least 30 months.

In one aspect, the modified OXM is administered in a pharmaceutical composition comprising about 0.1-20 mg/ml modified OXM.

In one aspect, the modified OXM is administered in a pharmaceutical composition comprising between 1 nmol/kg and 30 nmol/kg of body weight of the patient.

In one aspect, the pharmaceutical composition comprises about 2-15 mM phosphate buffer, about 2-25 mg/ml propylene glycol, about 1-18 mg/ml phenol, and has a pH in the range of 7.0-9.0.

In one aspect, disclosed is the use of a therapeutically effective amount of a compound of any of the modified OXM, or the pharmaceutical composition of the modified OXM, in the manufacture of a medicament for treating a subject with cardiometabolic and associated diseases.

In one aspect, disclosed is the use of a therapeutically effective amount of a compound of any one of the modified OXM, or the pharmaceutical composition of modified OXM, in the manufacture of a medicament for treating a subject in need thereof.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

The present disclosure of modified OXM and OXM analogs, both as to their generation and method of use, together with objects, features, and advantages thereof, may best be understood by reference to the following detailed description when read with the accompanying drawings in which:

FIG. 1 is adapted from Sand, Kine Marita Knudsen, et al. “Unraveling the interaction between FcRn and albumin: opportunities for design of albumin-based therapeutics.” Frontiers in immunology 5 (2015): 682, FIG. 2, and shows the crystal structure of human albumin. FIG. 1 shows the crystal structure of human albumin solved in the presence of saturating amounts of palmitic acid. The α-helical structures of the three domains (DI, DII, and DIII) are divided into subdomains (A and B) as indicated. Fatty acids binding sites are indicated via numbered circles (1-7) spread around the surface of the albumin three domains.

FIGS. 2A and 2B detail the methodology of two steps variant synthesis: FIG. 2A shows the general description of one of the productions of the mutated OXM backbone and the binder complex.

FIG. 2B shows the general description of the production of the acylated OXM analog.

FIG. 3 shows the synthesis steps for construction of fatty acid-peptide complex (Binder). Both the CTC-2 resin and the ivDde PG in FIG. 3 are not mandatory and therefore are only optional.

FIG. 4 shows the conjugation of a fatty acid-peptide complex (binder) to a mutated OXM backbone to produce an acylated OXM analog (SEQ ID NOs: 75 and 76, respectively, top to bottom).

FIG. 5 details the methodology of a one step variant synthesis (all on resin), where the mutated OXM backbone is synthesized on resin, following by a side chain elongation to synthesize the variant (SEQ ID NOs: 77-79, respectively, top to bottom).

FIG. 6 illustrates the dual agonism of the oxyntomodulin or oxyntomodulin analog. This Figure is adapted from Yang, Peng-Yu, et al. “New generation oxyntomodulin peptides with improved pharmacokinetic profiles exhibit weight reducing and anti-steatotic properties in mice.” Bioconjugate Chemistry 31.4 (2020): 1167-1176.

FIG. 7 shows the chromatogram for the OPK-88003 KPI, OPK-88003 FA mono conjugate, and the OPK-88003 combination of mono conjugates. Additionally, the purity of the OPK-88003 FA mono conjugate is shown.

FIGS. 8A and 8B show the CBA potency assays demonstrating the peptide potency of various OXM analogs and modified OXM analogs.

FIGS. 9A and 9B are CBA potency assays which qualitatively show the conjugate potential to bind HAS.

FIG. 10 shows the development of a second generation of long-acting acylated oxyntomodulin analogs to enhance potency and dosing regimen.

FIG. 11 shows the study outline of the experiment in Example 7(III).

FIG. 12A shows the body weight randomization, and FIG. 12B shows the fat mass randomization in Example 7.

FIG. 13A shows the absolute body weight and FIG. 13B shows relative body weight profiles.

FIG. 14A shows the discrete D12492 (60% HF) food intake, FIG. 14B shows the cumulative D12492 (60% HF) food intake, and FIG. 14C shows the comparative D12492 (60% HF) food intake.

FIG. 15 shows the cumulative food intake on day 28. The values were expressed as mean of n=8-10+SEM and compared using dunnett's test one-factor linear model.

FIG. 16A shows the absolute body weight at day 28 and FIG. 16B shows the relative body weight (day 28).

FIGS. 17A and 17B show body weight profile (FIG. 17A) and comparative food intake (FIG. 17B) following treatment with OPK-88604 and semaglutide in Example 7.

FIGS. 18A and 18B show body weight (FIG. 18A) and cumulative food intake (FIG. 18B) on day 28 with different treatments (Placebo, OPK-88604 and semaglutide) in Example 7.

FIG. 19A shows the absolute fat tissue mass at week 1, and FIG. 19B shows the absolute lean tissue mass at week 1.

FIG. 20A shows the relative fat tissue mass on week 1, and FIG. 20B shows the relative lean tissue mass on week 1.

FIG. 21A shows the absolute fat tissue mass at week 4, and FIG. 21B shows the absolute lean tissue mass at week 4.

FIG. 22A shows the relative fat tissue mass at week 4, and FIG. 22B shows relative lean tissue mass at week 4.

FIG. 23A shows the change in fat tissue mass from baseline, and FIG. 23B shows the change in lean tissue mass from baseline.

FIGS. 24A and 24B show relative fat mass change (FIG. 24A) and relative lean body mass change (FIG. 24B) from baseline on day 28 for different treatments (Placebo, OPK-88604 and semaglutide in Example 7.

FIG. 25A shows the relative liver cholesterol and FIG. 25B shows the total liver cholesterol.

FIG. 26A shows the relative liver triglyceride and FIG. 26B shows the total liver triglyceride.

FIG. 27A shows the relative liver glycogen and FIG. 27B shows total liver glycogen.

FIGS. 28A, 28B, and 28C show changes from baseline in liver cholesterol (FIG. 28A), liver triglyceride (FIG. 28B), and liver glycogen levels (FIG. 28C) on day 28 following different treatments (placebo, OPK-88604, semaglutide) in Example 7.

FIG. 29 shows liver H&E staining for lipid droplets following a 28 day treatment and contains representative images of liver morphology at termination.

FIGS. 30A, 30B, and 30C show liver H&E staining for lipid droplets following a 28 day treatment with placebo (FIG. 30A), OPK-88604 (FIG. 30B), and semaglutide (FIG. 30C) in Example 7.

FIG. 31A shows relative liver lipid and FIG. 31B shows total liver lipid.

FIG. 32A shows the plasma cholesterol at termination, and FIG. 32B shows the plasma triglyceride at termination.

FIGS. 33A and 33B show a comparison of plasma cholesterol levels (FIG. 33A) and plasma triglyceride levels (FIG. 33B) on day 28 with different treatments (placebo, OPK-88604 and semaglutide in Example 7.

FIG. 34A shows the Fasting Blood glucose at baseline and FIG. 34B shows the fasting blood glucose at week 4.

FIGS. 35A and 35B show the fasting glucose change from baseline (FIG. 35A) and fasting insulin change from baseline (FIG. 35B) on day 28 following treatment with placebo, OPK-88604 and semaglutide in Example 7.

FIG. 36A shows the glucose tolerance plasma insulin at week 1 and FIG. 36B shows the glucose tolerance plasma insulin at week 4.

FIG. 37A shows the oral glucose tolerance test profile and FIG. 37B shows the oral glucose tolerance test area under the curve (0-180 min).

FIGS. 38A and 38B show oral glucose tolerance test (FIG. 38A) and glucose area under the curve (AUC) (FIG. 38B) comparing different treatments with placebo, OPK-88604, and semaglutide in Example 7.

FIG. 39A shows liver weight at termination and FIG. 39B shows heart weight at termination.

FIGS. 40A and 40B show the full formula of the modified OXM disclosed herein as H(Aib)QGTFTSDYSKYLDSKKAQEFVQWLLN(Aib)GRNRNNIAC³⁸(Eicosanedioic-gGlu-(AEEA)₂-Lys(Ac)-Gly)-NH₂ (SEQ ID NO: 39). The molecular weight of the compound shown in FIGS. 40A and 40B is 5390.13, and its chemical formula is C₂₄₁H₃₈₀N₆₄O₇₄S. FIG. 40A shows half of the full chemical structure of the modified OXM starting with the N-terminus of the OXM. FIG. 40B shows the other half of the full chemical structure of the modified OXM ending with the C-terminus of the OXM.

FIGS. 41A and 41B show the effect of OPKO-88604 and OPK-88006 treatment on weight loss in the DIO Mouse Model.

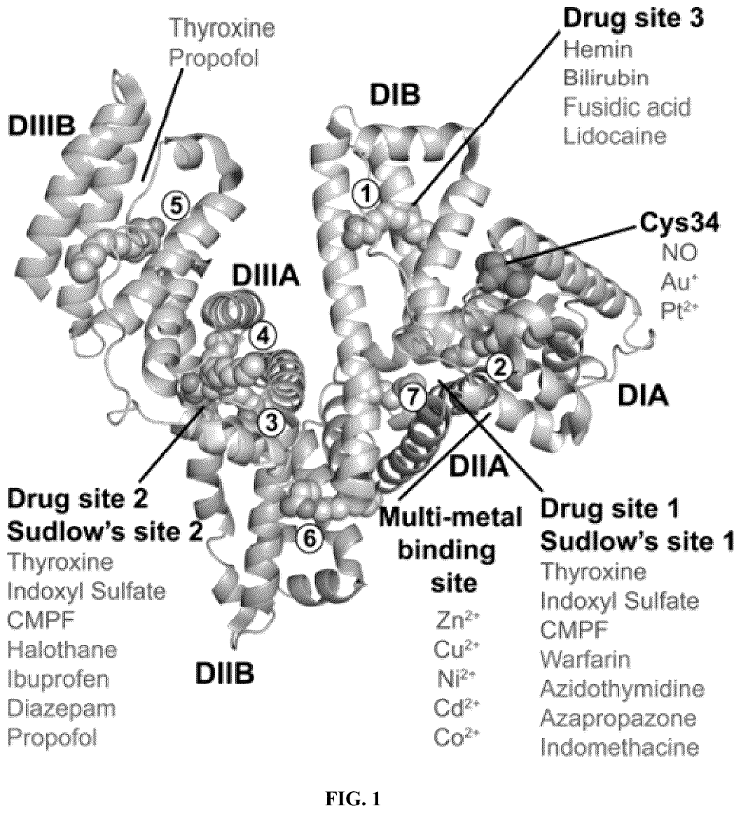

### DETAILED DESCRIPTION

In the following detailed description, numerous specific details are set forth in order to provide a thorough understanding of the OXM analogs and modified OXM or modified OXM analogs disclosed herein. However, it will be understood by those skilled in the art that preparation and uses of OXM analogs and modified OXM or modified OXM analogs disclosed herein may in certain cases be practiced without these specific details. In other instances, well-known methods, procedures, and components have not been described in detail so as not to obscure the disclosure presented herein.

Throughout this application, various references or publications are cited. Disclosures of these references or publications in their entireties are hereby incorporated by reference into this application in order to more fully describe the state of the art to which this invention pertains.

As used herein, the term “oxyntomodulin” may be used interchangeably with the term “OXM”, having all the same meanings and qualities.

As used herein, the term “oxyntomodulin analog” may be used interchangeably with the term “OXM analog”, “OXM mutant”, and “mutated OXM”, having all the same meanings and qualities.

As used herein, the term “wild type OXM” may be used interchangeably with the term “wild type oxyntomodulin”, “WT OXM”, “human wild type OXM”, “human wild type oxyntomodulin”, “native OXM”, “native oxyntomodulin”, “human native OXM”, “human native oxyntomodulin”, having all the same meanings and qualities.

### OXM and OXM Analogs

In one embodiment, the amino acid sequence of native human OXM comprises:

(SEQ ID NO: 1)
His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Lys-Arg-Asn-Arg-Asn-Asn-Ile-
Ala.

In one embodiment, the OXM analog comprises the amino acid sequence:

(SEQ ID NO: 2)
His-(D-Ser)-Gln-Gly-Thr-Phe-Thr-Ser-Asp-(1-Nal)-
Ser-Lys-Tyr-Leu-Asp-Glu-Lys-Ala-Ala-Gln-Glu-Phe-
Val-Gln-Trp-Leu-Leu-Asn-(Aib)-Ala-Arg-Asn-Arg-
Asn-Asn-Ile-Ala-Xaa₃₈-Xaa₃₉

wherein
Xaa₃₈ is Cys, Cys-PEG, Cys-PEG20, Lys, Gly, Val, or is absent, and
Xaa₃₉ is Cys, Cys-PEG, Cys-PEG20, Lys, Gly, Val, or is absent,
wherein the C-terminal amino acid is optionally amidated, is optionally OH, or is optionally NH₂.

In a related embodiment, the PEG in SEQ ID NO: 2 is a 20 kDa PEG. In another embodiment, the PEG at Xaa38 or Xaa39 in SEQ ID NO: 2 is 20 kDa PEG.

In one embodiment, the OXM analog comprises the amino acid sequence:

(SEQ ID NO: 3)
His-(Aib)-Gln-Gly-Thr-Phe-Thr-Ser-Asp-Tyr-Ser-
Lys-Tyr-Leu-Asp-Ser-Lys-Lys-Ala-Gln-Glu-Phe-Val-
Gln-Trp-Leu-Leu-Asn-(Aib)-Gly-Arg-Asn-Arg-Asn-
Asn-Ile-Ala-Xaa₃₈-Xaa₃₉

wherein
Xaa₃₈ is Cys, Cys-PEG, Cys-PEG20, Lys, Gly, Val, or is absent, and
Xaa₃₉ is Cys, Cys-PEG, Cys-PEG20, Lys, Gly, Val, or is absent,
wherein the C-terminal amino acid is optionally amidated, is optionally OH, or is optionally NH₂.

In a related embodiment, the PEG in SEQ ID NO: 3 is a 20 kDa PEG. In another embodiment, the PEG at Xaa38 or Xaa39 in SEQ ID NO: 3 is 20 kDa PEG.

In one embodiment, the OXM analog comprises the amino acid sequence:

(SEQ ID NO: 4)
Xaa1-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Xaa₃₉-Xaa₄₀-Xaa₄₁ is

wherein
Xaa₁ is H or is absent,
Xaa₃₉ is Cys, Cys-PEG, Lys, Gly, Val, or is absent, and
Xaa₄₀ is Cys, Cys-PEG, Lys, Gly, Val, or is absent,
Xaa₄₁ is OH, NH₂ or is absent.

In one embodiment, the OXM analog comprises the amino acid sequence:

(SEQ ID NO: 5)
Xaa₁-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Cys-Xaa₄₀

wherein
Xaa₁ is H, and
Xaa₄₀ is NH₂.

In one embodiment, the OXM analog comprises the amino acid sequence:

(SEQ ID NO: 6)
His-(Aib)-Gln-Gly-Thr-Phe-Thr-Ser-Asp-Tyr-Ser-
Lys-Tyr-Leu-Asp-Ser-Lys-Lys-Ala-Gln-Glu-Phe-Val-
Gln-Trp-Leu-Leu-Asn-(Aib)-Gly-Arg-Asn-Arg-Asn-
Asn-Ile-Ala-Cys-Xaa₃₉

wherein
Xaa₃₉ is NH₂.

In one embodiment, the OXM analog comprises the amino acid sequence:

(SEQ ID NO: 7)
His-(Aib)-Gln-Gly-Thr-Phe-Thr-Ser-Asp-Tyr-Ser-
Lys-Tyr-Leu-Asp-Ser-Lys-Lys-Ala-Gln-Glu-Phe-Val-
Gln-Trp-Leu-Leu-Asn-(Aib)-Gly-Arg-Asn-Arg-Asn-
Asn-Ile-Ala-Cys.

In one embodiment, the OXM analog comprises the amino acid sequence:

(SEQ ID NO: 8)
Xaa₁-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Lys-Gly-Xaa₄₁

wherein
Xaa₁ is H, and
Xaa₄₁ is NH₂.

In one embodiment, the OXM analog comprises the amino acid sequence:

(SEQ ID NO: 9)
His-(Aib)-Gln-Gly-Thr-Phe-Thr-Ser-Asp-Tyr-Ser-
Lys-Tyr-Leu-Asp-Ser-Lys-Lys-Ala-Gln-Glu-Phe-Val-
Gln-Trp-Leu-Leu-Asn-(Aib)-Gly-Arg-Asn-Arg-Asn-
Asn-Ile-Ala-Lys-Gly-Xaa₄₀

wherein
Xaa₄₀ is NH₂.

In one embodiment, the OXM analog comprises the amino acid sequence:

(SEQ ID NO: 10)
His-(Aib)-Gln-Gly-Thr-Phe-Thr-Ser-Asp-Tyr-Ser-Lys-
Tyr-Leu-Asp-Ser-Lys-Lys-Ala-Gln-Glu-Phe-Val-Gln-
Trp-Leu-Leu-Asn-(Aib)-Gly-Arg-Asn-Arg-Asn-Asn-Ile-
Ala-Lys-Gly.

In one embodiment, the OXM analog comprises the amino acid sequence:

(SEQ ID NO: 11)

Xaa₁-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Cys-Cys-Xaa₄₁

wherein
Xaa₁ is H, and
Xaa₄₁ is NH₂.

In one embodiment, the OXM analog comprises the amino acid sequence:

(SEQ ID NO: 12)
His-(Aib)-Gln-Gly-Thr-Phe-Thr-Ser-Asp-Tyr-Ser-Lys-
Tyr-Leu-Asp-Ser-Lys-Lys-Ala-Gln-Glu-Phe-Val-Gln-
Trp-Leu-Leu-Asn-(Aib)-Gly-Arg-Asn-Arg-Asn-Asn-Ile-
Ala-Cys-Cys-Xaa₄₀

wherein
Xaa₄₀ is NH₂.

In one embodiment, the OXM analog comprises the amino acid sequence:

(SEQ ID NO: 13)
His-(Aib)-Gln-Gly-Thr-Phe-Thr-Ser-Asp-Tyr-Ser-Lys-
Tyr-Leu-Asp-Ser-Lys-Lys-Ala-Gln-Glu-Phe-Val-Gln-
Trp-Leu-Leu-Asn-(Aib)-Gly-Arg-Asn-Arg-Asn-Asn-Ile-
Ala-Cys-Cys.

In one embodiment, the OXM analog comprises the amino acid sequence:

(SEQ ID NO: 14)

Xaa₁-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Lys-Xaa₄₀

wherein
Xaa₁ is H or is absent, and
Xaa₄₀ is NH₂, OH, or is absent.

In one embodiment, the OXM analog comprises the amino acid sequence:

(SEQ ID NO: 15)

Xaa₁-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Lys-Cys-Xaa₄₁

wherein
Xaa₁ is H or is absent, and
Xaa₄₁ is NH₂, OH, or is absent.

In one embodiment, the OXM analog comprises the amino acid sequence:

(SEQ ID NO: 16)

Xaa₁-His-(Aib)-Gln-Gly-Thr-Phe-Thr-Ser-Asp-Tyr-
Ser-Lys-Tyr-Leu-Asp-Ser-Lys-Lys-Ala-Gln-Glu-Phe-
Val-Gln-Trp-Leu-Leu-Asn-(Aib)-Gly-Arg-Asn-Arg-
Asn-Asn-Ile-Ala-Cys-Lys-Xaa₄₁

wherein
Xaa₁ is H or is absent, and
Xaa₄₁ is NH₂, OH, or is absent.

In one embodiment, the oxyntomodulin peptide analog is an active fragment of wild type OXM. In another embodiment, the OXM peptide analog is an active fragment of an OXM analog. In another embodiment, the OXM peptide analog is an active fragment of any one of SEQ ID NOs: 1 to 16. In one embodiment, the oxyntomodulin peptide analog comprises an active fragment of any one of SEQ ID NOs: 1 to 16.

In one embodiment, the active fragment of the OXM or OXM analog is a bio-active fragment. In another embodiment, the active fragment of the OXM or OXM analog is a bio-active fragment which retains the GCGR and GLP-1 R dual agonist activity of OXM.

In one embodiment, the active fragment of the OXM or OXM analog is a truncated fragment. In another embodiment, the active fragment of the OXM or OXM analog is a truncated fragment of any one of SEQ ID NOs: 1 to 16.

In one embodiment, the active OXM or OXM analog fragment is a C-terminal fragment. In another embodiment, the active OXM or OXM analog fragment is a C-terminal fragment of any of SEQ ID NOs: 1 to 16. In another embodiment, the active OXM or OXM analog fragment is defined as the composition starting at the 17th amino acid position from the N-terminus to the end of the C-terminus. In a preferred embodiment, the active OXM or OXM analog fragment encompasses the following positions: AA 22 from the N terminus to the C terminus, AA 25 from the N terminus to the C terminus, AA 28 from the N terminus to the C terminus, AA 30 from the N terminus to the C terminus, AA 32 from the N terminus to the C terminus, or AA 36 from the N terminus to the C terminus.

In one embodiment the OXM or OXM analog is a synthetic protein. In another embodiment, the OXM or OXM analog is produced by direct peptide synthesis. In another embodiment, the OXM or OXM analog is produced by solid-phase peptide synthesis. The amino acid sequence of the OXM, OXM analog, or any fragment thereof, may be produced by direct peptide synthesis using solid-phase techniques (See Stewart et al., Solid-Phase Peptide Synthesis (1969); and Merrifield, J. Am. Chem. Soc. 85:2149-54 (1963)).

The term “OXM analog”, “OXM mutant”, “mutated OXM”, or “mutated OXM analog” encompasses wild type human OXM comprising one or more amino acid substitutions, additions or deletions. OXM analogs of the present disclosure may be comprised of modifications with one or more natural amino acids in conjunction with one or more non-natural amino acid modification. Substitutions in a wide variety of amino acid positions in wild type OXM have been described, including but not limited to substitutions that modulate one or more of the biological activities of the OXM peptide, such as but not limited to, improved binding characteristics, increased activity, increased solubility of the polypeptide, increased albumin binding, increased chemical stability, or increased potency.

In one embodiment, the term “OXM analog” encompasses active fragments of wild type OXM or any OXM analogs.

In another aspect, the present application provides an oxyntomodulin peptide analog and formulations comprising the oxyntomodulin peptide analog and uses thereof in treating diabetes and/or obesity. U.S. Pat. Nos. 8,367,607, 8,415,296, and U.S. Application Number No. US 2020/0262887 hereby incorporated by reference, claim and disclose multiple oxyntomodulin analog PEGylated derivatives and disclose the syntheses thereof.

The OXM analogs or OXM described in the present application include glycosylated OXM or OXM analogs, such as but not limited to, polypeptides glycosylated at any amino acid position, N-linked or O-linked glycosylated forms of the polypeptide. Variants containing single nucleotide or amino acid changes are also considered as biologically active variants of OXM. In addition, splice variants are also included. The term “OXM polypeptide” or “OXM analog polypeptide” also includes heterodimers, homodimers, heteromultimers, or homomultimers of any one or more OXM polypeptides or any other polypeptide, protein, carbohydrate, polymer, small molecule, linker, ligand, or other biologically active molecule of any type, linked by chemical means or expressed as a fusion protein, as well as polypeptide analogs containing, for example, specific deletions or other modifications yet maintain biological activity.

In one embodiment, the OXM analog contains between 1 and 4 amino acid point mutations. In another embodiment, the OXM analog contains 1 amino acid mutation. In another embodiment, the OXM analog contains 2 amino acid mutations. In another embodiment, the OXM analog contains 3 amino acid mutations. In another embodiment, the OXM analog contains 4 amino acid mutations.

In one embodiment, the OXM analog contains between 4 and 8 amino acid point mutations. In another embodiment, the OXM analog contains 4 amino acid mutations. In another embodiment, the OXM analog contains 5 amino acid mutations. In another embodiment, the OXM analog contains 6 amino acid mutations. In another embodiment, the OXM analog contains 7 amino acid mutations. In another embodiment, the OXM analog contains 8 amino acid mutations.

In one embodiment, the OXM analog contains between 8 and 15 amino acid point mutations. In another embodiment, the OXM analog contains 8 amino acid mutations. In another embodiment, the OXM analog contains 9 amino acid mutations. In another embodiment, the OXM analog contains 10 amino acid mutations. In another embodiment, the OXM analog contains 11 amino acid mutations. In another embodiment, the OXM analog contains 12 amino acid mutations. In another embodiment, the OXM analog contains 13 amino acid mutations. In another embodiment, the OXM analog contains 14 amino acid mutations. In another embodiment, the OXM analog contains 15 amino acid mutations.

In another embodiment, the present application includes a homologue of a wild type OXM. In another embodiment, homologues e.g., polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a wild type OXM as determined using BlastP software of the National Center of Biotechnology Information (NCBI) using default parameters.

In another embodiment, “signal sequence” and “signal peptide” are used interchangeably herein having all the same qualities and meanings. In another embodiment, “sequence” when in reference to a polynucleotide molecule can refer to a coding portion.

In one embodiment, the OXM analog includes a signal peptide. In another embodiment, the OXM analog does not comprise a signal peptide.

### Acylated OXM Complexes

The terms acylated OXM, acylated OXM analog, acylated OXM complex, acylated OXM analog complex, modified OXM, modified OXM analog, modified OXM complex, and modified OXM analog complex, are synonymous in the present application.

In one embodiment, the acylated OXM includes a signal peptide. In another embodiment, the acylated OXM does not comprise a signal peptide.

In one embodiment, the OXM or OXM analogs disclosed herein are modified. In another embodiment, the OXM or OXM analogs disclosed herein are modified to increase albumin binding.

In another embodiment, the OXM or OXM analogs disclosed herein are modified to increase their half-life.

In one embodiment, an “acylated” amino acid is an amino acid comprising an acyl group which is non-native to a naturally occurring amino acid, regardless of how it is produced. Methods of producing acylated amino acids and acylated peptides are known in the art and include acylating an amino acid before inclusion in the peptide or peptide synthesis followed by chemical acylation of the peptide. In another embodiment, the acyl group causes the peptide to have one or more of (i) a prolonged half-life in circulation, (ii) a delayed onset of action, (iii) an improved resistance to proteases, or (iv) an extended duration of action.

In another embodiment, the OXM or OXM analogs disclosed herein are acylated. In another embodiment, the OXM or OXM analogs disclosed herein are acylated to increase albumin binding. In another embodiment, the OXM or OXM analogs disclosed herein are acylated to increase their half-life. In another embodiment, the OXM or OXM analogs disclosed herein are modified by being acylated.

In one embodiment, the OXM or OXM analogs disclosed herein are mono or multi-acylated. In another embodiment, the OXM or OXM analogs are multi-acylated and acylated on any amino acid positions present.

In one embodiment, the modified OXM or OXM analogs disclosed herein are mono or multi-acylated. In another embodiment, the modified OXM or OXM analogs are multi-acylated and acylated on any amino acid positions present. In a preferred embodiment, the multi-acylation occurs on the C-terminus of the modified OXM or OXM analog.

In one embodiment, at least one amino acid of the OXM is mutated to lysine. In a related embodiment, at least one amino acid of the OXM is mutated to lysine as a conjugation site for the binder complex. Specifically, at least one amino acid of the OXM is mutated to lysine and without interrupting the protein-receptor interaction.

In one embodiment, at least one amino acid of the OXM is mutated to cysteine. In a related embodiment, at least one amino acid of the OXM is mutated to cysteine as a conjugation site for the binder complex. Specifically, at least one amino acid of the OXM is mutated to cysteine and without interrupting the protein-receptor interaction.

In one embodiment, at least one amino acid of the OXM is mutated to glycine. In a related embodiment, at least one amino acid of the OXM is mutated to glycine as a conjugation site for the binder complex. Specifically, at least one amino acid of the OXM is mutated to glycine and without interrupting the protein-receptor interaction.

In one embodiment, at least one amino acid of the OXM is mutated to glycine to aid in the synthesis of the OXM analog. In another embodiment, at least one amino acid of the OXM is mutated to glycine to aid in the conjugation of the OXM analog to any of the binder complexes described herein. In one embodiment, the C-terminus of any of the OXM analogs is mutated to glycine to aid in the synthesis of the OXM analog. In another embodiment, the C-terminus of any of the OXM analogs is mutated to glycine to aid in the conjugation of the OXM analog to any of the binder complexes described herein.

In one embodiment, the G39 of SEQ ID NO: 2, the G39 of SEQ ID NO: 3, or the G40 of SEQ ID NO: 4 aids in the synthesis of the OXM analog. In another embodiment, the G39 of SEQ ID NO: 2, the G39 of SEQ ID NO: 3, or the G40 of SEQ ID NO: 4 aids in the conjugation of the OXM analog to any of the binder complexes described herein.

In one embodiment, at least one amino acid of the OXM is mutated to valine. In a related embodiment, at least one amino acid of the OXM is mutated to valine as a conjugation site for the binder complex. In another embodiment, the C terminus of an OXM analog, or fragment thereof, is mutated to valine. In another embodiment, the C terminus of an OXM analog, or fragment thereof, is mutated to valine as a conjugation site for the binder complex. In another embodiment, at least one amino acid of the OXM is mutated to valine and without interrupting the protein-receptor interaction.

In one embodiment, at least one amino acid of the OXM is mutated to valine to aid in the synthesis of the OXM analog. In another embodiment, at least one amino acid of the OXM is mutated to valine to aid in the conjugation of the OXM analog to any of the binder complexes described herein. In one embodiment, the C-terminus of any of the OXM analogs is mutated to valine to aid in the synthesis of the OXM analog. In another embodiment, the C-terminus of any of the OXM analogs is mutated to valine to aid in the conjugation of the OXM analog to any of the binder complexes described herein.

In one embodiment, the V39 of SEQ ID NO: 2, the V39 of SEQ ID NO: 3, or the V40 of SEQ ID NO: 4 aids in the synthesis of the OXM analog. In another embodiment, the V39 of SEQ ID NO: 2, the V39 of SEQ ID NO: 3, or the V40 of SEQ ID NO: 4 aids in the conjugation of the OXM analog to any of the binder complexes described herein.

In one embodiment, the Y of formula (I), V of formula (II), or Y of formula (III) is present and conjugated to any lysine, cysteine, or glycine residue present in said native OXM, an OXM analog, or an active fragment thereof.

In another embodiment, the Y of formula (I), V of formula (II), or Y of formula (III) is not present, and X is conjugated to any lysine, cysteine, or glycine residue present in said native OXM, an OXM analog, or an active fragment thereof.

In a related embodiment, C38 of the OXM analog of SEQ ID NO: 2 or SEQ ID NO: 3 is used as a conjugation site for the binder complex. In another embodiment, C38 of the OXM analog of SEQ ID NO: 2 or SEQ ID NO: 3 is used as a conjugation site for the binder complex without interrupting the protein-receptor interaction.

In a related embodiment, C39 of the OXM analog of SEQ ID NO: 2 or SEQ ID NO: 3 is used as a conjugation site for the binder complex. In another embodiment, C39 of the OXM analog of SEQ ID NO: 2 or SEQ ID NO: 3 is used as a conjugation site for the binder complex without interrupting the protein-receptor interaction.

In a related embodiment, C39 of the OXM analog of SEQ ID NO: 4 is used as a conjugation site for the binder complex. In another embodiment, C39 of the OXM analog of SEQ ID NO: 4 is used as a conjugation site for the binder complex without interrupting the protein-receptor interaction.

In a related embodiment, C40 of the OXM analog of SEQ ID NO: 4 is used as a conjugation site for the binder complex. In another embodiment, C40 of the OXM analog of SEQ ID NO: 4 is used as a conjugation site for the binder complex without interrupting the protein-receptor interaction.

In a preferred embodiment, the conjugation site for the binder complex occurs at the C-terminus of the modified OXM or OXM analog. In another preferred embodiment, the modified OXM or OXM analog is multi-acylated with multiple conjugation sites occurring near the C-terminus of the modified OXM or OXM analog.

In one embodiment, the mutated, C-terminal cystine residue in any of the OXM analogs described herein is used as a conjugation site for the binder complex. In another embodiment, either of the mutated, C-terminal cystine residues in any of the OXM analogs described herein is used as a conjugation site for the binder complex. In another embodiment, both of the mutated, C-terminal cystine residues in any of the OXM analogs described herein are used as a conjugation site for the binder complex.

In another embodiment, the last mutated cystine residue present at the C-terminus of any of the OXM analogs described herein is used as a conjugation site for the binder complex. In another embodiment, the second to last mutated cystine residue present at the C-terminus of any of the OXM analogs described herein is used as a conjugation site for the binder complex.

In a related embodiment, K38 of the OXM analog of SEQ ID NO: 2 or SEQ ID NO: 3 is used as a conjugation site for the binder complex. In another embodiment, K38 of the OXM analog of SEQ ID NO: 2 or SEQ ID NO: 3 is used as a conjugation site for the binder complex without interrupting the protein-receptor interaction.

In a related embodiment, G39 of the OXM analog of SEQ ID NO: 2 or SEQ ID NO: 3 is used as a conjugation site for the binder complex. In another embodiment, G39 of the OXM analog of SEQ ID NO: 2 or SEQ ID NO: 3 is used as a conjugation site for the binder complex without interrupting the protein-receptor interaction.

In a related embodiment, K39 of the OXM analog of SEQ ID NO: 4 is used as a conjugation site for the binder complex. In another embodiment, K39 of the OXM analog of SEQ ID NO: 4 is used as a conjugation site for the binder complex without interrupting the protein-receptor interaction.

In a related embodiment, G40 of the OXM analog of SEQ ID NO: 4 is used as a conjugation site for the binder complex. In another embodiment, G40 of the OXM analog of SEQ ID NO: 4 is used as a conjugation site for the binder complex without interrupting the protein-receptor interaction.

In one embodiment, the mutated, C-terminal lysine residue in any of the OXM analogs described herein is used as a conjugation site for the binder complex. In another embodiment, the mutated, C-terminal glycine residue in any of the OXM analogs described herein is used as a conjugation site for the binder complex. In another embodiment, the mutated, C-terminal valine residue in any of the OXM analogs described herein is used as a conjugation site for the binder complex.

In another embodiment, the last mutated lysine residue present at the C-terminus of any of the OXM analogs described herein is used as a conjugation site for the binder complex.

In one embodiment, the V of formula (II) is conjugated at the amino acid position 38 or position 39 of said OXM analog in SEQ ID NO: 2 or SEQ ID NO: 3, or any combination thereof.

Ine one embodiment, the V of formula (II) is conjugated at the amino acid position 39 or position 40 of said OXM analog in SEQ ID NO: 4, or any combination thereof.

In one embodiment, the V of formula (II) is conjugated at one or more positions between amino acid position numbers 17 to 37 of SEQ ID NO: 1. In another embodiment, the V of formula (II) is conjugated at one or more amino acid position numbers between: 22 to 37, 25 to 37, 28 to 37, 30 to 37, 32 to 37, or 36 to 37 of SEQ ID NO: 1. In another embodiment, the V of formula (II) is conjugated at one or more amino acid position numbers between 22 to 37 of SEQ ID NO: 1. In another embodiment, the V of formula (II) is conjugated at one or more amino acid position numbers between 25 to 37 of SEQ ID NO: 1. In another embodiment, the V of formula (II) is conjugated at one or more amino acid position numbers between 28 to 37 of SEQ ID NO: 1. In another embodiment, the V of formula (II) is conjugated at one or more amino acid position numbers between 30 to 37 of SEQ ID NO: 1. In another embodiment, the V of formula (II) is conjugated at one or more amino acid position numbers between 32 to 37 of SEQ ID NO: 1. In another embodiment, the V of formula (II) is conjugated at one or more amino acid position numbers between 36 to 37 of SEQ ID NO: 1.

In one embodiment, the V of formula (II) is conjugated at one or more positions between amino acid position numbers 17 to 37 of wild type OXM or an active fragment thereof. In another embodiment, the V of formula (II) is conjugated at one or more amino acid positions numbers between: 22 to 37, 25 to 37, 28 to 37, 30 to 37, 32 to 37, or 36 to 37 of wild type OXM or an active fragment thereof. In another embodiment, the V of formula (II) is conjugated at one or more amino acid positions numbers between 22 to 37 of wild type OXM or an active fragment thereof. In another embodiment, the V of formula (II) is conjugated at one or more amino acid positions numbers between 25 to 37 of wild type OXM or an active fragment thereof. In another embodiment, the V of formula (II) is conjugated at one or more amino acid positions numbers between 28 to 37 of wild type OXM or an active fragment thereof. In another embodiment, the V of formula (II) is conjugated at one or more amino acid positions numbers between 30 to 37 of wild type OXM or an active fragment thereof. In another embodiment, the V of formula (II) is conjugated at one or more amino acid positions numbers between 32 to 37 of wild type OXM or an active fragment thereof. In another embodiment, the V of formula (II) is conjugated at one or more amino acid positions numbers between 36 to 37 of wild type OXM or an active fragment thereof.

In one embodiment, the V of formula (II) is conjugated at one or more positions between amino acid position numbers 18 to 39 of SEQ ID NO: 5. In another embodiment, the V of formula (II) is conjugated at one or more amino acid position numbers between: 23 to 39, 26 to 39, 29 to 39, 31 to 39, 33 to 39, or 37 to 39 of SEQ ID NO: 5. In another embodiment, the V of formula (II) is conjugated at one or more amino acid position numbers between 23 to 39 of SEQ ID NO: 5. In another embodiment, the V of formula (II) is conjugated at one or more amino acid position numbers between 26 to 39 of SEQ ID NO: 5. In another embodiment, the V of formula (II) is conjugated at one or more amino acid position numbers between: 29 to 39 of SEQ ID NO: 5. In another embodiment, the V of formula (II) is conjugated at one or more amino acid position numbers between 31 to 39 of SEQ ID NO: 5. In another embodiment, the V of formula (II) is conjugated at one or more amino acid position numbers between 33 to 39 of SEQ ID NO: 5. In another embodiment, the V of formula (II) is conjugated at one or more amino acid position numbers between 37 to 39 of SEQ ID NO: 5.

In one embodiment, the V of formula (II) is conjugated at one or more positions between amino acid position numbers 18 to 39 of an OXM analog or an active fragment thereof. In another embodiment, the V of formula (II) is conjugated at one or more amino acid position numbers between: 23 to 39, 26 to 39, 29 to 39, 31 to 39, 33 to 39, or 37 to 39 of an OXM analog or an active fragment thereof. In another embodiment, the V of formula (II) is conjugated at one or more amino acid position numbers between 23 to 39 of an OXM analog or an active fragment thereof. In another embodiment, the V of formula (II) is conjugated at one or more amino acid position numbers between 26 to 39 OXM analog or an active fragment thereof. In another embodiment, the V of formula (II) is conjugated at one or more amino acid position numbers between: 29 to 39 of an OXM analog or an active fragment thereof. In another embodiment, the V of formula (II) is conjugated at one or more amino acid position numbers between 31 to 39 of an OXM analog or an active fragment thereof. In another embodiment, the V of formula (II) is conjugated at one or more amino acid position numbers between 33 to 39 of an OXM analog or an active fragment thereof. In another embodiment, the V of formula (II) is conjugated at one or more amino acid position numbers between 37 to 39 of an OXM analog or an active fragment thereof.

In one embodiment, the V of formula (II) is conjugated at one or more positions between amino acid position numbers 17 to 38 of SEQ ID NO: 6 or SEQ ID NO: 7. In another embodiment, the V of formula (II) is conjugated at one or more amino acid position numbers between: 22 to 38, 25 to 38, 28 to 38, 30 to 38, 32 to 38, or 36 to 38 of SEQ ID NO: 6 or SEQ ID NO: 7. In another embodiment, the V of formula (II) is conjugated at one or more amino acid position numbers between 22 to 38 of SEQ ID NO: 6 or SEQ ID NO: 7. In another embodiment, the V of formula (II) is conjugated at one or more amino acid position numbers between 25 to 38 of SEQ ID NO: 6 or SEQ ID NO: 7. In another embodiment, the V of formula (II) is conjugated at one or more amino acid position numbers between 28 to 38 of SEQ ID NO: 6 or SEQ ID NO: 7. In another embodiment, the V of formula (II) is conjugated at one or more amino acid position numbers between 30 to 38 of SEQ ID NO: 6 or SEQ ID NO: 7. In another embodiment, the V of formula (II) is conjugated at one or more amino acid position numbers between 32 to 38 of SEQ ID NO: 6 or SEQ ID NO: 7. In another embodiment, the V of formula (II) is conjugated at one or more amino acid position numbers between 36 to 38 of SEQ ID NO: 6 or SEQ ID NO: 7.

In one embodiment, the V of formula (II) is conjugated at one or more positions between amino acid position numbers 17 to 38 of an OXM analog or an active fragment thereof. In another embodiment, the V of formula (II) is conjugated at one or more amino acid position numbers between: 22 to 38, 25 to 38, 28 to 38, 30 to 38, 32 to 38, or 36 to 38 of an OXM analog or an active fragment thereof. In another embodiment, the V of formula (II) is conjugated at one or more amino acid position numbers between 22 to 38 of an OXM analog or an active fragment thereof. In another embodiment, the V of formula (II) is conjugated at one or more amino acid position numbers between 25 to 38 of an OXM analog or an active fragment thereof. In another embodiment, the V of formula (II) is conjugated at one or more amino acid position numbers between 28 to 38 of an OXM analog or an active fragment thereof. In another embodiment, the V of formula (II) is conjugated at one or more amino acid position numbers between 30 to 38 of an OXM analog or an active fragment thereof. In another embodiment, the V of formula (II) is conjugated at one or more amino acid position numbers between 32 to 38 of an OXM analog or an active fragment thereof. In another embodiment, the V of formula (II) is conjugated at one or more amino acid position numbers between 36 to 38 of an OXM analog or an active fragment thereof.

In one embodiment, the V of formula (II) is conjugated at one or more positions between amino acid position numbers 18 to 40 of SEQ ID NO: 4, SEQ ID NO: 8, or SEQ ID NO: 11. In another embodiment, the V of formula (II) is conjugated at one or more amino acid position numbers between: 23 to 40, 26 to 40, 29 to 40, 31 to 40, 33 to 40, or 37 to 40 of SEQ ID NO: 4, SEQ ID NO: 8, or SEQ ID NO: 11. In another embodiment, the V of formula (II) is conjugated at one or more amino acid position numbers between 23 to 40 of SEQ ID NO: 4, SEQ ID NO: 8, or SEQ ID NO: 11. In another embodiment, the V of formula (II) is conjugated at one or more amino acid position numbers between 26 to 40 of SEQ ID NO: 4, SEQ ID NO: 8, or SEQ ID NO: 11. In another embodiment, the V of formula (II) is conjugated at one or more amino acid position numbers between 29 to 40 of SEQ ID NO: 4, SEQ ID NO: 8, or SEQ ID NO: 11. In another embodiment, the V of formula (II) is conjugated at one or more amino acid position numbers between 31 to 40 of SEQ ID NO: 4, SEQ ID NO: 8, or SEQ ID NO: 11. In another embodiment, the V of formula (II) is conjugated at one or more amino acid position numbers between 33 to 40 of SEQ ID NO: 4, SEQ ID NO: 8, or SEQ ID NO: 11. In another embodiment, the V of formula (II) is conjugated at one or more amino acid position numbers between 37 to 40 of SEQ ID NO: 4, SEQ ID NO: 8, or SEQ ID NO: 11.

In one embodiment, the V of formula (II) is conjugated at one or more positions between amino acid position numbers 18 to 40 of an OXM analog or an active fragment thereof. In another embodiment, the V of formula (II) is conjugated at one or more amino acid position numbers between: 23 to 40, 26 to 40, 29 to 40, 31 to 40, 33 to 40, or 37 to 40 of an OXM analog or an active fragment thereof. In another embodiment, the V of formula (II) is conjugated at one or more amino acid position numbers between 23 to 40 of an OXM analog or an active fragment thereof. In another embodiment, the V of formula (II) is conjugated at one or more amino acid position numbers between 26 to 40 of an OXM analog or an active fragment thereof. In another embodiment, the V of formula (II) is conjugated at one or more amino acid position numbers between 29 to 40 of an OXM analog or an active fragment thereof. In another embodiment, the V of formula (II) is conjugated at one or more amino acid position numbers between 31 to 40 of an OXM analog or an active fragment thereof. In another embodiment, the V of formula (II) is conjugated at one or more amino acid position numbers between 33 to 40 of an OXM analog or an active fragment thereof. In another embodiment, the V of formula (II) is conjugated at one or more amino acid position numbers between 37 to 40 of an OXM analog or an active fragment thereof.

In one embodiment, the V of formula (II) is conjugated at one or more positions between amino acid position numbers 17 to 39 of SEQ ID NOs: 2, 3, 9, 10, 12, or 13. In another embodiment, the V of formula (II) is conjugated at one or more amino acid position numbers between: 22 to 39, 25 to 39, 28 to 39, 30 to 39, 32 to 39, or 36 to 39 of SEQ ID NO: 2, 3, 9, 10, 12, or 13. In another embodiment, the V of formula (II) is conjugated at one or more amino acid position numbers between 22 to 39 of SEQ ID NO: 2, 3, 9, 10, 12, or 13. In another embodiment, the V of formula (II) is conjugated at one or more amino acid position numbers between 25 to 39 of SEQ ID NO: 2, 3, 9, 10, 12, or 13. In another embodiment, the V of formula (II) is conjugated at one or more amino acid position numbers between 28 to 39 of SEQ ID NO: 2, 3, 9, 10, 12, or 13. In another embodiment, the V of formula (II) is conjugated at one or more amino acid position numbers between 30 to 39 of SEQ ID NO: 2, 3, 9, 10, 12, or 13. In another embodiment, the V of formula (II) is conjugated at one or more amino acid position numbers between 32 to 39 of SEQ ID NO: 2, 3, 9, 10, 12, or 13. In another embodiment, the V of formula (II) is conjugated at one or more amino acid position numbers between 36 to 39 of SEQ ID NO: 2, 3, 9, 10, 12, or 13.

In one embodiment, the V of formula (II) is conjugated at one or more positions between amino acid position numbers 17 to 39 of an OXM analog or an active fragment thereof. In another embodiment, the V of formula (II) is conjugated at one or more amino acid position numbers between: 22 to 39, 25 to 39, 28 to 39, 30 to 39, 32 to 39, or 36 to 39 of an OXM analog or an active fragment thereof. In another embodiment, the V of formula (II) is conjugated at one or more amino acid position numbers between 22 to 39 of an OXM analog or an active fragment thereof. In another embodiment, the V of formula (II) is conjugated at one or more amino acid position numbers between 25 to 39 of an OXM analog or an active fragment thereof. In another embodiment, the V of formula (II) is conjugated at one or more amino acid position numbers between 28 to 39 of an OXM analog or an active fragment thereof. In another embodiment, the V of formula (II) is conjugated at one or more amino acid position numbers between 30 to 39 of an OXM analog or an active fragment thereof. In another embodiment, the V of formula (II) is conjugated at one or more amino acid position numbers between 32 to 39 of an OXM analog or an active fragment thereof. In another embodiment, the V of formula (II) is conjugated at one or more amino acid position numbers between 36 to 39 of an OXM analog or an active fragment thereof.

In a related embodiment, C38 in the OXM analog shown in SEQ ID NO: 6 or SEQ ID NO: 7 is used as a conjugation site for the binder complex.

In a related embodiment, C39 in the OXM analog shown in SEQ ID NO: 5 is used as a conjugation site for the binder complex.

In a related embodiment, K39 in the OXM analog shown in SEQ ID NO: 14 is used as a conjugation site for the binder complex.

In a related embodiment, K38 in the OXM analog shown in SEQ ID NO: 9 or SEQ ID NO: 10 is used as a conjugation site for the binder complex.

In a related embodiment, G39 in the OXM analog shown in SEQ ID NO: 9 or SEQ ID NO: 10 is used as a conjugation site for the binder complex.

In a related embodiment, both K38 and G39 in the OXM analog shown in SEQ ID NO: 9 or SEQ ID NO: 10 are used as conjugation sites for the binder complex.

In one embodiment, K39 in the OXM analog shown in SEQ ID NO: 8 is used as a conjugation site for the binder complex.

In one embodiment, G40 in the OXM analog shown in SEQ ID NO: 8 is used as a conjugation site for the binder complex.

In a related embodiment, both K39 and G40 in the OXM analog shown in SEQ ID NO: 8 are used as conjugation sites for the binder complex.

In a related embodiment, C38 in the OXM analog shown in SEQ ID NO: 12 or SEQ ID NO: 13 is used as a conjugation site for the binder complex.

In a related embodiment, C39 in the OXM analog shown in SEQ ID NO: 12 or SEQ ID NO: 13 is used as a conjugation site for the binder complex.

In a related embodiment, both C38 and C39 in the OXM analog shown in SEQ ID NO: 12 or SEQ ID NO: 13 are used as conjugation sites for the binder complex.

In one embodiment, C39 in the OXM analog shown in SEQ ID NO: 11 is used as a conjugation site for the binder complex.

In one embodiment, C40 in the OXM analog shown in SEQ ID NO: 11 is used as a conjugation site for the binder complex.

In one embodiment, both C39 and C40 in the OXM analog shown in SEQ ID NO: 11 are used as conjugation sites for the binder complex.

In one embodiment, K39 in the OXM analog shown in SEQ ID NO: 15 is used as a conjugation site for the binder complex.

In one embodiment, C40 in the OXM analog shown in SEQ ID NO: 15 is used as a conjugation site for the binder complex.

In one embodiment, both K39 and C40 in the OXM analog shown in SEQ ID NO: 15 are used as conjugation sites for the binder complex.

In one embodiment, C39 in the OXM analog shown in SEQ ID NO: 16 is used as a conjugation site for the binder complex.

In one embodiment, K40 in the OXM analog shown in SEQ ID NO: 16 is used as a conjugation site for the binder complex.

In one embodiment, both C39 and K40 in the OXM analog shown in SEQ ID NO: 16 are used as conjugation sites for the binder complex.

In a related embodiment, the C terminus of the OXM or OXM analog is used as a conjugation site for the binder complex. In another embodiment, the C terminus of the OXM or OXM analog is used as a conjugation site for the binder complex without interrupting the protein-receptor interaction.

In a related embodiment, the primary amine located at the C terminus of the OXM or OXM analog is used as a conjugation site for the binder complex. In another embodiment, the primary amine located at the C terminus of the OXM or OXM analog is used as a conjugation site for the binder complex without interrupting the protein-receptor interaction.

In one embodiment, any OXM, OXM analogs, or fragment thereof disclosed herein are conjugated to any binder complexes disclosed. In one embodiment, any synthetic OXM, OXM analogs, or fragment thereof disclosed herein are conjugated to any binder complexes disclosed throughout. In another embodiment, any synthetic OXM, OXM analogs, or fragment thereof disclosed herein are conjugated to any binder complexes of V of Formula II disclosed throughout. In another embodiment, any OXM, OXM analogs, or fragment thereof disclosed herein are conjugated to any binder complexes of V of Formula II disclosed throughout.

In one embodiment, any OXM, OXM analogs, or fragment thereof disclosed herein are chemically conjugated to any binder complexes disclosed. In one embodiment, any synthetic OXM, OXM analogs, or fragment thereof disclosed herein are chemically conjugated to any binder complexes disclosed throughout. In another embodiment, any synthetic OXM, OXM analogs, or fragment thereof disclosed herein are chemically conjugated to any binder complexes of V of Formula II disclosed throughout. In another embodiment, any OXM, OXM analogs, or fragment thereof disclosed herein are chemically conjugated to any binder complexes of V of Formula II disclosed throughout.

In one embodiment, compounds that contain derivatized peptides or which contain non-peptide groups may be synthesized by well-known organic chemistry techniques. In a related embodiment, any of the binder complexes disclosed herein are synthetized by well-known organic chemistry techniques. In another embodiment, any of the elements within the binder complex are synthetized by well known organic chemistry techniques.

In another embodiment, the entire acylated OXM or acylated OXM analog is a synthetic protein. In another embodiment, the entire acylated OXM or acylated OXM analog is produced by direct peptide synthesis. In another embodiment, the entire acylated OXM or acylated OXM analog is produced by solid-phase peptide synthesis. In another embodiment, the entire acylated OXM or acylated OXM analog is produced by direct peptide synthesis using solid-phase techniques known in the art.

In another embodiment, the entire acylated OXM or acylated OXM analog is produced by an all on resin chain elongation methodology. In another embodiment, the entire acylated OXM or acylated OXM analog is produced by a one step variant synthesis (all on resin), where the mutated OXM backbone is synthesized on resin, following by a side chain elongation to synthesize the acylated OXM or OXM analog. In another embodiment, the all on resin chain elongation methodology for producing the entire acylated OXM or acylated OXM analog is detailed in FIG. 5.

The present application provides new compounds which are capable of binding albumin for use in extending the half-life of biologically active moieties to which they are attached. Specifically present application provides new compounds which are capable of binding albumin for use in extending the half-life of OXM or OXM analog to which they are attached. Thus, the present application provides albumin-binding compounds consisting essentially of the following elements: a binder, a spacer, a linker, and a biologically active moiety.

The present application provides new compounds which are capable of binding albumin for use in extending the half-life of biologically active moieties to which they are attached. Specifically present application provides new compounds which are capable of binding albumin for use in extending the half-life of the OXM or OXM analog to which they are attached. Thus, the present application provides albumin-binding compounds consisting essentially of the following elements: a binder, a spacer, and a biologically active moiety.

The present application provides new compounds which are capable of binding albumin for use in extending the half-life of biologically active moieties to which they are attached. Specifically present application provides new compounds which are capable of binding albumin for use in extending the half-life of OXM or OXM analog to which they are attached. Thus, the present application provides albumin-binding compounds consisting essentially of the following elements: a binder, a spacer, an optional linker, and a biologically active moiety.

The term “albumin binding moiety” as used herein means a residue which binds non-covalently to human serum albumin. In another embodiment, acylation with fatty acids delays clearance of the peptide it is attached to. In another embodiment, the OXM or OXM analog is acylated with an acyl group of sufficient size to bind serum albumin.

In one embodiment, the modified OXM or OXM analog consists of the following elements: a binder, a spacer, a linker, and OXM or OXM analog. In another embodiment, the modified OXM or OXM analog consists of the following elements: a binder, a spacer, a reversible linker, and OXM or OXM analog. In another embodiment, the modified OXM or OXM analog consists of the following elements: a binder, a spacer, a non-reversible linker, and OXM or OXM analog.

In another embodiment, the modified OXM or OXM analog consists of the following elements: a fatty acid chain, a spacer, a linker, and OXM or OXM analog. In another embodiment, the modified OXM or OXM analog consists of the following elements: a fatty acid chain, a spacer, a reversible linker, and OXM or OXM analog. In another embodiment, the modified OXM or OXM analog consists of the following elements: a fatty acid chain, a spacer, a non-reversible linker, and OXM or OXM analog.

In one embodiment, the modified OXM or OXM analog consists of the following elements: a binder, a spacer, and OXM or OXM analog. In another embodiment, the modified OXM or OXM analog consists of the following elements: a fatty acid chain, a spacer, and OXM or OXM analog.

In one embodiment, the modified OXM or OXM analog consists of the following elements: a binder, a spacer, an optional linker, and OXM or OXM analog. In another embodiment, the modified OXM or OXM analog consists of the following elements: a binder, a spacer, an optional reversible linker, and OXM or OXM analog. In another embodiment, the modified OXM or OXM analog consists of the following elements: a binder, a spacer, an optional non-reversible linker, and a OXM or OXM analog.

In another embodiment, the modified OXM or OXM analog consists of the following elements: a fatty acid chain, a spacer, an optional linker, and OXM or OXM analog. In another embodiment, the modified OXM or OXM analog consists of the following elements: a fatty acid chain, a spacer, an optional reversible linker, and OXM or OXM analog. In another embodiment, the modified OXM or OXM analog consists of the following elements: a fatty acid chain, a spacer, an optional non-reversible linker, and OXM or OXM analog.

In one embodiment, the present application provides modified an OXM or OXM analog consisting essentially of:

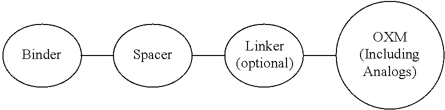

In one embodiment, the modified oxyntomodulin (“OXM”) or OXM analogs have the structure of formula (I):

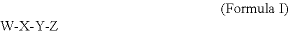

wherein:
W represents a binder;
X represents a spacer;
Y represents an optional linker;
Z represents native OXM, an OXM analog, or an active fragment thereof.

In one embodiment, the term “binder complex” refers to the binder, spacer, and an optional linker. In a related embodiment, the term “binder complex” refers to W, X, and Y of Formula I. In a related embodiment, the term “binder complex” refers to W and X of Formula I. In another embodiment, the term “binder complex” refers to a fatty acid chain, a spacer, and an optional linker. In another embodiment, the term “binder complex” refers to a fatty acid chain and a spacer.

In one embodiment, the term “binder complex” can be used interchangeably with any of the following terms: “probe”, “albumin binding probe”, or “albumin-binding probe”.

In one embodiment, the modified oxyntomodulin (“OXM”) disclosed herein has the structure of formula (II):

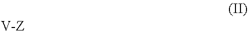

wherein:
   V represents a binder complex; and
   Z represents native OXM, an OXM analog, or an active fragment thereof.

In one embodiment, the V of formula II is:
Eicosanedioic-gGlu-(AEEA)ₙ-Lys(AcBr)-Gly-OH;
Octadecanedioic-gGlu-(AEEA)ₙ-Lys(AcBr)-Gly-OH;
Eicosanedioic-gGlu-Glu-(AEEA)ₙ-Lys(AcBr)-Gly-OH;
Octadecanedioic-gGlu-Glu-(AEEA)ₙ-Lys(AcBr)-Gly-OH;
Eicosanedioic-gGlu-(AEEA)ₙ; or
Octadecanedioic-gGlu-(AEEA)ₙ,
   wherein n is 1, 2 or 3.

In one embodiment, the V of formula II is:
Eicosanedioic-gGlu-(AEEA)₂-Lys(AcBr)-Gly-OH;
Octadecanedioic-gGlu-(AEEA)₂-Lys(AcBr)-Gly-OH;
Eicosanedioic-gGlu-Glu-(AEEA)₃-Lys(AcBr)-Gly-OH;
Octadecanedioic-gGlu-Glu-(AEEA)₃-Lys(AcBr)-Gly-OH;
Eicosanedioic-gGlu-(AEEA)₂; or
Octadecanedioic-gGlu-(AEEA)₂.

In another embodiment, the V of formula II is:
Hexadecanedioic-gGlu-(AEEA)ₙ-Cys-Gly-OH;
Octadecanedioic-gGlu-(AEEA)ₙ-Cys-Gly-OH;
Hexadecanedioic-gGlu-(AEEA)ₙ-Cys-Gly-OH; or
Octadecanedioic-gGlu-(AEEA)ₙ-Cys-Gly-OH,
   wherein n is 1, 2 or 3.

In another embodiment, the V of formula II is:
Hexadecanedioic-gGlu-AEEA-Cys-Gly-OH;
Octadecanedioic-gGlu-AEEA-Cys-Gly-OH;
Hexadecanedioic-gGlu-(AEEA)₂-Cys-Gly-OH; or
Octadecanedioic-gGlu-(AEEA)₂-Cys-Gly-OH.

In another embodiment, the V of formula II is:

(SEQ ID NO: 17)
H-Glu-Tyr-Glu-Lys(Octadecanedioic)-Glu-Tyr-Glu-
(AEEA)ₙ-Cys-Gly-OH;
(SEQ ID NO: 18)
H-Tyr-Glu-Lys(Octadecanedioic)-Glu-Tyr-(AEEA)ₙ-
Cys-Gly-OH;
(SEQ ID NO: 19)
H-Glu-Lys(Octadecanedioic)-Glu-(AEEA)ₙ-Cys-Gly-
OH;
(SEQ ID NO: 20)
H-Arg-Tyr-Arg-Lys(Octadecanedioic)-Arg-Tyr-Arg-
(AEEA)ₙ-Cys-Gly-OH;
or
(SEQ ID NO: 25)
Octadecanedioic-Glu-Tyr-Glu-Lys(Octadecanedioic)-
Glu-Tyr-Glu-(AEEA)ₙ-Cys-Gly-OH,

wherein n is 1, 2 or 3.

In another embodiment, the V of formula II is:

(SEQ ID NO: 21)
H-Glu-Tyr-Glu-Lys(Octadecanedioic)-Glu-Tyr-Glu-
AEEA-Cys-Gly-OH;
(SEQ ID NO: 22)
H-Tyr-Glu-Lys(Octadecanedioic)-Glu-Tyr-AEEA-Cys-
Gly-OH;
H-Glu-Lys(Octadecanedioic)-Glu-AEEA-Cys-Gly-OH;
(SEQ ID NO: 23)
H-Arg-Tyr-Arg-Lys(Octadecanedioic)-Arg-Tyr-Arg-
AEEA-Cys-Gly-OH;
or
(SEQ ID NO: 24)
Octadecanedioic-Glu-Tyr-Glu-Lys(Octadecanedioic)-
Glu-Tyr-Glu-AEEA-Cys-Gly-OH.

In another embodiment, the V of formula II is:

| (SEQ ID NO: 25) | |
|---|---|
| | H-Glu-Tyr-Glu-Lys(Eicosanedioic)-Glu-Tyr-Glu- |
| | (AEEA)ₙ-Cys-Gly-OH; |
| (SEQ ID NO: 26) | |
| | H-Tyr-Glu-Lys(Eicosanedioic)-Glu-Tyr-(AEEA)ₙ- |
| | Cys-Gly-OH; |
| | H-Glu-Lys(Eicosanedioic)-Glu-(AEEA)ₙ-Cys-Gly- |
| | OH; |
| (SEQ ID NO: 27) | |
| | H-Arg-Tyr-Arg-Lys(Eicosanedioic)-Arg-Tyr-Arg- |
| | (AEEA)ₙ-Cys-Gly-OH; |
| | or |
| (SEQ ID NO: 28) | |
| | Eicosanedioic -Glu-Tyr-Glu-Lys(Eicosanedioic)- |
| | Glu-Tyr-Glu-(AEEA)ₙ-Cys-Gly-OH,; |

In another embodiment, the V of formula II is:

(SEQ ID NO: 29)
H-Glu-Tyr-Glu-Lys(Eicosanedioic)-Glu-Tyr-Glu-AEEA-
Cys-Gly-OH;
(SEQ ID NO: 30)
H-Tyr-Glu-Lys(Eicosanedioic)-Glu-Tyr-AEEA-Cys-Gly-
OH;
H-Glu-Lys(Eicosanedioic)-Glu-AEEA-Cys-Gly-OH;
(SEQ ID NO: 31)
H-Arg-Tyr-Arg-Lys(Eicosanedioic)-Arg-Tyr-Arg-AEEA-
Cys-Gly-OH;
or
(SEQ ID NO: 32)
Eicosanedioic-Glu-Tyr-Glu-Lys(Eicosanedioic)-Glu-
Tyr-Glu-AEEA-Cys-Gly-OH.

In one embodiment, any of the binder complexes described herein are an albumin binding group. In one embodiment the binder complex V of Formula II is an albumin binding group.

In one embodiment, the binder complex increases the binding affinity of the native OXM, an OXM analog, or an active fragment thereof, to human serum albumin. In another embodiment, the binder complex V of Formula II increases the binding affinity of the native OXM, an OXM analog, or an active fragment thereof, to human serum albumin.

In one embodiment, the binder complex increases the binding affinity of the modified OXM to human serum albumin. In another embodiment, the binder complex V of Formula II increases the binding affinity of the modified OXM to human serum albumin.

In one embodiment, the V of Formula II comprises the structure of formula III:

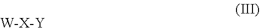

wherein:
   W represents a binder;
   X represents a spacer; and
   Y represents an optional linker.

In one embodiment, the W of Formula I or W of Formula III is a fatty acid chain. In another embodiment, the binder of Formula I or Formula III is a fatty acid chain.

As used herein, the term “fatty acid chain” refers to the hydrocarbon backbone of fatty acids (excluding the terminal acidic group) containing 2 to 40 carbon atoms. Fatty acid chain can refer to a octadecanedioic acid (C18 diacid) or eicosanedioic acid (C20 diacid).

In one embodiment, any of the binders described herein are an albumin binding group. In one embodiment the binder W of Formula III is an albumin binding group.

In one embodiment, the binder increases the binding affinity of the native OXM, an OXM analog, or an active fragment thereof, to human serum albumin. In another embodiment, the binder W of Formula III increases the binding affinity of the native OXM, an OXM analog, or an active fragment thereof, to human serum albumin.

In one embodiment, the binder increases the binding affinity of the modified OXM to human serum albumin. In another embodiment, the binder W of Formula III increases the binding affinity of the modified OXM to human serum albumin.

As used herein, the octadecanedioic acid (C18 diacid) fatty acid has the following structure of Formula IV:

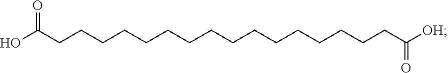 [SMILES: O=C(O)CCCCCCCCCCCCCCCCC(=O)O]

or has the following structure of Formula IV-A when linked to an amino or amine group to form an amide bond:

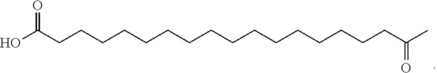 [SMILES: CC(=O)CCCCCCCCCCCCCCCCC(=O)O]

As used herein, the eicosanedioic acid (C20 diacid) fatty acid has the following structure of Formula V:

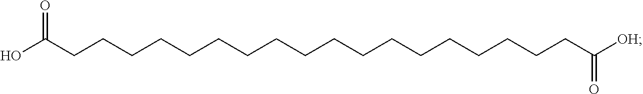 [SMILES: O=C(O)CCCCCCCCCCCCCCCCCCC(=O)O]

or has the following structure of Formula V-A when linked to an amino or amine group to form an amide bond:

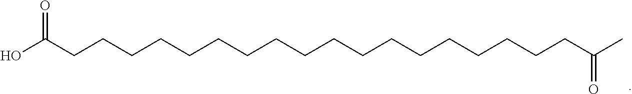 [SMILES: CC(=O)CCCCCCCCCCCCCCCCCCC(=O)O]

In one embodiment, the W of Formula I or Formula III is a hydrocarbon backbone of fatty acids containing 2 to 40 carbon atoms. In another embodiment, the binder of Formula I or Formula III is a hydrocarbon backbone of fatty acids containing 2 to 40 carbon atoms.

In a preferred embodiment, the W of Formula I or Formula III is a fatty acid containing 12 to 24 carbon atoms. In a preferred embodiment, the binder of Formula I or Formula III is a fatty acid containing 12 to 24 carbon atoms.

In one embodiment, the W of Formula I or Formula III is octadecanedioic acid (C18 diacid) or eicosanedioic acid (C20 diacid). In another embodiment, the binder of Formula I or Formula III is octadecanedioic acid (C18 diacid) or eicosanedioic acid (C20 diacid).

In one embodiment, the W of Formula I or Formula III is octadecanedioic acid (C18 diacid) and is represented by Formula IV:

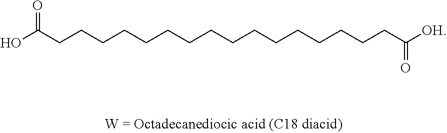 [SMILES: O=C(O)CCCCCCCCCCCCCCCCC(=O)O]

In another embodiment, the W of Formula I or Formula III is octadecanedioic acid (C18 diacid) and is represented by Formula IV-A when linked to an amino or amine group to form an amide bond:

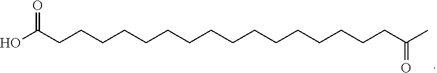 [SMILES: CC(=O)CCCCCCCCCCCCCCCCC(=O)O]

In one embodiment, the W of Formula I or Formula III is eicosanedioic acid (C20 diacid) and is represented by Formula V:

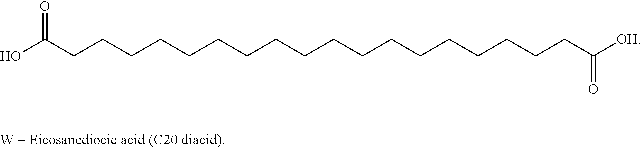 [SMILES: O=C(O)CCCCCCCCCCCCCCCCCCC(=O)O]

In another embodiment, the W of Formula I or Formula III is eicosanedioic acid (C20 diacid) and is represented by Formula V-A when linked to an amino or amine group to form an amide bond:

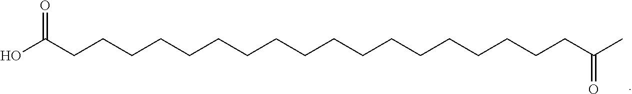 [SMILES: CC(=O)CCCCCCCCCCCCCCCCCCC(=O)O]

In one embodiment, the X of Formula I or Formula III is a spacer.

In one embodiment, the X of Formula I or Formula III is a natural AA, unnatural AA, PEG or PEG-like molecules, or other bio-polymers (e.g. poly-sugars). In another embodiment, the spacer of Formula I or Formula III is a natural AA, unnatural AA, PEG or PEG-like molecules, or other bio-polymers (e.g. poly-sugars).

In one embodiment, the X of Formula I or Formula III has a molecular weight between about 75 Da to 5 KDa. In another embodiment, the spacer of Formula I or Formula III has a molecular weight between about 75 Da to 5 KDa.

In one embodiment, the X of Formula I or Formula III is any one of gGlu-(AEEA)ₙ, gGlu-(AEEA)ₙ-Cys, or gGlu-(AEEA)ₙ-Cys-Gly, and n is 1, 2, or 3.

In one embodiment, the X of Formula I or Formula III is any one of gGlu-Gluₙ-(AEEA)ₘ-Cys-Glyₚ (SEQ ID NOs: 33 and 34), gGlu-Gluₙ-(AEEA)ₘ-Lys(AcBr)-Glyₚ (SEQ ID NOs: 35 and 36), or gGlu-Gluₙ-(AEEA)ₘ-Lys-Glyₚ (SEQ ID NOs: 37 and 38) and n is 1, 2, or 3; m is 1, 2, or 3; and p is 1, 2, or 3. In one embodiment, the X of Formula I or Formula III is any one of gGlu-Gluₙ-(AEEA)ₘ-Cys-Glyₚ (SEQ ID NOs: 33 and 34), gGlu-Gluₙ-(AEEA)ₘ-Lys(AcBr)-Gly (SEQ ID NOs: 35 and 36), or gGlu-Gluₙ-(AEEA)ₘ-Lys-Gly (SEQ ID NOs: 37 and 38) and n is 1, 2, or 3; m is 1, 2, or 3; and p is 1, 2, or 3.

In one embodiment, the X of Formula I or Formula III is any one of gGlu-Glu-(AEEA)ₘ-Cys-Gly, gGlu-Glu-(AEEA)ₘ-Lys(AcBr)-Gly, or gGlu-Glu-(AEEA)ₘ-Lys-Gly and m is 1, 2, or 3. In another embodiment, the X of Formula I or Formula III is any one of gGlu-Glu-(AEEA)ₘ-Cys-Gly, gGlu-Glu-(AEEA)ₘ-Lys(AcBr)-Gly, or gGlu-Glu-(AEEA)ₘ-Lys-Gly and m is 1. In another embodiment, the X of Formula I or Formula III is any one of gGlu-Glu-(AEEA)ₘ-Cys-Gly, gGlu-Glu-(AEEA)ₘ-Lys(AcBr)-Gly, or gGlu-Glu-(AEEA)ₘ-Lys-Gly and m is 2. In another embodiment, the X of Formula I or Formula III is any one of gGlu-Glu-(AEEA)ₘ-Cys-Gly, gGlu-Glu-(AEEA)ₘ-Lys(AcBr)-Gly, or gGlu-Glu-(AEEA)ₘ-Lys-Gly and m is 3.

In one embodiment, the X of Formula I or Formula III is represented by Formula VI:

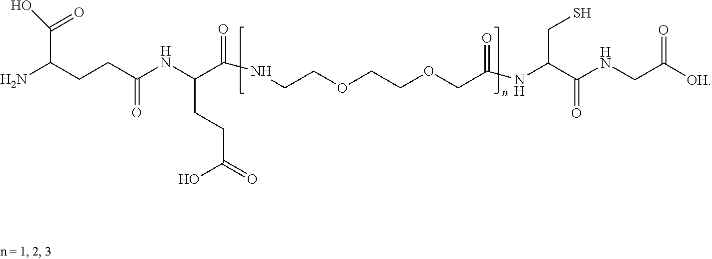

In one embodiment, the X of Formula I or Formula III is gGlu-Glu-(AEEA)ₘ-Cys-Gly, m is 1, 2, or 3, and is represented by Formula VII.

In one embodiment, the X of Formula I or Formula III is represented by Formula VI-A:

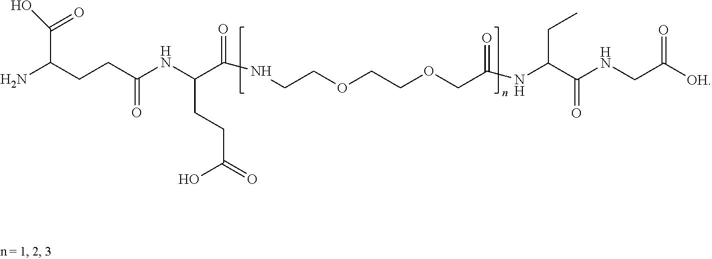

In one embodiment, the X of Formula I or Formula III is represented by Formula VI-B:

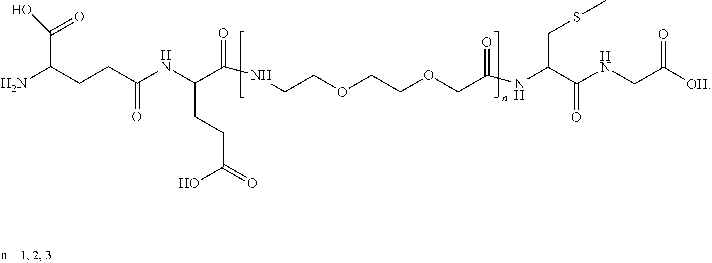

In one embodiment, the X of Formula I or Formula III is represented by Formula VI-B when linked to an adjoining moiety at the S bond. In another embodiment, the X of Formula I or Formula III is represented by Formula VI-B when linked to an adjoining moiety at the S bond at the C-terminus.

In one embodiment, the X of Formula I or Formula III is represented by Formula VI-C:

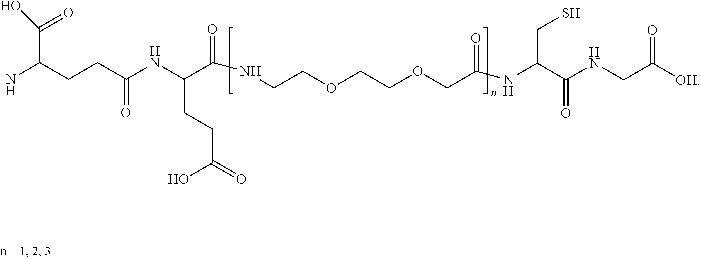

In one embodiment, the X of Formula I or Formula III is represented by Formula VI-C when linked to an amino or amine group to form an amide bond. In another embodiment, the X of Formula I or Formula III is represented by Formula VI-C when linked to an amino or amine group to form an amide bond at the N-terminus.

In one embodiment, the X of Formula I or Formula III is represented by Formula VI-D:

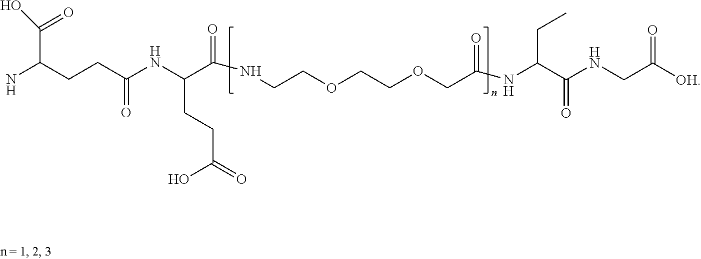

In one embodiment, the X of Formula I or Formula III is represented by Formula VI-E:

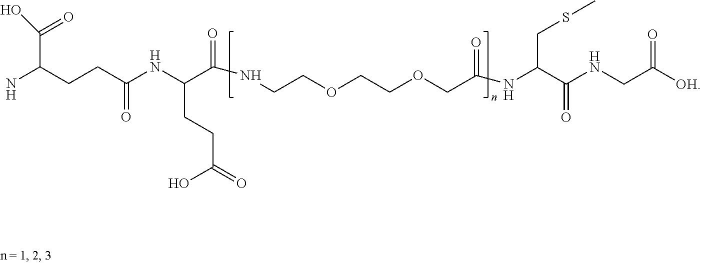

In one embodiment, the X of Formula I or Formula III is represented by Formula VI-E when linked to an adjoining moiety at the S bond and when linked to an amino or amine group to form an amide bond. In another embodiment, the X of Formula I or Formula III is represented by Formula VI-E when linked to an adjoining moiety at the S bond and when linked to an amino or amine group to form an amide bond at the N-terminus. In another embodiment, the X of Formula I or Formula III is represented by Formula VI-E when linked to an adjoining moiety at the S bond at the C-terminus and when linked to an amino or amine group to form an amide bond at the N-terminus.

In one embodiment, the X of Formula I or Formula III is represented by Formula VII:

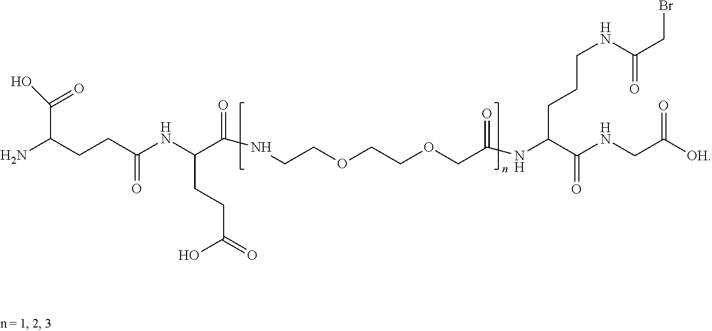

In one embodiment, the X of Formula I or Formula III is gGlu-Glu-(AEEA)ₘ-Lys(AcBr)-Gly, m is 1, 2, or 3, and is represented by Formula VII.

In one embodiment, the X of Formula I or Formula III is represented by Formula VII-A:

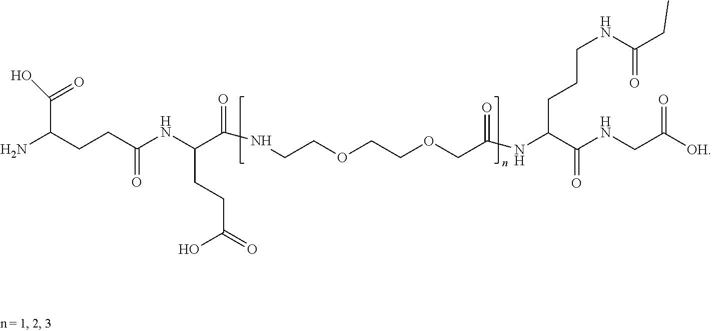

In one embodiment, the X of Formula I or Formula III is represented by Formula VII-A when linked to an adjoining moiety at the acyl carbon. In another embodiment, the X of Formula I or Formula III is represented by Formula VII-A when linked to an adjoining moiety at the acyl carbon at the C-terminus.

In one embodiment, the X of Formula I or Formula III is represented by Formula VII-B:

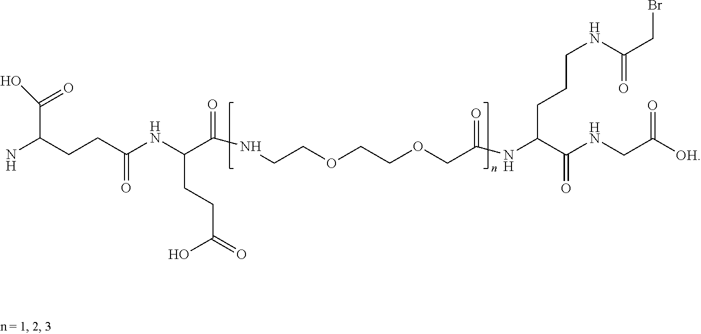

In one embodiment, the X of Formula I or Formula III is represented by Formula VII-B when linked to an amino or amine group to form an amide bond. In another embodiment, the X of Formula I or Formula III is represented by Formula VII-B when linked to an amino or amine group to form an amide bond at the N-terminus.

In one embodiment, the X of Formula I or Formula III is represented by Formula VII-C:

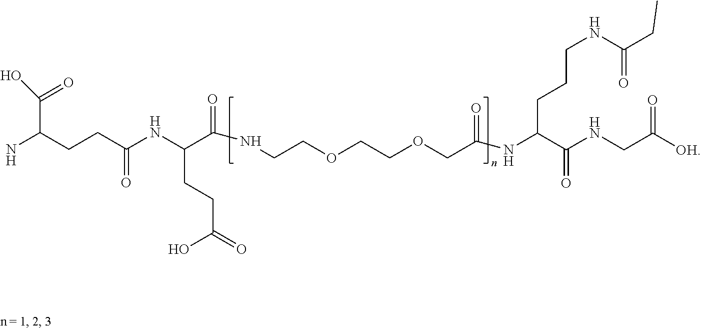

In one embodiment, the X of Formula I or Formula III is represented by Formula VII-C when linked to an amino or amine group to form an amide bond and when linked to an adjoining moiety at the acyl carbon. In another embodiment, the X of Formula I or Formula III is represented by Formula VII-C when linked to an amino or amine group to form an amide bond at the N-terminus and when linked to an adjoining moiety at the acyl carbon at the C-terminus.

In one embodiment, the X of Formula I or Formula III is represented by Formula VIII:

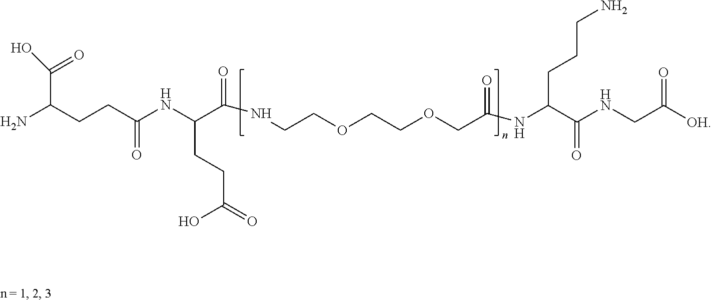

In one embodiment, the X of formula I or Formula III is X is gGlu-Glu-(AEEA)ₘ-Lys-Gly, m is 1, 2, or 3, and is represented by Formula VIII.

In one embodiment, the X of formula I or Formula III is represented by Formula VIII-A:

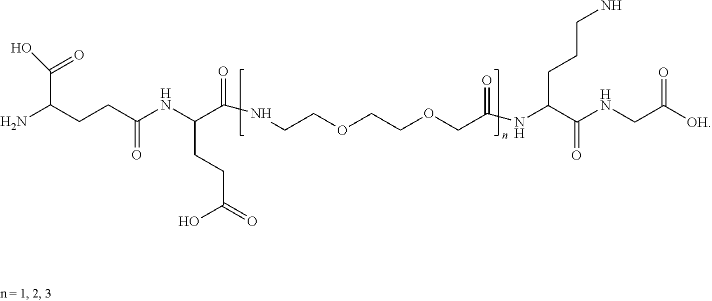

In one embodiment, the X of formula I or Formula III is represented by Formula VIII-A when linked through the lysine amino group. In another embodiment, the X of formula I or Formula III is represented by Formula VIII-A when linked through the lysine amino group at the C-terminus.

In one embodiment, the X of formula I or Formula III is represented by Formula VIII-B:

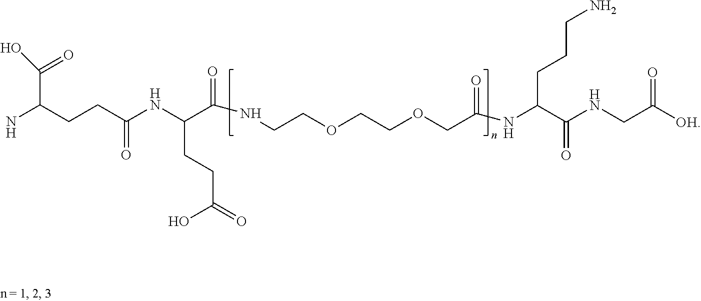

In one embodiment, the X of formula I or Formula III is represented by Formula VIII-B when linked to an amino or amine group to form an amide bond. In one embodiment, the X of formula I or Formula III is represented by Formula VIII-B when linked to an amino or amine group to form an amide bond at the N-terminus.

In one embodiment, the X of formula I or Formula III is represented by Formula VIII-C:

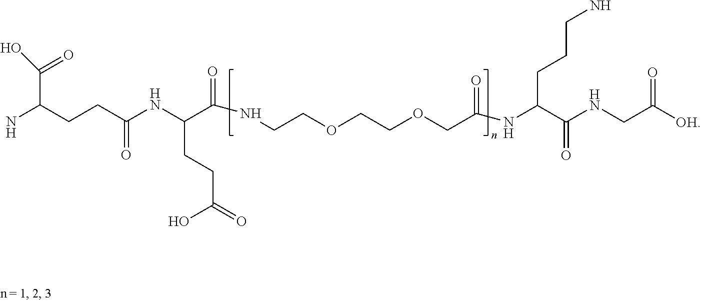

In one embodiment, the X of formula I or Formula III is represented by Formula VIII-D:

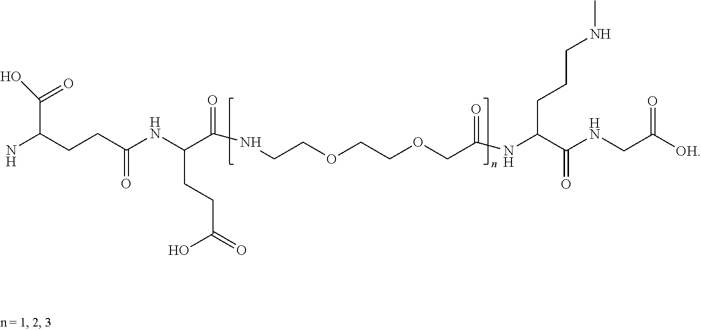

In one embodiment, the X of formula I or Formula III is represented by Formula VIII-D when linked to an amino or amine group to form an amide bond and when linked through the lysine amino group. In one embodiment, the X of formula I or Formula III is represented by Formula VIII-D when linked to an amino or amine group to form an amide bond at the N-terminus and when linked through the lysine amino group at the C-terminus.

In one embodiment, the Y of Formula I is a linker. In a related embodiment, the Y of Formula I is a linker and links W and Z of Formula I.

In one embodiment, the Y of Formula I or III is an optional linker.

In one embodiment, the Y of Formula I is not present. In a related embodiment, the Y of Formula I is not present, and Formula I then comprises W—X—Z.

In one embodiment, the Y of Formula I or III has a molecular weight between about 30 Da to about 600 Da. In another embodiment, the Y of Formula I or III has a molecular weight between about 43 Da to about 501 Da. In one embodiment, the linker of Formula I or III has a molecular weight between about 30 Da to about 600 Da. In another embodiment, the linker of Formula I or III has a molecular weight between about 43 Da to about 501 Da.

In one embodiment, the Y of Formula I or III is a bi-functional linker. In another embodiment, the Y of Formula I or III is Bis-dPEG®ₙ-NHS, where n is 1, 2, 3, 4, 5, 6, 7, 8, 9, or 10. In one embodiment, the linker of Formula I or III is a bi-functional linker. In another embodiment, the linker of Formula I or III is Bis-dPEG®ₙ-NHS, where n is 1, 2, 3, 4, 5, 6, 7, 8, 9, or 10.

In one embodiment, the Y of Formula I or III is a bi-functional linker. In another embodiment, the Y of Formula I or III is Bis-dPEG®ₙ-NHS ester, where n is 1, 2, 3, 4, 5, 6, 7, 8, 9, or 10. In one embodiment, the linker of Formula I or III is a bi-functional linker. In another embodiment, the linker of Formula I or III is Bis-dPEG®ₙ-NHS ester, where n is 1, 2, 3, 4, 5, 6, 7, 8, 9, or 10.

In one embodiment, the Y of Formula I or III is a bi-functional linker. In another embodiment, the Y of Formula I or III is Bis-PEGₙ-NHS, where n is 1, 2, 3, 4, 5, 6, 7, 8, 9, or 10. In one embodiment, the linker of Formula I or III is a bi-functional linker. In another embodiment, the linker of Formula I or III is Bis-PEGₙ-NHS, where n is 1, 2, 3, 4, 5, 6, 7, 8, 9, or 10.

In one embodiment, the Y of Formula I or III is a bi-functional linker with a mid molecular weight. In another embodiment, the Y of Formula I or III is a bi-functional linker with a mid molecular weight of approximately 2 kDa.

In one embodiment, the Y of Formula I or III is Chloropropane-2-one-Fmoc-Mal or Mal-NRFmoc-NHS. In another embodiment, the Y of Formula I or III is Chloropropane-2-one-Fmoc-Mal.

In another embodiment, the Y of Formula I or III is Mal-NRFmoc-NHS.

In one embodiment, the Y of Formula I or III is 2-(3-(2,5-dioxo-2H-pyrrol-1(5H)-yl)propanamido)-9H-fluoren-9-yl)methyl 3-chloro-2-oxopropylcarbamate.

In one embodiment, the Y of Formula I or III is a linker and is a stable covalent bond. In another embodiment, the Y of Formula I or III is a non-reversible linker. In another embodiment, the bond between the Z of Formula II and the linker of Formula I or III is a stable covalent bond. In another embodiment, the bond between Z and Y of Formula I or III is a stable covalent bond. In another embodiment, the bond between OXM and the linker of Formula I or III is a stable covalent bond. In another embodiment, the bond between OXM analog and the linker of Formula I or III is a stable covalent bond.

In one embodiment, the Y of Formula I or III is Mal-NRFmoc-NHS and is a stable covalent bond. In another embodiment, the Y of Formula I or III is Mal-NRFmoc-NHS and is a non-reversible linker. In another embodiment, the bond between the Z of Formula II and Mal-NRFmoc-NHS as the linker of Formula I or III is a stable covalent bond. In another embodiment, the bond between Z and Mal-NRFmoc-NHS is a stable covalent bond.

In one embodiment, the Y of Formula I or III is a linker and is a reversible covalent bond. In another embodiment, the Y or III of Formula I is a reversible linker. In another embodiment, the bond between the Z of Formula II and the linker of Formula I or III is a reversible covalent bond. In another embodiment, the bond between Z and Y of Formula I or Formula III is a reversible covalent bond. In another embodiment, the bond between the OXM of Formula II and the linker of Formula I or III is a reversible covalent bond. In another embodiment, the bond between the OXM analog of Formula II and the linker of Formula I or III is a reversible covalent bond.

In one embodiment, the Y of Formula I or III is Chloropropane-2-one-Fmoc-Mal and is a reversible covalent bond. In another embodiment, the Y of Formula I is Chloropropane-2-one-Fmoc-Mal and is a reversible linker. In another embodiment, the bond between the Z of Formula II and Chloropropane-2-one-Fmoc-Mal is a reversible covalent bond. In another embodiment, the bond between Z and Chloropropane-2-one-Fmoc-Mal is a reversible covalent bond.

In one embodiment, the Y of Formula I or III is Chloropropane-2-one-Fmoc-Mal and is represented by Formula IX (in another embodiment the Chloropropane can be substituted with Bromopropane or Iodopropane):

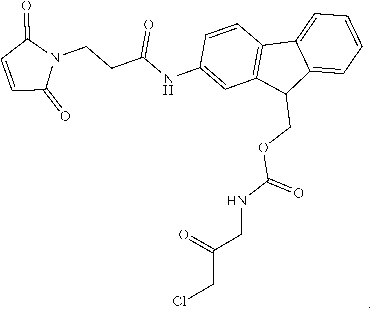 [SMILES: O=C(CCl)CNC(=O)OCC1c2ccccc2-c2ccc(NC(=O)CCN3C(=O)C=CC3=O)cc21]

In one embodiment, the Y or III of formula I is represented by Formula IX-A:

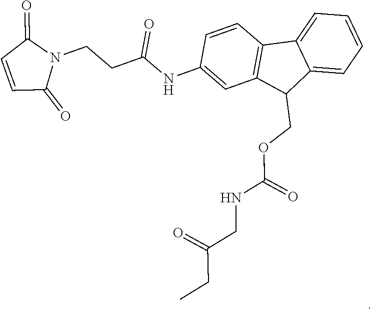 [SMILES: CCC(=O)CNC(=O)OCC1c2ccccc2-c2ccc(NC(=O)CCN3C(=O)C=CC3=O)cc21]

In one embodiment, the Y or III of formula I is represented by Formula IX-A when attached to an adjoining atom at the acyl carbon.

In one embodiment, the Y of formula I or III is represented by Formula IX-B:

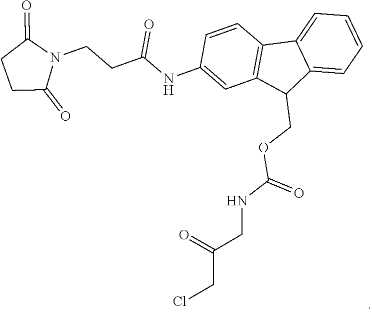 [SMILES: O=C(CCl)CNC(=O)OCC1c2ccccc2-c2ccc(NC(=O)CCN3C(=O)CCC3=O)cc21]

In one embodiment, the Y of formula I or III is represented by Formula IX-B when linked at the pyrrolidine ring to an adjoining atom.

In one embodiment, the Y of formula I or III is represented by Formula IX-C:

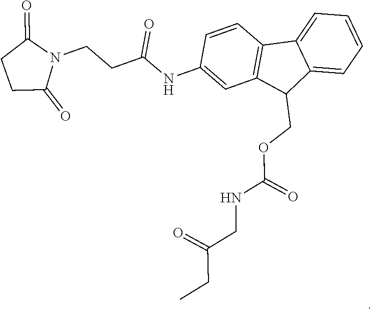 [SMILES: CCC(=O)CNC(=O)OCC1c2ccccc2-c2ccc(NC(=O)CCN3C(=O)CCC3=O)cc21]

In one embodiment, the Y of formula I or III is represented by Formula IX-B when linked at the pyrrolidine ring to an adjoining atom and when attached to an adjoining atom at the acyl carbon.

In another embodiment, the Y of Formula I or III is Chloropropane-2-one-Fmoc-Mal and is represented by Formula IX, and the Chloropropane can be substituted with Bromopropane or Iodopropane.

In another embodiment, the Y of Formula I or III is Chloropropane-2-one-Fmoc-Mal and the Chloropropane can be substituted with Bromopropane or Iodopropane.

In one embodiment, the Y of Formula I or III is Mal-Fmoc-Propyl bromide.

In another embodiment, the Y of Formula I or III is (2-(3-(2,5-dioxo-2H-pyrrol-1(5H)-yl)propanamido)-9H-fluoren-9-yl) methyl 3-bromo-2-oxopropylcarbamate.

In one embodiment, the Y of formula I or III is represented by Formula X:

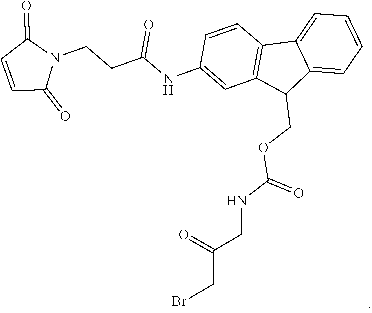 [SMILES: O=C(CBr)CNC(=O)OCC1c2ccccc2-c2ccc(NC(=O)CCN3C(=O)C=CC3=O)cc21]

In another embodiment, the Y of formula I or III is Mal-Fmoc-Propyl bromide and is represented by Formula X.

In one embodiment, the Y of formula I or III is Bromopropane-2-one-Fmoc-Mal. In another embodiment, the Y of formula I or III is Bromopropane-2-one-Fmoc-Mal and is represented by Formula X.

In one embodiment, the Y of Formula I or III is Mal-Fmoc-Propyl Chloride.

In another embodiment, the Y of Formula I or III is (2-(3-(2,5-dioxo-2H-pyrrol-1(5H)-yl)propanamido)-9H-fluoren-9-yl) methyl 3-chloro-2-oxopropylcarbamate.

In one embodiment, the Y of Formula I or III is represented by Formula XI:

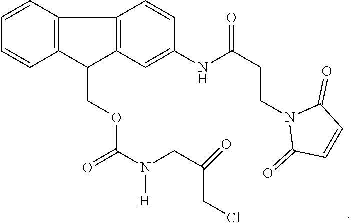 [SMILES: O=C(CCl)CNC(=O)OCC1c2ccccc2-c2ccc(NC(=O)CCN3C(=O)C=CC3=O)cc21]

In one embodiment, the Y of Formula I or III is Mal-Fmoc-Propyl Chloride and is represented by Formula XI.

In one embodiment, the Y of Formula I or III is Mal-Fmoc-Propyl Iodide.

In another embodiment, the Y of Formula I or III is (2-(3-(2,5-dioxo-2H-pyrrol-1(5H)-yl)propanamido)-9H-fluoren-9-yl)methyl 3-Iodo-2-oxopropylcarbamate.

In one embodiment, the Y of Formula I or III is represented by Formula XII:

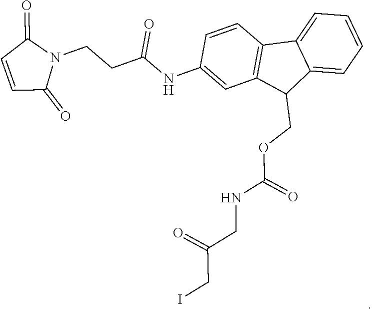 [SMILES: O=C(CI)CNC(=O)OCC1c2ccccc2-c2ccc(NC(=O)CCN3C(=O)C=CC3=O)cc21]

In another embodiment, the Y of Formula I or III is Mal-Fmoc-Propyl Iodide and is represented by Formula XII.

In one embodiment, the Y of formula I or III is Iodopropane-2-one-Fmoc-Mal. In another embodiment, the Y of formula I or III is Iodopropane-2-one-Fmoc-Mal and is represented by Formula XII.

In one embodiment, the Y of Formula I or III is Mal-NRFmoc-NHS and is represented by Formula XIII:

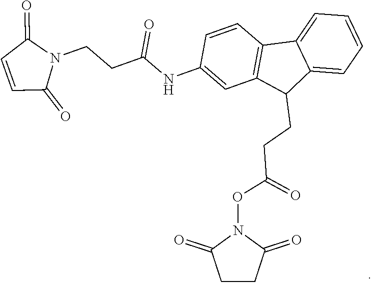 [SMILES: O=C(CCN1C(=O)C=CC1=O)Nc1ccc2c(c1)C(CCC(=O)ON1C(=O)CCC1=O)c1ccccc1-2]

In one embodiment, the Y of formula I or III is represented by Formula XIII-A:

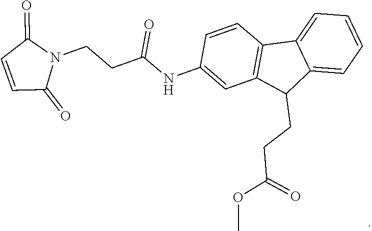 [SMILES: COC(=O)CCC1c2ccccc2-c2ccc(NC(=O)CCN3C(=O)C=CC3=O)cc21]

In one embodiment, the Y of formula I or III is represented by Formula XIII-A when linked at the oxygen to an adjoining atom.

In one embodiment, the Y of formula I or III is represented by Formula XIII-B:

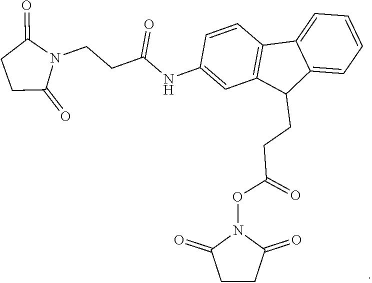 [SMILES: O=C(CCN1C(=O)CCC1=O)Nc1ccc2c(c1)C(CCC(=O)ON1C(=O)CCC1=O)c1ccccc1-2]

In one embodiment, the Y of formula I or III is represented by Formula XIII-B when linked at the pyrrolidine ring to an adjoining atom.

In one embodiment, the Y of formula I or III is represented by Formula XIII-C:

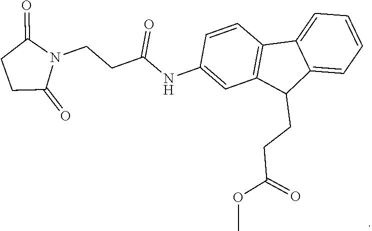 [SMILES: COC(=O)CCC1c2ccccc2-c2ccc(NC(=O)CCN3C(=O)CCC3=O)cc21]

In one embodiment, the Y of formula I or III is represented by Formula XIII-C when linked at the pyrrolidine ring to an adjoining atom and when linked at the oxygen to an adjoining atom.

In one aspect, the modified OXM disclosed herein comprises the following formula:

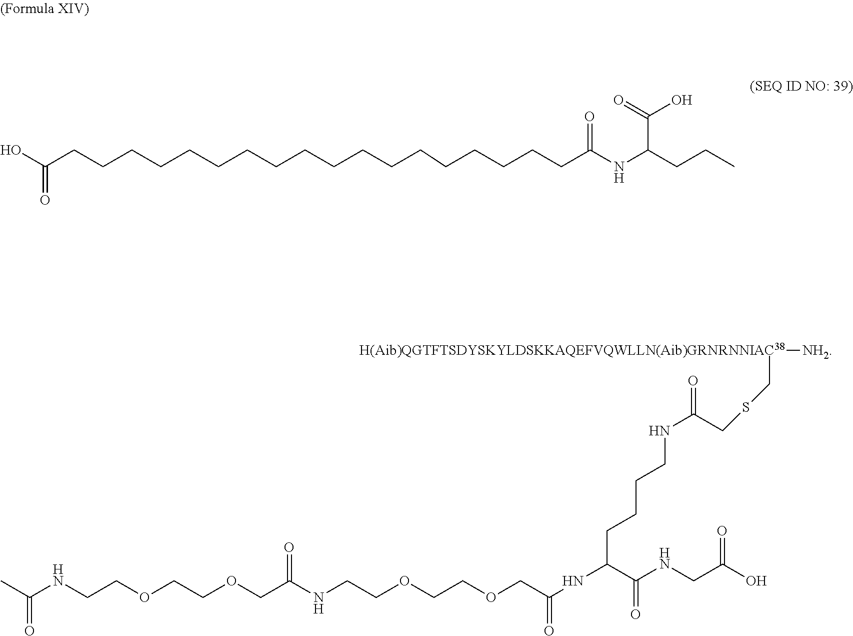

In one aspect, the modified OXM disclosed herein is H(Aib)QGTFTSDYSKYLDSKKAQEFVQWLLN(Aib)GRNRNNIAC³⁸(Eicosanedioic-gGlu-(AEEA)₂-Lys(Ac)-Gly)-NH₂ (SEQ ID NO: 39).

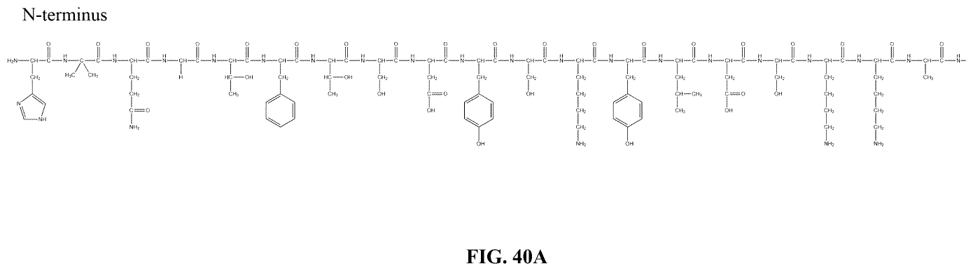

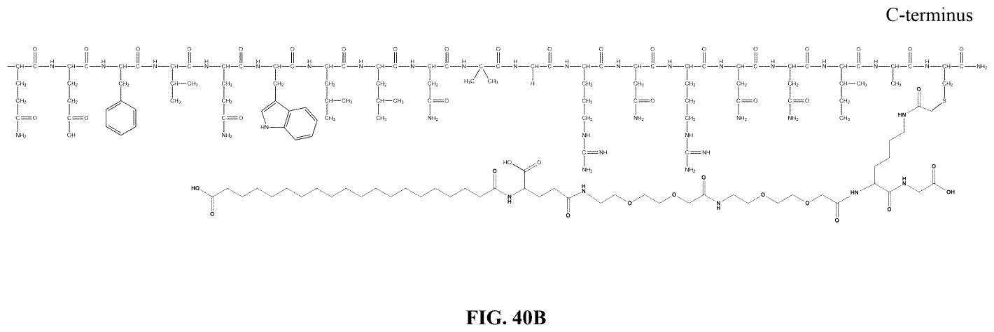

In one aspect, the full formula of the modified OXM disclosed herein as H(Aib)QGTFTSDYSKYLDSKKAQEFVQWLLN(Aib)GRNRNNIAC³⁸(Eicosanedioic-gGlu-(AEEA)₂-Lys(Ac)-Gly)-NH₂ (SEQ ID NO: 39) is shown in FIGS. 40A and 40B. The molecular weight of the compound shown in FIGS. 40A and 40B is 5390.13, and its chemical formula is C₂₄₁H₃₈₀N₆₄O₇₄S. FIG. 40A shows half of the full chemical structure of the modified OXM starting with the N-terminus of the OXM. FIG. 40B shows the other half of the full chemical structure of the modified OXM ending with the C-terminus of the OXM.

In one aspect, the modified OXM disclosed herein comprises the following formula:

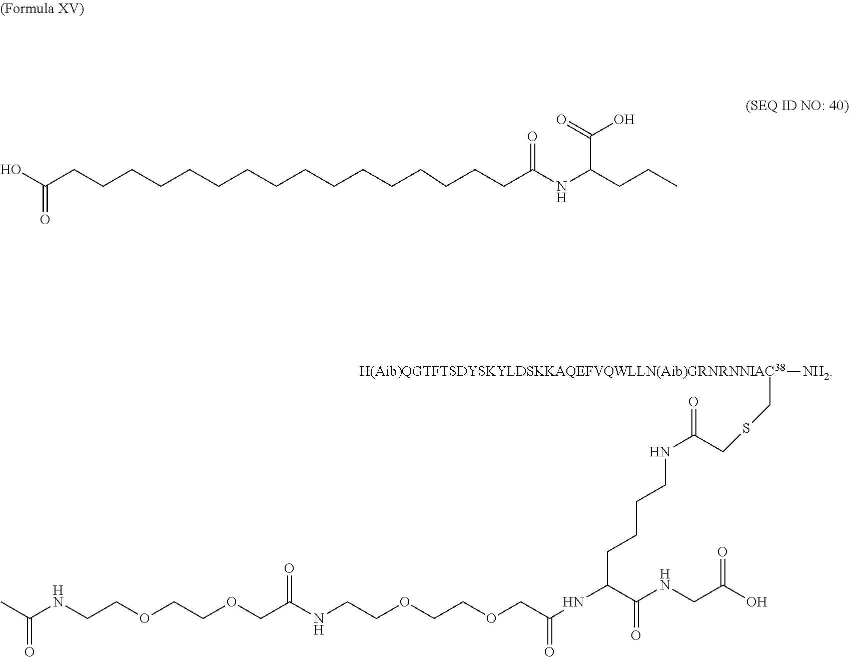

In one aspect, the modified OXM disclosed herein is H(Aib)QGTFTSDYSKYLDSKKAQEFVQWLLN(Aib)GRNRNNIAC³⁸(Octadecanedioic-gGlu-(AEEA)₂-Lys(Ac)-Gly)-NH₂ (SEQ ID NO: 40).

In one aspect, the modified OXM disclosed herein comprises the following formula:

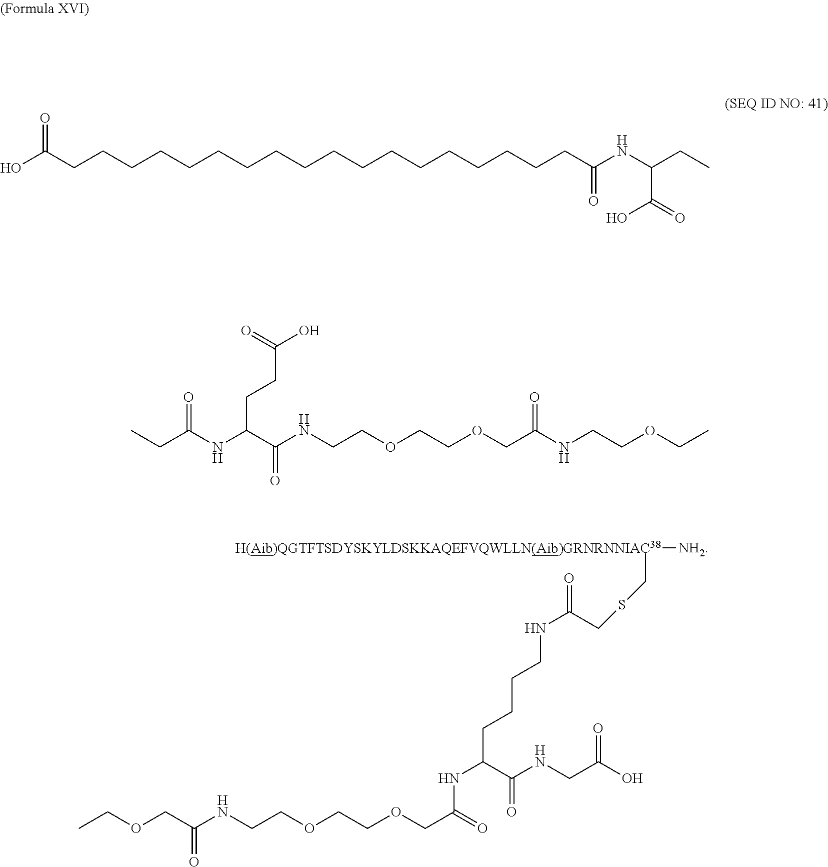

In one aspect, the modified OXM disclosed herein is H(Aib)QGTFTSDYSKYLDSKKAQEFVQWLLN(Aib)GRNRNNIAC³⁸(Eicosanedioic-gGlu-Glu-(AEEA)₃-Lys(Ac)-Gly)-NH₂ (SEQ ID NO: 41).

In one aspect, the modified OXM disclosed herein comprises the following formula:

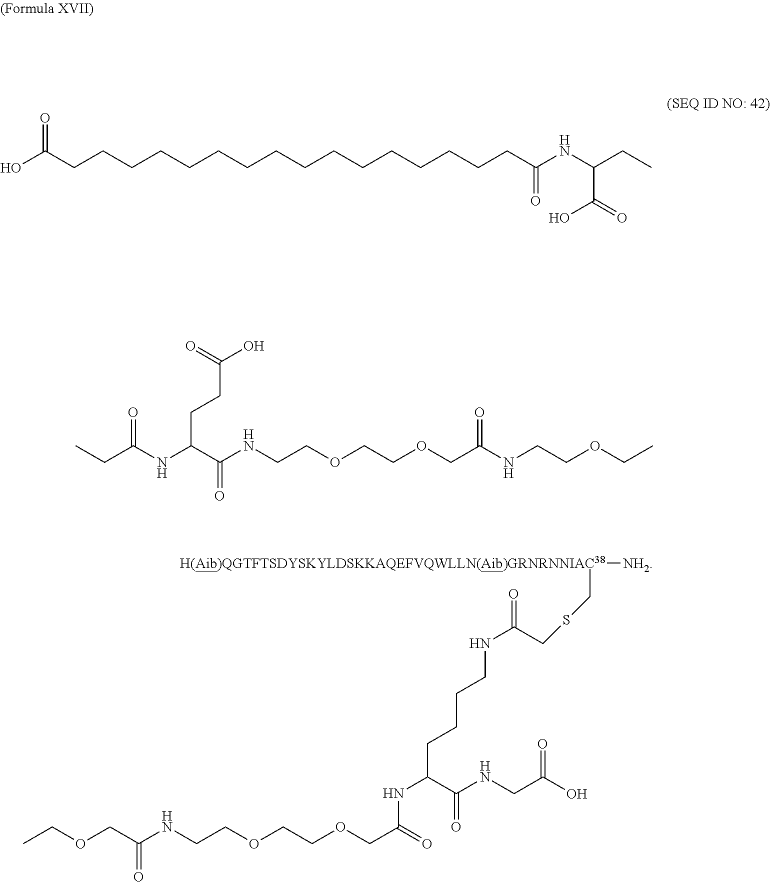

In one aspect, the modified OXM disclosed herein is H(Aib)QGTFTSDYSKYLDSKKAQEFVQWLLN(Aib)GRNRNNIAC³⁸(Octadecanedioic-gGlu-Glu-(AEEA)₃-Lys(Ac)-Gly)-NH₂ (SEQ ID NO: 42).

In one aspect, the modified OXM disclosed herein comprises the following formula:

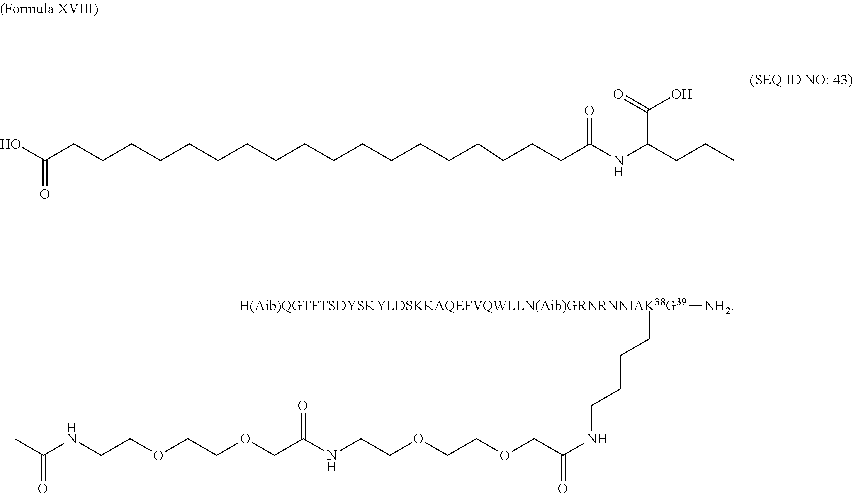

In one aspect, the modified OXM disclosed herein is H(Aib)QGTFTSDYSKYLDSKKAQEFVQWLLN(Aib)GRNRNNIAK³⁸(Eicosanedioic-gGlu-(AEEA)₂)G³⁹-NH₂ (SEQ ID NO: 43).

In one aspect, the modified OXM disclosed herein comprises the following formula:

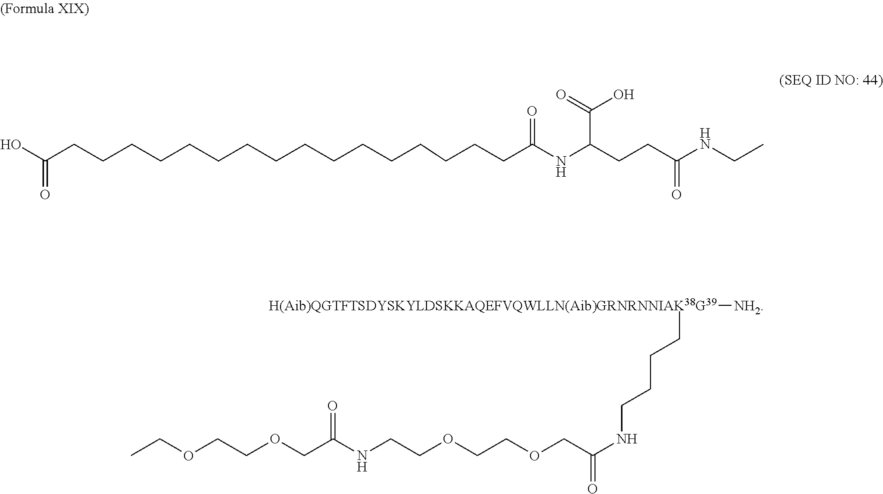

In one aspect, the modified OXM disclosed herein is H(Aib)QGTFTSDYSKYLDSKKAQEFVQWLLN(Aib)GRNRNNIAK³⁸(Octadecanedioic-gGlu-(AEEA)₂)G³⁹-NH₂ (SEQ ID NO: 44).

In one aspect, the modified OXM disclosed herein comprises the following formula

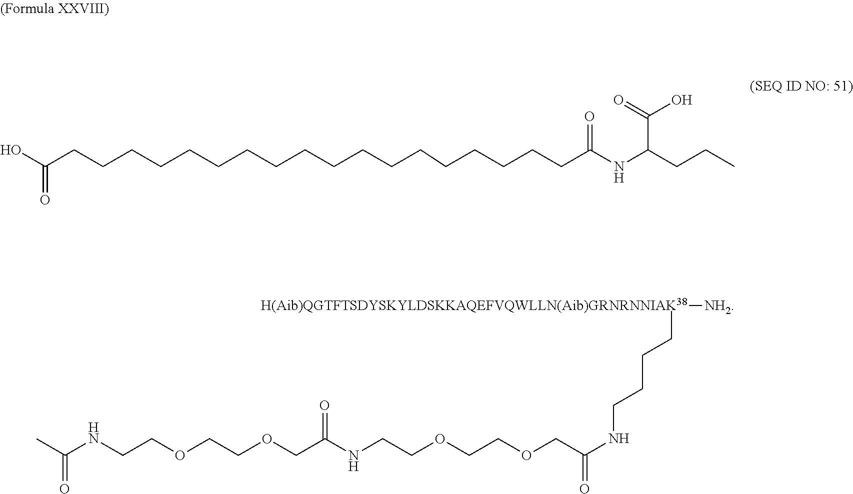

In one aspect, the modified OXM disclosed herein is H(Aib)QGTFTSDYSKYLDSKKAQEFVQWLLN(Aib)GRNRNNIAK³⁸(Eicosanedioic-gGlu-(AEEA)₂)-NH₂. (SEQ ID NO: 51)

In one aspect, the modified OXM disclosed herein comprises the following formula

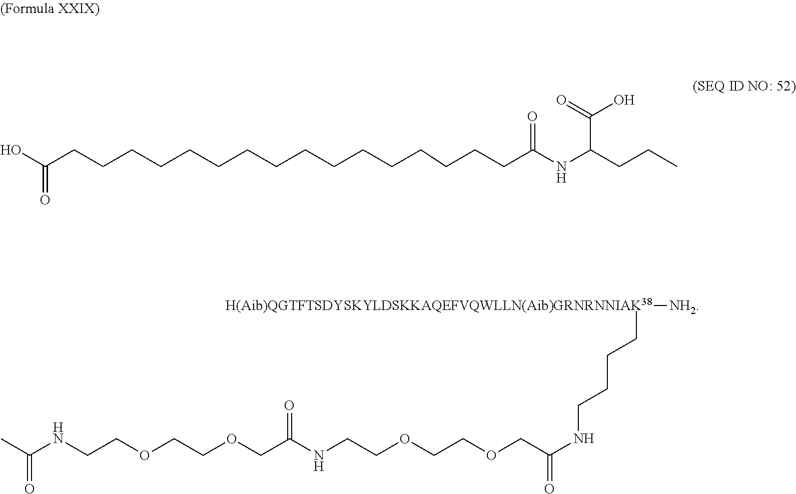

In one aspect, the modified OXM disclosed herein is H(Aib)QGTFTSDYSKYLDSKKAQEFVQWLLN(Aib)GRNRNNIAK³⁸(Octadecanedioic-gGlu-(AEEA)₂)-NH₂ (SEQ ID NO: 52).

In one aspect, the modified OXM disclosed herein comprises the following formula:

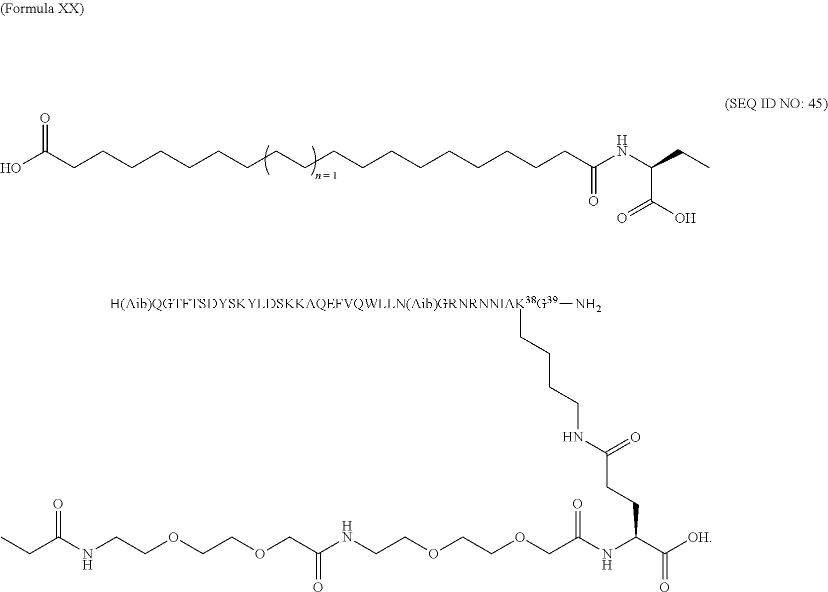

In one aspect, the modified OXM disclosed herein is H(Aib)QGTFTSDYSKYLDSKKAQEFVQWLLN(Aib)GRNRNNIAK³⁸(Eicosanedioic-gGlu-(AEEA)₂-gGlu)G³⁹-NH₂ (SEQ ID NO: 45).

In one aspect, the modified OXM disclosed herein comprises the following formula:

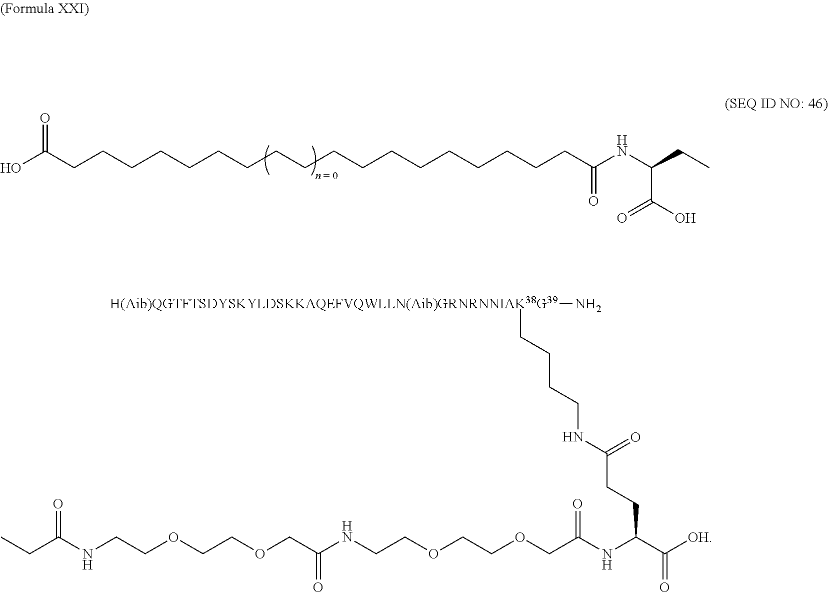

In one aspect, the modified OXM disclosed herein is H(Aib)QGTFTSDYSKYLDSKKAQEFVQWLLN(Aib)GRNRNNIAK³⁸(Octadecanedioic-gGlu-(AEEA)₂-gGlu)G³⁹-NH₂ (SEQ ID NO: 46).

In one aspect, the modified OXM disclosed herein comprises the following formula:

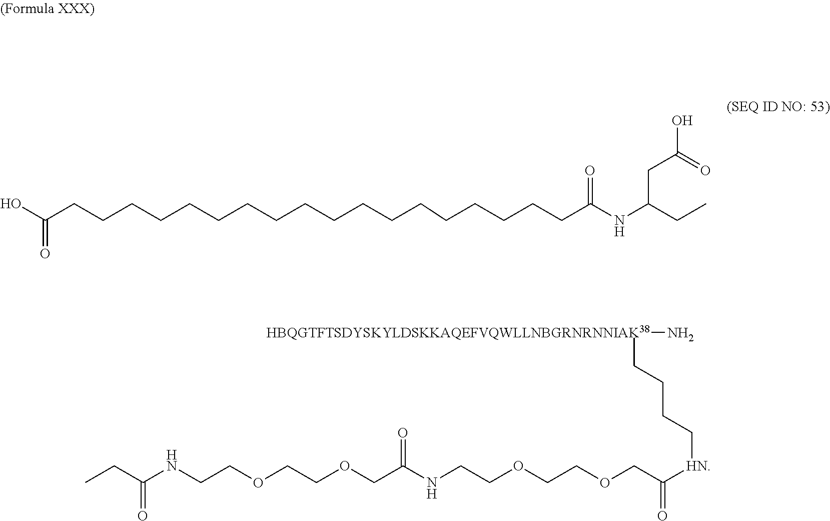

In one aspect, the modified OXM disclosed herein is H(Aib)QGTFTSDYSKYLDSKKAQEFVQWLLN(Aib)GRNRNNIAK³⁸(Eicosanedioic-gGlu-Glu-(AEEA)₂)-NH₂. (SEQ ID NO: 53)

In one aspect, the modified OXM disclosed herein comprises the following formula:

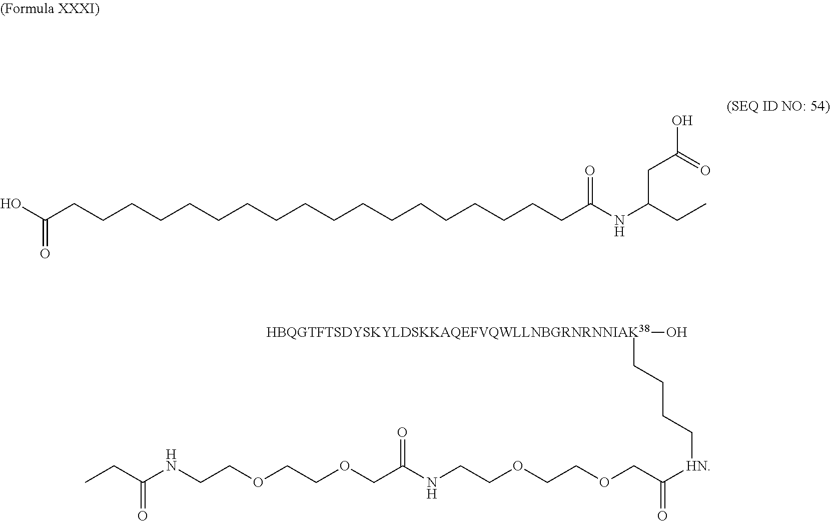

In one aspect, the modified OXM disclosed herein is H(Aib)QGTFTSDYSKYLDSKKAQEFVQWLLN(Aib)GRNRNNIAK³⁸(Eicosanedioic-gGlu-Glu-(AEEA)₂)-OH (SEQ ID NO: 54).

In one aspect, the modified OXM disclosed herein comprises the following formula:

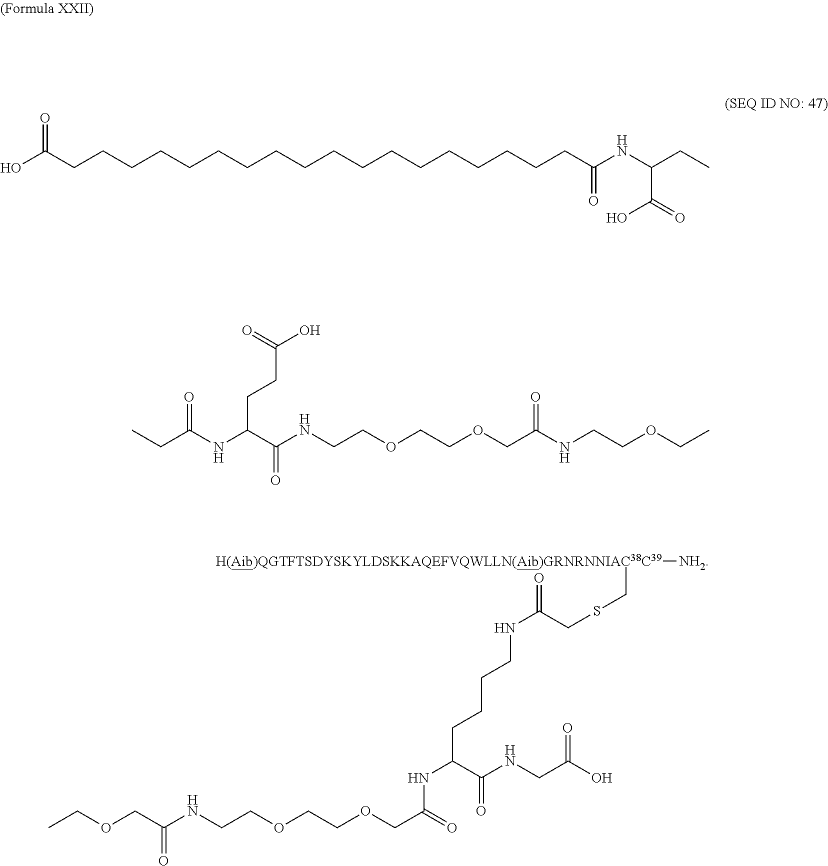

In one aspect, the modified OXM disclosed herein is Eicosanedioic-gGlu-Glu-AEEA-AEEA-AEEA-Lys(Ac-Cys³⁸mut.OXM (C³⁸C³⁹))-Gly-OH (SEQ ID NO: 47).

In one aspect, the modified OXM disclosed herein comprises the following formula:

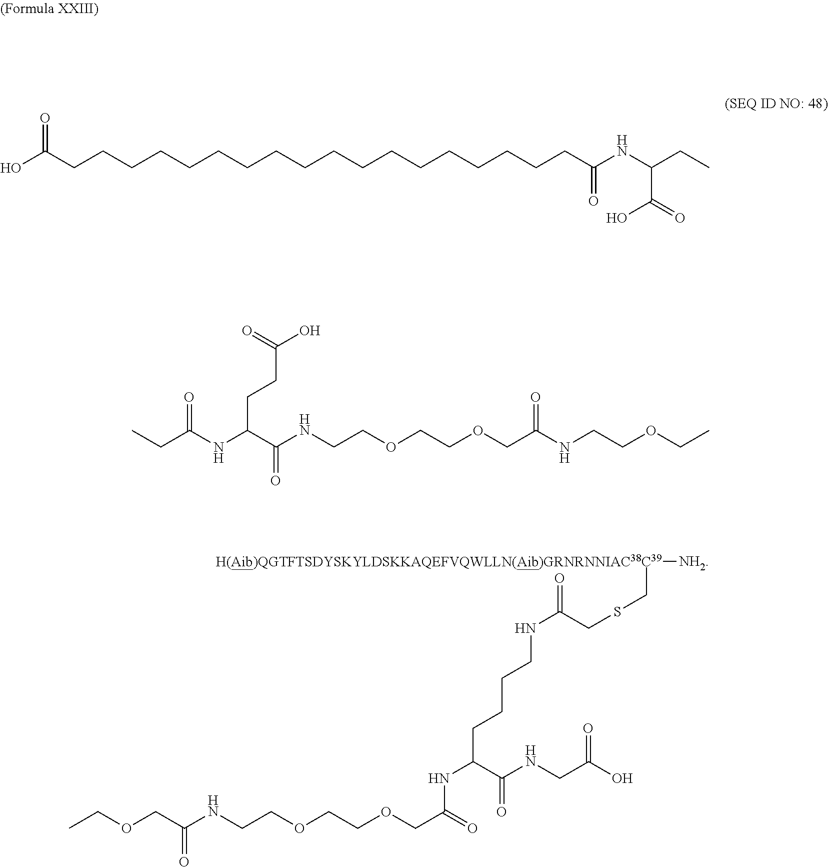

In one aspect, the modified OXM disclosed herein is Eicosanedioic-gGlu-Glu-AEEA-AEEA-AEEA-Lys(Ac-Cys³⁹mut.OXM (C³⁸C³⁹))-Gly-OH (SEQ ID NO: 48).

In one aspect, the modified OXM disclosed herein comprises the following formula:

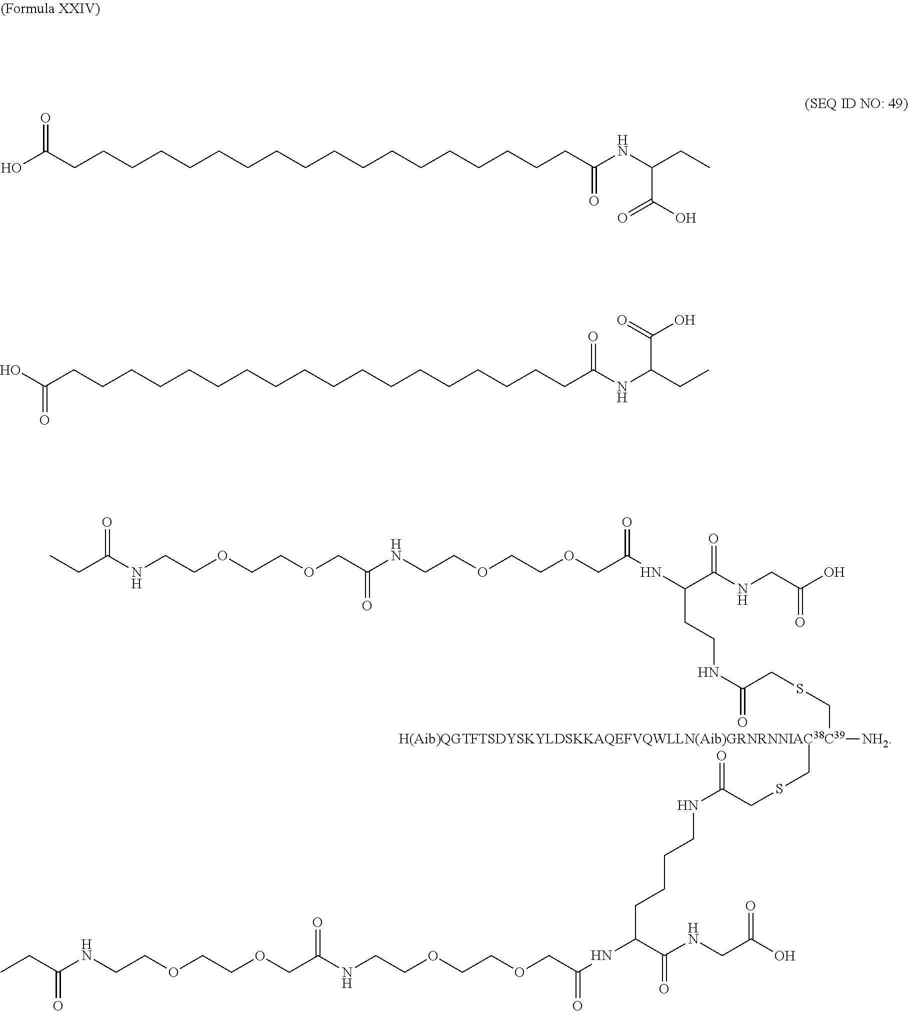

In one aspect, the modified OXM disclosed herein is H(Aib)QGTFTSDYSKYLDSKKAQEFVQWLLN(Aib)GRNRNNIAC³⁸(Eicosanedioic-gGlu-(AEEA)₂-Lys(Ac)-Gly)C³⁹(Eicosanedioic-gGlu-(AEEA)₂-Lys(Ac)-Gly)-NH₂ (SEQ ID NO: 49).

Ine one aspect, the modified OXM disclosed herein comprises the following formula:

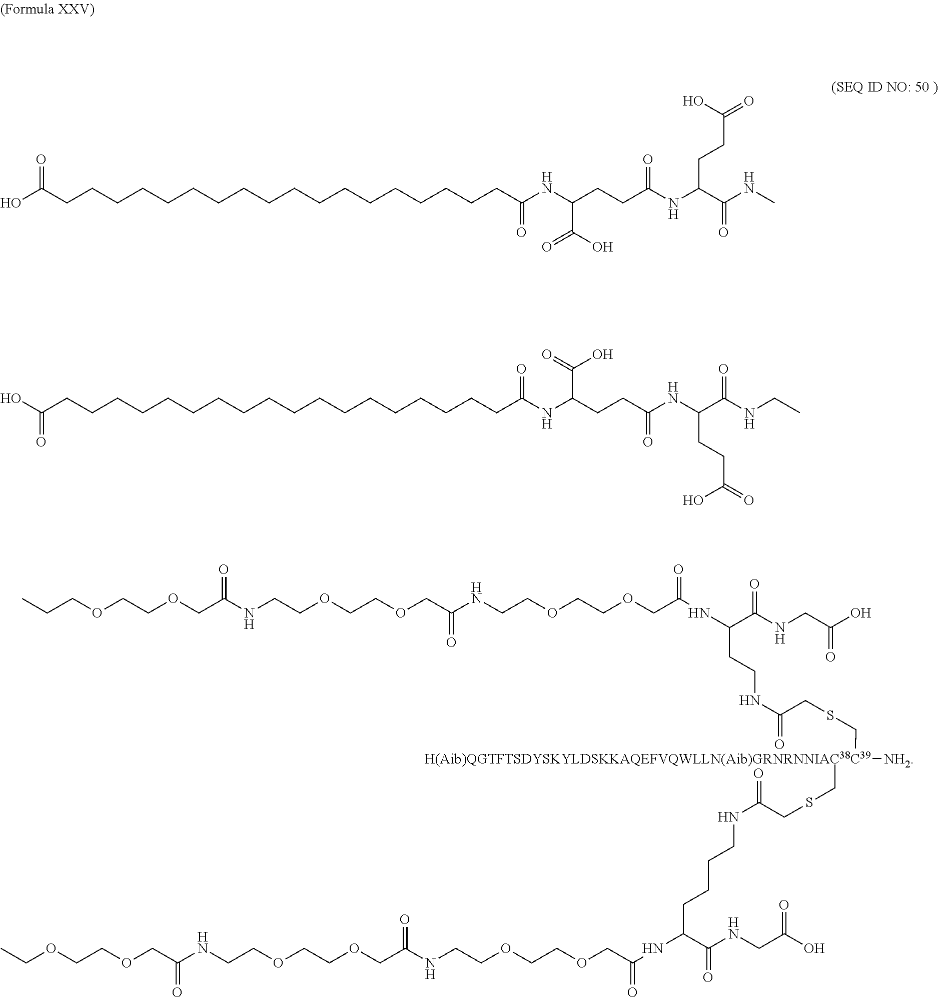

In one aspect, the modified OXM disclosed herein is H(Aib)QGTFTSDYSKYLDSKKAQEFVQWLLN(Aib)GRNRNNIAC³⁸(Eicosanedioic-gGlu-Glu-(AEEA)₃-Lys(Ac)-Gly)C³⁹(Eicosanedioic-gGlu-Glu-(AEEA)₃-Lys(Ac)-Gly)-NH₂ (SEQ ID NO: 50).

In one embodiment, the modified OXM disclosed herein is H(Aib)QGTFTSDYSKYLDSKKAQEFVQWLLN(Aib)GRNRNNIAC³⁸(Octadecanedioic-gGlu-(AEEA)₂-Lys(Ac)-Gly)C³⁹(Octadecanedioic-gGlu-(AEEA)₂-Lys(Ac)-Gly)-NH₂ (SEQ ID NO: 55)

In one embodiment, the modified OXM disclosed herein is H(Aib)QGTFTSDYSKYLDSKKAQEFVQWLLN(Aib)GRNRNNIAC³⁸(Octadecanedioic-gGlu-Glu-(AEEA)₃-Lys(Ac)-Gly)C³⁹(Octadecanedioic-gGlu-Glu-(AEEA)₃-Lys(Ac)-Gly)-NH₂ (SEQ ID NO: 56).

In one embodiment, the modified OXM disclosed herein is H(Aib)QGTFTSDYSKYLDSKKAQEFVQWLLN(Aib)GRNRNNIAC³⁸(Octadecanedioic-gGlu-(AEEA)₃-Lys(Ac)-Gly)C³⁹(Octadecanedioic-gGlu-(AEEA)₂-Lys(Ac)-Gly)-NH₂(SEQ ID NO: 57).

In one embodiment, the modified OXM disclosed herein is H(Aib)QGTFTSDYSKYLDSKKAQEFVQWLLN(Aib)GRNRNNIAC³⁸(Octadecanedioic-gGlu-(AEEA)₂-Lys(Ac)-Gly)C³⁹(Octadecanedioic-gGlu-(AEEA)₃-Lys(Ac)-Gly)-NH₂ (SEQ ID NO: 58).

In one embodiment, the modified OXM disclosed herein is H(Aib)QGTFTSDYSKYLDSKKAQEFVQWLLN(Aib)GRNRNNIAC³⁸(Eicosanedioic-gGlu-(AEEA)₃-Lys(Ac)-Gly)C³⁹(Eicosanedioic-gGlu-(AEEA)₂-Lys(Ac)-Gly)-NH₂ (SEQ ID NO: 59).

In one embodiment, the modified OXM disclosed herein is H(Aib)QGTFTSDYSKYLDSKKAQEFVQWLLN(Aib)GRNRNNIAC³⁸(Eicosanedioic-gGlu-(AEEA)₂-Lys(Ac)-Gly)C³⁹(Eicosanedioic-gGlu-(AEEA)₃-Lys(Ac)-Gly)-NH₂(SEQ ID NO: 60).

In one embodiment, the modified OXM disclosed herein is H(Aib)QGTFTSDYSKYLDSKKAQEFVQWLLN(Aib)GRNRNNIAC³⁸(Eicosanedioic-gGlu-(AEEA)₂-Lys(Ac)-Gly)C³⁹(Octadecanedioic-gGlu-(AEEA)₂-Lys(Ac)-Gly)-NH₂(SEQ ID NO: 61).

In one embodiment, the modified OXM disclosed herein is H(Aib)QGTFTSDYSKYLDSKKAQEFVQWLLN(Aib)GRNRNNIAC³⁸(Octadecanedioic-gGlu-(AEEA)₂-Lys(Ac)-Gly)C³⁹(Eicosanedioic-gGlu-(AEEA)₂-Lys(Ac)-Gly)-NH₂(SEQ ID NO: 62).

In one embodiment, the modified OXM disclosed herein is H(Aib)QGTFTSDYSKYLDSKKAQEFVQWLLN(Aib)GRNRNNIAC³⁸(Eicosanedioic-gGlu-Glu-(AEEA)₃-Lys(Ac)-Gly)C³⁹(Octadecanedioic-gGlu-Glu-(AEEA)₃-Lys(Ac)-Gly)-NH₂ (SEQ ID NO: 63).

In one embodiment, the modified OXM disclosed herein is H(Aib)QGTFTSDYSKYLDSKKAQEFVQWLLN(Aib)GRNRNNIAC³⁸(Octadecanedioic-gGlu-Glu-(AEEA)₃-Lys(Ac)-Gly)C³⁹(Eicosanedioic-gGlu-Glu-(AEEA)₃-Lys(Ac)-Gly)-NH₂ (SEQ ID NO: 64).

In one embodiment, the modified OXM disclosed herein is H(Aib)QGTFTSDYSKYLDSKKAQEFVQWLLN(Aib)GRNRNNIAC³⁸(Eicosanedioic-gGlu-(AEEA)₃-Lys(Ac)-Gly)C³⁹(Octadecanedioic-gGlu-(AEEA)₂-Lys(Ac)-Gly)-NH₂(SEQ ID NO: 65).

In one embodiment, the modified OXM disclosed herein is H(Aib)QGTFTSDYSKYLDSKKAQEFVQWLLN(Aib)GRNRNNIAC³⁸(Octadecanedioic-gGlu-(AEEA)₃-Lys(Ac)-Gly)C³⁹ (Eicosanedioic-gGlu-(AEEA)₂-Lys(Ac)-Gly)-NH₂(SEQ ID NO: 66).

In one embodiment, the modified OXM disclosed herein is H(Aib)QGTFTSDYSKYLDSKKAQEFVQWLLN(Aib)GRNRNNIAC³⁸(Eicosanedioic-gGlu-(AEEA)₂-Lys(Ac)-Gly)C³⁹(Octadecanedioic-gGlu-(AEEA)₃-Lys(Ac)-Gly)-NH₂(SEQ ID NO: 67).

In one embodiment, the modified OXM disclosed herein is H(Aib)QGTFTSDYSKYLDSKKAQEFVQWLLN(Aib)GRNRNNIAC³⁸(Octadecanedioic-gGlu-(AEEA)₂-Lys(Ac)-Gly)C³⁹(Eicosanedioic-gGlu-(AEEA)₃-Lys(Ac)-Gly)-NH₂ (SEQ ID NO: 68)

In one embodiment, the modified OXM disclosed herein is Eicosanedioic-gGlu-AEEA-AEEA-Lys(Ac-Cys³⁸mut.OXM (C³⁸C³⁹))-Gly-OH.

In one embodiment, the modified OXM disclosed herein is Eicosanedioic-gGlu-AEEA-AEEA-Lys(Ac-Cys³⁹mut.OXM (C³⁸C³⁹))-Gly-OH.

In one embodiment, the OXM in the modified OXM disclosed throughout includes any of the OXM analogs previously disclosed. In a related embodiment, the OXM analog in the modified OXM disclosed throughout include any of SEQ ID NO: 1 through SEQ ID NO: 16.

In one embodiment, disclosed is a composition comprising a mixture of two polypeptides. In a related embodiment, the mixture is combination of any of the modified OXM or modified OXM analogs described through the present application. In one embodiment, disclosed is a composition comprising a mixture of two polypeptides where the first polypeptide comprises any of the modified OXMs or modified OXM analogs described herein and the second polypeptide comprises any of the modified OXMs or modified OXM analogs described herein.

In another embodiment, the mixture has a first polypeptide which is Eicosanedioic-gGlu-AEEA-AEEA-Lys(Ac-Cys39mut.OXM (C38C39))-Gly-OH or the composition of Formula XXIV and the second polypeptide is Eicosanedioic-gGlu-AEEA-AEEA-Lys(Ac-Cys³⁸mut.OXM (C³⁸C³⁹))-Gly-OH or the composition of Formula XXIII.

In another embodiment, the mixture has a first polypeptide which is Octadecanedioic-gGlu-AEEA-AEEA-Lys(Ac-Cys³⁹mut.OXM (C³⁸C³⁹))-Gly-OH and the second polypeptide is Octadecanedioic-gGlu-AEEA-AEEA-Lys(Ac-Cys³⁸mut.OXM (C³⁸C³⁹))-Gly-OH.

In another embodiment, the first polypeptide in the mixture described herein is present in the mixture between about 30% to about 60%. In another embodiment, the first polypeptide in the mixture described herein is present in the mixture between about 20% to about 70%. In another embodiment, the first polypeptide in the mixture described herein is present in the mixture between about 10% to about 80%. In another embodiment, the first polypeptide in the mixture described herein is present in the mixture between about 5% to about 90%.

In another embodiment, the first polypeptide in the mixture described herein is present in the mixture about 50%.

In another embodiment, the first polypeptide in the mixture described herein is present in the mixture between about 30% to about 60% by weight. In another embodiment, the first polypeptide in the mixture described herein is present in the mixture between about 20% to about 70% by weight. In another embodiment, the first polypeptide in the mixture described herein is present in the mixture between about 10% to about 80% by weight. In another embodiment, the first polypeptide in the mixture described herein is present in the mixture between about 5% to about 90% by weight.

In another embodiment, the first polypeptide in the mixture described herein is present in the mixture about 50% by weight.

In one embodiment, the composition comprising a mixture of any of the modified OXM or modified OXM analogs described through the present application is in the form of a pharmaceutically acceptable salt.

In one embodiment, the modified OXM or modified OXM analogs disclosed throughout the application includes pharmaceutically acceptable salts thereof.

### Pharmaceutical Compositions

Furthermore, pharmaceutical compositions of GLP-1 peptides and solid pharmaceutical compositions of GLP-1 are described in International Patent Application No. WO 2013/139694 A1. See also U.S. Pat. Nos. 8,129,343; 8,536,122; 9,278,123; 10,086,047; 10,278,923; 10,933,120; 10,960,052; 11,382,957; 11,759,501; 11,759,502; and 11,759,503; which are incorporated herein by reference in its entirety.

In one embodiment, disclosed is a pharmaceutical composition comprising the compound according to any one of the modified OXM or modified OXM analogs, and a pharmaceutically acceptable excipient.

An embodiment provides a pharmaceutical composition comprising a compound according to any one of the embodiments above, and a pharmaceutically acceptable excipient.

In some embodiments the composition comprises at least one pharmaceutically acceptable excipient. The term “excipient” as used herein broadly refers to any component other than the active therapeutic ingredient(s). The excipient may be an inert substance, an inactive substance, and/or a not medicinally active substance. The excipient may serve various purposes, e.g. as a carrier, vehicle, filler, binder, lubricant, glidant, disintegrant, flow control agents, crystallization retarders, solubilizers, stabilizer, colouring agent, flavouring agent, surfactant, emulsifier and/or to improve administration, and/or absorption of the active substance. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005). In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization retarders such as Povidone, etc.; solubilizers such as Pluronic, Povidone, etc.; colouring agents, including dyes and pigments such as Iron Oxide Red or Yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

In one embodiment is a pharmaceutical formulation comprising a modified OXM or modified OXM analog disclosed herein which is present in a concentration from 0.1 mg/ml to 25 mg/ml, and wherein said formulation has a pH from 3.0 to 9.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In one embodiment, the dose of the modified OXM or modified OXM analog disclosed herein, or pharmaceutically acceptable salt thereof, can range, for example, from 50 nmoles/kg to 3000 nmoles/kg of patient body weight, 50 nmoles/kg to 2000 nmoles/kg, 50 nmoles/kg to 1000 nmoles/kg, 50 nmoles/kg to 900 nmoles/kg, 50 nmoles/kg to 800 nmoles/kg, 50 nmoles/kg to 700 nmoles/kg, 50 nmoles/kg to 600 nmoles/kg, 50 nmoles/kg to 500 nmoles/kg, 50 nmoles/kg to 400 nmoles/kg, 50 nmoles/kg to 300 nmoles/kg, 50 nmoles/kg to 200 nmoles/kg, 50 nmoles/kg to 100 nmoles/kg, 100 nmoles/kg to 300 nmoles/kg, 100 nmoles/kg to 500 nmoles/kg, 100 nmoles/kg to 1000 nmoles/kg, 100 nmoles/kg to 2000 nmoles/kg of patient body weight. In other embodiments, the dose may be 1 nmoles/kg, 5 nmoles/kg, 10 nmoles/kg, 20 nmoles kg, 25 nmoles/kg, 30 nmoles/kg, 40 nmoles/kg, 50 nmoles/kg, 60 nmoles/kg, 70 nmoles/kg, 80 nmoles/kg, 90 nmoles/kg, 100 nmoles/kg, 150 nmoles/kg, 200 nmoles/kg, 250 nmoles/kg, 300 nmoles/kg, 350 nmoles/kg, 400 nmoles/kg, 450 nmoles/kg, 500 nmoles/kg, 600 nmoles/kg, 700 nmoles/kg, 800 nmoles/kg, 900 nmoles/kg, 1000 nmoles/kg, 2000 nmoles/kg, 2500 nmoles/kg or 3000 nmoles/kg of body weight of the patient. In yet other embodiments, the dose may be 0.1 nmoles/kg, 0.2 nmoles/kg, 0.3 nmoles/kg, 0.4 nmoles kg, or 0.5 nmoles/kg, 0.1 nmoles/kg to 1000 nmoles/kg, 0.1 nmoles/kg to 900 nmoles/kg, 0.1 nmoles/kg to 850 nmoles/kg, 0.1 nmoles/kg to 800 nmoles/kg, 0.1 nmoles/kg to 700 nmoles/kg, 0.1 nmoles/kg to 600 nmoles/kg, 0.1 nmoles/kg to 500 nmoles/kg, 0.1 nmoles/kg to 400 nmoles/kg, 0.1 nmoles/kg to 300 nmoles/kg, 0.1 nmoles/kg to 200 nmoles/kg, 0.1 nmoles/kg to 100 nmoles/kg, 0.1 nmoles/kg to 50 nmoles/kg, 0.1 nmoles/kg to 10 nmoles/kg, or 0.1 nmoles/kg to 1 nmoles/kg of body weight of the patient. In other embodiments, the dose may be 0.3 nmoles/kg to 1000 nmoles/kg, 0.3 nmoles/kg to 900 nmoles/kg, 0.3 nmoles/kg to 850 nmoles/kg, 0.3 nmoles/kg to 800 nmoles/kg, 0.3 nmoles/kg to 700 nmoles/kg, 0.3 nmoles/kg to 600 nmoles/kg, 0.3 nmoles/kg to 500 nmoles/kg, 0.3 nmoles/kg to 400 nmoles/kg, 0.3 nmoles/kg to 300 nmoles/kg, 0.3 nmoles/kg to 200 nmoles/kg, 0.3 nmoles/kg to 100 nmoles/kg, 0.3 nmoles/kg to 50 nmoles/kg, 0.3 nmoles/kg to 10 nmoles/kg, or 0.3 nmoles/kg to 1 nmoles/kg of body weight of the patient. In these embodiments, “kg” is kilograms of body weight of the patient.

In one embodiment, the dose of the modified OXM or modified OXM analog disclosed herein, or pharmaceutically acceptable salt thereof, can range, for example, from 10 nmoles/kg to 10000 nmoles/kg, from 10 nmoles/kg to 5000 nmoles/kg, from 10 nmoles/kg to 3000 nmoles/kg, 10 nmoles/kg to 2500 nmoles/kg, 10 nmoles/kg to 2000 nmoles/kg, 10 nmoles/kg to 1000 nmoles/kg, 10 nmoles/kg to 900 nmoles/kg, 10 nmoles/kg to 800 nmoles/kg, 10 nmoles/kg to 700 nmoles/kg, 10 nmoles/kg to 600 nmoles/kg, 10 nmoles/kg to 500 nmoles/kg, 10 nmoles/kg to 400 nmoles/kg, 10 nmoles/kg to 300 nmoles/kg, 10 nmoles/kg to 200 nmoles/kg, 10 nmoles/kg to 150 nmoles/kg, 10 nmoles/kg to 100 nmoles/kg, 10 nmoles/kg to 90 nmoles/kg, 10 nmoles/kg to 80 nmoles/kg, 10 nmoles/kg to 70 nmoles/kg, 10 nmoles/kg to 60 nmoles/kg, 10 nmoles/kg to 50 nmoles/kg, 10 nmoles/kg to 40 nmoles/kg, 10 nmoles/kg to 30 nmoles/kg, 10 nmoles/kg to 20 nmoles/kg, 200 nmoles/kg to 900 nmoles/kg, 200 nmoles/kg to 800 nmoles/kg, 200 nmoles/kg to 700 nmoles/kg, 200 nmoles/kg to 600 nmoles/kg, 200 nmoles/kg to 500 nmoles/kg, 250 nmoles/kg to 600 nmoles/kg, 300 nmoles/kg to 600 nmoles/kg, 300 nmoles/kg to 500 nmoles/kg, or 400 nmoles/kg to 600 nmoles/kg, of body weight of the patient. In various other embodiments, the dose of the compound, or the pharmaceutically acceptable salt thereof, may range from, for example, 1 nmoles/kg to 10000 nmoles/kg, from 1 nmoles/kg to 5000 nmoles/kg, from 1 nmoles/kg to 3000 nmoles/kg, 1 nmoles/kg to 2500 nmoles/kg, 1 nmoles/kg to 2000 nmoles/kg, 1 nmoles/kg to 1000 nmoles/kg, 1 nmoles/kg to 900 nmoles/kg, 1 nmoles/kg to 800 nmoles/kg, 1 nmoles/kg to 700 nmoles/kg, 1 nmoles/kg to 600 nmoles/kg, 1 nmoles/kg to 500 nmoles/kg, 1 nmoles/kg to 400 nmoles/kg, 1 nmoles/kg to 300 nmoles/kg, 1 nmoles/kg to 200 nmoles/kg, 1 nmoles/kg to 150 nmoles/kg, 1 nmoles/kg to 100 nmoles/kg, 1 nmoles/kg to 90 nmoles/kg, 1 nmoles/kg to 80 nmoles/kg, 1 nmoles/kg to 70 nmoles/kg, 1 nmoles/kg to 60 nmoles/kg, 1 nmoles/kg to 50 nmoles/kg, 1 nmoles/kg to 40 nmoles/kg, 1 nmoles/kg to 30 nmoles/kg, 1 nmoles/kg to 20 nmoles/kg, 1 nmoles/kg to 10 nmoles/kg, or 1 nmoles/kg to 5 nmoles/kg. In these embodiments, “kg” is kilograms of body weight of the patient. In one aspect, a single dose or multiple doses of the compound, or pharmaceutically acceptable salt thereof, may be administered to the patient.

In one embodiment, the dose of the modified OXM or modified OXM analog disclosed herein, or pharmaceutically acceptable salt thereof, is 1 nmoles/kg, 2 nmoles/kg, 3 nmoles/kg, 5 nmoles/kg, 6 nmoles/kg, nmoles/kg, 7 nmoles/kg, 8 nmoles/kg, 9 nmoles/kg, 10 nmoles/kg, 11 nmoles/kg, 12 nmoles/kg, 13 nmoles/kg, 14 nmoles/kg, 15 nmoles/kg, 16 nmoles/kg, 17 nmoles/kg, 18 nmoles/kg, 19 nmoles/kg, 20 nmoles/kg, 21 nmoles/kg, 22 nmoles/kg, 23 nmoles/kg, 24 nmoles/kg, 25 nmoles/kg, 26 nmoles/kg, 27 nmoles/kg, 28 nmoles/kg, 29 nmoles/kg, or 30 nmoles/kg of body weight of the patient. In another embodiment, the dose of the modified OXM or modified OXM analog disclosed herein, or pharmaceutically acceptable salt thereof, is 2 nmoles/kg, 5 nmoles/kg, 7 nmoles/kg, 10 nmoles/kg, 15 nmoles/kg, 20 nmoles/kg, 25 nmoles/kg, or 30 nmoles/kg of body weight of the patient.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In another embodiment is a pharmaceutical formulation comprising an aqueous solution of a modified OXM or modified OXM analog disclosed herein and a buffer, wherein said modified OXM or modified OXM analog is present in a concentration from 0.1 mg/ml or above, and wherein said formulation has a pH from about 3.0 to about 9.0.

In another embodiment the pH of the formulation is from about 7.0 to about 9.5. In another embodiment the pH of the formulation is from about 3.0 to about 7.0. In another embodiment the pH of the formulation is from about 5.0 to about 7.5. In another embodiment the pH of the formulation is from about 7.5 to about 9.0. In another embodiment the pH of the formulation is from about 7.5 to about 8.5. In another embodiment the pH of the formulation is from about 6.0 to about 7.5. In another embodiment the pH of the formulation is from about 6.0 to about 7.0. In another embodiment the pharmaceutical formulation is from 8.0 to 8.5.

In some embodiments the pharmaceutical composition comprises a phosphate buffer, such as a sodium phosphate buffer, e.g. disodium phosphate. In some embodiments the pharmaceutical composition comprises an isotonic agent, such as propylene glycol. In some embodiments the pharmaceutical composition comprises a preservative, such as phenol.

The pharmaceutical composition may be in the form of a solution or a suspension. In some embodiments the pharmaceutical composition is aqueous composition, such as an aqueous solution or an aqueous suspension. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water. An aqueous composition may comprise at least 50% w/w water, or at least 60%, 70%, 80%, or even at least 90% w/w of water. In some embodiments the pharmaceutical composition has a pH in the range of 7.0-9.0, such as 7.0-8.5.

In some embodiments the modified OXM or modified OXM analog is administered in the form of a pharmaceutical composition comprising about 0.1-20 mg/ml modified OXM or modified OXM analog, about 2-15 mM phosphate buffer, about 2-25 mg/ml propylene glycol, about 1-18 mg/ml phenol, and has a pH in the range of 7.0-9.0. In some embodiments the modified OXM or modified OXM analog is administered in the form of a pharmaceutical composition comprising about 1.34 mg/ml modified OXM or modified OXM analog, about 1.42 mg/ml disodium phosphate dihydrate, about 14.0 mg/ml propylene glycol, about 5.5 mg/ml phenol, and has pH of about 7.4. In some embodiments the modified OXM or modified OXM analog is administered in the form of a pharmaceutical composition comprising 1.34 mg/ml modified OXM or modified OXM analog, 1.42 mg/ml disodium phosphate dihydrate, 14.0 mg/ml propylene glycol, 5.5 mg/ml phenol, and has pH of 7.4.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative.

In a further embodiment, the formulation further comprises a pharmaceutically acceptable preservative. In a further embodiment the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In an embodiment the preservative is phenol or m-cresol. In a further embodiment the preservative is present in a concentration from 0.1 mg/ml to 20 mg/ml. In a further embodiment the preservative is present in a concentration from 0.1 mg/ml to 5 mg/ml. In a further embodiment the preservative is present in a concentration from 5 mg/ml to 10 mg/ml. In a further embodiment the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995.

In a further embodiment the formulation further comprises an isotonic agent. In a further embodiment the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. In an embodiment the isotoncity agent is propyleneglycol. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment the isotonic agent is present in a concentration from 1 mg/ml to 50 mg/ml. In a further embodiment the isotonic agent is present in a concentration from 1 mg/ml to 7 mg/ml. In an embodiment the isotonic agent is present in a concentration from 5 mg/ml to 7 mg/ml. In a further embodiment the isotonic agent is present in a concentration from 8 mg/ml to 24 mg/ml. In a further embodiment the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19th edition, 1995.

In a further embodiment, the formulation further comprises a chelating agent. In a further embodiment the chelating agent is selected from salts of ethylenediaminetetraacetic acid (EDTA), citric acid, and aspartic acid, and mixtures thereof. In a further embodiment the chelating agent is present in a concentration from 0.1 mg/ml to 5 mg/ml. In a further embodiment the chelating agent is present in a concentration from 0.1 mg/ml to 2 mg/ml. In a further embodiment the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19th edition, 1995.

In a further embodiment the formulation further comprises a stabilizer. The use of a stabilizer in pharmaceutical compositions is well-known to the skilled person. For convenience reference is made to Remington: The Science and Practice of Pharmacy, 19th edition, 1995.

In one embodiment, the pharmaceutical compositions disclosed herein are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment, methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment, the formulation further comprises a stabilizer selected from the group of high molecular weight polymers or low molecular compounds. In a further embodiment the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment, the formulation further comprises a surfactant. In another embodiment the pharmaceutical composition comprises two different surfactants. The term “Surfactant” as used herein refers to any molecules or ions that are comprised of a water-soluble (hydrophilic) part, the head, and a fat-soluble (lipophilic) segment. Surfactants accumulate preferably at interfaces, which the hydrophilic part is orientated towards the water (hydrophilic phase) and the lipophilic part towards the oil- or hydrophobic phase (i.e. glass, air, oil etc.). The concentration at which surfactants begin to form micelles is known as the critical micelle concentration or CMC. Furthermore, surfactants lower the surface tension of a liquid. Surfactants are also known as amphipathic compounds. The term “Detergent” is a synonym used for surfactants in general.

Anionic surfactants may be selected from the group of: Chenodeoxycholic acid, Chenodeoxycholic acid sodium salt, Cholic acid, Dehydrocholic acid, Deoxycholic acid, Deoxycholic acid methyl ester, Digitonin, Digitoxigenin, N,N-Dimethyldodecylamine N-oxide, Docusate sodium, Glycochenodeoxycholic acid sodium, Glycocholic acid hydrate, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine sodium salt, N-Lauroylsarcosine, N-Lauroylsarcosine, Lithium dodecyl sulfate, Lugol, 1-Octanesulfonic acid sodium salt, 1-Octanesulfonic acid sodium salt, Sodium 1-butanesulfonate, Sodium 1-decanesulfonate, Sodium 1-dodecanesulfonate, Sodium 1-heptanesulfonate, Sodium 1-heptanesulfonate, Sodium 1-nonanesulfonate, Sodium 1-propanesulfonate monohydrate, Sodium 2-bromoethanesulfonate, Sodium cholate hydrate, ox or sheep bile, Sodium cholate hydrate, Sodium choleate, Sodium deoxycholate, Sodium dodecyl sulfate, Sodium dodecyl sulfate, Sodium hexanesulfonate, Sodium octyl sulfate, Sodium pentanesulfonate, Sodium taurocholate, Taurochenodeoxycholic acid sodium salt, Taurodeoxycholic acid sodium salt monohydrate, Taurolithocholic acid 3-sulfate disodium salt, Tauroursodeoxycholic acid sodium salt, Trizma® dodecyl sulfate, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), Dodecylphosphocholine (FOS-Choline-12), Decylphosphocholine (FOS-Choline-10), Nonylphosphocholine (FOS-Choline-9), dipalmitoyl phosphatidic acid, sodium caprylate, and/or Ursodeoxycholic acid.

Cationic surfactants may be selected from the group of: Alkyltrimethylammonium bromide, Benzalkonium chloride, Benzalkonium chloride, Benzyldimethylhexadecylammonium chloride, Benzyldimethyltetradecylammonium chloride, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Hexadecyltrimethylammonium bromide, Hexadecyltrimethylammonium bromide, Polyoxyethylene(10)-N-tallow-1,3-diaminopropane, Thonzonium bromide, and/or Trimethyl(tetradecyl)ammonium bromide.

Nonionic surfactants may be selected from the group of: BigCHAP, Bis(polyethylene glycol bis[imidazoyl carbonyl]), block copolymers as polyethyleneoxide/polypropyleneoxide block copolymers such as poloxamers, poloxamer 188 and poloxamer 407, Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Dodecanoyl-N-methylglucamide, alkyl-polyglucosides, ethoxylated castor oil, Heptaethylene glycol monodecyl ether, Heptaethylene glycol monododecyl ether, Heptaethylene glycol monotetradecyl ether, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-beta-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-β-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span®60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tetradecyl-β-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165 solution, Triton® X-305 solution, Triton® X-405, Triton® X-45, Triton® X-705-70, TWEEN® 20, TWEEN® 40, TWEEN® 60, TWEEN® 6, TWEEN® 65, TWEEN® 80, TWEEN® 81, TWEEN® 85, Tyloxapol, sphingophospholipids (sphingomyelin), and sphingoglycolipids (ceramides, gangliosides), phospholipids, and/or n-Undecyl β-D-glucopyranoside.

Zwitterionic surfactants may be selected from the group of: CHAPS, CHAPSO, 3-(Decyldimethylammonio)propanesulfonate inner salt, 3-(Dodecyldimethylammonio)-propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)-propanesulfonate, 3-(N,N-Dimethyloctylammonio)propanesulfonate inner salt, 3-(N,N-Dimethylpalmitylammonio)propanesulfonate, N-alkyl-N,N-dimethylammonio-1-propanesulfonates, 3-cholamido-1-propyldimethylammonio-1-propanesulfonate, Dodecylphosphocholine, myristoyl lysophosphatidylcholine, Zwittergent 3-12 (N-dodecyl-N,N-dimethyl-3-ammonio-1-propanesulfonate), Zwittergent 3-10 (3-(Decyldimethylammonio)-propanesulfonate inner salt), Zwittergent 3-08 (3-(Octyldimethylammonio)pro-panesulfonate), glycerophospholipids (lecithins, kephalins, phosphatidyl serine), glyceroglycolipids (galactopyranoside), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, lysophosphatidylserine and lysophosphatidylthreonine, acylcarnitines and derivatives, Nbeta-acylated derivatives of lysine, arginine or histidine, or side-chain acylated derivatives of lysine or arginine, Nbeta-acylated derivatives of dipeptides comprising any combination of lysine, arginine or histidine and a neutral or acidic amino acid, Nbeta-acylated derivative of a tripeptide comprising any combination of a neutral amino acid and two charged amino acids, or the surfactant may be selected from the group of imidazoline derivatives, long-chain fatty acids and salts thereof C6-C12 (eg. oleic acid and caprylic acid), N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), or mixtures thereof.

In a further embodiment, the formulation further comprises protease inhibitors such as EDTA (ethylenediamine tetraacetic acid) and benzamidineHCl, but other commercially available protease inhibitors may also be used. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present disclosure.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

In some embodiments the composition comprises 0.1-10% (w/w), such as 0.2-4% (w/w) or 0.5-3% (w/w), of binder. In some embodiments the composition comprises 1% (w/w) or 2% (w/w) of binder. The composition may comprise a binder, such as povidone; starches; celluloses and derivatives thereof, such as microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be selected from the group consisting of dry binders and/or wet granulation binders. Suitable dry binders are, e.g., cellulose powder and microcrystalline cellulose, such as Avicel PH 102 and Avicel PH 200. In some embodiments the composition comprises avicel, such as avicel PH 102. Suitable binders for wet granulation or dry granulation are corn starch, polyvinyl pyrrolidone (povidone), vinylpyrrolidone-vinylacetate copolymer (copovidone) and cellulose derivatives like hydroxymethylcellulose, hydroxyethylcellulose, hydroxypropylcellulose and hydroxyl-propylmethylcellulose. In some embodiments the composition comprises povidone.

In one embodiment, the solid composition for oral administration is a modified OXM or modified OXM analog, a salt of N-(8-(2-hydroxybenzoyl)amino)caprylic acid, a lubricant, a binder, and a filler.

In one embodiment, the solid composition for oral administration is a modified OXM or modified OXM analog, a salt of N-(8-(2-hydroxybenzoyl)amino)caprylic acid, a lubricant, a binder, and a filler, wherein the composition comprises at least 60% (w/w) of said salt of N-(8-(2-hydroxybenzoyl)amino)caprylic acid, wherein the composition comprises 1-3.5% (w/w) of the magnesium stearate, wherein the composition comprises 0.5-3% (w/w) of the povidone, and wherein the composition comprises 5-25% (w/w) of the microcrystalline cellulose.

In one embodiment, the solid composition for oral administration is a modified OXM or modified OXM analog, a salt of N-(8-(2-hydroxybenzoyl)amino)caprylic acid, a lubricant, a binder, and a filler, wherein the composition comprises at least 60% (w/w) of said salt of N-(8-(2-hydroxybenzoyl)amino)caprylic acid.

In one embodiment, the solid composition for oral administration is a modified OXM or modified OXM analog, a salt of N-(8-(2-hydroxybenzoyl)amino)caprylic acid, a lubricant, a binder, and a filler, wherein the composition comprises 1-3.5% (w/w) of the magnesium stearate.

In one embodiment, the solid composition for oral administration is a modified OXM or modified OXM analog, a salt of N-(8-(2-hydroxybenzoyl)amino)caprylic acid, a lubricant, a binder, and a filler, wherein the composition comprises 0.5-3% (w/w) of the povidone.

In one embodiment, the solid composition for oral administration is a modified OXM or modified OXM analog, a salt of N-(8-(2-hydroxybenzoyl)amino)caprylic acid, a lubricant, a binder, and a filler, wherein the composition comprises 5-25% (w/w) of the microcrystalline cellulose.

Pharmaceutical Composition with Two Granules

In some embodiments the present disclosure relates to a pharmaceutical composition comprising a first type and a second type of granules, wherein said first type of granules comprises a salt of N-(8-(2-hydroxybenzoyl)amino)caprylic acid and said second type of granules comprises a modified OXM or modified OXM analog. In some embodiments the first type of granules further comprises a lubricant, such as magnesium stearate. In some embodiments the first type of granules further comprises a filler, such as microcrystalline cellulose. Accordingly, the first type of granules may further comprise a lubricant and optionally a filler. In some embodiments the second type of granules further comprises a filler, such as microcrystalline cellulose. In some embodiments the second type of granules further comprises a binder, such as povidone. Accordingly, the second type of granules may further comprise a filler and optionally a binder. In some embodiments the composition further comprises an extragranular lubricant, such as magnesium stearate.

In some embodiments N-(8-(2-hydroxybenzoyl)amino)caprylic acid is referred to as “NAC”.

In some embodiments the first type of granules comprising a salt of N-(8-(2-hydroxybenzoyl)amino)caprylic acid does not contain a modified OXM or modified OXM analog. In some embodiments the second type of granules comprising a modified OXM or modified OXM analog does not contain a salt of N-(8-(2-hydroxybenzoyl)amino)caprylic acid. In some embodiments the pharmaceutical composition comprises a first type and a second type of granules, wherein said first type of granules comprises a salt of N-(8-(2-hydroxybenzoyl)amino)caprylic acid and no modified OXM or modified OXM analog, and wherein said second type of granules comprises a modified OXM or modified OXM analog and no salt of N-(8-(2-hydroxybenzoyl)amino)caprylic acid.

In some embodiments the term “granule” refers to particles gathered into larger particles.

In some embodiments the present disclosure relates to a pharmaceutical composition comprising a salt of N-(8-(2-hydroxybenzoyl)amino)caprylic acid and a modified OXM or modified OXM analog, wherein the release of said salt of N-(8-(2-hydroxybenzoyl)amino)caprylic acid is simultaneous with or faster than the release of said modified OXM or modified OXM analog. In some embodiments the present disclosure relates to a pharmaceutical composition comprising a salt of N-(8-(2-hydroxybenzoyl)amino)caprylic acid and a modified OXM or modified OXM analog, wherein the release of said salt of N-(8-(2-hydroxybenzoyl)amino)caprylic acid is simultaneous with or faster than the release of said modified OXM or modified OXM analog.

In some embodiments the composition comprises granules which have been manufactured by dry granulation. In some embodiments the composition comprises granules which have been manufactured by roller compaction. In some embodiments the moldings from the roller compactions process are comminuted into granules. As used herein the term “composition” refers to a pharmaceutical composition.

In some embodiments the composition is in the form of a solid dosage form. In some embodiments the composition is in the form of a tablet. In some embodiments the composition is in the form of a capsule. In some embodiments the composition is in the form of a sachet.

The composition may be administered in several dosage forms, for example as a tablet; a coated tablet; a chewing gum; a capsule such as hard or soft gelatine capsules or a powder. The composition may further be compounded in a drug carrier or drug delivery system, e.g. in order to improve stability and/or solubility or further improve bioavailability. The composition may be a freeze-dried or spray-dried composition. A composition may also be used in the formulation of site specific, controlled, sustained, protracted, prolonged, delayed, pulsatile, retarded, and/or slow release drug delivery systems.

In some embodiments the composition or granule comprises at least one pharmaceutically acceptable excipient. The term “excipient” as used herein broadly refers to any component other than the active therapeutic ingredient(s). The excipient may be an inert substance, which is inert in the sense that it substantially does not have any therapeutic and/or prophylactic effect per se. The excipient may serve various purposes, e.g. as a delivery agent, absorption enhancer, vehicle, filler (also known as diluents), binder, lubricant, glidant, disintegrant, crystallization retarders, acidifying agent, alkalizing agent, preservative, antioxidant, buffering agent, chelating agent, complexing agents, surfactant agent, emulsifying and/or solubilizing agents, sweetening agents, wetting agents stabilizing agent, colouring agent, flavouring agent, and/or to improve administration, and/or absorption of the active substance. A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In some embodiments the composition or granule comprises a filler, such as lactose (e.g. spray-dried lactose, α-lactose, β-lactose, Tabletose®, various grades of Pharmatose®, Microtose® or Fast-FloC®), microcrystalline cellulose (various grades of Avicel®, Elcema®, Vivacel®, Ming Tai® or Solka-Floc®), other cellulose derivatives, sucrose, sorbitol, mannitol, dextrins, dextrans, maltodextrins, dextrose, fructose, kaolin, mannitol, sorbitol, sucrose, sugar, starches or modified starches (including potato starch, maize starch and rice starch), calcium phosphate (e.g. basic calcium phosphate, calcium hydrogen phosphate, dicalcium phosphate hydrate), calcium sulphate, calcium carbonate, or sodium alginate. In some embodiments the filler is microcrystalline cellulose, such as Avicel PH 101.

In some embodiments the composition or granule comprises a binder, such as lactose (e.g. spray-dried lactose, α-lactose, β-lactose, Tabletose®, various grades of Pharmatose®, Microtose® or Fast-FloC®), microcrystalline cellulose (various grades of Avicel®, Elcema®, Vivacel®, Ming Tai® or Solka-Floc®), hydroxypropylcellulose, L-hydroxypropylcellulose (low-substituted), hypromellose (HPMC) (e.g. Methocel E, F and K, Metolose SH of Shin-Etsu, Ltd, such as, e.g., the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, ethylcellulose, sodium carboxymethylcellulose, other cellulose derivatives, sucrose, dextrins, maltodextrins, starches or modified starches (including potato starch, maize starch and rice starch), calcium lactate, calcium carbonate, acacia, sodium alginate, agar, carrageenan, gelatin, guar gum, pectin, PEG, or povidone. In some embodiments the binder is povidone, such as povidone K 90.

In some embodiments the composition or granule comprises a disintegrant, such as alginic acid, alginates, microcrystalline cellulose, hydroxypropyl cellulose, other cellulose derivatives, croscarmellose sodium, crospovidone, polacrillin potassium, sodium starch glycolate, starch, pregelatinized starch, or carboxymethyl starch (e.g. Primogel® and Explotab®).

In some embodiments the composition or granule comprises a lubricant, such as stearic acid, magnesium stearate, calcium stearate or other metallic stearate, talc, waxes, glycerides, light mineral oil, glyceryl behenate, hydrogenated vegetable oils, sodium stearyl fumarate, polyethylene glycols, alkyl sulfates, or sodium benzoate. In some embodiments the composition or granule comprises a lubricant, such as magnesium silicate, talc, or colloidal silica. In some embodiments the lubricant is magnesium stearate. In some embodiments the composition comprises a lubricant and/or a glidant, such as talc, magnesium stearate, calcium stearate, zinc stearate, glyceryl behenate, polyethylene oxide polymers, sodium lauryl sulfate, magnesium lauryl sulfate, sodium oleate, sodium stearyl fumarate, stearic acid, hydrogenated vegetable oils, silicon dioxide and/or polyethylene glycol. In some embodiments the composition comprises magnesium stearate.

In some embodiments the composition comprises a disintegrant, such as sodium starch glycolate, polacrilin potassium, sodium starch glycolate, crospovidon, croscarmellose, sodium carboxymethylcellulose or dried corn starch.

The composition may comprise one or more surfactants, for example a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants.

Still further, the composition may be formulated as is known in the art of oral formulations of insulinotropic compounds, e.g. using any one or more of the formulations described in WO 2008/145728.

In some embodiments the composition or granule comprises one or more excipients selected from crystallization retarders, such as Povidone, etc.; solubilizing agents (also known as surfactants), such as anionic surfactants (e.g. Pluronic or Povidone), cationic surfactants, nonionic surfactants, and/or zwitterionic surfactants; colouring agents, including dyes and pigments, such as Iron Oxide Red or Yellow, titanium dioxide, and/or talc; and/or pH control agents, such as citric acid, tartaric acid, fumaric acid, sodium citrate, dibasic calcium phosphate, and/or dibasic sodium phosphate.

In some embodiments the composition comprises at least 60% (w/w) delivery agent, less than 10% (w/w) binder, 5-40% (w/w) filler, and less than 10% (w/w) lubricant or glidant.

In some embodiments the composition comprises at least 60% (w/w), such as 65-75% (w/w), 60-80% (w/w), or 50-90% (w/w), delivery agent. In some embodiments the composition comprises at least 70% (w/w), such as 70-80% (w/w), delivery agent.

In some embodiments the composition comprises 0.1-10% (w/w), such as 0.2-4% (w/w) or 0.5-3% (w/w), binder. In some embodiments the composition comprises 1.5-2.5% (w/w), such as 1.7-2.3% (w/w), 1.8-2.2% (w/w), or 1.9-2.1% (w/w), binder. In some embodiments the composition comprises 1% (w/w) or 2% (w/w) binder.

In some embodiments the composition comprises 5-40% (w/w), such as 10-30% (w/w) or 5-25% (w/w), filler. In some embodiments the composition comprises 10-25% (w/w), such as 17-23% (w/w), 18-22% (w/w), or 19-21% (w/w), filler. In some embodiments the composition comprises 10.9% (w/w) or 18% (w/w) filler, or comprises 19.5% (w/w) or 20.5 (w/w) filler.

The filler may be selected from lactose, mannitol, erythritol, sucrose, sorbitol, calcium phosphate, such as calciumhydrogen phosphate, microcrystalline cellulose, powdered cellulose, confectioner's sugar, compressible sugar, dextrates, dextrin and dextrose. In some embodiments the composition comprises microcrystalline cellulose, such as Avicel PH 102 or Avicel PH 200.

In some embodiments the composition comprises 0.1-10% (w/w) or 0.5-5% (w/w), such as 1-3.5% (w/w) or 1% (w/w), lubricant. In some embodiments the composition comprises 1.5-3% (w/w), such as 2.1-2.7% (w/w), 2.2-2.6% (w/w) or 2.3-2.5% (w/w), lubricant.

Still further, the composition or granule of the present disclosure may be formulated as is known in the art of oral formulations of insulinotropic compounds.

The composition or granule may be administered in several dosage forms, for example as a tablet; a capsule such as hard capsules, sachet or a powder. The composition or granule may further be compounded in a drug carrier or drug delivery system, e.g. in order to improve stability and/or solubility or further improve bioavailability.

In some embodiments the weight of the tablet is in the range of 150 mg to 1000 mg, such as in the range of 300-600 mg or 350-450 mg.

In some embodiments the present disclosure relates to a first granule comprising at least 75% (w/w) delivery agent, less than 10% (w/w) lubricant, and optionally less than 20% filler and no modified OXM or modified OXM analog. In some embodiments the present disclosure relates to a first granule comprising at least 80% (w/w) delivery agent, less than 10% (w/w) lubricant, and optionally less than 20% filler and no modified OXM or modified OXM analog. In some embodiments the first granule comprises 75-90% (w/w), such as 78-88% (w/w), 80-86% (w/w) or 82-84% (w/w), delivery agent. In some embodiments the first granule comprises less than 10% (w/w), such as 1-3% (w/w), 1.5-2.5% (w/w) or 1.9-2.3% (w/w), lubricant, In some embodiments the first granule comprises less than 20%, such as 10-20% (w/w), 12-18% (w/w) or 14-17% (w/w), filler. In some embodiments the first granule comprises no modified OXM or modified OXM analog. In some embodiments the granule comprises at least 80% (w/w) delivery agent, less than 10% (w/w) lubricant, and optionally less than 20% filler.

In some embodiments the present disclosure relates to a second granule comprising a modified OXM or modified OXM analog, at least 15% (w/w) filler and less than 40% (w/w) binder and no salt of N-(8-(2-hydroxybenzoyl)amino)caprylic acid. In some embodiments the second granule comprises at least 1%, such as 1-70% (w/w), 2-40% (w/w) or 4-30% (w/w), modified OXM or modified OXM analog. In some embodiments the second granule comprises at least 20%, such as 40-80% (w/w) or 50-75% (w/w), filler. In some embodiments the second granule comprises less than 30%, such as 5-30% (w/w), 10-28% (w/w) or 15-25% (w/w), binder. In some embodiments the second granule comprises no salt of N-(8-(2-hydroxybenzoyl)amino)caprylic acid. In some embodiments the granule comprises a modified OXM or modified OXM analog, at least 15% (w/w) filler and less than 40% (w/w) binder. In some embodiments the granule comprises a modified OXM or modified OXM analog, at least 50% (w/w) filler and less than 40% (w/w) binder.

In some embodiments the present disclosure relates to a composition comprising a first and a second type of granules, wherein the first type of granule comprises at least 75% (w/w) delivery agent, less than 10% (w/w) lubricant, optionally less than 20% filler and no modified OXM or modified OXM analog, and wherein the second type of granule comprises a modified OXM or modified OXM analog, at least 15% (w/w) filler, less than 40% (w/w) binder and no salt of N-(8-(2-hydroxybenzoyl)amino)caprylic acid. In some embodiments the present disclosure relates to a composition comprising a first and a second type of granules, wherein the first type of granule comprises at least

75% (w/w) delivery agent, less than 10% (w/w) lubricant, less than 20% filler and no modified OXM or modified OXM analog, and wherein the second type of granule comprises a modified OXM or modified OXM analog, at least 15% (w/w) filler, less than 40% (w/w) binder and no salt of N-(8-(2-hydroxybenzoyl)amino)caprylic acid. In some embodiments the present disclosure relates to a composition comprising a first and a second type of granules, wherein the first type of granule comprises at least 75% (w/w) delivery agent, less than 10% (w/w) lubricant, and no modified OXM or modified OXM analog, and wherein the second type of granule comprises a modified OXM or modified OXM analog, at least 15% (w/w) filler, less than 40% (w/w) binder and no salt of N-(8-(2-hydroxybenzoyl)amino)caprylic acid.

The delivery agent used in the present invention is a salt of N-(8-(2-hydroxybenzoyl)amino)caprylic acid. In some embodiments the delivery agent is an absorption enhancer. The structural formula of N-(8-(2-hydroxybenzoyl)amino)caprylate is shown Formula XXVII.

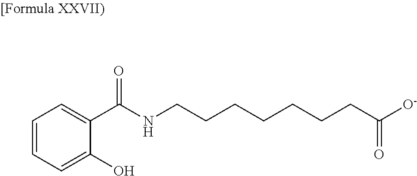 [SMILES: O=C([O-])CCCCCCCNC(=O)c1ccccc1O]

In some embodiments the salt of N-(8-(2-hydroxybenzoyl)amino)caprylic acid is in the caprylic acid form and/or the capraylate form. In some embodiments the salt of N-(8-(2-hydroxybenzoyl)amino)caprylic acid comprises one monovalent cation, two monovalent cations or one divalent cation. In some embodiments the salt of N-(8-(2-hydroxybenzoyl)amino)caprylic acid is selected from the group consisting of the sodium salt, potassium salt and calcium salt of N-(8-(2-hydroxybenzoyl)amino)caprylic acid.

Salts of N-(8-(2-hydroxybenzoyl)amino)caprylate may be prepared using the method described in e.g. WO96/030036, WO00/046182, WO01/092206 or WO2008/028859.

The salt of N-(8-(2-hydroxybenzoyl)amino)caprylic acid may be crystalline and/or amorphous. In some embodiments the delivery agent comprises the anhydrate, monohydrate, dihydrate, trihydrate, a solvate or one third of a hydrate of the salt of N-(8-(2-hydroxybenzoyl)amino) caprylic acid as well as combinations thereof. In some embodiments the delivery agent is a salt of N-(8-(2-hydroxybenzoyl)amino)caprylic acid as described in WO2007/121318. The salt of N-(8-(2-hydroxybenzoyl)amino)caprylic acid may be any polymorph thereof.

In some embodiments the delivery agent is sodium N-(8-(2-hydroxybenzoyl)amino)caprylate (referred to as “SNAC” herein), also known as sodium 8-(salicyloylamino) octanoate.

In some embodiments the amount of the salt of N-(8-(2-hydroxybenzoyl) amino)caprylic acid in the composition is in the range of 0.6-3.5 mmol. In some embodiments the amount of the salt of N-(8-(2-hydroxybenzoyl) amino)caprylic acid in the composition is at least 0.6 mmol, such as selected from the group at least 0.8 mmol or at least 0.9 mmol. In some embodiments the amount of the salt of N-(8-(2-hydroxybenzoyl)amino)caprylic acid in the composition is up to 2.5 mmol. In some embodiments the amount of the salt of N-(8-(2-hydroxybenzoyl)amino)caprylic acid in the composition is 0.6-2.0 mmol. In some embodiments the amount of the salt of N-(8-(2-hydroxybenzoyl)amino)caprylic acid is 1 mmol, such as 1.08 mmol.

In some embodiments the amount of SNAC or salt of NAC in the composition is in the range of 100-1000 mg. In some embodiments the amount of SNAC or salt of NAC in the composition is at least 150 mg or at least 250 mg. In some embodiments the amount of SNAC or salt of NAC in the composition is up to 800 mg, such as up to 700 mg or up to 600 mg. In some embodiments the amount of SNAC or salt of NAC in the composition is 300 mg.

In some embodiments the molar ratio between modified OXM or modified OXM analog and delivery agent in the composition is less than 10, such as less than 5 or less than 1. In some embodiments the molar ratio between modified OXM or modified OXM analog and delivery agent in the composition is less than 1/10, such as less than 1/100 or less than 5/1000.

The composition of the invention may be prepared as is known in the art. In some embodiments the composition or the granule may be prepared as described in International Patent Application No. WO 2013/139694 A1.

In some embodiments the amount of the salt of NAC in the composition is at least 0.6 mmol, such as selected from the group consisting of at least 0.65 mmol, at least 0.7 mmol, at least 0.75 mmol, at least 0.8 mmol, at least 0.8 mmol, at least 0.9 mmol, at least 0.95 mmol and at least 1 mmol. In some embodiments the amount of the salt of NAC in the composition is in the range of 0.6-2.1 mmol or 0.6-1.9 mmol. In some embodiments the amount of the salt of NAC in the composition is in the range of 0.7-1.7 mmol or 0.8-1.3 mmol. In some embodiments the amount of the salt of NAC in the composition is up to 2.1 mmol, such as selected from the group consisting of up to 2.1 mmol, up to 2 mmol, up to 1.9 mmol, up to 1.8 mmol, up to 1.7 mmol, up to 1.6 mmol, up to 1.5 mmol, up to 1.4 mmol, up to 1.3 mmol, up to 1.2 mmol and up to 1.1 mmol. In some embodiments the amount of the salt of NAC is 1 mmol, such as 1.08 mmol.

In some embodiments the dosage of modified OXM or modified OXM analog is in the range of 0.01 mg to 100 mg. In some embodiments the composition comprises an amount of a modified OXM or modified OXM analog in the range of 0.1 to 40 mg or 1 to 20 mg. In some embodiments the composition comprises an amount of a modified OXM or modified OXM analog in the range of 5 to 20 mg, such as in the range of 5 to 15 mg, such as 5 mg, such as 10 mg, such as 15 mg, such as 20 mg. In some embodiments the composition comprises an amount of a modified OXM or modified OXM analog in the range of 3 to 14 mg, such as 3 mg, such as 7 mg, such as 14 mg.

In some embodiments the composition comprises an amount of a modified OXM or modified OXM analog in the range of 0.05 to 25 mol, such as in the range of 0.5 to 2.5 μmol.

In some embodiments the composition comprises 1-100 mg modified OXM or modified OXM analog.

In some embodiments the composition comprises 1-100 mg modified OXM or modified OXM analog and 100-500 mg or 50-90% (w/w) of a salt of NAC. In some embodiments the composition comprises 1-100 mg modified OXM or modified OXM analog and 100-500 mg of a salt of NAC. In some embodiments the composition comprises 1-100 mg modified OXM or modified OXM analog and 50-90% (w/w) of a salt of NAC.

In some embodiments the composition comprises 1-100 mg modified OXM or modified OXM analog and 100-500 mg or 50-90% (w/w) SNAC. In some embodiments the composition comprises 1-100 mg modified OXM or modified OXM analog and 100-500 mg SNAC. In some embodiments the composition comprises 1-100 mg modified OXM or modified OXM analog and 50-90% (w/w) SNAC.

In one embodiment, the pharmaceutical composition is administered at between 1 mg and 20 mg. In another embodiment, the pharmaceutical composition is administered at between 3 mg and 14 mg. In another embodiment, the pharmaceutical composition is administered at 3 mg, 7, mg, or 14 mg.

In some embodiments the tablet of the invention co-releases the active ingredients and the delivery agent by surface erosion; hence, the tablets becomes smaller and smaller with time by dissolution primarily from the surface from non-disintegrated tablets. Concurrent release: In some embodiments the compositions show concurrent release of the GLP-1 agonist and the delivery agent from the surface of the tablet. This can be tested by visual inspection during the disintegration test; the tablets do not have concurrent release of the GLP-1 agonist and the delivery agent from the surface of the tablet if the tablet breaks into smaller parts during the first 8 minutes of the disintegration test.

The treatment with a composition according to the present invention may also be combined with one or more additional pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), Gastric Inhibitory Polypeptides (GIP analogs), compounds lowering food intake, RXR agonists and agents acting on the ATP-dependent potassium channel of the 3-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, PYY agonists, Y2 receptor agonists, Y4 receptor agonists, mixed Y2/Y4 receptor agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, oxyntomodulin and analogues, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, Gastric Inhibitory Polypeptide agonists or antagonists (GIP analogs), gastrin and gastrin analogs. In addition, vitamins including prescribed vitamins such as vitamin D metabolites (e.g. calcifediol) may be administered in combination with the disclosed OXM molecules. The combination products may be in different dosage forms or combined in a single dosage form having two or more active ingredients.

### Methods of Treatment

In another aspect, the present disclosure provides a method of treating cardiometabolic and associated diseases comprising administering to a subject in need of such treatment a therapeutically effective amount of any one of the modified OXM or modified OXM analogs.

In one embodiment, disclosed is a method of treating a disease for which an agonist of glucagon-like peptide-1 receptor (GLP-1 R) is indicated, in a subject in need of such prevention and/or treatment, comprising administering to the subject a therapeutically effective amount of any of the modified OXM or modified OXM analogs disclosed, or a pharmaceutically acceptable salt thereof. In another embodiment, disclosed is the use of any of the modified OXM or modified OXM analogs disclosed, or a pharmaceutically acceptable salt thereof, for the manufacture of a medicament for treating a disease or condition for which an agonist of the GLP-1 R is indicated. In a related embodiment, disclosed is any of the modified OXM or modified OXM analogs for use in the treatment of a disease or condition for which an agonist of GLP-1 R is indicated. In another embodiment, disclosed is a pharmaceutical composition comprising any of the modified OXM or modified OXM analogs for the treatment of a disease or condition for which an agonist of the GLP-1 R is indicated.

In one embodiment, disclosed is a method of treating a disease for which an agonist of glucagon receptor (GCGR) is indicated, in a subject in need of such prevention and/or treatment, comprising administering to the subject a therapeutically effective amount of any of the modified OXM or modified OXM analogs disclosed, or a pharmaceutically acceptable salt thereof. In another embodiment, disclosed is the use of any of the modified OXM or modified OXM analogs disclosed, or a pharmaceutically acceptable salt thereof, for the manufacture of a medicament for treating a disease or condition for which an agonist of the GCGR is indicated. In a related embodiment, disclosed is any of the modified OXM or modified OXM analogs for use in the treatment of a disease or condition for which an agonist of GCGR is indicated. In another embodiment, disclosed is a pharmaceutical composition comprising any of the modified OXM or modified OXM analogs for the treatment of a disease or condition for which an agonist of the GCGR is indicated.

In one embodiment, disclosed is a method of treating a disease for which a dual agonist of GCGR and GLP-1 R is indicated, in a subject in need of such prevention and/or treatment, comprising administering to the subject a therapeutically effective amount of any of the modified OXM or modified OXM analogs disclosed, or a pharmaceutically acceptable salt thereof. In another embodiment, disclosed is the use of any of the modified OXM or modified OXM analogs disclosed, or a pharmaceutically acceptable salt thereof, for the manufacture of a medicament for treating a disease or condition for which a dual agonist of the GCGR and GLP-1 R is indicated. In a related embodiment, disclosed is any of the modified OXM or modified OXM analogs for use in the treatment of a disease or condition for which a dual agonist of GCGR and GLP-1 R is indicated. In another embodiment, disclosed is a pharmaceutical composition comprising any of the modified OXM or modified OXM analogs for the treatment of a disease or condition for which a duel agonist of the GCGR and GLP-1 R is indicated.

In another embodiment, disclosed is a method of treating cardiometabolic and associated diseases comprising administering to a subject in need of such treatment a therapeutically effective amount of any one of the modified OXM or modified OXM analogs, wherein the disease is T1D, T2DM, pre-diabetes, idiopathic T1D (Type 1b), LADA (latent autoimmune diabetes in adults), EOD (early-onset T2DM), YOAD (youth-onset atypical diabetes), MODY (maturity onset diabetes of the young), malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), excessive sugar craving, dyslipidemia, hyperinsulinemia, NAFLD (including related diseases such as steatosis, NASH, fibrosis, cirrhosis, and hepatocellular carcinoma), NASH, fibrosis, cirrhosis, hepatocellular carcinoma, cardiovascular disease, atherosclerosis (including coronary artery disease), coronary artery disease, peripheral vascular disease, hypertension, endothelial dysfunction, impaired vascular compliance, congestive heart failure, myocardial infarction (e.g., necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome Crohn's disease, colitis, irritable bowel syndrome, prevention or treatment of Polycystic Ovary Syndrome and treatment of addiction (e.g., alcohol and/or drug abuse).

In another embodiment, disclosed is the use of a therapeutically effective amount of the compound of any one of the modified OXM or modified OXM analogs in the manufacture of a medicament for treating a subject in need. In another embodiment, disclosed is the use of a therapeutically effective amount of the compound of any one of the modified OXM or modified OXM analogs in the manufacture of a medicament for treating a subject with cardiometabolic and associated diseases. In another embodiment, In another embodiment, disclosed is the use of a therapeutically effective amount of the compound of any one of the modified OXM or modified OXM analogs in the manufacture of a medicament for treating a subject with any one or more of the following cardiometabolic and associated diseases: T1D, T2DM, pre-diabetes, idiopathic T1D (Type 1b), LADA (latent autoimmune diabetes in adults), EOD (early-onset T2DM), YOAD (youth-onset atypical diabetes), MODY (maturity onset diabetes of the young), malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), excessive sugar craving, dyslipidemia, hyperinsulinemia, NAFLD (including related diseases such as steatosis, NASH, fibrosis, cirrhosis, and hepatocellular carcinoma), NASH, fibrosis, cirrhosis, hepatocellular carcinoma, cardiovascular disease, atherosclerosis (including coronary artery disease), coronary artery disease, peripheral vascular disease, hypertension, endothelial dysfunction, impaired vascular compliance, congestive heart failure, myocardial infarction (e.g., necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome Crohn's disease, colitis, irritable bowel syndrome, prevention or treatment of Polycystic Ovary Syndrome and treatment of addiction (e.g., alcohol and/or drug abuse).

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood.

In any of the embodiments described herein, the cardiovascular disease is cardiomyopathy, heart failure, or high blood pressure. In any of the embodiments described herein, the cardiovascular disease is cardiomyopathy. In any of the embodiments described herein, the cardiovascular disease is heart failure. In any of the embodiments described herein, the cardiovascular disease is high blood pressure.

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

In some embodiments, this disclosure provides methods of reducing the risk of cardiovascular (CV) disease in a human being. In one embodiment, this disclosure provides methods of reducing the risk of cardiovascular (CV) disease in a human being, the method comprising administering modified OXM or modified OXM analogs disclosed herein.

In some embodiments the present disclosure relates to a method of treating type 2 diabetes, comprising administering modified OXM or modified OXM analogs in a therapeutically effective amount to a subject in need thereof, wherein said subject has clinical evidence of cardiovascular disease and/or subclinical evidence of cardiovascular disease; wherein said method reduces or delays a major adverse cardiovascular event (MACE).

In some embodiments MACE is events selected from the group consisting of cardiovascular (CV) death, non-fatal MI, non-fatal stroke, revascularisation, hospitalisation for unstable angina pectoris, and hospitalisation for heart failure. The term “non-fatal MI” as used herein refers to non-fatal myocardial infarction. In some embodiments MACE is events selected from the group consisting of CV death, non-fatal MI, and non-fatal stroke.

In some embodiments the method reduces or delays a major adverse cardiovascular event (MACE). In some embodiments the method reduces the risk of said subject developing a major adverse cardiovascular event (MACE). In some embodiments the method reduces the risk of said subject developing its first MACE. Thus, in some embodiments the MACE referred to herein is first MACE, e.g. after initiating administration of modified OXM or modified OXM analogs. The term “first MACE” as used herein refers to the first MACE event of a subject after initiation of modified OXM or modified OXM analogs administration.

In some embodiments MACE is selected from the group consisting of CV death, non-fatal MI, non-fatal stroke, revascularisation, hospitalisation for heart failure, and hospitalisation for unstable angina pectoris. In some embodiments MACE (e.g. selected from the group consisting of CV death, non-fatal MI, non-fatal stroke, revascularisation, hospitalisation for heart failure, and hospitalisation for unstable angina pectoris) is reduced or delayed by at least 1% compared to placebo. In some embodiments MACE (e.g. selected from the group consisting of CV death, non-fatal MI, non-fatal stroke, revascularisation, hospitalisation for heart failure, and hospitalisation for unstable angina pectoris) is reduced or delayed by from about 20% to about 35% compared to placebo. In some embodiments MACE (e.g. selected from the group consisting of CV death, non-fatal MI, non-fatal stroke, revascularisation, hospitalisation for heart failure, and hospitalisation for unstable angina pectoris) is reduced about 27% compared to placebo. In some embodiments the first MACE (e.g. selected from the group consisting of CV death, non-fatal MI, non-fatal stroke, revascularisation, hospitalisation for heart failure, and hospitalisation for unstable angina pectoris) is reduced or delayed by at least 1% compared to placebo. In some embodiments the first MACE (e.g. selected from the group consisting of CV death, non-fatal MI, non-fatal stroke, revascularisation, hospitalisation for heart failure, and hospitalisation for unstable angina pectoris) is reduced or delayed by from about 20% to about 27% compared to placebo. In some embodiments the first MACE (e.g. selected from the group consisting of CV death, non-fatal MI, non-fatal stroke, revascularisation, hospitalisation for heart failure, and hospitalisation for unstable angina pectoris) is reduced about 27% compared to placebo.

In some embodiments MACE is selected from the group consisting of CV death, non-fatal MI, and non-fatal stroke. In some embodiments MACE (e.g. selected from the group consisting of CV death, non-fatal MI, and non-fatal stroke) is reduced or delayed by at least 10% compared to placebo. In some embodiments MACE (e.g. selected from the group consisting of CV death, non-fatal MI, and non-fatal stroke) is reduced or delayed by from about 20% to about 30% compared to placebo.

In some embodiments the MACE is revascularisation. Revascularisation may be coronary revascularisation or peripheral revascularisation.

In some embodiments the MACE is hospitalisation for unstable angina pectoris.

In some embodiments the administration of modified OXM or modified OXM analogs is a chronic treatment in which the modified OXM or modified OXM analogs is administered for at least 16 months (such as at least 30 months, and optionally up to 54 months), and wherein said method reduces or delays non-fatal myocardial infarction (MI).

In some embodiments the administration of modified OXM or modified OXM analogs is a chronic treatment in which the modified OXM or modified OXM analogs is administered for at least 18 months (such as at least 30 months, and optionally up to 54 months), and wherein said method reduces the need or risk of requiring revascularisation. In some embodiments the MACE is CV death.

In some embodiments, this disclosure provides methods of reducing the risk of cardiovascular (CV) disease in a human being, wherein at least one risk factor associated with CV disease is reduced and/or eliminated, the at least one risk factor being selected from the group consisting of excessive body weight, high serum lipids, cholesterol, triglycerides, LDL and/or VLDL particle concentration and/or diameter, phosphatidylethanolamines, phosphatidylcholines, lysophosphatidylethanolamines, sphingolipids and/or lysophosphatidylcholines in serum of the human being; optionally wherein the human being is overweight, obese, has a body mass index (BMI kg/m2) of greater than or equal to 25, and/or has Type II diabetes. In one embodiment, this disclosure provides methods of reducing the risk of cardiovascular (CV) disease in a human being, the method comprising administering modified OXM or modified OXM analogs disclosed herein, wherein at least one risk factor associated with CV disease is reduced and/or eliminated, the at least one risk factor being selected from the group consisting of excessive body weight, high serum lipids, cholesterol, triglycerides, LDL and/or VLDL particle concentration and/or diameter, phosphatidylethanolamines, phosphatidylcholines, lysophosphatidylethanolamines, sphingolipids and/or lysophosphatidylcholines in serum of the human being; optionally wherein the human being is overweight, obese, has a body mass index (BMI kg/m2) of greater than or equal to 25, and/or has Type II diabetes.

In one embodiment, administration of the modified OXM or modified OXM analogs disclosed herein induces a significant reduction in atherogenic lipids and/or lipoprotein number as compared to baseline.

In one embodiment, disclosed herein is a method of treating cardiovascular (CV) disease risk factors comprising: administering a pharmaceutical composition comprising a therapeutically effective amount of the modified OXM or modified OXM analogs disclosed herein, or a pharmaceutically acceptable salt thereof to a patient in need thereof, wherein the patient is overweight, obese, has a body mass index (BMI kg/m2) of greater than or equal to 25, and/or has Type II diabetes.

In one embodiment, disclosed herein is a method of treating cardiovascular (CV) disease risk factors comprising: administering a pharmaceutical composition comprising a therapeutically effective amount of the modified OXM or modified OXM analogs disclosed herein, or a pharmaceutically acceptable salt thereof to a patient in need thereof, wherein the patient is overweight, obese, has a body mass index (BMI kg/m2) of greater than or equal to 25, and/or has Type II diabetes and wherein the method reduces at least one of plasma triglycerides, total plasma cholesterol, plasma LDL-cholesterol or plasma HDL-cholesterol levels by about any of 3%, 5% 7%, or 10% from baseline.

In one embodiment, disclosed herein is a method of treating cardiovascular (CV) disease risk factors comprising: administering a pharmaceutical composition comprising a therapeutically effective amount of the modified OXM or modified OXM analogs disclosed herein, or a pharmaceutically acceptable salt thereof to a patient in need thereof, wherein the patient is overweight, obese, has a body mass index (BMI kg/m2) of greater than or equal to 25, and/or has Type II diabetes and wherein the method reduces at least one of plasma triglycerides, total plasma cholesterol, plasma LDL-cholesterol or plasma HDL-cholesterol levels.

In one embodiment, disclosed herein is a method of treating cardiovascular (CV) disease risk factors comprising: administering a pharmaceutical composition comprising a therapeutically effective amount of the modified OXM or modified OXM analogs disclosed herein, or a pharmaceutically acceptable salt thereof to a patient in need thereof, and wherein the method reduces at least one of plasma triglycerides, total plasma cholesterol, plasma LDL-cholesterol or plasma HDL-cholesterol levels by about any of 3%, 5% 7%, or 10% from baseline. In another embodiment, disclosed herein is a method of treating cardiovascular (CV) disease risk factors comprising: administering a pharmaceutical composition comprising a therapeutically effective amount of the modified OXM or modified OXM analogs disclosed herein, or a pharmaceutically acceptable salt thereof to a patient in need thereof, and wherein the method reduces at least one of plasma triglycerides, total plasma cholesterol, plasma LDL-cholesterol or plasma HDL-cholesterol levels.

In one embodiment, disclosed herein is a method of reducing plasma lipids in a patient in need thereof, comprising administering a pharmaceutical composition comprising a therapeutically effective amount the modified OXM or modified OXM analogs disclosed herein, or a pharmaceutically acceptable salt thereof to the patient in need thereof, wherein the patient has established cardiovascular disease and wherein the method reduces at least one of plasma triglycerides, total plasma cholesterol, plasma LDL-cholesterol or plasma HDL-cholesterol levels by 3%, 5%, 7%, or 10% from baseline. In another embodiment, disclosed herein is a method of reducing plasma lipids in a patient in need thereof, comprising administering a pharmaceutical composition comprising a therapeutically effective amount the modified OXM or modified OXM analogs disclosed herein, or a pharmaceutically acceptable salt thereof to the patient in need thereof, wherein the patient has established cardiovascular disease and wherein the method reduces at least one of plasma triglycerides, total plasma cholesterol, plasma LDL-cholesterol or plasma HDL-cholesterol levels.

In one embodiment, disclosed herein is a method of reducing plasma lipids in a patient in need thereof, comprising administering a pharmaceutical composition comprising a therapeutically effective amount the modified OXM or modified OXM analogs disclosed herein, or a pharmaceutically acceptable salt thereof to the patient in need thereof, and wherein the method reduces at least one of plasma triglycerides, total plasma cholesterol, plasma LDL-cholesterol or plasma HDL-cholesterol levels by 3%, 5%, 7%, or 10% from baseline. In another In one embodiment, disclosed herein is a method of reducing plasma lipids in a patient in need thereof, comprising administering a pharmaceutical composition comprising a therapeutically effective amount the modified OXM or modified OXM analogs disclosed herein, or a pharmaceutically acceptable salt thereof to the patient in need thereof, and wherein the method reduces at least one of plasma triglycerides, total plasma cholesterol, plasma LDL-cholesterol or plasma HDL-cholesterol levels.

In one embodiment, disclosed herein is a method of reducing plasma lipids in a patient in need thereof, comprising administering a pharmaceutical composition comprising a therapeutically effective amount the modified OXM or modified OXM analogs disclosed herein, or a pharmaceutically acceptable salt thereof to the patient in need thereof, wherein the patient has established cardiovascular disease.

In some embodiments the subject to receive administration of modified OXM or modified OXM analogs according to the methods of the present disclosure has type 2 diabetes as well as (i) clinical evidence of cardiovascular disease, and/or (ii) subclinical evidence of cardiovascular disease. These cardiovascular diseases may be referred to as concomitant, i.e. one or more cardiovascular diseases are present in the subject at the same time as type 2 diabetes.

“Clinical evidence of cardiovascular disease” may be present when the subject fulfils at least one criterion selected from the group consisting of
a) prior myocardial infarction,
b) prior stroke or transient ischaemic attack (TIA),
c) prior coronary, carotid or peripheral arterial revascularisation,
d) >50% stenosis on angiography or imaging of coronary, carotid or lower extremity arteries,
e) history of symptomatic coronary heart disease (e.g documented by eg positive exercise stress test or any cardiac imaging or unstable angina with ECG changes),
f) asymptomatic cardiac ischemia (e.g. documented by positive nuclear imaging test or exercise test or stress echo or any cardiac imaging),
g) heart failure New York Heart Association (NYHA) class and
h) chronic renal impairment (e.g. documented (prior to screening) by estimated glomerular filtration rate (eGFR)<60 mL/min/1.73 m2 per MDRD).

In some embodiments clinical evidence of cardiovascular disease is prior myocardial infarction. In some embodiments clinical evidence of cardiovascular disease is prior stroke or transient ischaemic attack (TIA). In some embodiments clinical evidence of cardiovascular disease is prior coronary, carotid or peripheral arterial revascularisation. In some embodiments clinical evidence of cardiovascular disease is >50% stenosis on angiography or imaging of coronary, carotid or lower extremity arteries. In some embodiments clinical evidence of cardiovascular disease is history of symptomatic coronary heart disease (e.g. documented by positive exercise stress test or any cardiac imaging or unstable angina with ECG changes). In some embodiments clinical evidence of cardiovascular disease is asymptomatic cardiac ischemia (e.g. documented by positive nuclear imaging test or exercise test or stress echo or any cardiac imaging). In some embodiments clinical evidence of cardiovascular disease is heart failure New York Heart Association (NYHA) class In some embodiments clinical evidence of cardiovascular disease is chronic renal impairment (e.g., documented (prior to screening) by estimated glomerular filtration rate (eGFR)<60 mL/min/1.73 m2 per MDRD.

“Subclinical evidence of cardiovascular disease” may be present when the subject fulfils at least one criterion selected from the group consisting of
i) persistent microalbuminuria (e.g. 30-299 mg/g) or proteinuria,
j) hypertension and left ventricular hypertrophy by ECG or imaging,
k) left ventricular systolic or diastolic dysfunction (e.g. by imaging), and
l) ankle/brachial index <0.9.

In some embodiments subclinical evidence of cardiovascular disease is persistent microalbuminuria (30-299 mg/g) or proteinuria. In some embodiments subclinical evidence of cardiovascular disease is hypertension and left ventricular hypertrophy by ECG or imaging. In some embodiments subclinical evidence of cardiovascular disease is left ventricular systolic or diastolic dysfunction by imaging. In some embodiments subclinical evidence of cardiovascular disease is ankle/brachial index <0.9.

In some embodiments the term “prior” refers to before initiating administration of modified OXM or modified OXM analogs.

In one embodiment, disclosed is a method of reducing the risk of a major adverse cardiovascular event (MACE), comprising: administering modified OXM or modified OXM analogs in a therapeutically effective amount to a subject in need thereof, wherein the subject has type 2 diabetes and cardiovascular disease.

Disclosed herein is a pharmaceutical composition or a granule for use as a medicament. In one embodiment the composition or the granule is administered orally. In another embodiment the composition or the granule is suitable for parenteral administration. In another embodiment the composition or the granule is administered parenterally.

Administration of pharmaceutical compositions disclosed herein may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current disclosure are useful in the formulation of solids, semisolids, powder and solutions for pulmonary administration of compounds of the present disclosure, using, for example a metered dose inhaler, dry powder inhaler and a nebulizer, all being devices well known to those skilled in the art.

Compositions disclosed herein are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. In another embodiment, disclosed are controlled release and sustained release systems administered subcutaneously. Without limiting the scope of the invention, examples of useful controlled release system and compositions are hydrogels, oleaginous gels, liquid crystals, polymeric micelles, microspheres, nanoparticles. Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension or a powder for the administration of any compounds disclosed herein in the form of a nasal or pulmonal liquid or powder spray. As a still further option, the pharmaceutical compositions containing the compound can also be adapted to transdermal administration, e.g. by needle-free injection or from a patch, optionally an iontophoretic patch, or transmucosal, e.g. buccal, administration.

The compounds of the present disclosure can be administered via the pulmonary route in a vehicle, as a solution, suspension or dry powder using any of known types of devices suitable for pulmonary drug delivery. Examples of these comprise, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit Rev Ther Drug Carr Sys 14(4) (1997) 395-453).

It should be understood that any embodiment of the present disclosure, including those described only in the Examples or claims, or only in one section of the specification, can be combined with one or more additional embodiments of the present disclosure, to the extent that such combinations are not expressly disclaimed or are improper.

### Definitions

As used herein, the singular form “a”, “an” and “the” include plural references unless the context clearly dictates otherwise. For example, the term “a compound” or “at least one compound” may include a plurality of compounds, including mixtures thereof.

A “non-naturally encoded amino acid” refers to an amino acid that is not one of the common amino acids or pyrrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof. The term “non-naturally encoded amino acid” also includes, but is not limited to, amino acids that occur by modification (e.g. post-translational modifications) of a naturally encoded amino acid (including but not limited to, the 20 common amino acids or pyrrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

Throughout this application, various embodiments of this present disclosure may be presented in a range format. It should be understood that the description in range format is merely for convenience and brevity and should not be construed as an inflexible limitation on the scope of the present disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

A skilled artisan would appreciate that the term “about”, may encompass a deviance of between 0.0001-5% from the indicated number or range of numbers. In some instances, the term “about”, may encompass a deviance of between 1-10% from the indicated number or range of numbers. In some instances, the term “about”, encompasses a deviance of up to 25% from the indicated number or range of numbers.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount”, or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “patient” or “subject” refers to a living mammalian organism, such as a human, monkey, cow, horse, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo [2.2.2]oct-2-ene-1-carboxylic acid, acetic acid, aliphatic mono-anddicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this present disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

### EXAMPLES

### Example 1

### Synthesis of OXM Analogs

The OXM analogs according to SEQ ID NO: 3 (where Xaa38 is absent and Xaa39 is absent) and SEQ ID NO: 13 of the present application were generated by solid-phase peptide synthesis on a Protein Technologies Inc. Symphony or Applied Biosystems 433 A automated peptide synthesizer. Synthesis was performed on Fmoc-Rink amide polystyrene resin (Rapp Polymere Tubingen, Germany) with substitution approximately 0.7 mmol/g. The synthesis was performed using the Fmoc main-chain protecting group strategy. Amino acid side-chain derivatives used were: Arg(Pbf), Asn(Trt), Asp(OtBu), Cys(Trt), Gln(Trt), Glu(OtBu), His(Trt), Lys(Boc), Ser(OtBu), Thr(OtBu), Trp(Boc), and Tyr(OtBu). Coupling is carried out with approximately 10 equivalents of amino acid activated with diisopropylcarbodiimide (DIC) and hydroxybenzotriazole (HOBt) (1:1:1 molar ratio) in dimethylformamide (DMF) or N-methyl pyrrolidinone (NMP). Coupling was carried out for 45 to 90 minutes at room temperature.

Concomitant cleavage from the resin and side chain protecting group removal were carried out in a solution containing trifluoroacetic acid (TFA):triisopropylsilane:3,6-dioxa-1,8-octane-dithiol:methanol:anisole 90:4:2:2:2 (v/v) for 1.5 to 2 hr at room temperature. The solution was filtered and concentrated to <2 mL, and peptides were precipitated with cold diethyl ether, redissolved in 30-40 mL of 10% acetonitrile and purified on a Cis reversed-phase high performance liquid chromatography (HPLC) column (typically a Waters SymmetryPrep 7 μm, 19×300 mm) at a flow rate of 12-15 mL/min. Samples were eluted with a two-stage linear AB gradient of 0 to 25% B over 20 minutes followed by 25 to 75% B over 100 minutes where A=0.05% TFA/water and B=0.05% TFA/acetonitrile. The product generally eluted at 30-35% acetonitrile. Peptide purity and molecular weight was confirmed on an Agilent 1 100 Series liquid chromatography-mass spectrometry (LC-MS) system with a single quadrupole MS detector. Analytical HPLC separation was done on a Zorbax Eclipse XDB-C8, 5 micron, 4.6 mm i.d.×15 cm column with a linear AB gradient of 6 to 60% B over 15 minutes in which A=0.05% TFA/H20 and B=0.05% TFA/acetonitrile and the flow rate is 1 ml/min. The peptide analogue was purified to >95% purity and was confirmed to have molecular weight corresponding to the calculated value within 1 atomic mass unit (amu).

### Example 2

### Design of Modified OXM Complexes

The goal of the present experiment is to design an albumin binding moiety of an OXM analog. This is intended to increase the longevity of the OXM analog, increase the OXM analog's weight loss effect, and increase the OXM analog's longevity.

Instead of modifying the OXM analog with PEGylation, a lower molecular weight molecule was utilized in order to overcome PEG-related limitations and allow optimal dosing to harness maximal efficacy of the OXM analog.

Three principles were prioritized when designing a long-acting prodrug employing an albumin-binding probe. The first principle is that following its conjugation, the probe introduced into the drug doesn't interfere with the drug ability to properly interact with a target receptor. Secondly, the conjugated drug should have sufficient affinity to albumin to manifest prolonged action in vivo.

Third, most importantly, and many times overlooked, is the ability of the Albumin/conjugate complex to bind the FcRn receptor, that sequentially enables the recycle mechanism. The principles listed above were highlighted and considered to be the most important aspect during the designing the different albumin binding moieties disclosed below.

In particular, having the acylation at the C terminal of the OXM variants which were not shortened by reducing the number of amino acids in oxyntomodulin achieved maximum binding affinity on both receptors while also permitting effective albumin binding. In an embodiment, the present disclosure comprises an acylated OXM variant having at least 37 amino acids wherein the acylation occurs at the C terminal of the OXM variant polypeptide. In a preferred embodiment, the present disclosure comprises an acylated OXM variant having 37, 38 and/or 39 amino acids and wherein the acylation occurs at a position on the C terminal of the OXM variant.

The general design of these newly developed acylated OXM analogs utilizing an albumin binding technology to increase the longevity of the hormone is shown below. This design includes the protein design, OXM analog, and the binder complex design, which is the binder, spacer, and

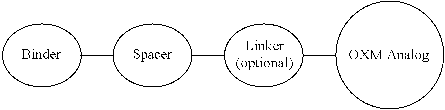

optional linker.

The acylated OXM analogs discussed in this example are also referred to as MOD-604, long-acting OXM, long-acting OXM analogs, modified OXM or modified OXM analogs.

### Protein Design

An oxyntomodulin analog was selected as the target protein. The OXM analogs selected are any one of SEQ ID NOs: 2 to 16.

### Binder Complex

There were multiple design considerations to produce a pharmaceutically effective, long acting OXM analog. First, a long fatty acid chain was selected in order to effectively bind to human serum albumin. Secondly, in some embodiments, a diacid form of the fatty acid was found to be very important for the binding affinity to albumin. Thirdly, the linker moiety was discovered to be important for the overall properties of the claimed molecules. Fourthly, maintaining the integrity of the amino acid length of oxyntomodulin was determined to be a key element while, lastly, linking the acylated moiety to the C terminal was also determined to be important.

A table describing the binder complexes that were designed is shown in Table 1 below.

TABLE 1
Binder Complex

Eicosanedioic-gGlu-(AEEA)₂-Lys(AcBr)-Gly-OH
Octadecanedioic-gGlu-(AEEA)₂-Lys(AcBr)-Gly-OH
Eicosanedioic-gGlu-Glu-(AEEA)₃-Lys(AcBr)-Gly-OH
Octadecanedioic-gGlu-Glu-(AEEA)₃-Lys(AcBr)-Gly-OH
Eicosanedioic-gGlu-(AEEA)₂
Octadecanedioic-gGlu-(AEEA)₂

A table describing the full modified OXM analog complexes that were designed is shown in Table 2 below.

TABLE 2
Modified OXM analog complex Design

| | | | Corresponding |
|---|---|---|---|
| | | | Formula |
| Variant | OXM | | for Full |
| # | Backbone | Full Modified OXM Analog | Modified OXM |
| 1 | H(Aib)QG | H(Aib)QGTFTSDYSKYLDSKK | Formula XIV |
| | TFTSDYSK | AQEFVQWLLN(Aib)GRNRNNI | |
| | YLDSKKAQ | AC³⁸(Eicosanedioic-gGlu- | |
| | EFVQWLLN | (AEEA)₂-Lys(Ac)-Gly)-NH₂ | |
| | (Aib)GRN | (SEQ ID NO: 39) | |
| | RNNIAC³⁸- | | |
| | NH₂ (SEQ | | |
| | ID NO: | | |
| | 69) | | |
| 2 | | H(Aib)QGTFTSDYSKYLDSKK | Formula XV |
| | | AQEFVQWLLN(Aib)GRNRNNI | |
| | | AC³⁸(Octadecanedioic-gGlu- | |
| | | (AEEA)₂-Lys(Ac)-Gly)-NH₂ | |
| | | (SEQ ID NO: 40) | |
| 3 | | H(Aib)QGTFTSDYSKYLDSKK | Formula XVI |
| | | AQEFVQWLLN(Aib)GRNRNNI | |
| | | AC³⁸(Eicosanedioic-gGlu- | |
| | | Glu-(AEEA)₃-Lys(Ac)-Gly)- | |
| | | NH₂ (SEQ ID NO: 41) | |
| 4 | | H(Aib)QGTFTSDYSKYLDSKK | Formula XVII |
| | | AQEFVQWLLN(Aib)GRNRNNI | |
| | | AC³⁸(Octadecanedioic-gGlu- | |
| | | Glu-(AEEA)₃-Lys(Ac)-Gly)- | |
| | | NH₂ (SEQ ID NO: 42) | |
| 5 | H(Aib)QG | H(Aib)QGTFTSDYSKYLDSKK | Formula XVIII |
| | TFTSDYSK | AQEFVQWLLN(Aib)GRNRNNI | |
| | YLDSKKAQ | AK³⁸(Eicosanedioic-gGlu- | |
| | EFVQWLLN | (AEEA)₂)G³⁹-NH₂ (SEQ ID NO: | |
| | (Aib)GRN | 43) | |
| | RNNIAK³⁸ | | |
| | G³⁹-NH₂ | | |
| 6 | | H(Aib)QGTFTSDYSKYLDSKK | Formula XIX |
| | | AQEFVQWLLN(Aib)GRNRNNI | |
| | | AK³⁸(Octadecanedioic-gGlu- | |
| | | (AEEA)₂)G³⁹-NH₂ (SEQ ID NO: | |
| | | 44) | |
| 7 | H(Aib)QG | Eicosanedioic-gGlu-Glu-AEEA- | Formula XXII |
| | TFTSDYSK | AEEA-AEEA-Lys(Ac- | |
| | YLDSKKAQ | Cys³⁸mut.OXM (C³⁸C³⁹))- | |
| | EFVQWLLN | Gly-OH (SEQ ID NO: 47) | |
| | (Aib)GRN | | |
| | RNNIAC³⁸ | | |
| | C³⁹-NH₂ | | |
| 8 | | Eicosanedioic-gGlu-Glu-AEEA- | Formula XXIII |
| | | AEEA-AEEA-Lys(Ac- | |
| | | Cys³⁹mut.OXM (C³⁸C³⁹))-Gly- | |
| | | OH (SEQ ID NO: 48) | |
| 9 | | H(Aib)QGTFTSDYSKYLDSKK | Formula XXV |
| | | AQEFVQWLLN(Aib)GRNRNNI | |
| | | AC³⁸(Eicosanedioic-gGlu- | |
| | | Glu-(AEEA)₃-Lys(Ac)- | |
| | | Gly)C³⁹(Eicosanedioic-gGlu- | |
| | | Glu-(AEEA)₃-Lys(Ac)-Gly)-NH₂ | |
| | | (SEQ ID NO: 50) | |

### Example 3

### Synthesis of Acylated OXM Analogs

### 3.1: Overview

The present Example details the specifications for the synthesis of an acylated OXM analog, which is a long acting oxyntomodulin (OXM) analog possessing albumin binding property and GLP-1 and glucagon activities.

The OXM analogue is constructed from two key materials: (1) a mutated OXM peptide backbone (“KPI”), and (2) a fatty acid-peptide complex (“Binder” or “Binder Complex”).

The construction of the acylated OXM analogue is obtained via a conjugation step between the binder, and a specific site (cysteine) on the mutated OXM peptide. The acylated OXM structure is shown in Formula XXVI below. This acylated OXM structure is also referred to as OPK-88604.

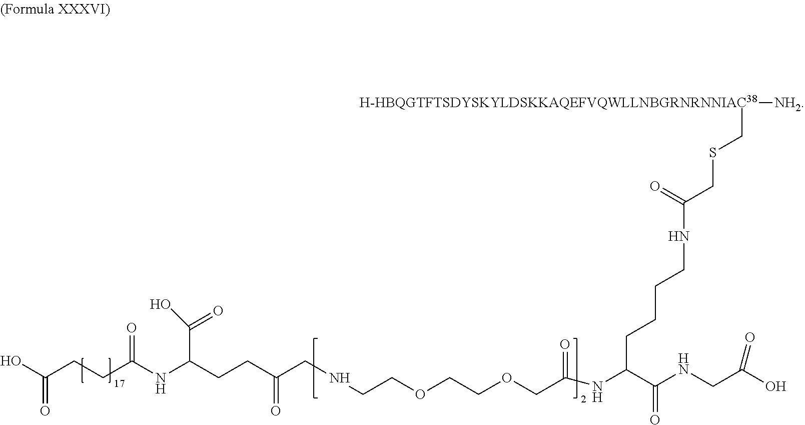

The structure and characteristics of the mutated OXM backbone are detailed in Table 3 below.

TABLE 3
Mutated OXM Backbone Structure

| | |
|---|---|
| Sequence and structure* | H-His-Aib-Gln-Gly-Thr-Phe-Thr-Ser-Asp-Tyr-Ser-Lys-Tyr- |
| | Leu-Asp-Ser-Lys-Lys-Ala-Gln-Glu-Phe-Val-Gln-Trp-Leu- |
| | Leu-Asn-Aib-Gly-Arg-Asn-Arg-Asn-Asn-Ile-Ala-Cys-NH₂ |
| | (SEQ ID NO: 73) |
| Molecular Formula | C₁₉₄H₂₉₈N₅₈O₅₈S |
| Molecular weight | 4402.93Da (calculated) |
| Theoretical EC (280 nm) | 8670 M⁻¹ cm⁻¹ or 1.969 Abs 0.1% (1 g/l) |

*All mutations with respect to wild type OXM are shown in bold

The structure and characteristics of the binder complex are detailed in Table 4 below.

TABLE 4
Binder Complex Structure

| | |
|---|---|
| Sequence and | Eicosanedioic-□Glu-(AEEA)₂-Lys(Ac-Br)-Gly- |
| structure | OH |
| Molecular Formula | C₄₈H₈₅BrN₆O₁₆ |
| Molecular weight | 1082.14 Da |
| Solubility data | Water-based buffer at pHs 6.5-7.5 |

The structure and characteristics of the building blocks of the binder complex are detailed in Table 5 below.

TABLE 5
Binder Building Blocks

| Building Block Unit | CAS | | Molecular |
|---|---|---|---|
| Name | Number | MW | Formula |
| tBu-Eicosanedioic acid | 683239-16-9 | 398.62 | C₂₄H₄₆O₄ |
| Fmoc-Glu-OtBu | 84793-07-7 | 425.47 | C₂₄H₂₇NO₆ |
| Fmoc-Glu(OtBu)-OH | 71989-18-9 | 425.47 | C₂₄H₂₇NO₆ |
| Fmoc-AEEA-OH | 166108-71-0 | 385.4 | C₂₁H₂₃NO₆ |
| Fmoc-Lys(Mtt)-OH | 167393-62-6 | 624.77 | C₄₁H₄₀N₂O₄ |
| Fmoc-Lys(ivDde)-OH | 204777-78-6 | 574.71 | C₃₄H₄₂N₂O₆ |
| Fmoc-Lys(Boc)-OH | 71989-26-9 | 468.54 | C₂₆H₃₂N₂O₆ |
| Bromoacetic acid | 79-08-3 | 138.95 | BrCH₂COOH |
| Bromoacetic anhydride | 13094-51-4 | 259.88 | (BrCH₂CO)₂O |
| Fmoc-Gly-OH | 29022-11-5 | 297.31 | C₁₇H₁₅NO₄ |
| Fmoc-Gly-Wang Resin | N/A | N/A | N/A |

The structure and characteristics of the acylated OXM analog are detailed in Table 6 below.

TABLE 6
Acylated OXM Analog Structure

| | |
|---|---|
| Sequence | H(Aib)QGTFTSDYSKYLDSKKAQEFVQWLLN(Aib) |
| and | GRNRNNIAC³⁸(Eicosanedioic-gGlu- |
| structure | (AEEA)₂-Lys(Ac)-Gly)-NH₂ (SEQ ID NO: |
| | 74) |
| Molecular | C₂₄₂H₃₈₂N₆₄O₇₄S |
| Formula | |
| Molecular | 5404.15 Da (calculated) |
| weight | |
| Theoretical | 8670 M⁻¹ cm⁻¹, or 1.604 Abs 0.1% |
| EC | (1 g/l) |
| (280 nm) | |

### 3.2: General Production Process

Several methodologies can be applied for the synthesis of the acylated OXM analogs. Each of such methodologies is governed by the different coupling chemistry required for the analogue synthesis.

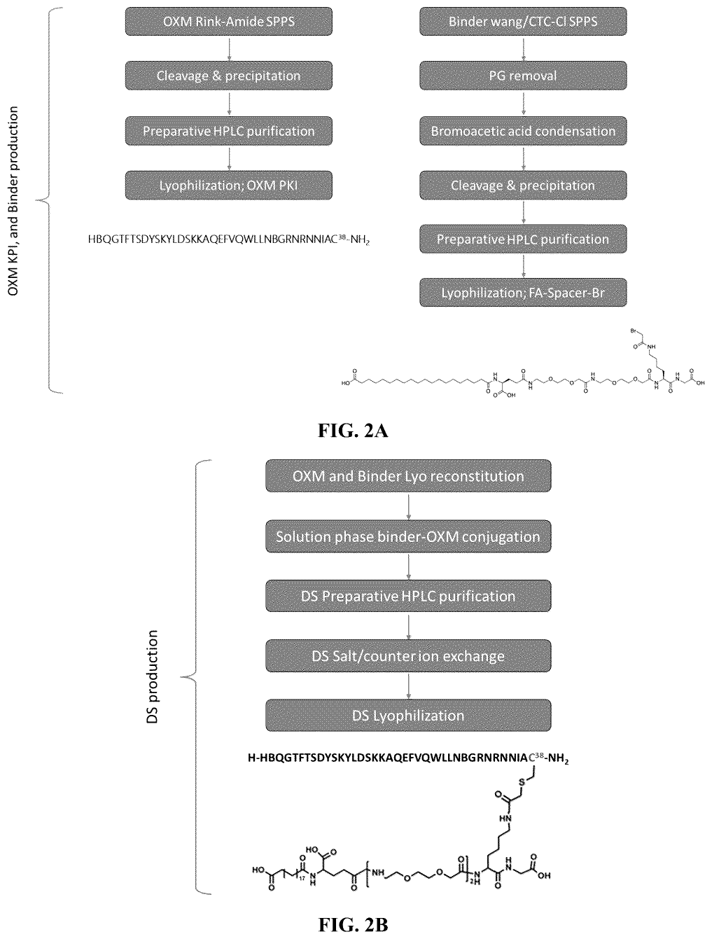

FIGS. 2A and 2B detail the methodology of two steps variant synthesis. FIG. 2A shows the general description of the production of the mutated OXM backbone and the binder complex. FIG. 2B shows the general description of the production of the acylated OXM analog.

The mutated OXM backbone is to be synthesized using solid-phase peptide synthesis (SPPS) methodology.

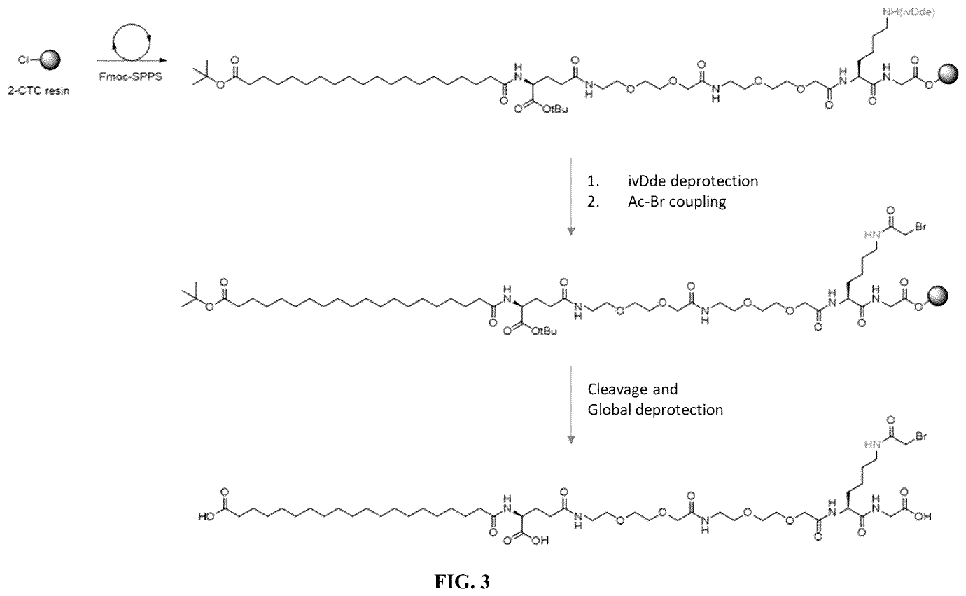

The fatty acid-peptide binder complex is to be synthesized using SPPS methodology, as suggested in FIG. 3, which shows the synthesis steps for construction of fatty acid-peptide complex (Binder). Both the CTC-2 resin and the ivDde PG in FIG. 3 are not mandatory and therefore are only optional.

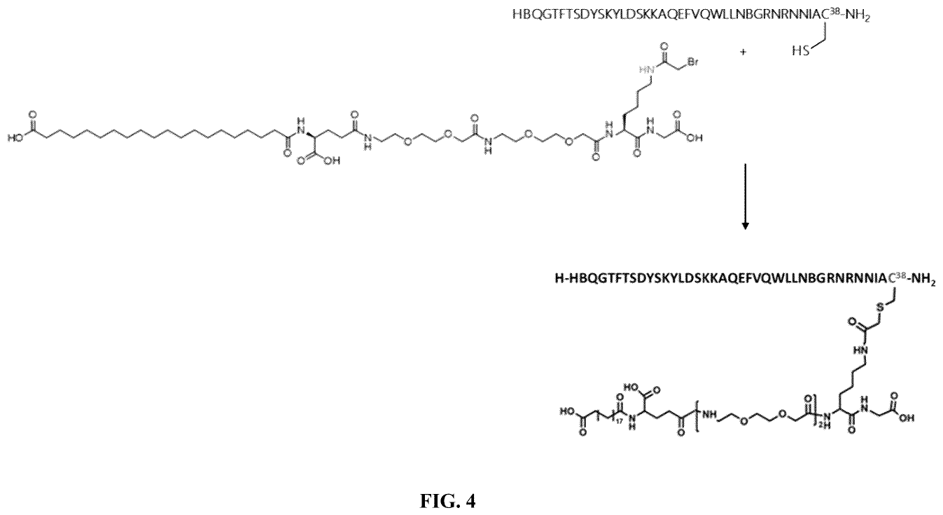

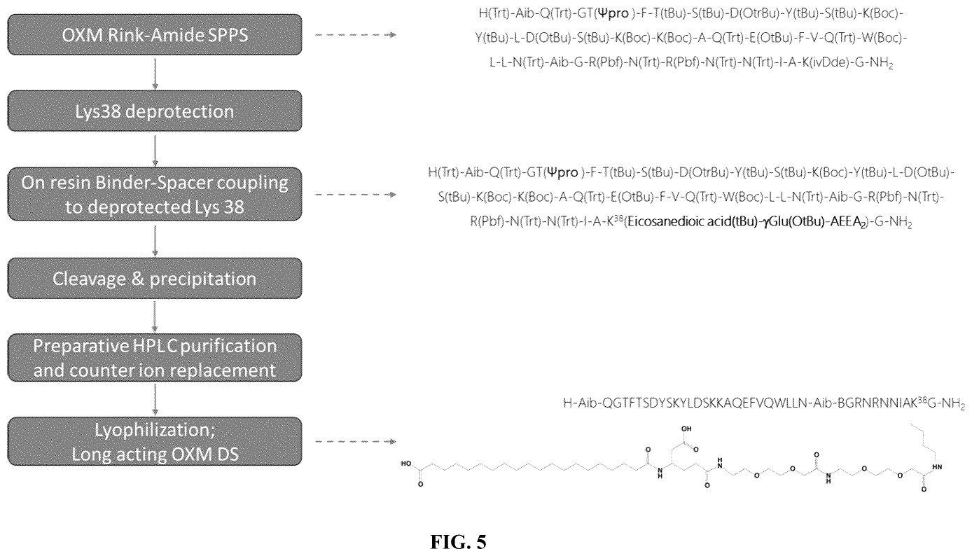

The conjugation of the Binder to the mutated OXM backbone is an SN2 reaction involving the thiol side chain of an OXM backbone cysteine residue (Cys38) and the Bromo acetamide of the binder. FIG. 4 shows the conjugation of a fatty acid-peptide complex (binder) to a mutated OXM backbone to produce an acylated OXM analog.

Alternatively to the conjugation step between the OXM backbone and the binder complex, an all on resin chain elongation methodology can also be applied, in order to synthesize the long acting OXM variants. FIG. 5 exemplifies such “all on resin” synthesis route, and consequent purification steps.

### 3.3: Specific Production Process

### Binder Synthesis

The binder backbone synthesis was performed with a 3.2 eq. excess of AA, Oxymapure and DIC, on Fmoc-Gly-Wang resin (preloaded-0.62 mmol/gr). Acetylation was performed after each coupling with acetic anhydride, and Fmoc deprotection was performed with 20% Piperidine in DMF. The acetylation was precautionary and therefore could be optional.

### Binder Cleavage

After the backbone was obtained, the peptide was cleaved from the resin with a cleavage cocktail containing TFA: TIS: H₂O, and precipitated with pre-cold IPE. Evaporation of the cocktail cleavage solution (˜80% from initial volume) increase precipitation yields.

### Acetyl Bromide Conjugation

The binder pellet was reconstituted in DMF, and bromoacetic anhydride was added to obtain the final binder structure. This conjugation can be performed with Bromoacetic acid on resin, while using orthogonal protecting groups on the Lys side chain.

### Purification

RP-HPLC was performed to obtain the purified binder.

Purification was performed on Luna C18(2) 10 um, 100 A, 2.12*25 cm column with 0.1% TFA in H₂O as mobile phase A and 0.1% TFA in ACN as mobile phase B. The reaction mixture was diluted with 30% ACN:70% H₂O until ˜3% of DMF were reached, filtered and loaded onto the column. The binder elutes from the column at about 60% MPB.

The fractions with purity >90% were pooled and evaporated until a concentration of ˜50% ACN is reached, in order to decrease the ACN content and to increase the pool concentration.

### Lyophilization

The fractions with purity >90% were pooled and evaporated until a concentration of ˜50% ACN is reached, in order to decrease the ACN content and to increase the pool concentration.

The final pool was filtered through a 0.22□m PVDF filter before lyophilization. The concentration and purity before and after filtration were similar.

### Analytical Methods in Use

Method parameters for Analytical HPLC and for similar variant and binder analysis are presented below in Table 7 and Table 8, respectively.

TABLE 7
Analytical HPLC Conditions for variant analysis

| | | | |
|---|---|---|---|
| Column | Acquity UPLC PST CSH C18, 50 × | Flow | 0.4 |
| | 2.1 mm, 1.7 um | | ml/min |
| MPA | 0.1% TFA/H2O | Column | 45° C. |
| | | Temp. | |
| MPB | 0.1% TFA/CAN | Wave | 220 nm |
| | | Length | |
| Gradient | 10% →10% in 0.2 min → 30% in | | |
| (MPB) | 0.3 min → 60% in 7 min | | |

TABLE 8
Analytical HPLC Conditions for binder analysis

| | | | |
|---|---|---|---|
| Column | Acquity UPLC CSH C18, 50 × | Flow | 0.4 |
| | 2.1 mm, 1.7 um | | ml/min |
| MPA | 0.1% TFA/H₂O | Column | 45° C. |
| | | Temp. | |
| MPB | 0.1% TFA/ACN | Wave | 220 nm |
| | | Length | |
| Gradient | 5% → 5% in 0.2 min → 20% in | | |
| (MPB) | 0.3 min → 90% in 7 min | | |

### Example 4

### Comparative Characteristics of Modified OXM Analog

The modified OXM analogs are a considerable improvement over existing GLP-1 agonists and prior incarnations of the OXM analog alone (SEQ ID NO: 3 where Xaa38=Cys and Xaa39=Cys) or the PEGylated OXM analog (SEQ ID NO: 3 where Xaa38=Cys-PEG20 and Xaa39=Cys-PEG20).

In prior studies, the OXM analog of SEQ ID NO: 3 demonstrated good efficacy and safety profile, achieving good HbA1c reduction, weight loss, and improved lipid profile. However, because of a dose limitation, it was not able to achieve the weight loss observed with the leading GLP-1 therapies such as semaglutide and tirzepatide.

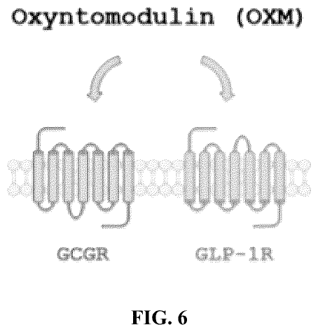

The potencies of the modified OXM analogs newly disclosed in the present application, the OXM Analog alone, and the PEGylated OXM analog were examined and compared by measuring the EC₅₀ of both receptors, glucagon-like peptide-1 (GLP1) and glucagon (GCGR). FIG. 6 illustrates the dual agonism of OXM or an OXM analog.

The OXM analog alone (SEQ ID NO: 3 without any PEGylation) had a GLP-1 activity (EC₅₀=0.105) that was similar to semaglutide (0.11 nM) and superior to tirzepatide (0.9 nM).

PEGylation of the OXM analog inhibited GLP-1 activity (EC₅₀=2.75) by over 20-fold and increased the molecular weight by approximately 10-fold requiring a significant increase in the dose needed to achieve competitive weight loss.

PEGylation of the OXM peptide also produced high viscosity of the formulated product limiting the maximal dose to 70 mg, preventing the use of higher doses to compete with the leading GLP-1 therapies.

The newly designed and disclosed modified OXM analogs replace PEGylation with a lower molecular weight molecule that overcomes the PEG-related limitations and allow the optimal dosing to harness maximal efficacy of the OXM analog.

Table 9 and Table 10 below show the comparison of the characteristics of the newly designed modified OXM analogs, the OXM analog alone, the PEGylated OXM analog, and comparable GLP-1 therapeutics.

TABLE 9

| | | | Approx. | Est. Human | |
|---|---|---|---|---|---|
| Composition | Formulation | EC₅₀ (nM) | MW | Dose | |
| OXM Analog | Non-pegylated | 0.105 | 4.4K | N/A | |
| (SEQ ID NO: 3) | (SEQ ID NO: 3 where | | | | |
| | Xaa38 = Cys and Xaa39 = | | | | |
| | Cys) | | | | |
| | PEGylated | 2.75 | 42-50K | >70 | mg |
| | (SEQ ID NO: 3 where | | | | |
| | Xaa38 = Cys-PEG20 and | | | | |
| | Xaa39 = Cys-PEG20) | | | | |
| Modified OXM Analogs | Non-formulated | 0.13 | 4.4K | N/A | |
| | (SEQ ID NO: 5 to SEQ ID | | | | |
| | NO: 7) | | | | |
| | Variant 1 | 0.09 | 5.8K | 2-6 | mg |
| | (Formula XIV) | | | | |
| | Variant 2 | 0.10 | 4.7K | 2-6 | mg |
| | (Formula XV) | | | | |
| | Variant 3 | 0.15 | 5.66K | 2-6 | mg |
| | (Formula XVI) | | | | |
| | FA-conjugated | 0.21 | 5.76K | | |
| | (Mixture of Formula XXII | | | | |
| | and Formula XXIII) | | | | |

| | | | | |
|---|---|---|---|---|
| Semaglutide (Wegovy ®, Ozempic ®) | 0.11 | 4.1K | 2.4-6.2 | mg |
| Tirzepatide (Mounjaro ®, Zepbound ®) | 0.9 | 4.8K | 15-22 | mg |

The levels of cAMP (cyclic adenosine monophosphate) will be measured in CHO-K1 cells that overexpress GLP-1 or GCGR in future studies. Camp levels will be measured by Camp Hunter assay (DiscoverX™) based on enzyme fragment complementation of 2 fragments of b-gal enzyme (ED and EA. Free cAMP from cell lysates will compete for antibody binding against cAMP (ED-cAMP conjugate). Unbound ED-cAMP will be free to complement EA to form active b-gal enzyme, which will subsequently hydrolyze the substrate to produce signal. A positive chemiluminescent signal generated will be directly proportional to the amount of free cAMP bound by the antibody.

### Example 5

### Potency and Purity of Modified OXM Analogs

The purpose of this experiment is to study the influence of fatty acid and/or binder complex conjugation on the potency of particular OXM analog backbones.

### Compositions

“OPK-88003 KPI” is defined as SEQ ID NO: 3 where Xaa38=Cys and Xaa39=Cys.

“OPK-88003 API” is defined as SEQ ID NO: 3 where Xaa38=Cys-PEG20 and Xaa39=Cys-PEG20.

“OPK-88003 FA mono conjugate” is defined as either the modified OXM of Formula XXII or the modified OXM of Formula XXIII. Either of these compositions is also referred to as “KPI+1*Binder”.

“OPK-88003 combination of mono conjugates” is defined as the modified OXM of Formula XXV. This composition is also referred to as “KPI+2*-Kinder”.

“MOD-604” is defined as the mutated OXM4 analog of any one of SEQ TD NOs: 5 to 7.

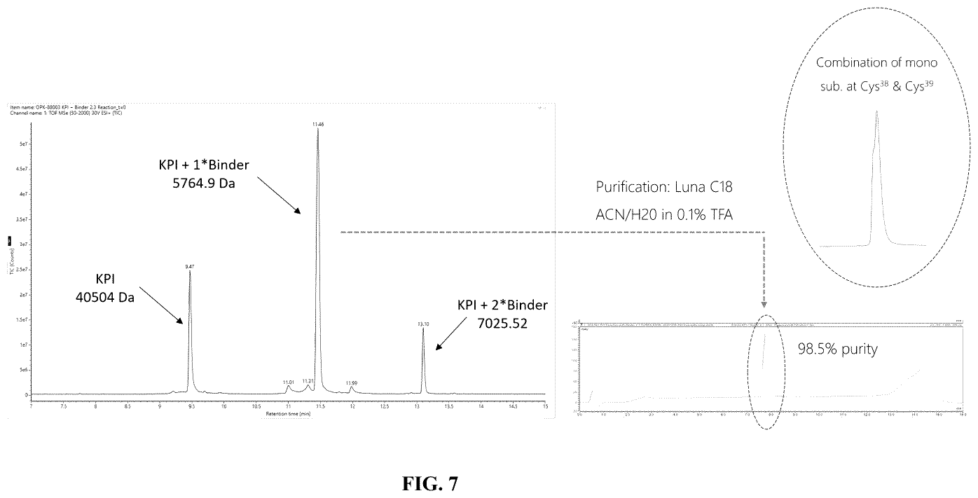

FIG. 7 shows the chromatogram for the OPK-88003 KPI, OPK-88003 FA mono conjugate, and the OPK-88003 combination of mono conjugates. Additionally, the purity of the OPK-88003 FA mono conjugate is shown.

The impact of the longevity-enabling molecule (PEG Vs. FA-peptide/binder complex) on the peptide potency was studied.

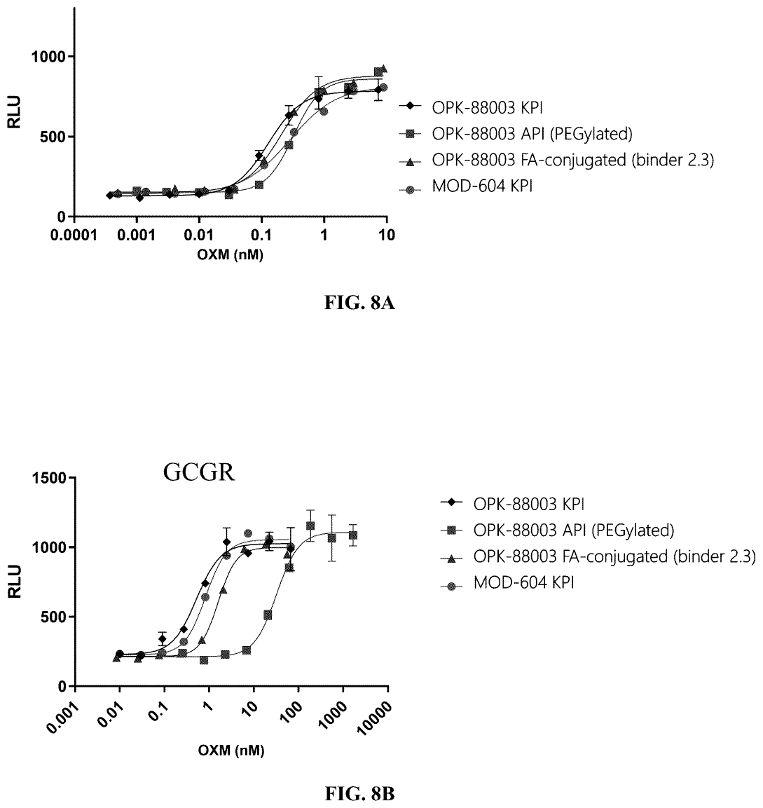

FIGS. 8A and 8B show the CBA potency assays demonstrating the peptide potency of various OXM analogs and modified OXM analogs. Table 10 below shows the quantitative data from these assays

TABLE 10

| | | | | | | | Activity fold | |
|---|---|---|---|---|---|---|---|---|
| | | | | | | | reduction | |
| | | | | EC50 ratio | | | compared to | |
| | | EC50 [nM] | EC50 [nM] | (GCGR\GLP- | % EC50 | | OPK-88003 | |
| Product | Identification | GLP1 | GCGR | 1R) | compare to KPI | | KPI | |
| OPK-88003 | KPI | 0.127 | 0.60 | 4.7 | 100 | | | |
| OPK-88003 | API | 1.18 | 32.1 | 27.2 | 10.8 | (GLP1) | 14 | (GLP1) |
| | (PEGylated) | | | | 1.9 | (GCGR) | 53.5 | (GCGR) |
| OPK-88003 | FA-conjugate | 0.21 | 1.60 | 7.6 | 60 | (GLP1) | 1.65 | (GLP1) |
| | | | | | 38 | (GCGR) | 2.65 | (GCGR) |
| MOD-604 | KPI | 0.282 | 0.83 | 2.9 | 45 | (GLP1) | 2.2 | (GLP1) |
| | | | | | 72 | (GCGR) | 1.4 | (GCGR) |

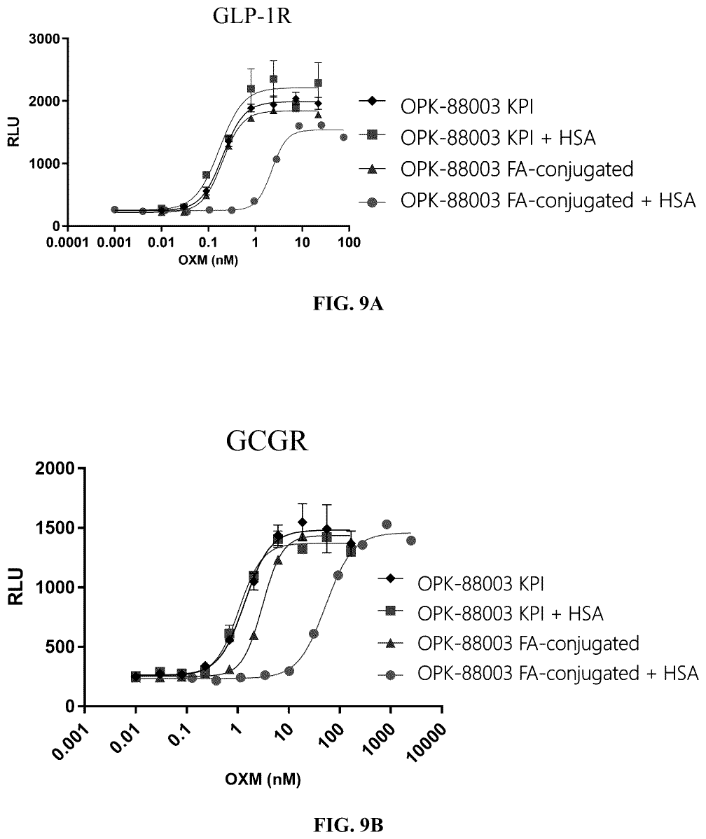

FIGS. 9A and 9B are CBA potency assays which qualitatively show the conjugate potential to bind HSA. Table 11 below shows the quantitative data from these assays. Significant reduction of the modified OXM potency in the presence of HSA will imply on the FA interaction with Albumin.

TABLE 11

| | | | | | | | Activity fold | |
|---|---|---|---|---|---|---|---|---|
| | | | | | | | reduction | |
| | | | | EC50 ratio | | | compared to | |
| | | EC50 [nM] | EC50 [nM] | (GCGR\GLP- | % EC50 | | OPK-88003 | |
| Product | identification | GLP1 | GCGR | 1R) | compare to KPI | | KPI | |
| OPK-88003 | KPI | 0.201 | 1.37 | 6.8 | 100 | | | |
| OPK-88003 | KPI + HSA | 0.187 | 1.06 | 5.7 | 107 | (GLP1) | | |
| | | | | | 129 | (GCGR) | | |
| OPK-88003 | FA-conjugate | 0.2 | 3.04 | 15.2 | 100 | (GLP1) | non | (GLP1) |
| | | | | | 45 | (GCGR) | 2.2 | (GCGR) |
| OPK-88003 | FA-conjugate + | 2.27 | 52.9 | 23.3 | 8.9 | (GLP1) | 11.2 | (GLP1) |
| | HSA | | | | 2.6 | (GCGR) | 38.6 | (GCGR) |

### Example 6

### Development of Second Generation of Long-Acting Acylated Oxyntomodulin Analogs to Enhance Potency and Dosing Regimen

A second generation of long-acting oxyntomodulin was developed for the treatment of obesity, type-2 diabetes, cardiovascular disease, and NASH.

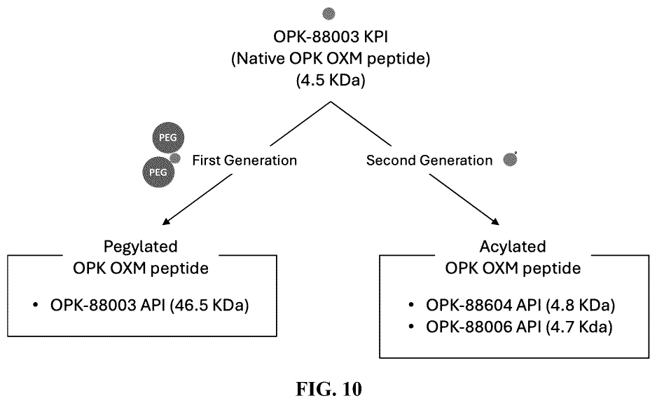

FIG. 10 shows the development of a second generation of long-acting acylated oxyntomodulin analogs to enhance potency and dosing regimen. The potential benefits of novel acylated OXM products relative to the PEGylated OXM peptide were evaluated for GLP-1 and Glucagon activity in vitro, molecular weight, dosing, and efficacy in a DIO Mouse model.

Table 12 shows the comparison of PEGylated and acylated OXM peptide analogs for molecular weight, GLP-1 and glucagon activity, and ratio of EC50 activity.

TABLE 12
Comparison of PEGylated and acylated OXM peptide analogs

| | EC50 (nM) | | GCGR/ | |
|---|---|---|---|---|
| | GLP- | | GLP- | Molecular |
| Formulation | 1R | GCGR | 1R | Weight |
| OPK-88003 KPI (Native OPK | 0.0168 | 0.0731 | 4.4 | 4.5 KDa |
| OXM peptide) | | | | |
| OPK-88003 API (Pegylated | 0.267 | 16.8 | 62.9 | 46.5 KDa |
| OPK OXM peptide) | | | | |
| OPK-88604 (2nd Gen. | 0.0159 | 0.164 | 10.3 | 4.8 KDa |
| Acylated OPK OXM peptide) | | | | |
| OPK-88006 (2nd Gen. | 0.019 | 0.31 | 16.3 | 4.7 KDa |
| Acylated OPK OXM peptide) | | | | |

“OPK-88604” is defined as the modified OXM of Formula XIV.

“OPK-88006” is defined as the modified OXM of Formula XVIII.

Novel and acylated long-acting OXM peptide analogs were synthesized and identified with targeted GLP-1 and Glucagon activities. The potency of the acylated OXM peptides was compared with the native OXM peptide alone and Pegylated OXM peptide to evaluate the impact of acylation on GLP-1 and Glucagon activities.

PEGylation of the OXM peptide decreased both GLP-1 activity (by 10-15-fold) and glucagon activity (by over 20-fold) in OPK-88003. PEGylation also preferentially decreased glucagon activity relative to the glucagon activity as reflected in the EC50 GCGR/GLP-1 ratio. Further PEGylation also increased the molecular weight of the OXM peptide by over 40 KDa which impacts the amount and volume needed for dosing in a therapeutic setting. In contrast, acylation of the native OXM peptide resulted in only a modest decrease in GLP-1 and Glucagon activity, and little impact on the ratio of GCGR/GLP-1 activity relative to the pegylated API, OPK-88003. In comparison, acylation of OXM peptide increased the molecular weight only slightly by 0.2-0.3 KDa, thus reducing the amount and volume of OXM peptide that would be required for dosing in a therapeutic regimen.

### Conclusions

In summary, the overall benefits of acylation over PEGylation of the OXM peptide included: Reduced molecular weight by approximately 10-fold; Reduced volume and viscosity for administration; Increased potency of the OXM peptide which allows for higher dosing and efficacy.

The acylated OXM peptides have more acceptable weekly exposure by binding to endogenous serum albumin compared to the PEGylated of OXM peptide.

### Example 7

Effect of Treatment with Long-Acting Acylated Oxyntomodulin Analogs on Metabolic Parameters and Glycemic Control in Dio Mice

### I: OVERVIEW

### II. METHODS

II(A) Summary of Methods: DIO mice (45-50 g) were treated daily with placebo, 15 nmol/kg of OPK-88604, or 15 nmol/kg of semaglutide for a period of 28 days, or were treated with placebo, 30 nmoles/kg of OPK-88604, or 30 nmoles/kg of OPK-88006 for 4 weeks. The animals were individually weighed and analyzed for lipid and cholesterol levels prior to treatment and following completion of the study. The levels of fasting glucose and insulin levels were analyzed, and oral glucose tolerance test was performed and compared between groups The levels of plasma triglycerides and cholesterol were evaluated at the end of study and compared to base levels prior to treatment. Levels of total cholesterol, triglycerides and glycogen were also examined at the end of study. Histology of liver was used to analyze for lipid droplets in the liver. All tests were conducted using routine methods known in the art.

### II(B) Statistical Analysis

Comparison of data and significant differences for multiple testing were graphically visualized when p<0.05.

For single-timepoint continuous data, the data was fitted to a one-factor linear regression model with the treatment groups as categorical, independent (predictor) variables, and Dunnett's test used to compare treatments to control.

For continuous data with repeated sample collection over time, the data was fitted to a two-factor linear regression model with the treatment groups and time points as categorical independent (predictor) variables with interaction. Dunnett's test was used to compare treatments to control for each timepoint.

Data from categorical endpoints, such as histopathological scoring values, were partitioned into a 2×2 contingency table containing responders and non-responders in control and treatment groups. Fisher's exact test was employed and assumes that contingency table row and column totals were fixed, sampling was random, and observations can be classified only into one cell. The reported p-values from all pairwise comparisons were adjusted using the Bonferroni correction.

### II(C)Diet-Induced Animal Models

The mice used in the studies were the gubra diet-induced obese (DIO) mouse model. This mouse model is fed a high fat diet that results in rapid weight gain. The DIO mice were based on male C57BI/6J mice and ready for experiment after 18 weeks on a 60% high fat diet.

### II(D) Assessment of the Body Loss

To ensure accurate monitoring of weight changes over the course of the study, the animals were weighed daily with precision, prior to dosing at 7:00 AM. Body weight data was recorded in two formats: (1) Absolute measurements: The direct weight of the animal each day, and (2) Relative measurements: The weight expressed as a percentage relative to the body weight recorded on Day 1.

### II(E) Blood Sampling and Plasma Preparation

For in vivo blood samples, tail-vein, tongue or cheek blood was collected in a Microvette tube of appropriate dimensions with anticoagulant and mixed by inversion 5 times. Blood was placed at 4° C. until centrifuged at 3000 g for 10 minutes. The plasma supernatants were transferred to new tubes and immediately frozen on dry ice. The samples were stored at −70° C.

For termination blood samples, during anesthesia with isoflurane, the abdominal cavity was opened, and cardiac blood was drawn with a syringe into a Microvette/Vacuette of appropriate dimensions with anticoagulant and mixed by inversion 5 times. Blood was placed at 4° C. until centrifuged at 3000 g for 10 minutes. The plasma supernatants were transferred to new tubes and immediately frozen on dry ice. The samples were stored at −70° C.

### II(F) Tissue Sampling of Liver (NASH)

After termination, the liver was collected and weighed. Specific liver samples and biopsies were dissected and processed as specified in table “Termination samples” in the protocol and further described below.

The liver was divided into a left lateral lobe, medial lobe, right lateral lobe, and caudate lobe. The remaining lobes were not used unless specified in the protocol.

The Post-biopsy piece (˜200 mg, less than 0.7×0.5 cm) was cut from the left lateral lobe, 4 mm from the prebiopsy site with an edge. The tissue was collected in 10% neutral buffered formalin and fixated for 24 hours at room temperature.

The Liver Sponsor piece (˜150 mg) qA dissected from the left medial lobe, collected in tubes and frozen in liquid nitrogen. The samples were stored at −70° C. The size of the sponsor piece varied according to the size of the left medial lobe.

The Liver TG TC piece (25±5 mg) was dissected from the right medial lobe, with no edges, on the opposite side of the sponsor piece. The samples were weighed individually, collected in tubes and frozen in liquid nitrogen. The samples were stored at −70° C.

The Liver Frozen piece for Oil-Red-O staining (˜150 mg) was dissected from the left lateral lobe, collected in tubes and frozen in liquid nitrogen. The samples were stored at −70° C.

The Liver Extra piece (˜100-300 mg) was dissected from the right lateral lobe, collected in tubes and frozen in liquid nitrogen. This sample can be used as backup tissue from the study and can be used for re-analysis if necessary. The samples were stored at −70° C.

### Glucose Tolerance Test

Animals were housed in a clean cage and fasted for 4 hours prior to oral or intraperitoneal glucose administration. At timepoint 0, mice receive an oral or intraperitoneal dose of 10 ml/kg of 200 mg/ml standard glucose solution (Fresenius Kabi, DK). At timepoint 0, mice received an oral or intraperitoneal dose of 4 ml/kg of standard 500 mg/ml glucose solution (Fresenius Kabi, DK). Blood samples (10 μl) were collected at various timepoints defined in the study protocol into heparinized glass capillary tubes and immediately suspended in glucose/lactate system solution buffer (EKF-diagnostics, Germany). Blood glucose (BG) was measured using a BIOSEN c-Line glucose meter (EKF-diagnostics, Germany) according to the manufacturer's instructions. After the last blood sample and/or drug dose, the animals were returned to the normal feeding schedule.

### II(G) Histological Staining Procedures

In summary, glass slides with paraffin embedded sections were deparaffinated in xylene and rehydrated in series of graded ethanol.

Slides treated with histochemical and immunohistochemical stains were scanned using a 20× objective in a ScanScope AT slide scanner (Aperio).

Slides treated with fluorescence stains were scanned using a 20× objective on an Olympus VS120 slide scanner (Leica), equipped with the appropriate fluorescent filters.

Hematoxylin & Eosin (H&E) staining: Sections of FFPE tissue were incubated in Mayer's Hematoxylin (Dako), washed in tap water, stained in Eosin Y solution (Sigma-Aldrich), dehydrated in graded ethanol and cover slipped.

### II(H) Blood and Plasma Assays

Alanine transaminase (ALT), Aspartate transaminase (AST), Triglycerides (TG) and Total Cholesterol (TC): Blood samples were collected in heparinized tubes and plasma was separated and stored at −70° C. until analysis. Samples were measured using commercial kits (Roche Diagnostics), on the cobas c 501 autoanalyzer.

Insulin: Blood samples were collected in heparinized tubes and plasma was separated and stored at −70° C. until analysis. Insulin was measured using the commercial MSD platform (Meso Scale Diagnostics).

### II(I) Tissue Assays

Liver Triglycerides (TG) and Total cholesterol (TC): The liver content of triglyceride and total cholesterol was extracted from the liver samples into 5% NP-40, in a homogenization process followed by a heating step, where the samples were heated twice to 90° C. The samples were then centrifuged, and the supernatant was collected. The TG and TC levels were then determined by measurement on the cobas c 501 autoanalyzer with the relevant commercial kits from Roche Diagnostics.

### II(J) EchoMRI Body Composition for Body Composition Analyses

The body composition of mice was assessed by an EchoMRI 3 in 1 Body composition analyzer (EchoMRI, US). Non-anaesthetized animals were placed in a plastic tube inside the MRI scanner for approximately 80 seconds. The body composition was reported as fat mass and fat free mass (lean mass).

### III. EFFECT OF TREATMENT WITH OPK-88604 ON METABOLIC PARAMETERS AND GLYCEMIC CONTROL IN MALE DIO MICE

The aim of this experiment (Example 7(III)) was to investigate the effect of 4 weeks of daily treatment with OPK-88604 on metabolic parameters and glycemic control in male DIG mice.

Sixty C57BL/6JRj male mice were fed a high fat diet (60% HFD, D12492—Ssniff) for 20 weeks before treatment start. The animals were randomized by body weight and fat mass into 6 groups (n=10 per group) and treated with daily subcutaneous administration of vehicle, OPK-88604 low (2 nmol/kg), OPK-88604 mid (5 nmol/kg), OPK-88604 high (15 nmol/kg), Semaglutide (15 nmol/kg) and Tirzepatide (15 nmol/kg) for 4 weeks. Body weight and food intake (24 h) were measured daily. In week 3, an OGTT study was performed. Baseline and preterminal fasting blood glucose and plasma insulin were assessed. Preterminal plasma TC and TG were assessed. Terminal endpoints included liver and heart weight, liver TC, TG, glucagon and lipid content (HE staining).

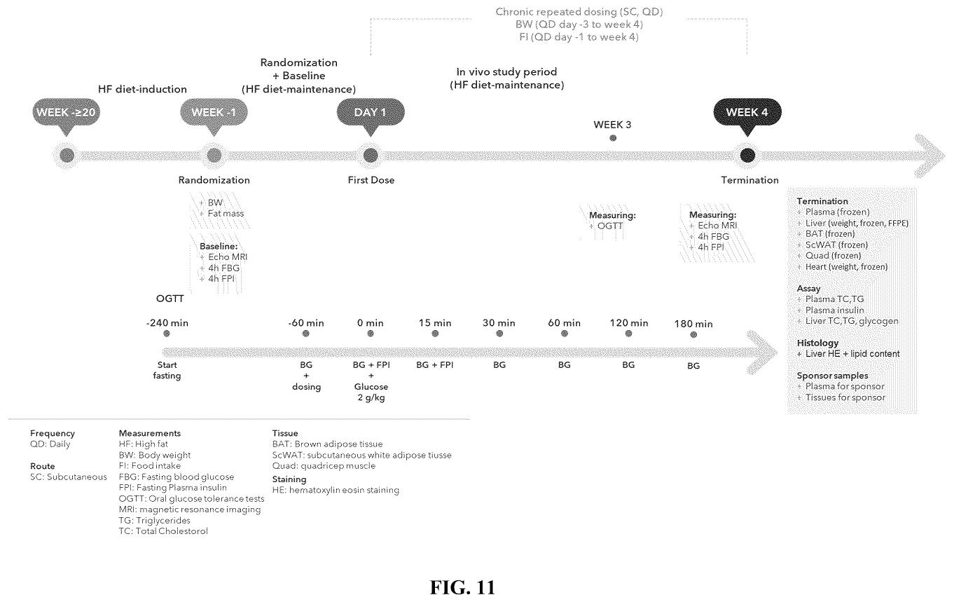

FIG. 11 shows the study outline of the experiment in Example 7(III). Table 13 below shows the study group summary for the experiment in Example 7(III).

TABLE 13
Study Group Summary

| | | n in | | Dose | Route of | Volume | Dosing |
|---|---|---|---|---|---|---|---|
| # | Group name | group | Animal model | (nmol/kg) | administration | (ml/kg) | Frequency |
| 1 | Vehicle | 10 | DIO | NA | SC | 5 | QD |
| 2 | OPK-88604 | 10 | DIO | 2 | SC | 5 | QD |
| | low | | | | | | |
| 3 | OPK-88604 | 10 | DIO | 5 | SC | 5 | QD |
| | mid | | | | | | |
| 4 | OPK-88604 | 10 | DIO | 15 | SC | 5 | QD |
| | high | | | | | | |
| 5 | Semaglutide | 10 | DIO | 15 | SC | 5 | QD |
| 6 | Tirzepatide | 9 | DIO | 15 | SC | 5 | QD |

### Results

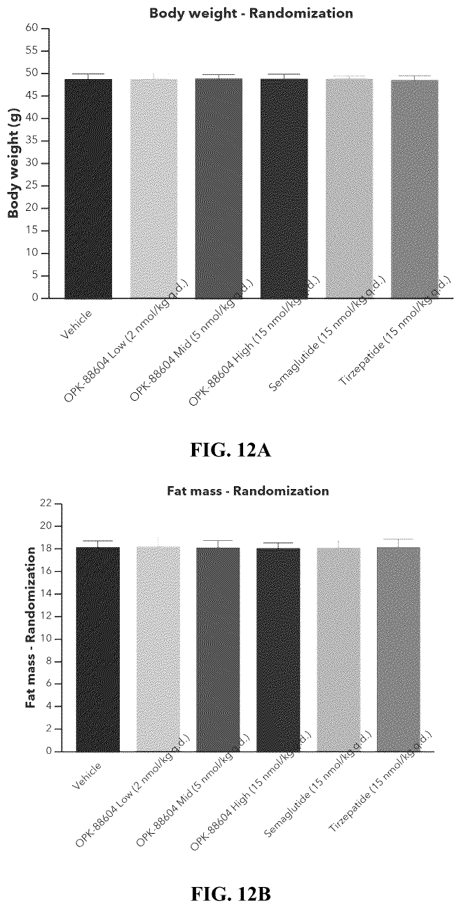

FIG. 12A shows the body weight randomization, and FIG. 12B shows the fat mass randomization in the present study. The values were expressed as mean of n=10+SEM and compared using Dunnett's test one-factor linear model. There were no differences at significance level 0.05.

### III(A): Body Weight Profile and Food Intake

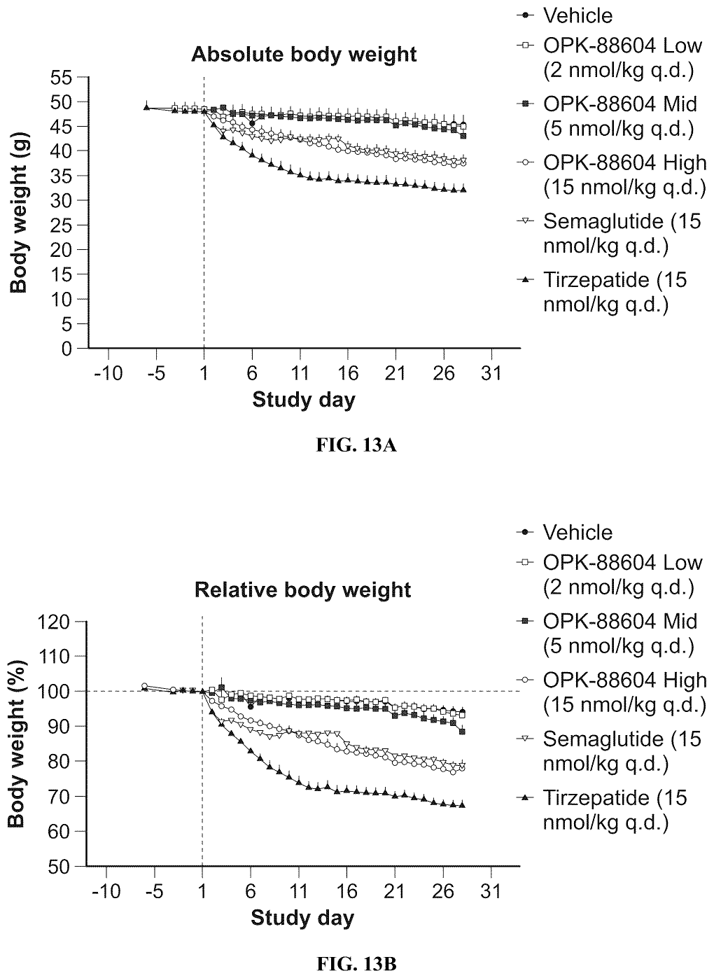

FIG. 13A shows the absolute body weight and FIG. 13B shows relative body weight profiles. The values were expressed as mean of n=8-10+SEM.

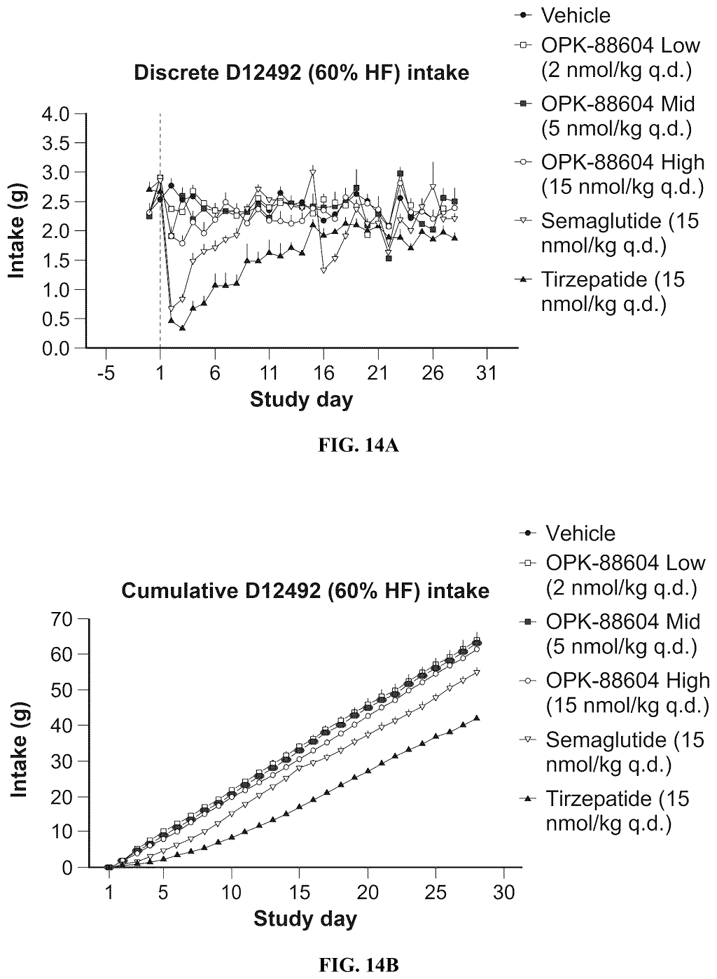

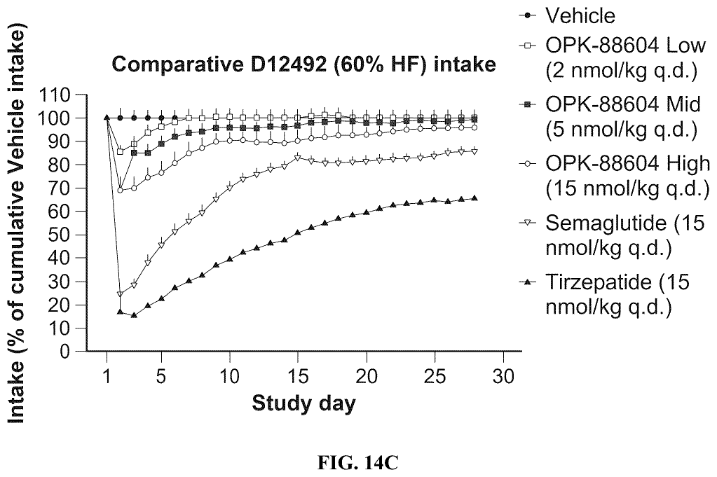

FIG. 14A shows the discrete D12492 (60% HF) food intake, FIG. 14B shows the cumulative D12492 (60% HF) food intake, and FIG. 14C shows the comparative D12492 (60% HF) food intake. The values were expressed as mean of n=8-10+SEM.

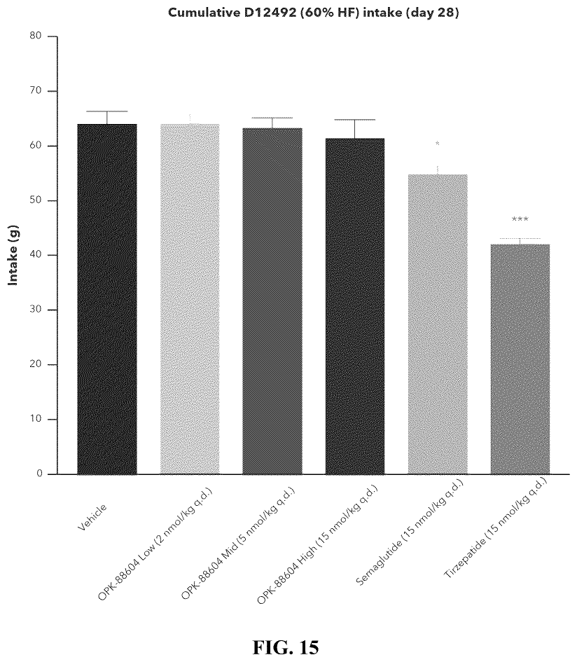

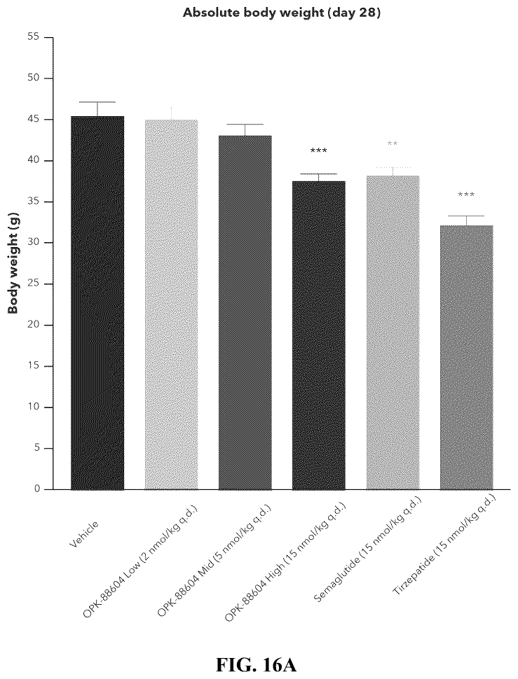

FIG. 15 shows the cumulative food intake on day 28. The values were expressed as mean of n=8-10+SEM and compared using dunnett's test one-factor linear model. For semaglutide (15 nmol/kg q.d.), P<0.05 compared to Vehicle. For tirzepatide (15 nmol/kg), P<0.001 compared to Vehicle.

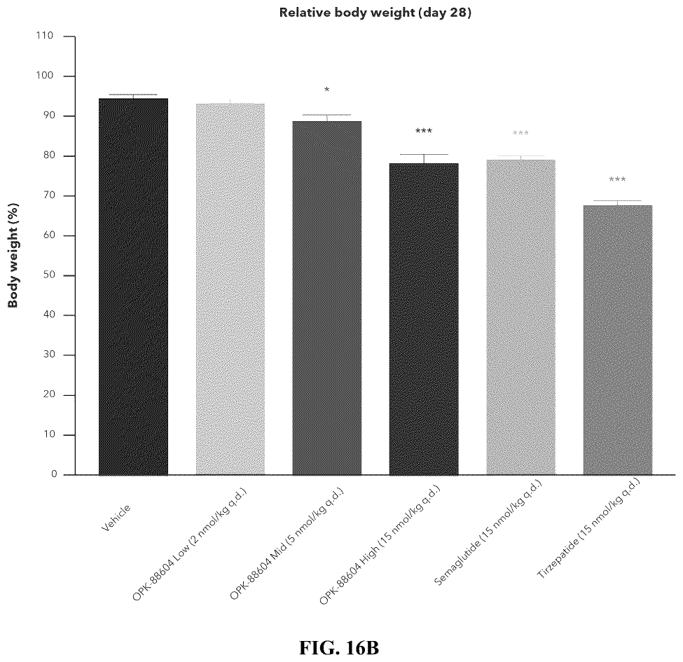

FIG. 16A shows the absolute body weight at day 28 and FIG. 16B shows the relative body weight (day 28). Values were expressed as mean of n=8-10+SEM and compared using Dunnett's test one-factor linear model. In FIG. 16A, semaglutide (15 nmol/kg) had P<0.01 compared to Vehicle, and OPK-88604 High (15 nmol/kg) and Tirzepatide (15 nmol/kg) had P<0.001 compared to Vehicle. In FIG. 16B, OPK-88604 Mid (5 nmol/kg) had P<0.05 compared to Vehicle and OPK-88604 High (15 nmol/kg), Semaglutide (15 nmol/kg), and Tirzepatide (15 nmol/kg) had P<0.001 compared to Vehicle.

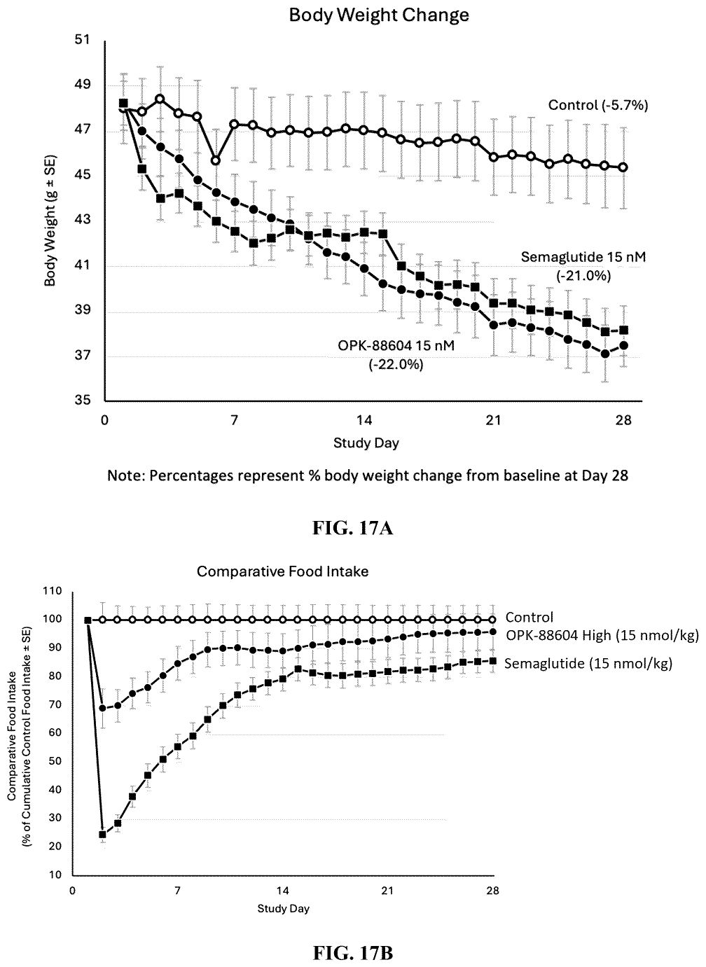

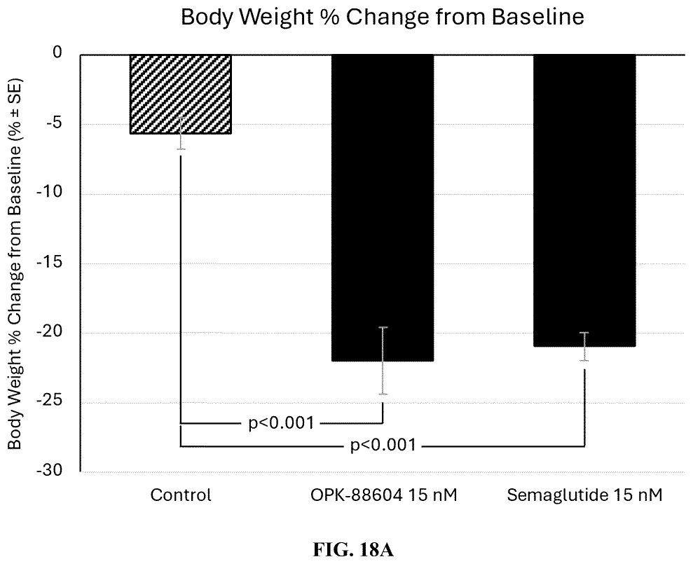

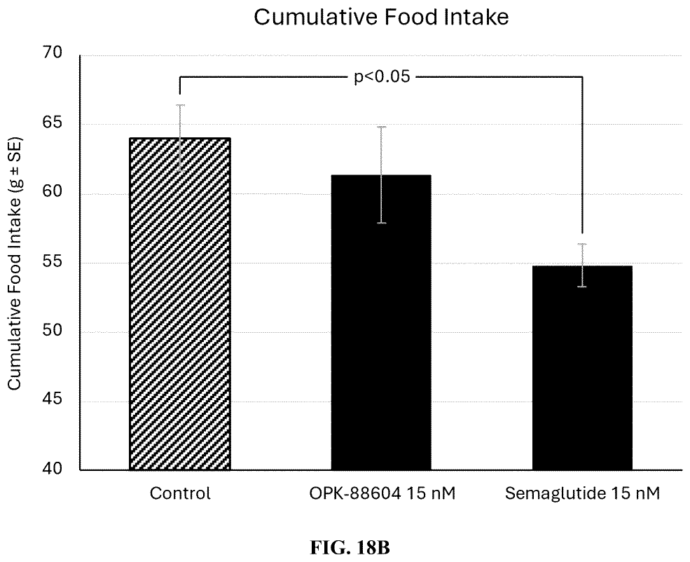

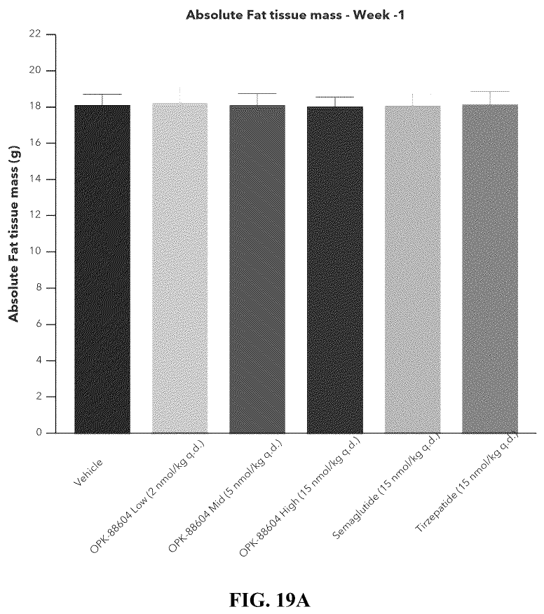

FIGS. 17A and 17B focuses on the body weight profile (FIG. 17A) and comparative food intake (FIG. 17B) following treatment with OPK-88604 and semaglutide. Treatment with acylated OPK-88604 and semaglutide over a 28 day period resulted in similar weight loss from baseline compared to placebo. Semaglutide reduced food consumption significantly more than OPK-88604 treatment, suggesting potentially a role for a separate mechanism of action such as an increase in energy expenditure that may contribute to weight loss with OPK-8860 treatment. For these studies the animals in each group were weighed daily and the amount of food consumed daily was determined.

FIGS. 18A and 18B show body weight (FIG. 12A) and cumulative food intake (FIG. 12B) on day 28 with different treatments (Placebo, OPK-88604, and semaglutide). The body weight and cumulative food intake over 28 days was determined for each group of animals treated with placebo, 15 nmol/kg of OPK-88604 and 15 nmol/kg of semaglutide. The body weight loss at the end of the study was similar for OPK-88604 and semaglutide treatments. The cumulative food intake over the period of the study was significantly decreased with semaglutide compared to acylated OPK-88604. These data suggest that OPK-88604 treatment may increase energy expenditure contributing to weight loss.

### III(B): Fat Mass and Lean Body Mass Change

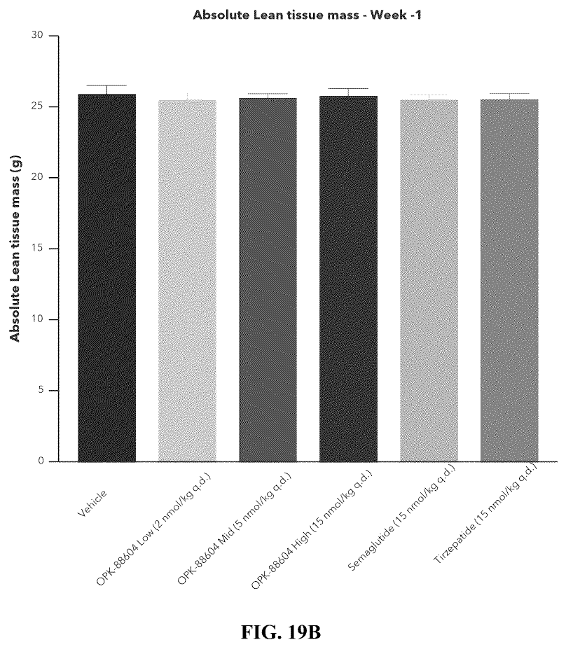

FIG. 19A shows the absolute fat tissue mass at week 1, and FIG. 19B shows the absolute lean tissue mass at week 1. The values were expressed as mean of n=10+SEM and compared using Dunnett's test one-factor linear model. There were no differences at significance level 0.05.

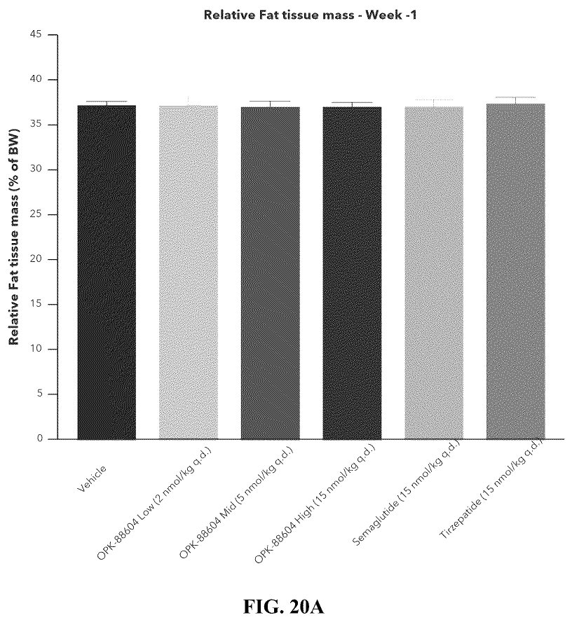

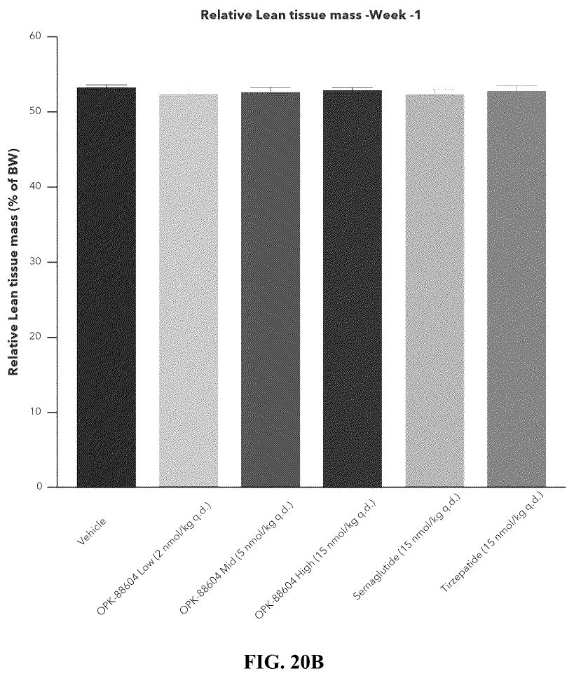

FIG. 20A shows the relative fat tissue mass on week 1, and FIG. 20B shows the relative lean tissue mass on week 1. The values were expressed as mean of n=10+SEM and compared using Dunnett's test one-factor linear model. There were no differences at significance level 0.05.

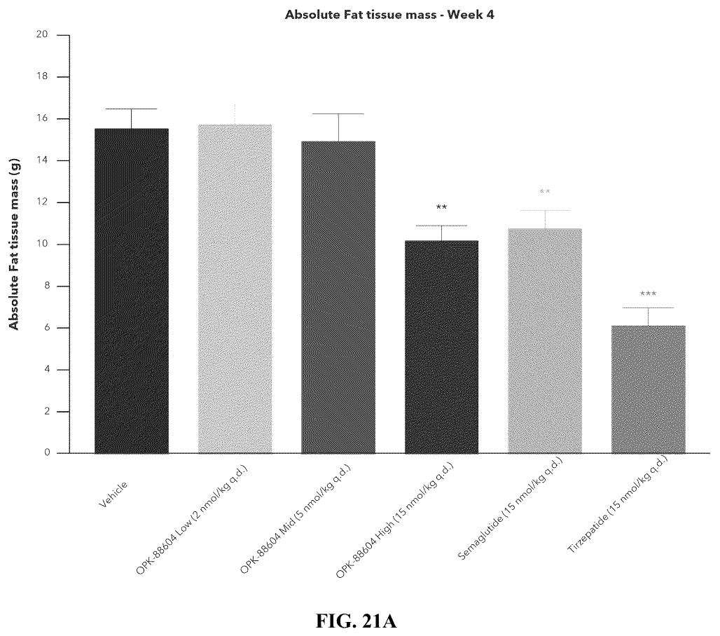

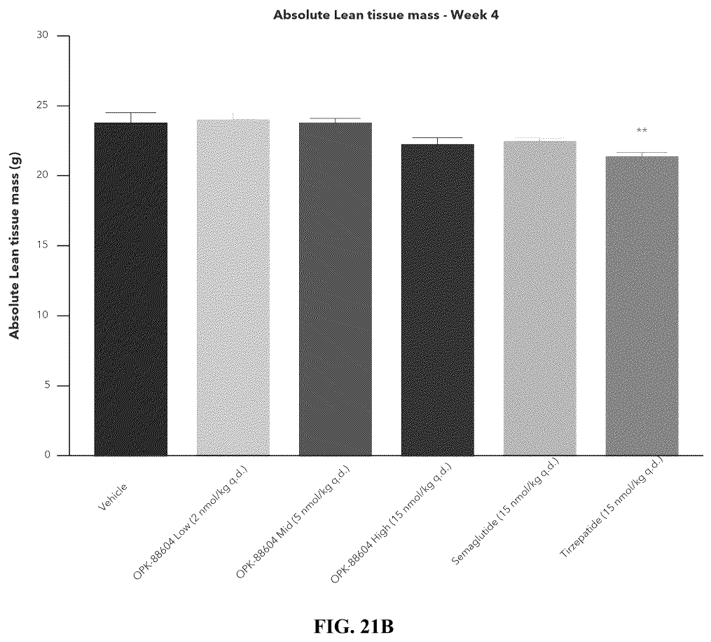

FIG. 21A shows the absolute fat tissue mass at week 4, and FIG. 21B shows the absolute lean tissue mass at week 4. The values were expressed as mean of n=9-10+SEM and compared using Dunnett's test one-factor linear model. In FIG. 21A, OPK-88604 High (15 nmol/kg) and Semaglutide (15 nmol/kg) had P<0.01 compared to Vehicle, and Tirzepatide (15 nmol/kg) had P<0.001 compared to Vehicle. In FIG. 21B, Tirzepatide (15 nmol/kg) had P<0.01 compared to Vehicle.

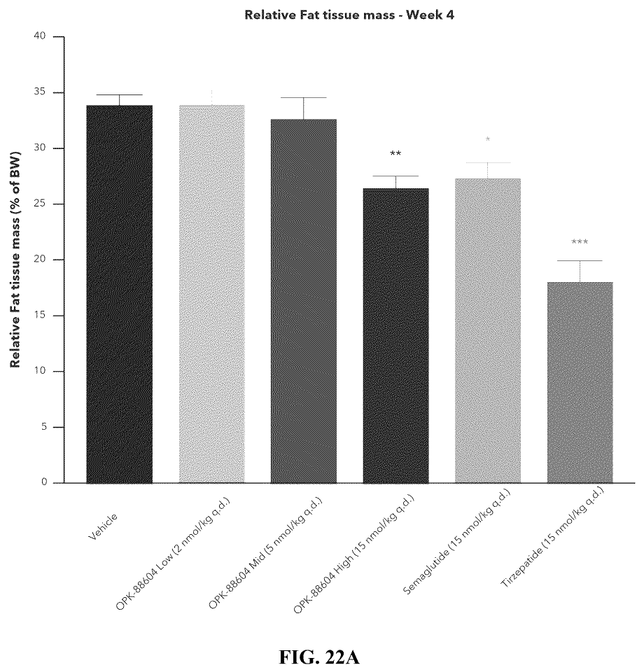

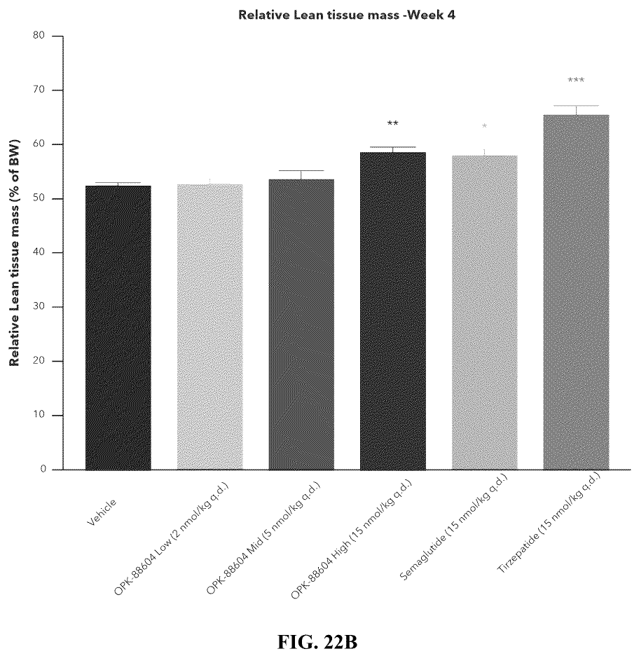

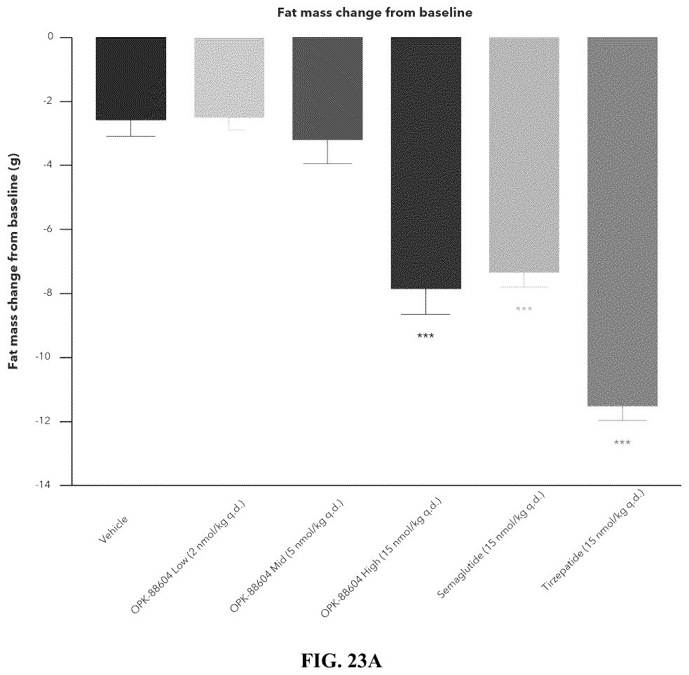

FIG. 22A shows the relative fat tissue mass at week 4, and FIG. 22B shows relative lean tissue mass at week 4. The values were expressed as mean of n=9-10+SEM and compared using Dunnett's test one-factor linear model. Semaglutide (15 nmol/kg) had P<0.05 compared to Vehicle, OPK-88604 High (15 nmol/kg) had P<0.01 compared to Vehicle, and Tirzepatide (15 nmol/kg) had P<0.001 compared to Vehicle.

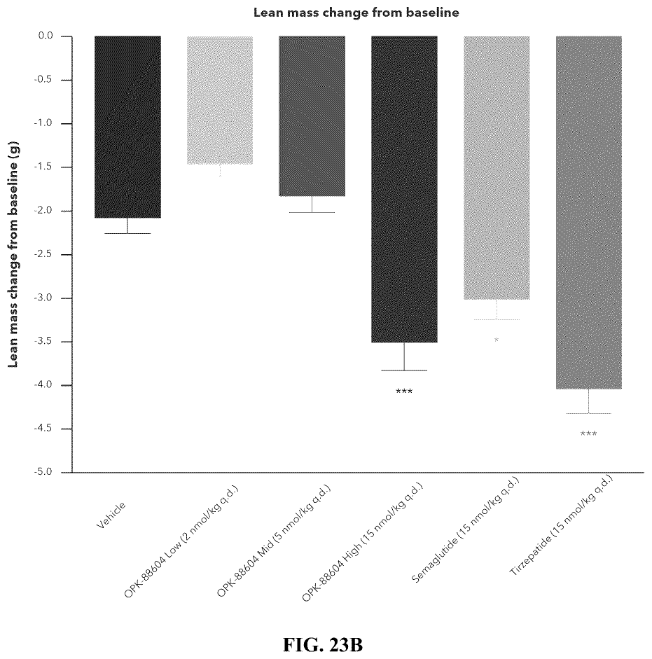

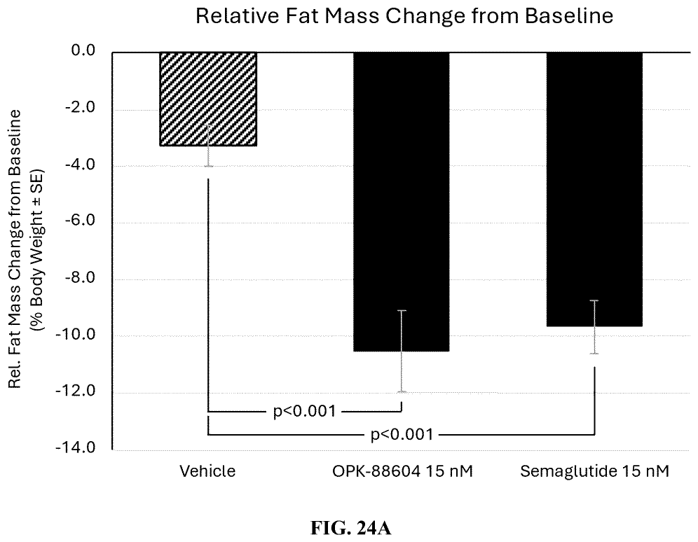

FIG. 23A shows the change in fat tissue mass from baseline, and FIG. 23B shows the change in lean tissue mass from baseline. The values were expressed as mean of n=9-10+SEM and compared using Dunnett's test one-factor linear model. In FIG. 23A, OPK-88604 High (15 nmol/kg), Semaglutide (15 nmol/kg), and Tirzepatide (15 nmol/kg) had P<0.001 compared to Vehicle. In FIG. 23B, Semaglutide (15 nmol/kg) had P<0.05, ***: P<0.001 compared to Vehicle.

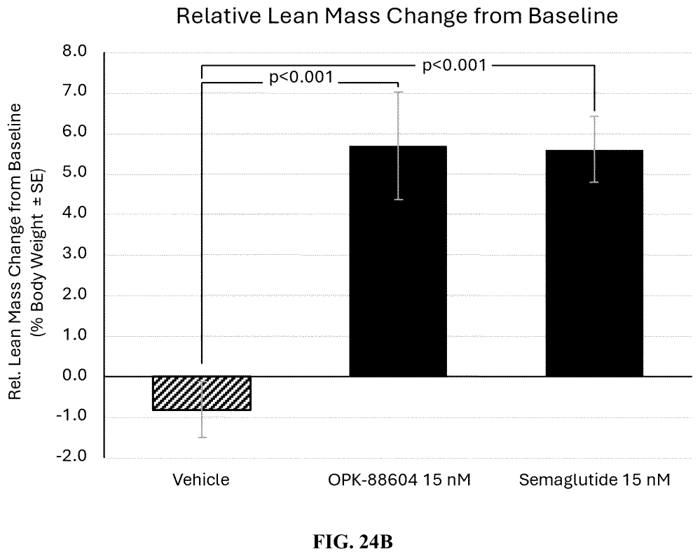

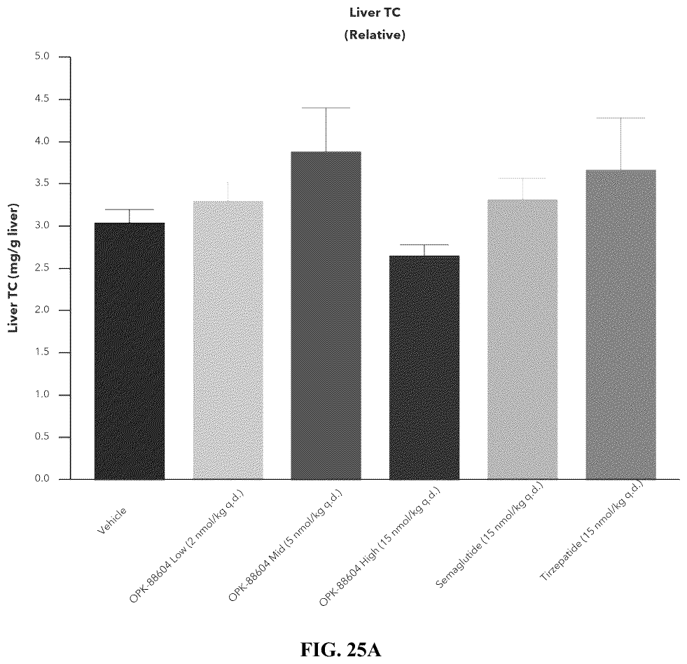

FIGS. 24A and 24B focuses on relative fat mass change (FIG. 24A) and relative lean body mass change (FIG. 24B) from baseline on day 28 for different treatments (Placebo, OPK-88604, and semaglutide). At baseline and after a 28 day treatment with placebo, OKP88604 or semaglutide, the animals in each group were evaluated for lean body mass and fat mass using DEXA and according to routine methods known in the art. The data shows the relative amount of lean body mass and fat mass change over 28 days with treatment with OPK-88604 and semaglutide was statistically similar.

### III(C): Liver Profiles

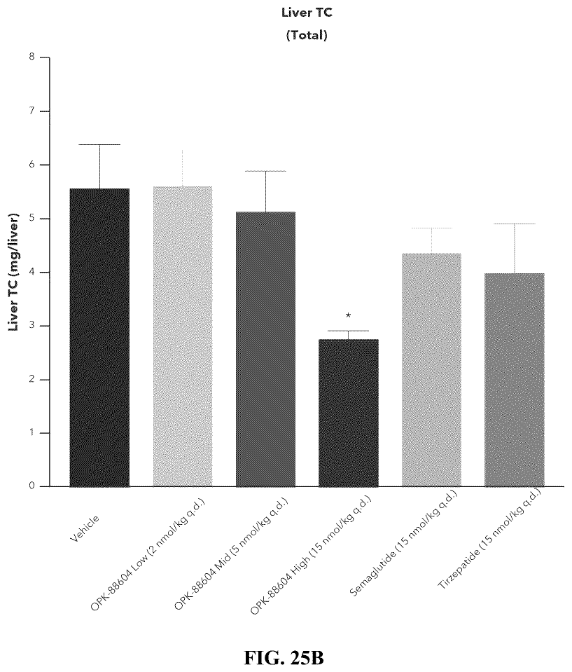

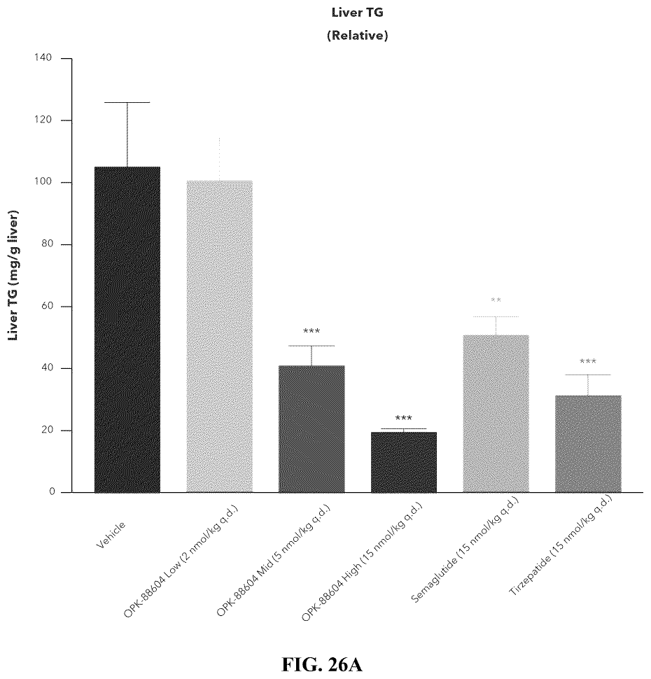

FIG. 25A shows the relative liver cholesterol and FIG. 25B shows the total liver cholesterol. The values were expressed as mean of n=9-10+SEM and compared using Dunnett's test one-factor linear model. In FIG. 25A, there were no differences at significance level 0.05. In FIG. 25B, OPK-88604 High (15 nmol/kg) had P<0.05 compared to Vehicle.

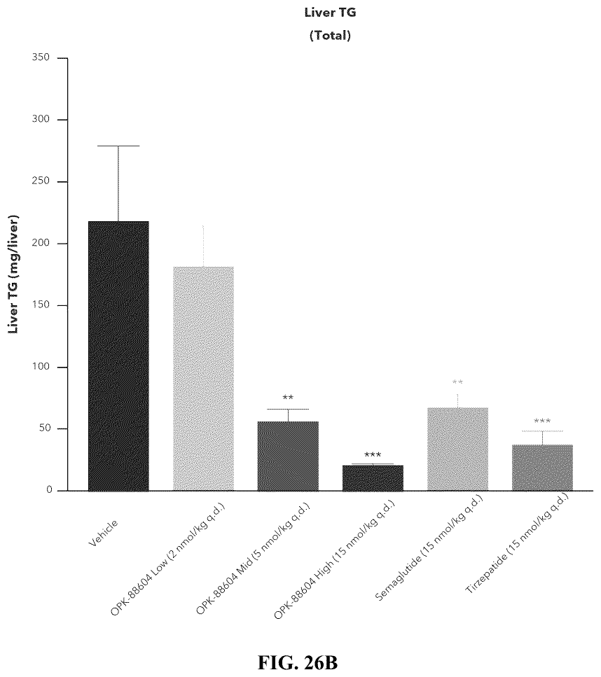

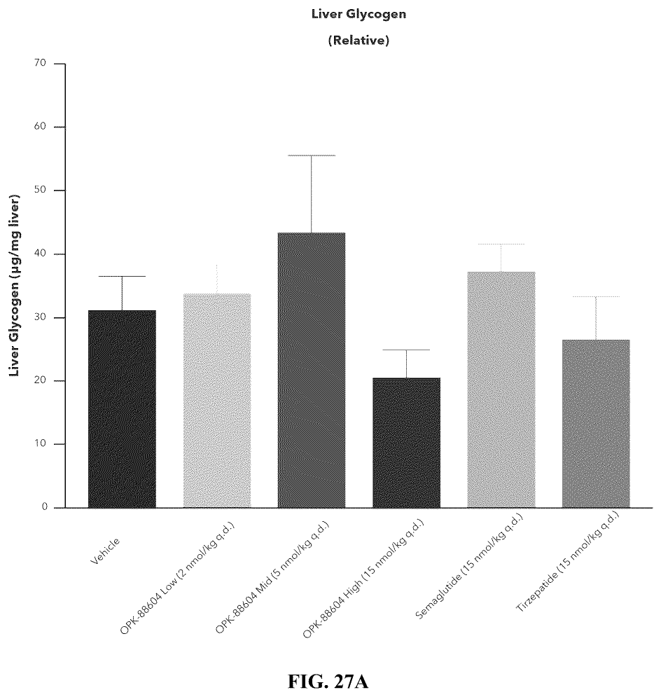

FIG. 26A shows the relative liver triglyceride and FIG. 26B shows the total liver triglyceride. The values were expressed as mean of n=9-10+SEM and compared using Dunnett's test one-factor linear model. In FIG. 26A, Semaglutide (15 nmol/kg) had P<0.01 compared to Vehicle and OPK-88604 Mid (5 nmol/kg), OPK-88604 High (15 nmol/kg), and Tirzepatide (15 nmol/kg) had P<0.001 compared to Vehicle. In FIG. 26B, OPK-88604 Mid (5 nmol/kg) and Semaglutide (15 nmol/kg) had P<0.01 compared to Vehicle, and OPK-88604 High (15 nmol/kg) and Tirzepatide (15 nmol/kg) had P<0.001 compared to Vehicle.

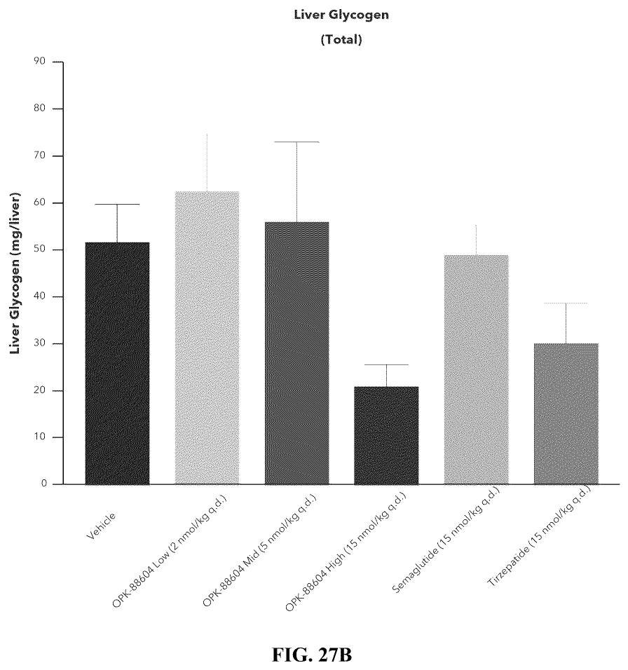

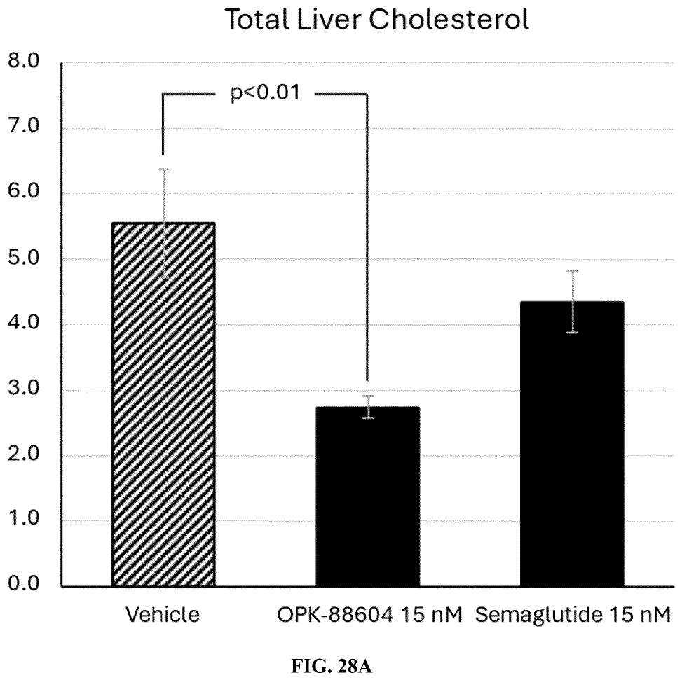

FIG. 27A shows the relative liver glycogen and FIG. 27B shows total liver glycogen. Values were expressed as mean of n=7-9+SEM and compared using Dunnett's test one-factor linear model. There were no differences at significance level 0.05.

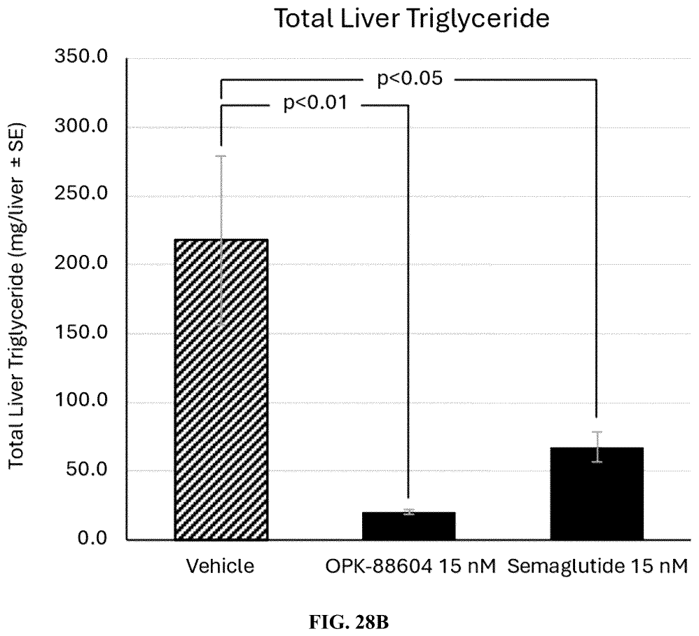

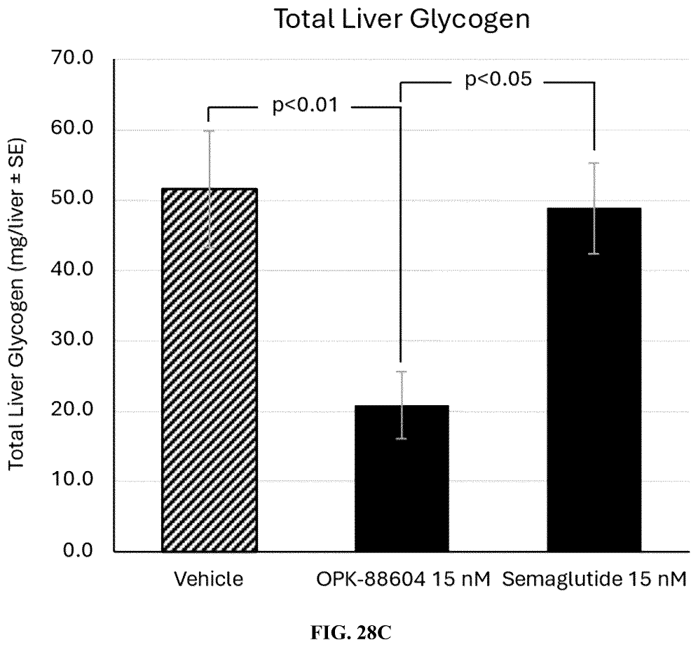

FIGS. 28A, 28B, and 28C show changes from baseline in liver cholesterol (FIG. 28A), liver triglyceride (FIG. 28B), and liver glycogen levels (FIG. 28C) on day 28 following different treatments (placebo, OPK-88604, semaglutide). DIO mice were treated with placebo, OPK-88604 and semaglutide for 28 days. The mice in each group were assessed at baseline and following a 28 day treatment with placebo, OPK-88604 or semaglutide. The data show that treatment with acylated OPK-88604 was significantly more effective in reducing total liver cholesterol, triglycerides, and glycogen. The increased reduction in cholesterol and triglyceride levels may provide additional benefits in weight loss and associated indications such as cardiovascular disease. The increased reduction in liver cholesterol with OPK-88604 may be associated with increased energy expenditure, whereas this energy expenditure is not as apparent with semaglutide treatment.

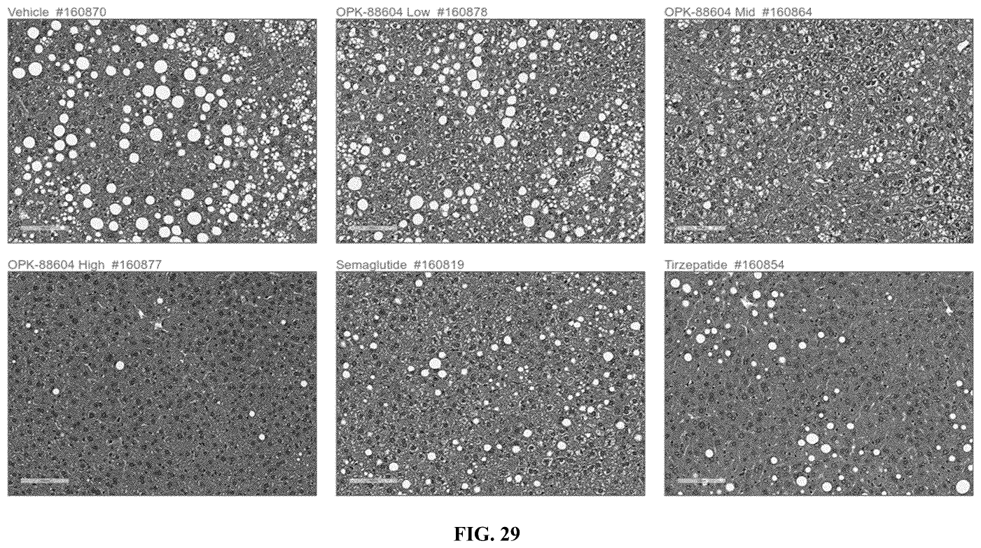

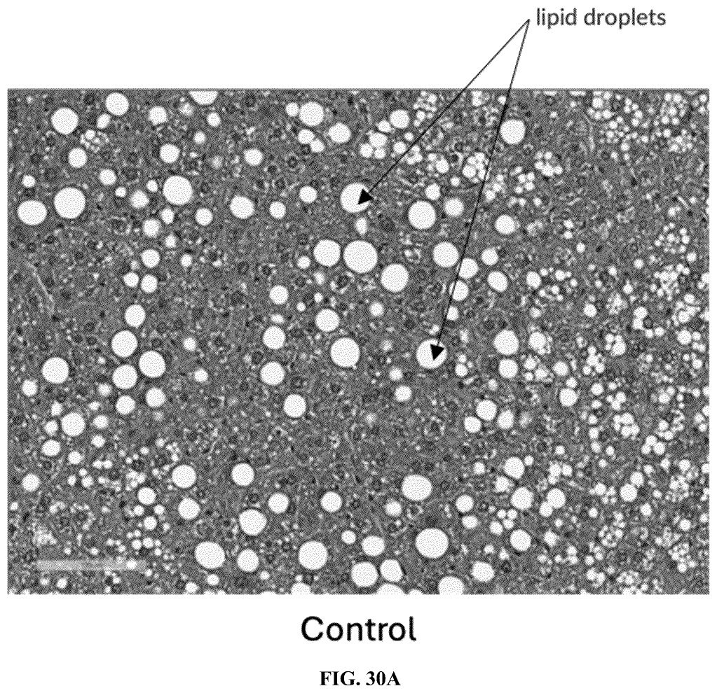

FIG. 29 shows liver H&E staining for lipid droplets following a 28 day treatment and contains representative images of liver morphology at termination (magnification 20×, scale bar=100 m).

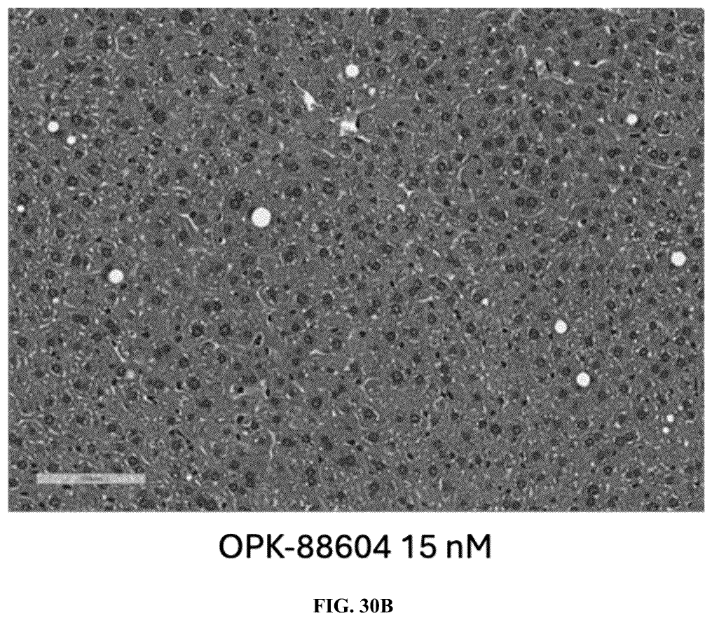

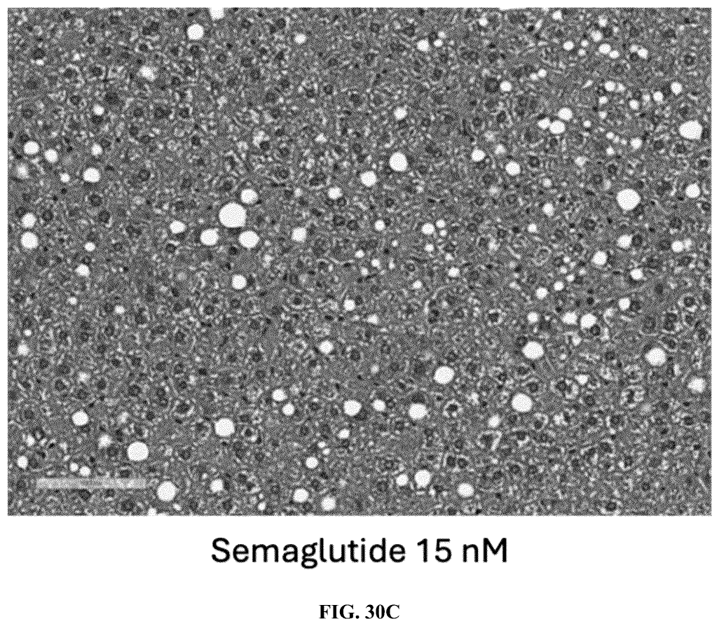

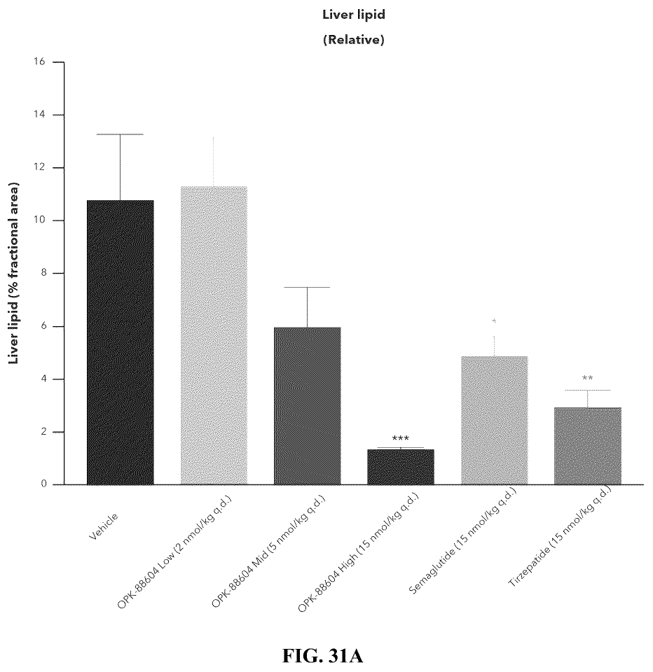

FIGS. 30A, 30B, and 30C show liver H&E staining for lipid droplets following a 28 day treatment with placebo (FIG. 30A), OPK-88604 (FIG. 30B), and semaglutide (FIG. 30C). Slides with liver tissue from DIO mice treated with placebo, OPK-88604 or semaglutide for 28 days were fixed and stained with H&E to evaluate the presence of lipid droplets according to routine methods known in the art. The data shows that treatment with OPK-88604 reduced the amount of lipid droplets in liver compared to placebo and animals treated with semaglutide. The decrease in liver droplets observed in the histological data was consistent with the total decrease in liver cholesterol and triglyceride levels analyzed in liver.

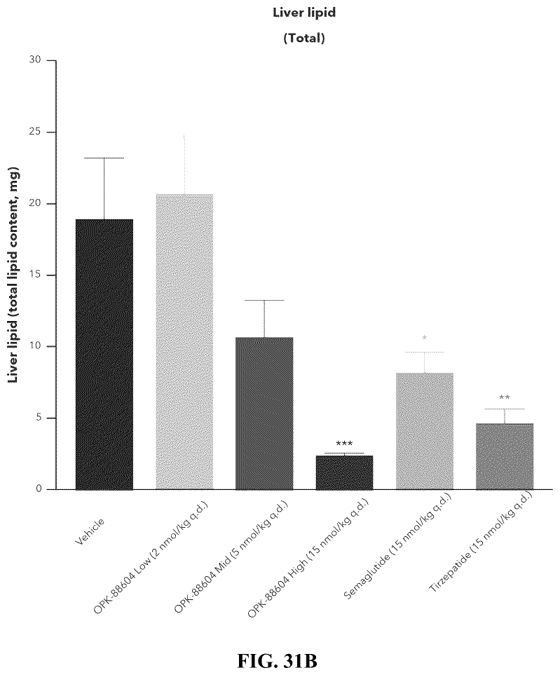

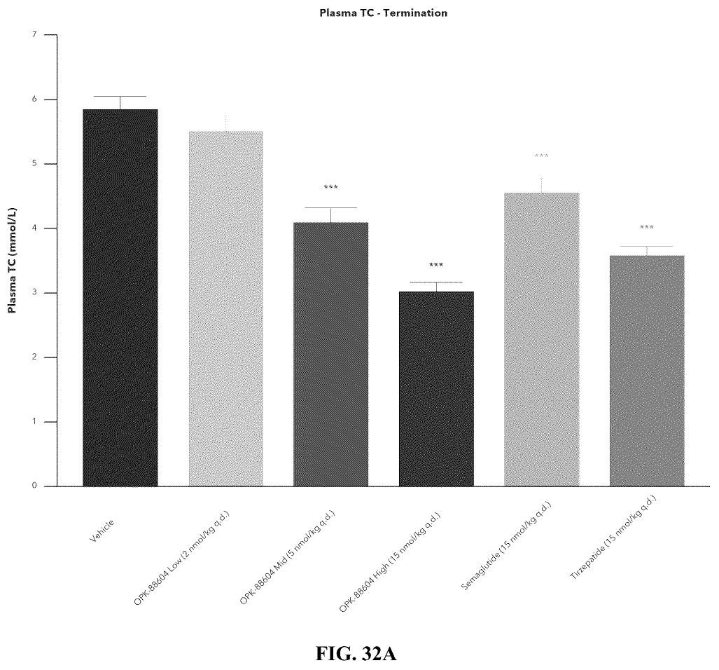

FIG. 31A shows relative liver lipid and FIG. 31B shows total liver lipid. The values were expressed as mean of n=9-10+SEM and compared using Dunnett's test one-factor linear model. Semaglutide (15 nmol/kg) had P<0.05 compared to Vehicle, Tirzepatide (15 nmol/kg) had P<0.01 compared to Vehicle, and OPK-88604 High (15 nmol/kg) had P<0.001 compared to Vehicle.

### III(D): Glucose and Insulin Profiles

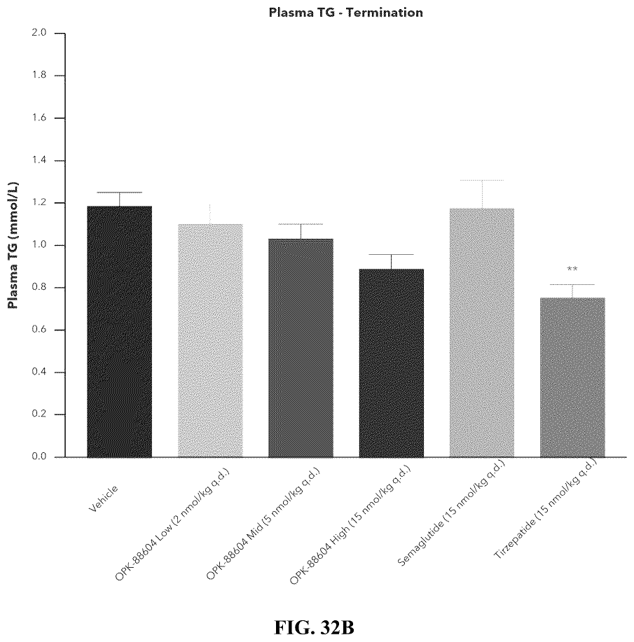

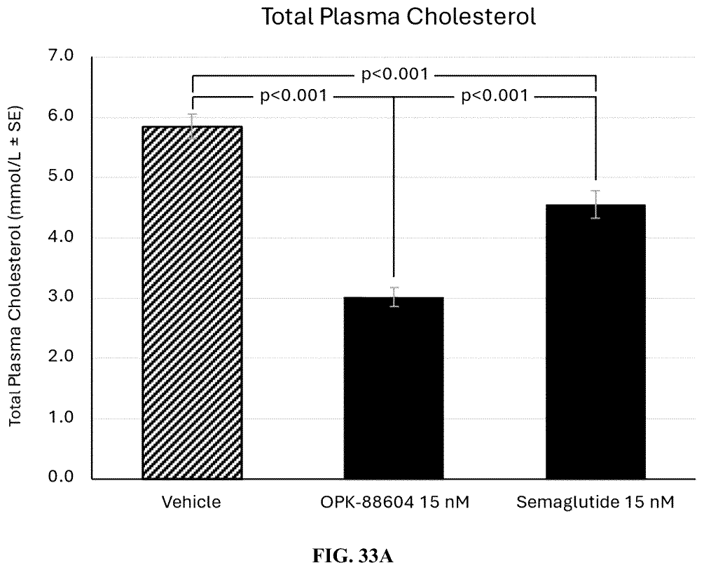

FIG. 32A shows the plasma cholesterol at termination, and FIG. 32B shows the plasma triglyceride at termination. The values were expressed as mean of n=9-10+SEM and compared using Dunnett's test one-factor linear model. In FIG. 32A, OPK-88604 Mid (5 nmol/kg q.d.), OPK-88604 High (15 nmol/kg q.d.), Semaglutide (15 nmol/kg q.d.), and Tirzepatide (15 nmol/kg q.d.) had P<0.001 compared to Vehicle. In FIG. 32B, Tirzepatide (15 nmol/kg q.d.) had P<0.01 compared to Vehicle.

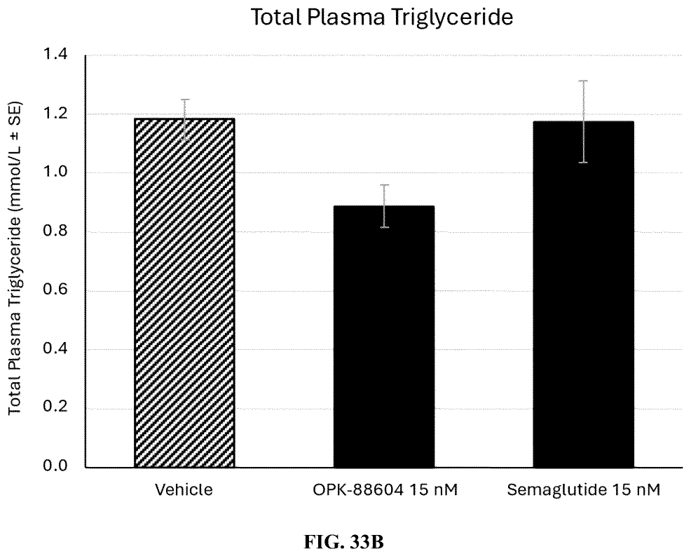

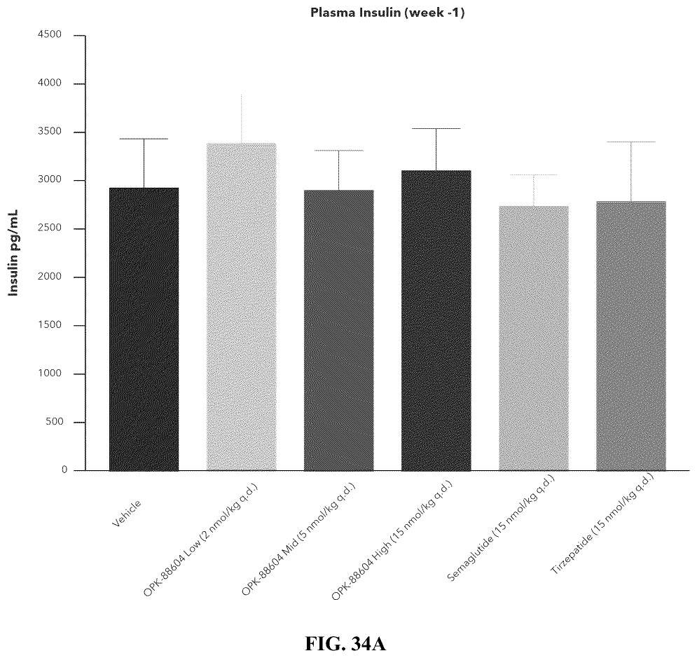

FIGS. 33A and 33B show a comparison of plasma cholesterol levels (FIG. 33A) and plasma triglyceride levels (FIG. 33B) on day 28 with different treatments (placebo, OPK-88604, and semaglutide). DIO mice were treated with placebo, OPK-88604 and semaglutide for 28 days. The serum and plasma were collected prior to and following the different treatments on day 28. The data shows that OPK-88604 treatment was significantly more effective in reducing total plasma cholesterol and triglycerides compared to semaglutide. This finding is a key differentiating property of the OPK-88604 compared to semaglutide.

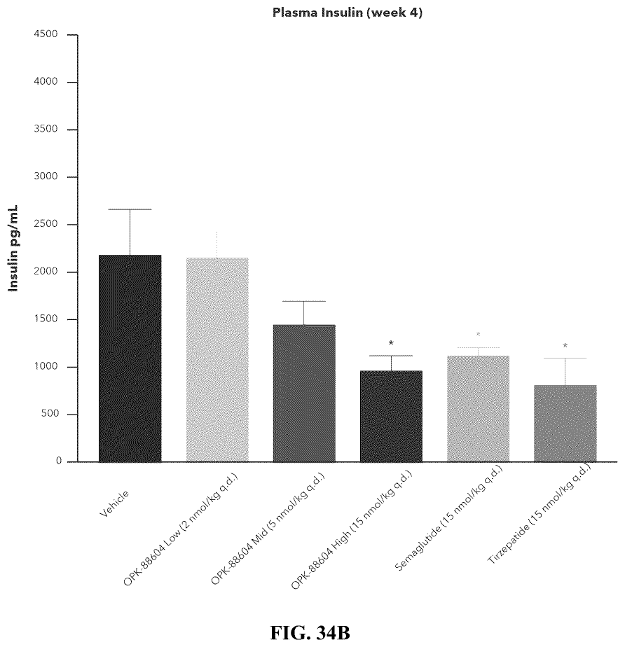

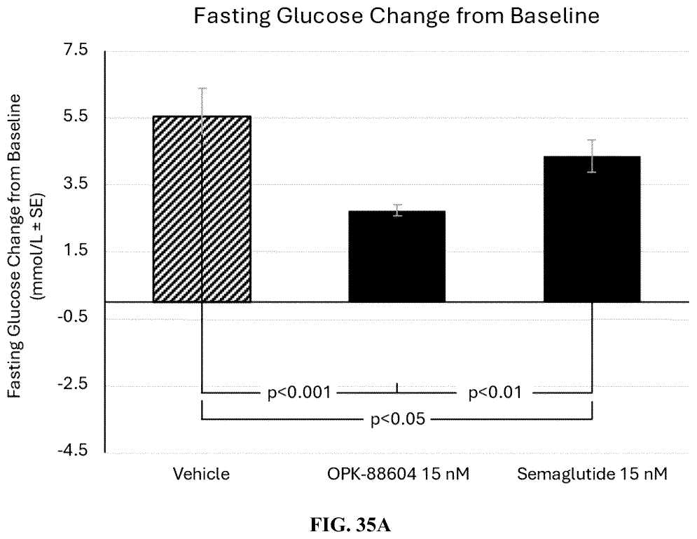

FIG. 34A shows the Fasting Blood glucose at baseline and FIG. 34B shows the fasting blood glucose at week 4. In FIG. 34A, the Values were expressed as mean of n=10+SEM, compared using Dunnett's test one-factor linear model, and there were differences at significance level 0.05. In FIG. 34B, the values were expressed as mean of n=8-10+SEM and compared using Dunnett's test one-factor linear model. In FIG. 34B, OPK-88604 High (15 nmol/kg q.d.), Semaglutide (15 nmol/kg q.d.), and Tirzepatide (15 nmol/kg q.d.) had P<0.05 compared to Vehicle.

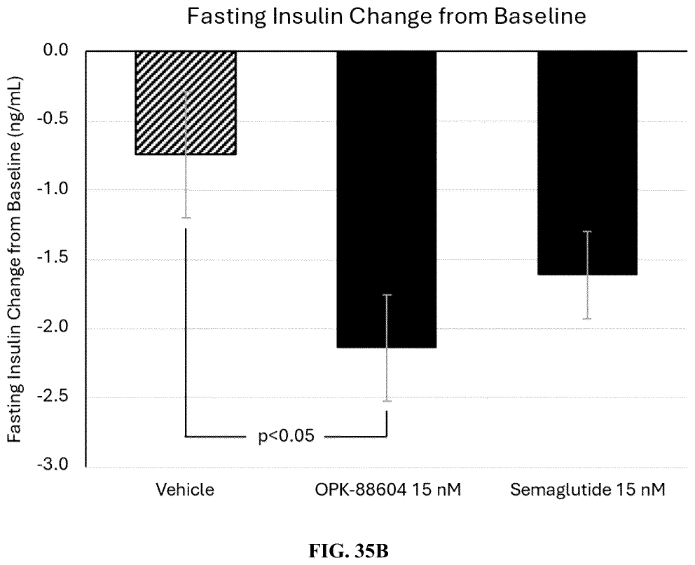

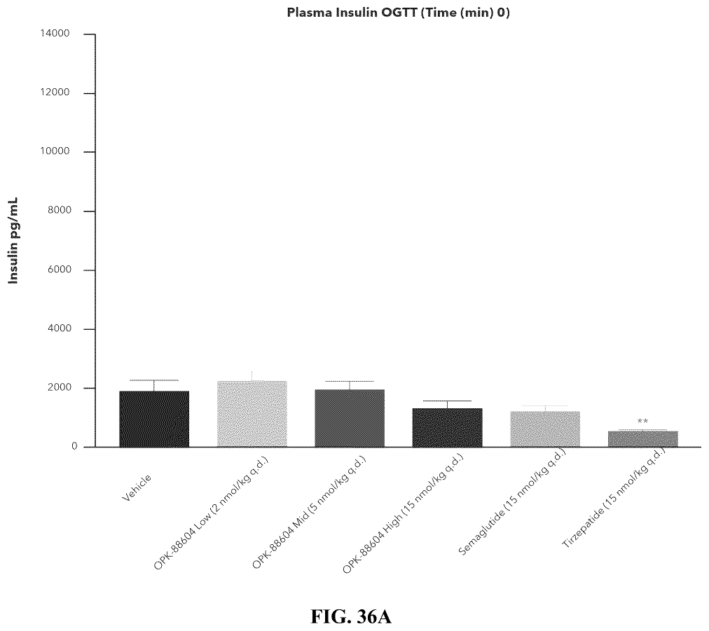

FIG. 357A and FIG. 35B show the fasting glucose change from baseline (FIG. 35A) and fasting insulin change from baseline (FIG. 35B) on day 28 following treatment with placebo, OPK-88604, and semaglutide. DIO mice treated for 28 days with placebo, OPK-88604 or Semaglutide were analyzed for fasting blood glucose and insulin change from baseline. The data shows that treatment with OPK-88604 resulted in a significant decrease in fasting glucose levels compared to semaglutide treatment. Similarly, OPK-88604 treatment resulted in a marked increase in insulin levels from baseline compared to semaglutide treatment. These data suggest that OPK-88604 may provide better glucose control compared to semaglutide by increasing insulin levels.

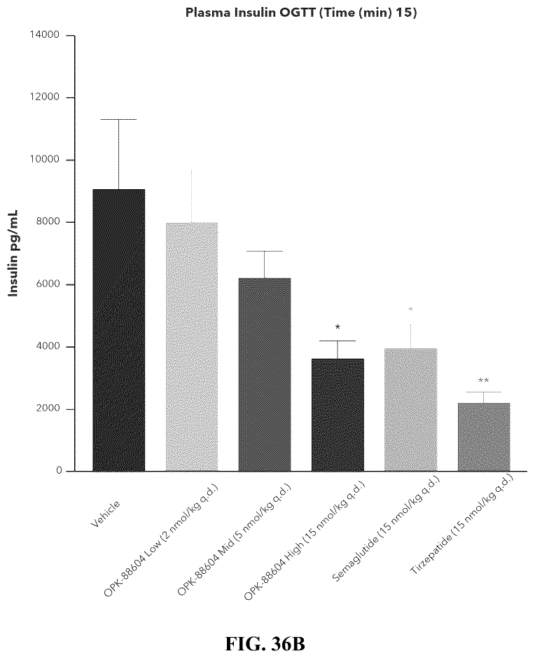

FIG. 36A shows the glucose tolerance plasma insulin at week 1 and FIG. 36B shows the glucose tolerance plasma insulin at week 4. The values were expressed as mean of n=9-10+SEM and compared using Dunnett's test one-factor linear model. In FIG. 36A, Tirzepatide (15 nmol/kg q.d.) had P<0.01 compared to Vehicle. In FIG. 36B, OPK-88604 High (15 nmol/kg q.d.) and Semaglutide (15 nmol/kg q.d.) had P<0.05 compared to Vehicle, and Tirzepatide (15 nmol/kg q.d.) had P<0.01 compared to Vehicle.

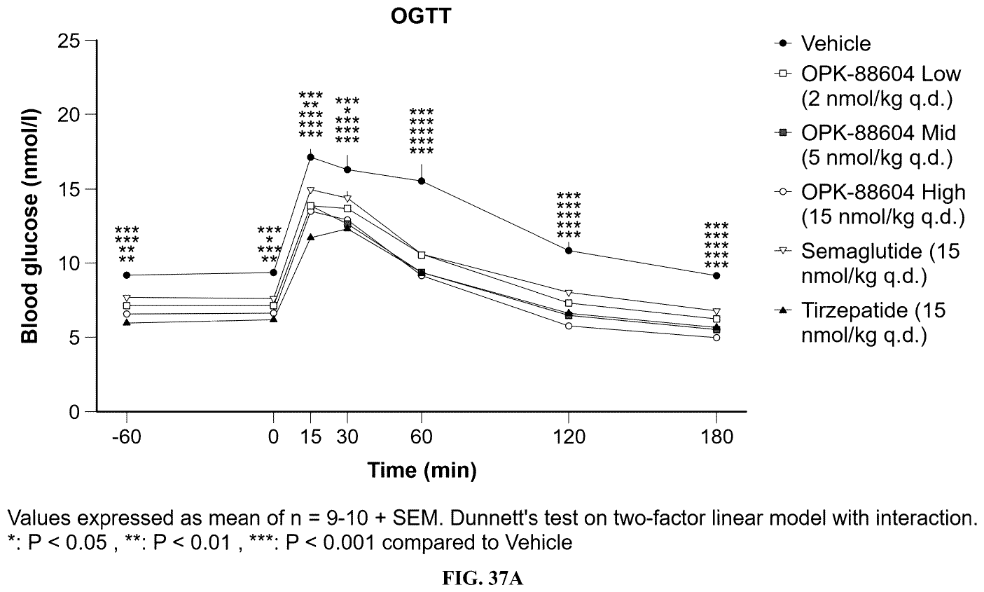

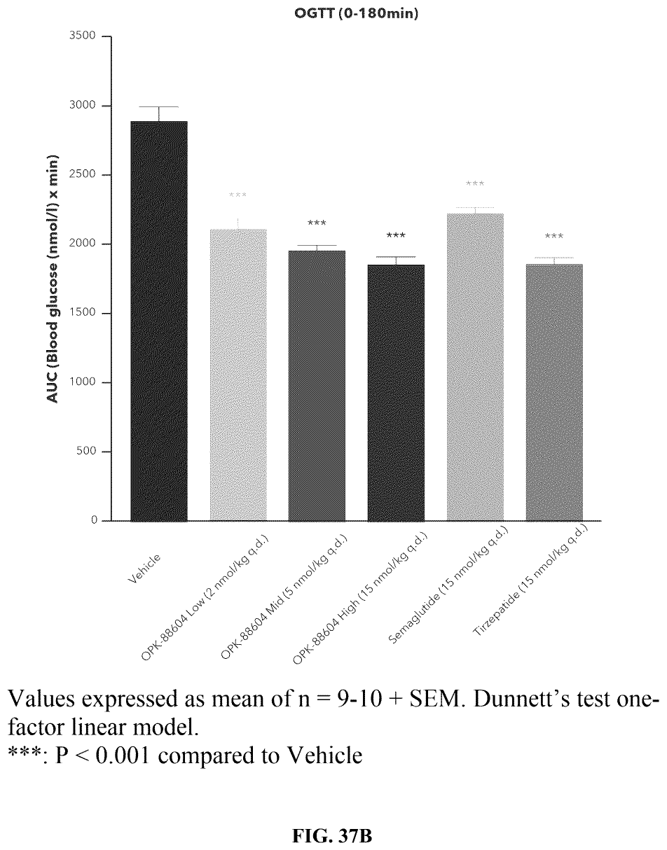

FIG. 37A shows the oral glucose tolerance test profile and FIG. 37B shows the oral glucose tolerance test area under the curve (0-180 min).

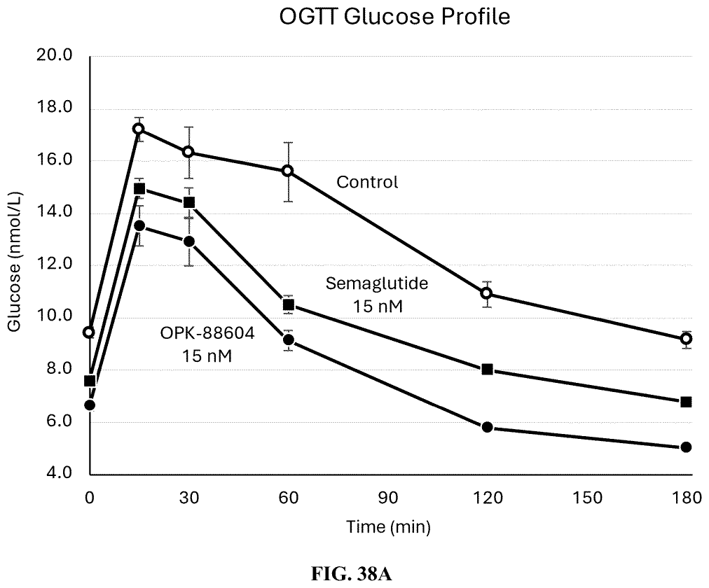

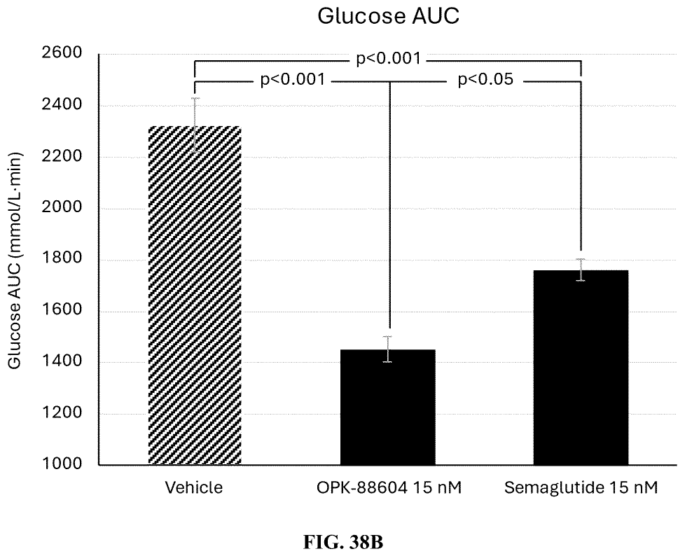

FIG. 38A and FIG. 38B show oral glucose tolerance test (FIG. 38A) and glucose area under the curve (AUC) (FIG. 38B) comparing different treatments with placebo, OPK-88604, and semaglutide. DIO mice were treated with placebo, OPK-88604 and semaglutide for 3 weeks and were assessed with an oral glucose tolerance test (OGTT) according to routine methods known in the art.

The data showed that treatment with OPK-88604 was more effective in reducing glucose levels in an Oral Glucose Tolerance test compared to semaglutide. The glucose AUC was also statistically lower in the OPK-88604 treated group compared to semaglutide.

### III(D): Organ Weights

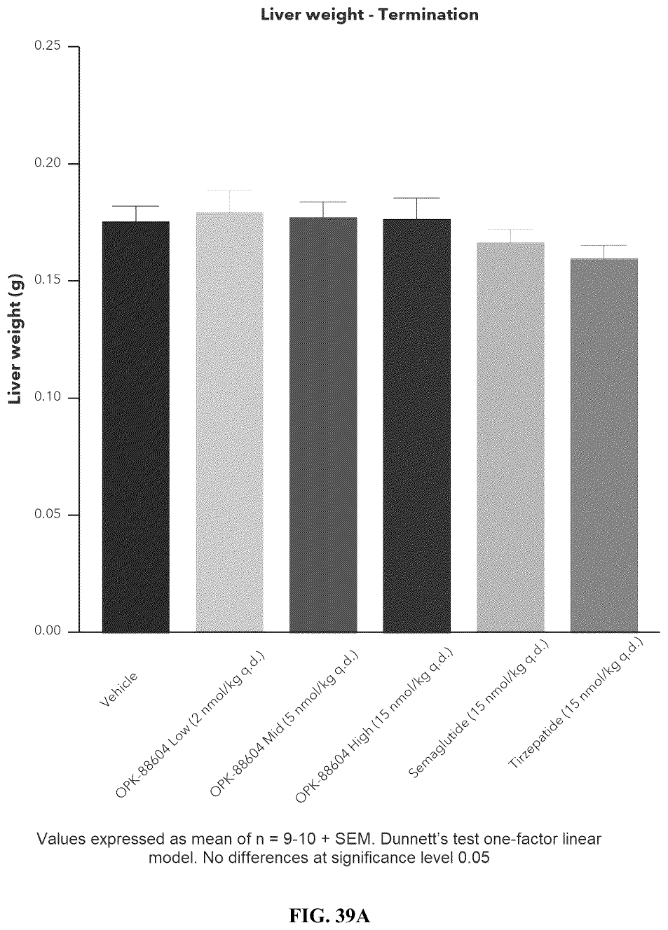

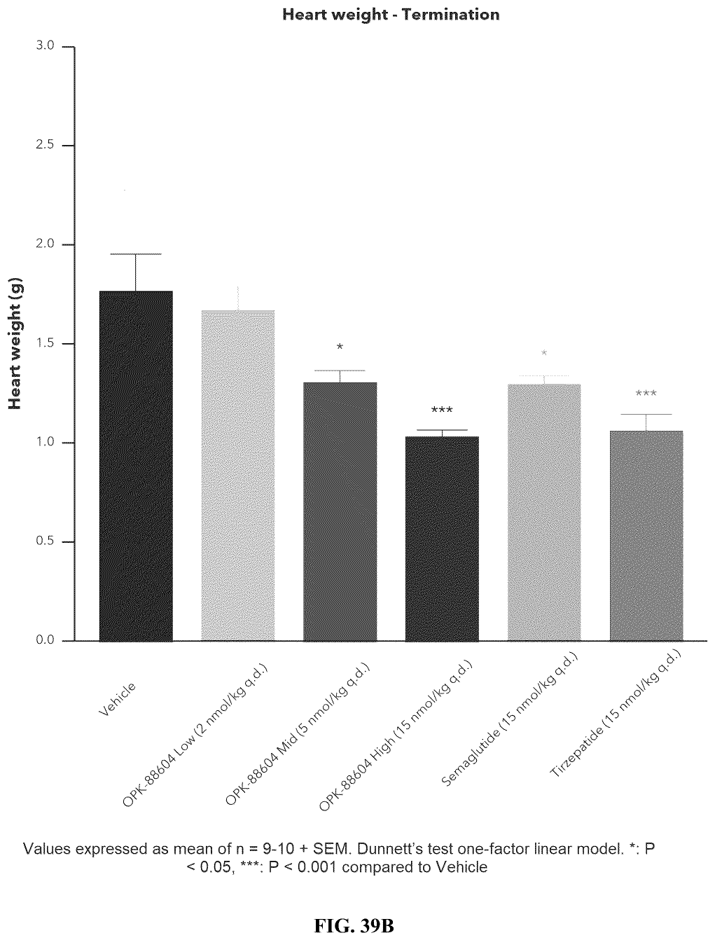

FIG. 39A shows liver weight at termination and FIG. 39B shows heart weight at termination.

### III(E): Conclusion

Four weeks treatment with OPK-88604 high (15 nmol/kg) reduced both absolute and relative body weight and fat mass, while treatment with OPK-88604 mid (5 nmol/kg) reduced relative body weight, as compared to the vehicle group. In response to an oral glucose bolus all groups treated with OPK-88604 displayed reduced blood glucose compared to vehicle controls. During the glucose tolerance test, as well as preterminal, plasma insulin levels were decreased following treatment with OPK-88604 high. Treatment with OPK-88604 (all doses) reduced blood glucose levels at study week 4. Plasma TC levels at termination were reduced by OPK-88604 mid and high treatment. Heart weight and liver TG (total and relative) were reduced by OPK-88604 mid and high. Total liver TC and Liver lipid content (total and relative) were reduced by OPK-88604 high.

### IV. EFFECT OF TREATMENT WITH OPK-88604 AND OPK-88006 ON WEIGHT LOSS IN MALE DIO MICE

### Results

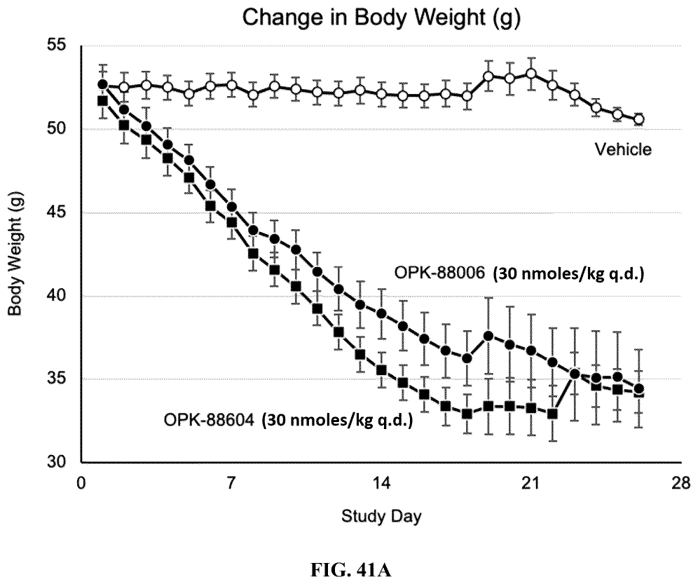

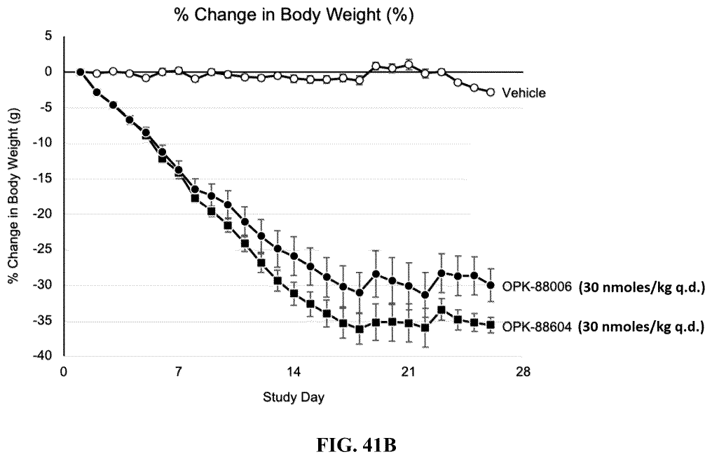

FIGS. 41A and 41B show the effect of OPKO-88604 and OPK-88006 treatment on weight loss in the DIO Mouse Model and are discussed below.

DIO mice with weights of around 55 grams were divided into three groups and treated with placebo, 30 nmoles/kg of OPK-88604, or 30 nmoles/kg of OPK-88006 for 4 weeks. The animals were weighed daily for the period of treatment.

The data showed that both OPK-88604 and OPK-88006 treatment resulted in significant weight loss over 4 weeks. The loss in weight was similar for both compounds.

### IV: CONCLUSIONS

The long-acting acylated oxyntomodulin analogs show advantages over the leading GLP-1 therapies currently available.

Currently, two long-acting acylated OXM peptide analogs, OPK-88006 and OPK-88604 have been selected and synthesized based on their GLP-1 and glucagon activities and are expected to be an improvement over semaglutide and other GLP-1 therapies. The acylated OXM peptide analogs significantly reduced the loss of GLP-1 and glucagon activity, retained an acceptable ratio of GLP-1 and glucagon activity, and reduced the molecular weight, viscosity and amount of drug and volume used for dosing relative to the PEGylated OXM peptide, OPK-88003.

The DIO mouse model studies showed that treatment with acylated OXM peptide analog, OPK-88604: Resulted in similar or slightly improved weight loss compared to Semaglutide; Reduced food consumption significantly less than treatment with semaglutide, suggesting that increases in energy expenditure may play a role in weight loss with OPK-88604; Had similar relative loss of lean body mass and fat mass compared to Semaglutide; Significantly reduced the amount of lipid droplets in liver compared to Semaglutide, consistent with the total decrease in liver cholesterol and triglyceride levels in liver and plasma; Was significantly more effective in lowering fasting glucose levels and increased insulin levels compared to Semaglutide; Was more effective in reducing glucose levels in an Oral Glucose Tolerance test compared to Semaglutide; and resulted in significant weight loss over 4 weeks compared to placebo.

1. A modified oxyntomodulin (“OXM”) having the structure of formula (II): 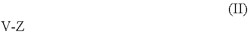
wherein:
V represents a binder complex comprising the structure of formula (III): 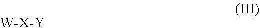
wherein:
W represents a fatty acid binder;
X represents a spacer; and
Y represents an optional linker; and
Z represents native OXM, an OXM analog, or an active fragment thereof comprising any one of SEQ ID NOs: 1 to 16.

2. (canceled)

3. (canceled)

4. The modified OXM of claim 1, wherein said V of formula II comprises:
Eicosanedioic-gGlu-(AEEA)ₙ-Lys(AcBr)-Gly-OH;
Octadecanedioic-gGlu-(AEEA)ₙ-Lys(AcBr)-Gly-OH;
Eicosanedioic-gGlu-Glu-(AEEA)ₙ-Lys(AcBr)-Gly-OH;
Octadecanedioic-gGlu-Glu-(AEEA)-Lys(AcBr)-Gly-OH;
Eicosanedioic-gGlu-(AEEA)ₙ;
Octadecanedioic-gGlu-(AEEA)ₙ;
Hexadecanedioic-gGlu-AEEA-Cys-Gly-OH;
Octadecanedioic-gGlu-AEEA-Cys-Gly-OH;
Hexadecanedioic-gGlu-(AEEA)₂-Cys-Gly-OH;
Octadecanedioic-gGlu-(AEEA)₂-Cys-Gly-OH.
H-Glu-Tyr-Glu-Lys(Octadecanedioic)-Glu-Tyr-Glu-AEEA-Cys-Gly-OH (SEQ ID NO: 21);
H-Tyr-Glu-Lys(Octadecanedioic)-Glu-Tyr-AEEA-Cys-Gly-OH (SEQ ID NO: 22);
H-Glu-Lys(Octadecanedioic)-Glu-AEEA-Cys-Gly-OH;
H-Arg-Tyr-Arg-Lys(Octadecanedioic)-Arg-Tyr-Arg-AEEA-Cys-Gly-OH (SEQ ID NO: 23);
Octadecanedioic-Glu-Tyr-Glu-Lys(Octadecanedioic)-Glu-Tyr-Glu-AEEA-Cys-Gly-OH (SEQ ID NO: 24);
H-Glu-Tyr-Glu-Lys(Eicosanedioic)-Glu-Tyr-Glu-AEEA-Cys-Gly-OH (SEQ ID NO: 29);
H-Tyr-Glu-Lys(Eicosanedioic)-Glu-Tyr-AEEA-Cys-Gly-OH (SEQ ID NO: 30);
H-Glu-Lys(Eicosanedioic)-Glu-AEEA-Cys-Gly-OH;
H-Arg-Tyr-Arg-Lys(Eicosanedioic)-Arg-Tyr-Arg-AEEA-Cys-Gly-OH (SEQ ID NO: 31); or
Eicosanedioic-Glu-Tyr-Glu-Lys(Eicosanedioic)-Glu-Tyr-Glu-AEEA-Cys-Gly-OH (SEQ ID NO: 32),
wherein n is 1, 2 or 3.

5. The modified OXM of claim 4, wherein the V of formula (II), the X of formula (III), or the Y of formula (III) is conjugated:
i. to any lysine, cysteine, or glycine residue present in said native OXM, an OXM analog, or an active fragment thereof,
ii. at one or more positions between amino acid position numbers: 17 to 37 of SEQ ID NO: 1; 18 to 39 of SEQ ID NO: 5 or SEQ ID NO: 14; 17 to 38 of SEQ ID NO: 6 or SEQ ID NO: 7; 18 to 40 of SEQ ID NO: 4, SEQ ID NO: 8, SEQ ID NO: 11, SEQ ID NO: 15, or SEQ ID NO: 16; or 17 to 39 of SEQ ID NOs: 2, 3, 9, 10, 12, or 13;
iii. at one or more amino acid position numbers between:
22 to 37, 25 to 37, 28 to 37, 30 to 37, 32 to 37, or 36 to 37 of SEQ ID NO: 1;
23 to 39, 26 to 39, 29 to 39, 31 to 39, 33 to 39, or 37 to 39 of SEQ ID NO: 5;
22 to 38, 25 to 38, 28 to 38, 30 to 38, 32 to 38, or 36 to 38 of SEQ ID NO: 6 or SEQ ID NO: 7;
23 to 40, 26 to 40, 29 to 40, 31 to 40, 33 to 40, or 37 to 40 of SEQ ID NO: 4, SEQ ID NO: 8, or SEQ ID NO: 11; or
22 to 39, 25 to 39, 28 to 39, 30 to 39, 32 to 39, or 36 to 39 of SEQ ID NO: 2, 3, 9, 10, 12, or 13
iv. at the C³⁸, C³⁹, K38, G39, of said OXM analog, or any combination thereof;
v. at the C³⁸, C³⁹, K38, G39, of said OXM analog in SEQ ID NO: 2 or SEQ ID NO: 3, or any combination thereof;
vi. at the amino acid position 38 or position 39 of said OXM analog in SEQ ID NO: 2 or SEQ ID NO: 3, or any combination thereof; or
vii. at the amino acid position 39 or position 40 of said OXM analog in SEQ ID NO: 4, or any combination thereof.

6. (canceled)

7. (canceled)

8. (canceled)

9. (canceled)

10. (canceled)

11. (canceled)

12. (canceled)

13. (canceled)

14. (canceled)

15. (canceled)

16. The modified OXM of claim 1, wherein W is:
i. octadecanedioic acid (C18 diacid) or is eicosanedioic acid (C20 diacid);
ii. octadecanedioic acid (C18 diacid) and is represented by Formula IV: 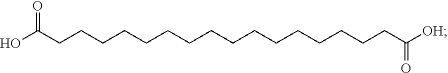 [SMILES: O=C(O)CCCCCCCCCCCCCCCCC(=O)O]
iii. octadecanedioic acid (C18 diacid) and is represented by Formula IV-A when linked to an amino group to form an amide bond 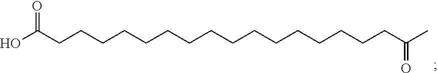 [SMILES: CC(=O)CCCCCCCCCCCCCCCCC(=O)O]
iv. eicosanedioic acid (C20 diacid) and is represented by Formula V: 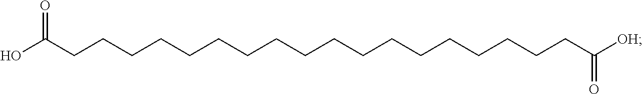 [SMILES: O=C(O)CCCCCCCCCCCCCCCCCCC(=O)O]
v. eicosanedioic acid (C20 diacid) and is represented by Formula IV-A when linked to an amine to form an amide bond:

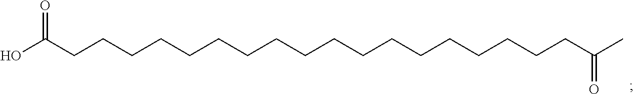 [SMILES: CC(=O)CCCCCCCCCCCCCCCCCCC(=O)O]

17. (canceled)

18. (canceled)

19. The modified OXM of claim 1, wherein X is:
i. gGlu-Gluₙ-(AEEA)ₘ-Cys-Glyₚ (SEQ ID NOs: 33 and 34), gGlu-Gluₙ-(AEEA)ₘ-Lys(AcBr)-Glyₚ (SEQ ID NOs: 35 and 36), or gGlu-Gluₙ-(AEEA)ₘ-Lys-Glyₚ (SEQ ID NOs: 37 and 38) and n is 1, 2, or 3; m is 1, 2, or 3; and p is 1, 2, or 3;
ii. gGlu-Glu-(AEEA)ₘ-Cys-Gly, m is 1, 2, or 3, and is represented by Formula VI: 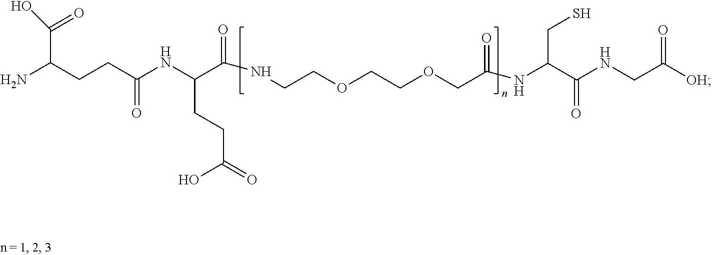
iii. gGlu-Glu-(AEEA)ₘ-Cys-Gly, m is 1, 2, or 3, and is represented by Formula VI-E when linked to an adjoining moiety at the S bond: 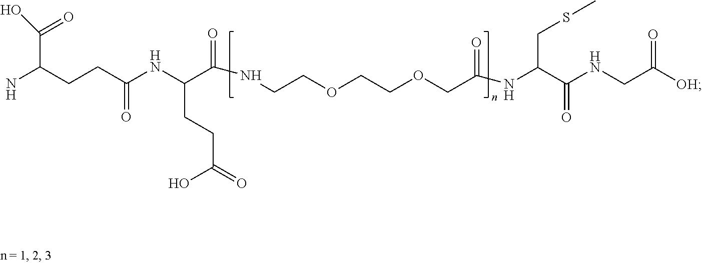
iv. gGlu-Glu-(AEEA)ₘ-Lys(AcBr)-Gly, m is 1, 2, or 3, and is represented by Formula VII: 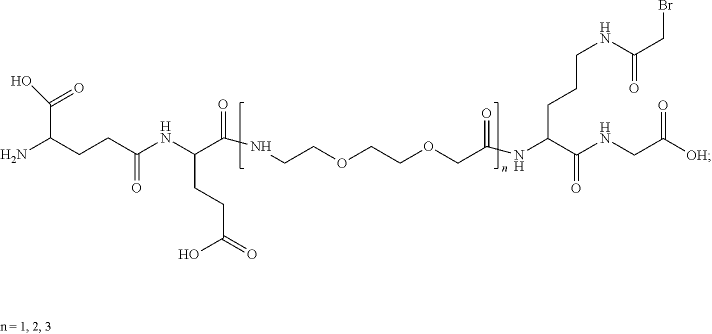
v. gGlu-Glu-(AEEA)ₘ-Lys(AcBr)-Gly, m is 1, 2 or 3 and is represented by Formula VII-C when linked to an adjoining moiety at the acyl carbon: 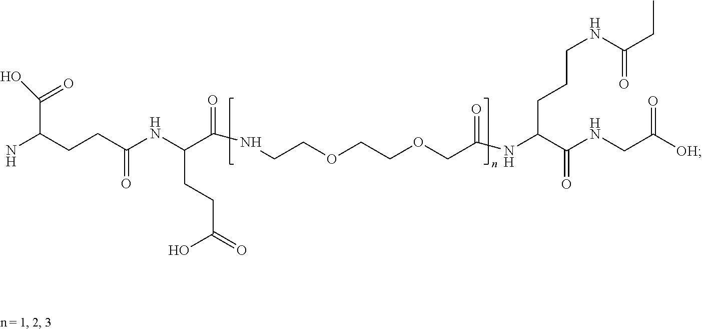
vi. gGlu-Glu-(AEEA)ₘ-Lys-Gly, m is 1, 2, or 3, and is represented by Formula VIII: 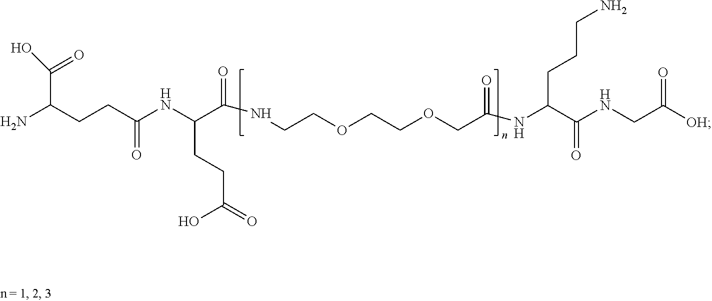
vii. gGlu-Glu-(AEEA)ₘ-Lys-Gly, m is 1, 2, or 3, and is represented by Formula VIII-D when linked through the lysine amino group:

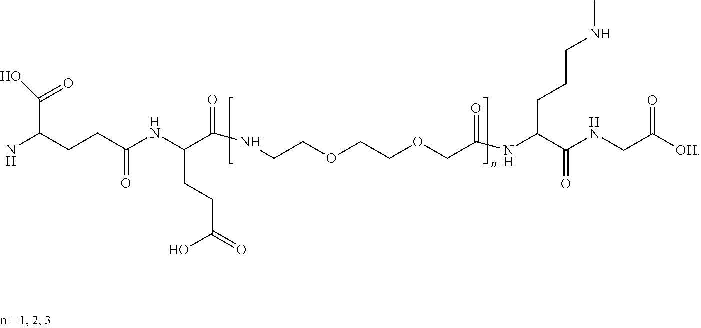

20. (canceled)

21. (canceled)

22. (canceled)

23. The modified OXM of claim 1, wherein Y is:
i. Chloropropane-2-one-Fmoc-Mal;
ii. Chloropropane-2-one-Fmoc-Mal and is represented by Formula IX: 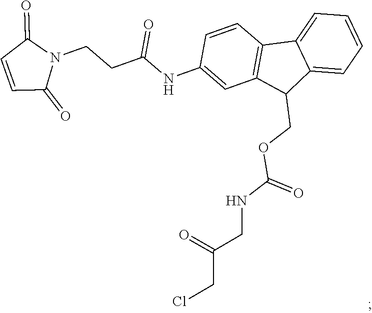 [SMILES: O=C(CCl)CNC(=O)OCC1c2ccccc2-c2ccc(NC(=O)CCN3C(=O)C=CC3=O)cc21]
iii. Chloropropane-2-one-Fmoc-Mal and is represented by Formula IX-C when attached to an adjoining atom at the acyl carbon: 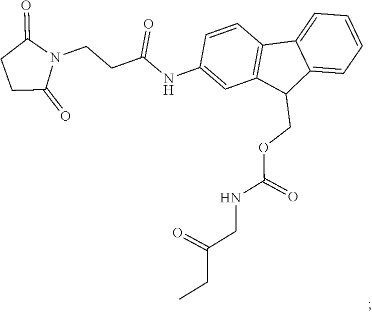 [SMILES: CCC(=O)CNC(=O)OCC1c2ccccc2-c2ccc(NC(=O)CCN3C(=O)CCC3=O)cc21]
iv. Mal-NRFmoc-NHS;
v. Mal-NRFmoc-NHS and is represented by Formula XIII: 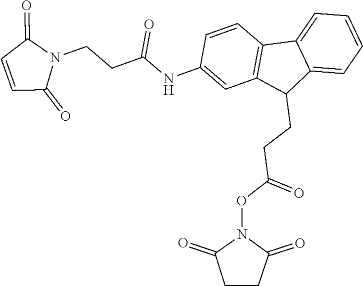 [SMILES: O=C(CCN1C(=O)C=CC1=O)Nc1ccc2c(c1)C(CCC(=O)ON1C(=O)CCC1=O)c1ccccc1-2]
v. Mal-NRFmoc-NHS and is represented by Formula XIII-C when linked at the oxygen to an adjoining atom:

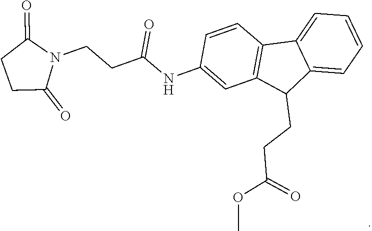 [SMILES: COC(=O)CCC1c2ccccc2-c2ccc(NC(=O)CCN3C(=O)CCC3=O)cc21]

24. (canceled)

25. (canceled)

26. (canceled)

27. The modified OXM of claim 1, wherein the bond between the Z of Formula II and the linker is a stable covalent bond or is a reversible covalent bond.

28. (canceled)

29. The modified OXM of claim 1, wherein the modified OXM comprises the following formula:

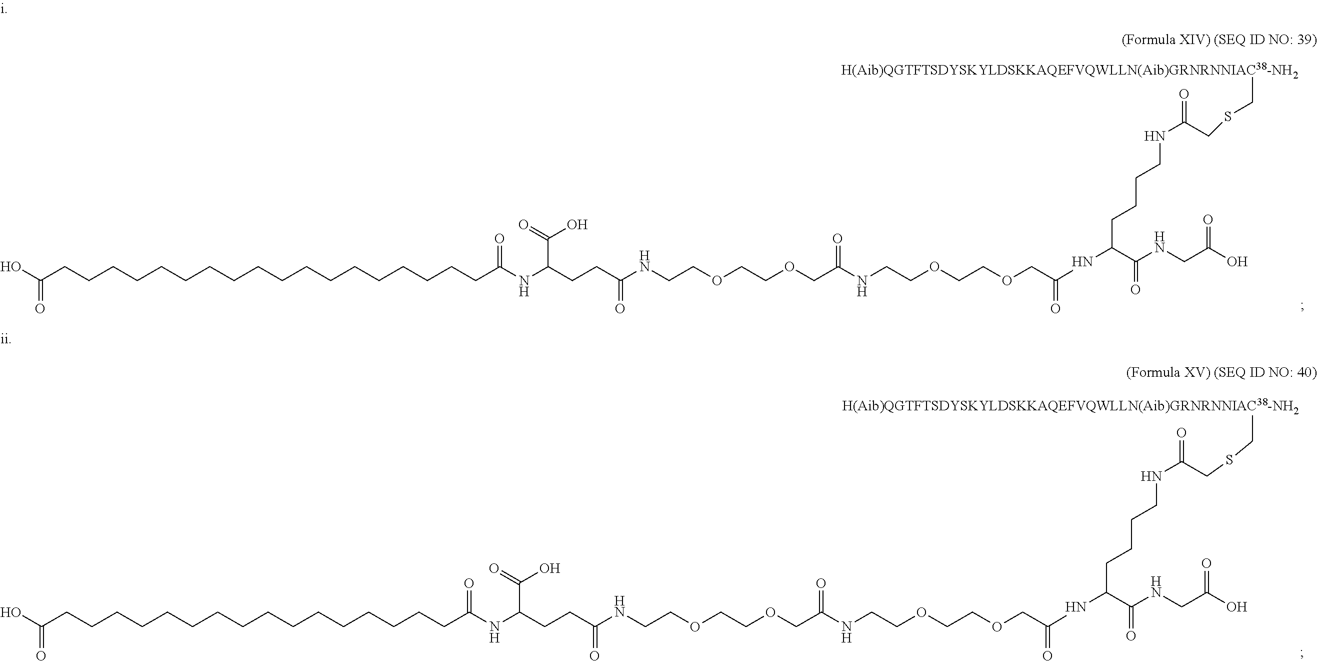 [SMILES: "H(Aib)QGTFTSDYSKYLDSKKAQEFVQWLLN(Aib)GRNRNNIAC38-NH2" ; CCSCC(=O)NCCCCC(NC(=O)COCCOCCNC(=O)COCCOCCNC(=O)CCC(NC(=O)CCCCCCCCCCCCCCCCCCC(=O)O)C(=O)O)C(=O)NCC(=O)O ; "H(Aib)QGTFTSDYSKYLDSKKAQEFVQWLLN(Aib)GRNRNNIAC38-NH2" ; CCSCC(=O)NCCCCC(NC(=O)COCCOCCNC(=O)COCCOCCNC(=O)CCC(NC(=O)CCCCCCCCCCCCCCCCC(=O)O)C(=O)O)C(=O)NCC(=O)O]

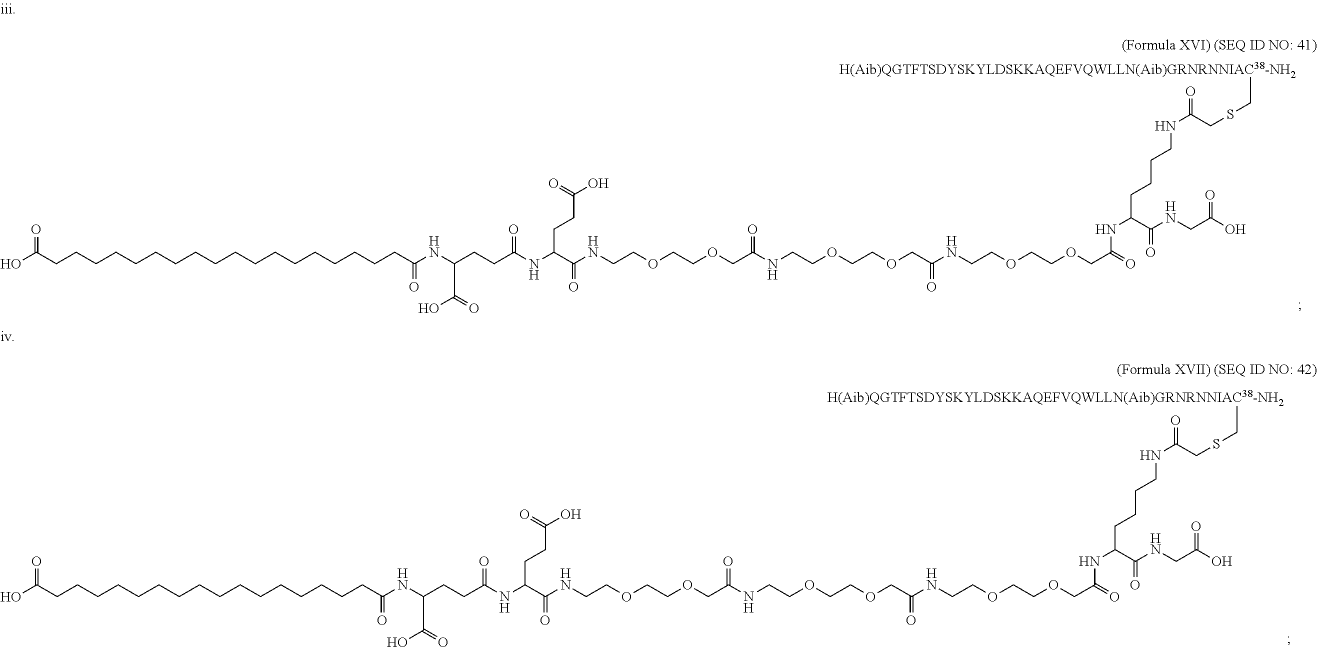 [SMILES: "H(Aib)QGTFTSDYSKYLDSKKAQEFVQWLLN(Aib)GRNRNNIAC38-NH2" ; CCSCC(=O)NCCCCC(NC(=O)COCCOCCNC(=O)COCCOCCNC(=O)COCCOCCNC(=O)C(CCC(=O)O)NC(=O)CCC(NC(=O)CCCCCCCCCCCCCCCCCCC(=O)O)C(=O)O)C(=O)NCC(=O)O ; "H(Aib)QGTFTSDYSKYLDSKKAQEFVQWLLN(Aib)GRNRNNIAC38-NH2" ; CCSCC(=O)NCCCCC(NC(=O)COCCOCCNC(=O)COCCOCCNC(=O)COCCOCCNC(=O)C(CCC(=O)O)NC(=O)CCC(NC(=O)CCCCCCCCCCCCCCCCC(=O)O)C(=O)O)C(=O)NCC(=O)O]

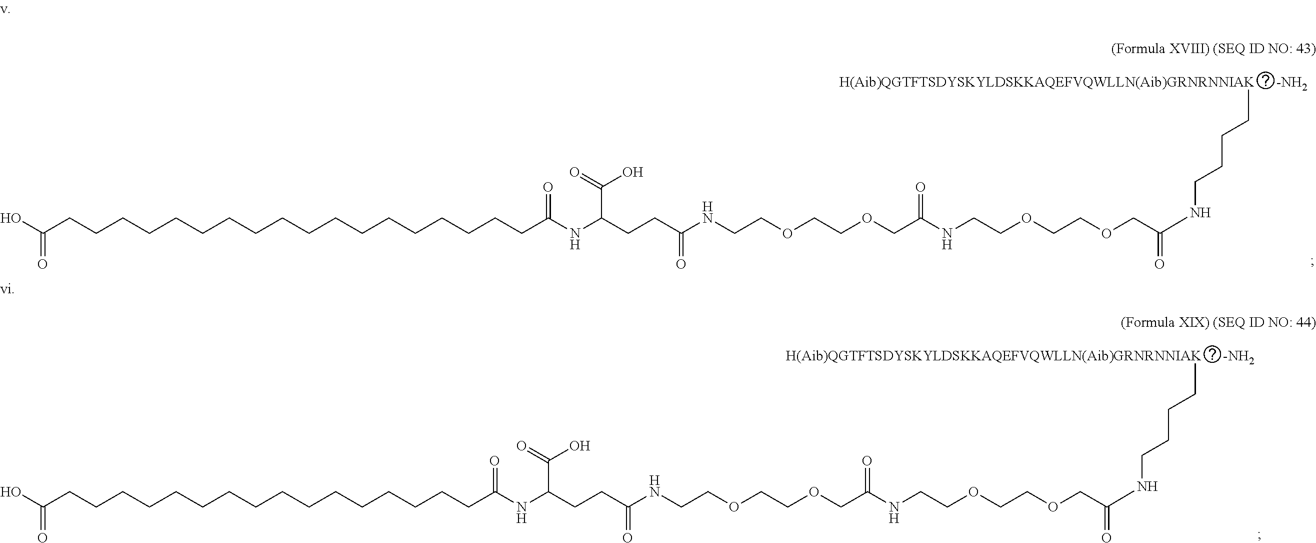 [SMILES: "H(Aib)QGTFTSDYSKYLDSKKAQEFVQWLLN(Aib)GRNRNNIAK        -NH2" ; CCCCCNC(=O)COCCOCCNC(=O)COCCOCCNC(=O)CCC(NC(=O)CCCCCCCCCCCCCCCCCCC(=O)O)C(=O)O ; "H(Aib)QGTFTSDYSKYLDSKKAQEFVQWLLN(Aib)GRNRNNIAK        -NH2" ; CCCCCNC(=O)COCCOCCNC(=O)COCCOCCNC(=O)CCC(NC(=O)CCCCCCCCCCCCCCCCC(=O)O)C(=O)O]

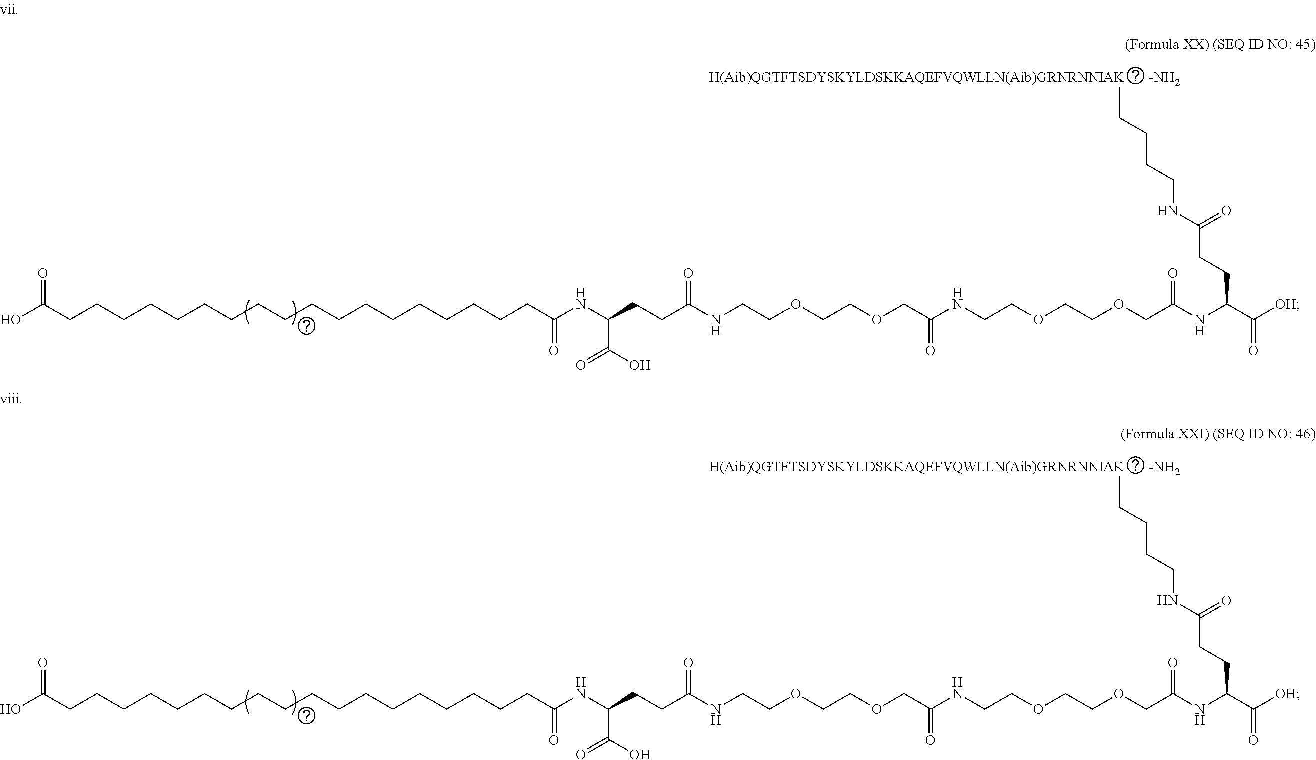

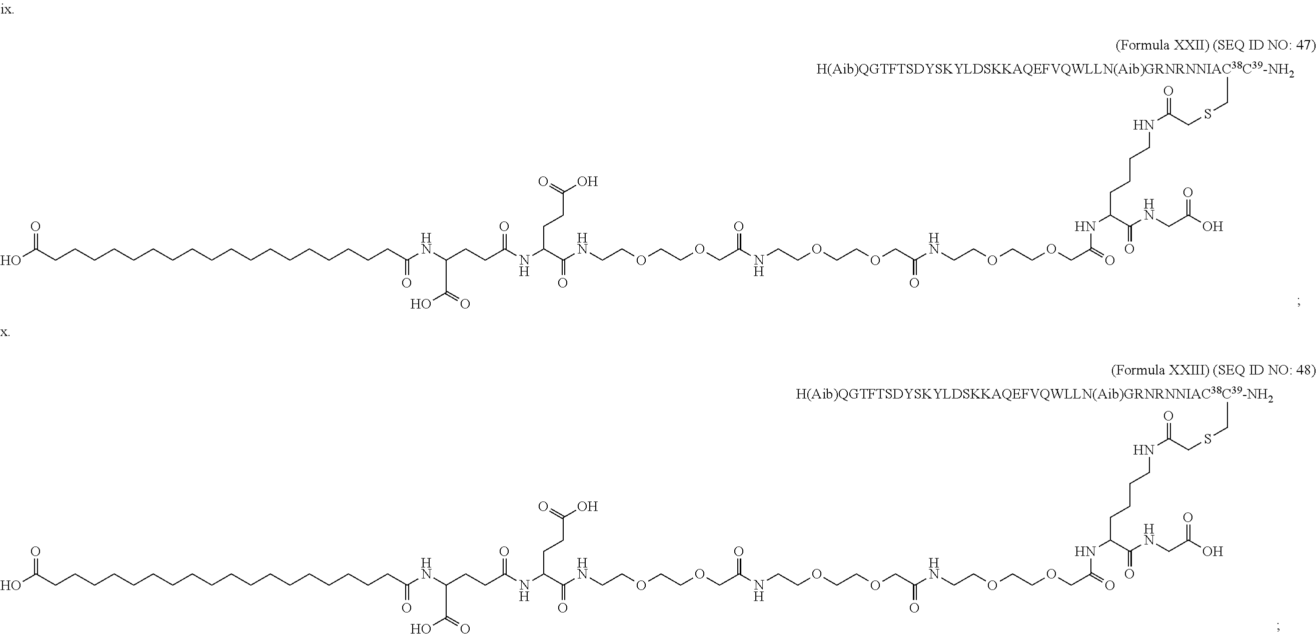 [SMILES: "H(Aib)QGTFTSDYSKYLDSKKAQEFVQWLLN(Aib)GRNRNNIAC38C39-NH2" ; CCSCC(=O)NCCCCC(NC(=O)COCCOCCNC(=O)COCCOCCNC(=O)COCCOCCNC(=O)C(CCC(=O)O)NC(=O)CCC(NC(=O)CCCCCCCCCCCCCCCCCCC(=O)O)C(=O)O)C(=O)NCC(=O)O ; "H(Aib)QGTFTSDYSKYLDSKKAQEFVQWLLN(Aib)GRNRNNIAC38C39-NH2" ; CCSCC(=O)NCCCCC(NC(=O)COCCOCCNC(=O)COCCOCCNC(=O)COCCOCCNC(=O)C(CCC(=O)O)NC(=O)CCC(NC(=O)CCCCCCCCCCCCCCCCCCC(=O)O)C(=O)O)C(=O)NCC(=O)O]

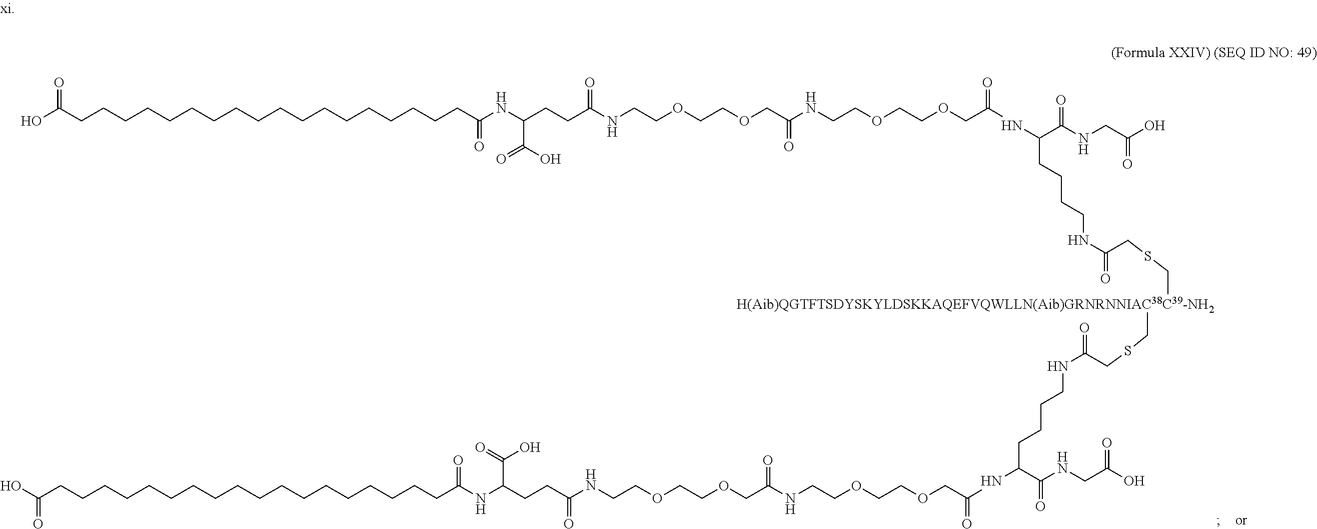

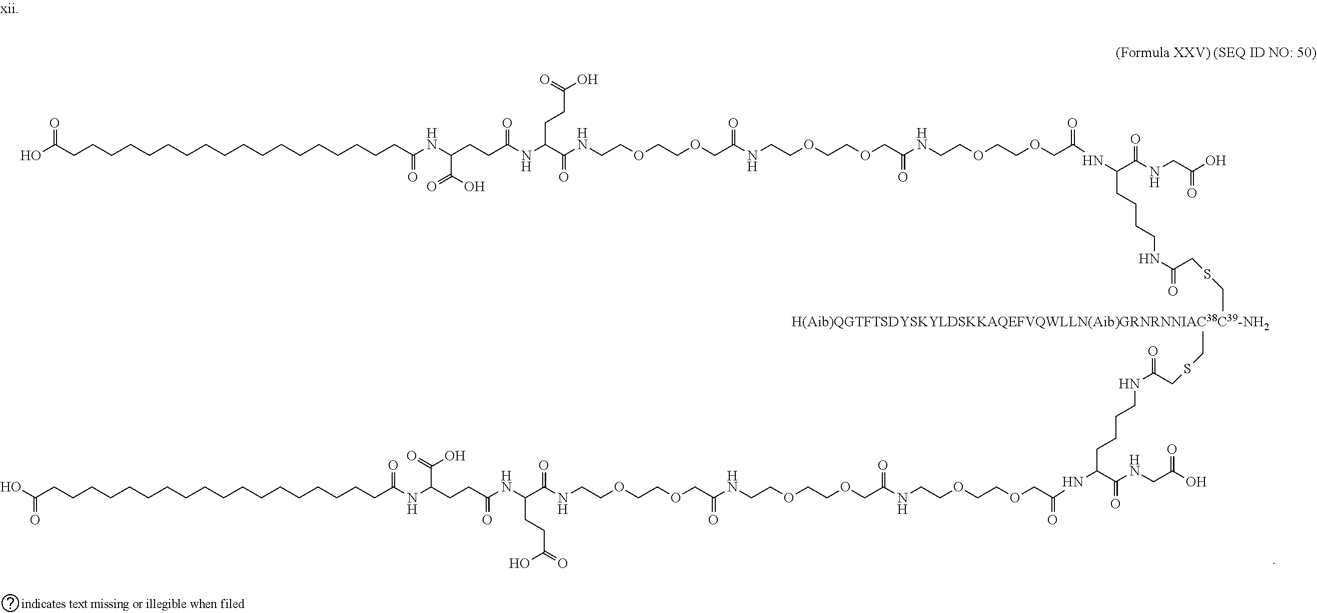

30. The modified OXM of claim 1, wherein the modified OXM comprises:
i.
(SEQ ID NO: 39)
H(Aib)QGTFTSDYSKYLDSKKAQEFVQWLLN(Aib)GRNRNNIAC³⁸
(Eicosanedioic-gGlu-(AEEA)₂-Lys(Ac)-Gly)-NH₂;
ii.
(SEQ ID NO: 40)
H(Aib)QGTFTSDYSKYLDSKKAQEFVQWLLN(Aib)GRNRNNIAC³⁸
(Octadecanedioic-gGlu-(AEEA)₂-Lys(Ac)-Gly)-NH₂;
iii.
(SEQ ID NO: 41)
H(Aib)QGTFTSDYSKYLDSKKAQEFVQWLLN(Aib)GRNRNNIAC³⁸
(Eicosanedioic-gGlu-Glu-(AEEA)₃-Lys(Ac)-Gly)-
NH₂;
iv.
(SEQ ID NO: 42)
H(Aib)QGTFTSDYSKYLDSKKAQEFVQWLLN(Aib)GRNRNNIAC³⁸
(Octadecanedioic-gGlu-Glu-(AEEA)₃-Lys(Ac)-Gly)-
NH₂;
v.
(SEQ ID NO: 43)
H(Aib)QGTFTSDYSKYLDSKKAQEFVQWLLN(Aib)GRNRNNIAK³⁸
(Eicosanedioic-gGlu-(AEEA)₂)G³⁹-NH₂;
vi.
(SEQ ID NO: 44)
H(Aib)QGTFTSDYSKYLDSKKAQEFVQWLLN(Aib)GRNRNNIAK³⁸
(Octadecanedioic-gGlu-(AEEA)₂)G³⁹-NH₂;
vii.
(SEQ ID NO: 45)
H(Aib)QGTFTSDYSKYLDSKKAQEFVQWLLN(Aib)GRNRNNIAK³⁸
(Eicosanedioic-gGlu-(AEEA)₂-gGlu)G³⁹-NH₂;
viii.
(SEQ ID NO: 46)
H(Aib)QGTFTSDYSKYLDSKKAQEFVQWLLN(Aib)GRNRNNIAK³⁸
(Octadecanedioic-gGlu-(AEEA)₂-gGlu)G³⁹-NH₂;
ix.
(SEQ ID NO: 47)
Eicosanedioic-gGlu-Glu-AEEA-AEEA-AEEA-Lys(Ac-
Cys³⁸mut.OXM(C³⁸C³⁹))-Gly-OH;
x.
(SEQ ID NO: 48)
Eicosanedioic-gGlu-Glu-AEEA-AEEA-AEEA-Lys(Ac-
Cys³⁹mut.OXM(C³⁸C³⁹))-Gly-OH;
xi.
(SEQ ID NO: 49)
H(Aib)QGTFTSDYSKYLDSKKAQEFVQWLLN(Aib)GRNRNNIAC³⁸
(Eicosanedioic-gGlu-(AEEA)₂-Lys(Ac)-Gly)C³⁹
(Eicosanedioic-gGlu-(AEEA)₂-Lys(Ac)-Gly)-NH₂;
or
xii.
(SEQ ID NO: 50)
H(Aib)QGTFTSDYSKYLDSKKAQEFVQWLLN(Aib)GRNRNNIAC³⁸
(Eicosanedioic-gGlu-Glu-(AEEA)₃-Lys(Ac)-Gly)C³⁹
(Eicosanedioic-gGlu-Glu-(AEEA)₃-Lys(Ac)-Gly)-
NH₂.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. The modified OXM of claim 1, wherein (i) the binder complex V of Formula II or the W of Formula III is an albumin binding group or (ii) the binder complex V of Formula II or the W of Formula III is an albumin binding group and increases the binding affinity of the native OXM, OXM analog, or active fragment thereof, to human serum albumin.

54. (canceled)

55. (canceled)

56. A composition comprising a mixture of two polypeptides, a first polypeptide comprising any one of the modified OXM of claim 30 and a second polypeptide comprising any one of the modified OXM of claim 30.

57. (canceled)

58. (canceled)

59. (canceled)

60. The modified OXM of claim 1, further comprising a pharmaceutically acceptable salt thereof.

61. A pharmaceutical composition comprising the compound of claim 1, or a pharmaceutically acceptable salt, stereoisomer, solvate, or hydrate thereof, and a pharmaceutically acceptable excipient.

62. A method of treating cardiometabolic and associated diseases comprising administering to a subject in need of such treatment a therapeutically effective amount of the modified OXM of claim 1 or a pharmaceutically acceptable salt, or the pharmaceutical composition of claim 61.

63. The method of claim 62, wherein the disease is T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome Crohn's disease, colitis, irritable bowel syndrome, prevention or treatment of Polycystic Ovary Syndrome and treatment of addiction.

64. A method of reducing the risk of a major adverse cardiovascular event (MACE), comprising:
administering the modified OXM of claim 1, or the pharmaceutical composition of claim 61, in a therapeutically effective amount to a subject in need thereof, wherein the subject has type 2 diabetes and cardiovascular disease.

65. The method of claim 64, wherein MACE is selected from the group consisting of CV death, non-fatal (myocardial infarction) MI, non-fatal stroke, revascularisation, hospitalisation for heart failure, and hospitalisation for unstable angina pectoris.

66. The method of claim 64, wherein the cardiovascular disease is selected from the group consisting of clinical evidence of cardiovascular disease and subclinical evidence of cardiovascular disease;
wherein the clinical evidence of cardiovascular disease is selected from the group consisting of prior myocardial infarction; prior stroke or transient ischaemic attack; prior coronary, carotid, or peripheral arterial revascularization; >50% stenosis on angiography or imaging of coronary, carotid, or lower extremity arteries; history of symptomatic coronary heart disease; asymptomatic cardiac ischemia; heart failure; and chronic renal impairment by estimated glomerular filtration rate <60 mL/min/1.73 m² per MDRD; and
wherein the subclinical evidence of cardiovascular disease is selected from the group consisting of persistent microalbuminuria or proteinuria; hypertension and left ventricular hypertrophy by ECG or imaging; left ventricular systolic or diastolic dysfunction; and ankle/brachial index <0.9.

67. The method according to claim 64, wherein:
i. the subject has a BMI of no more than 30 kg/m²;
ii. the modified OXM is administered for at least 30 months;
ii. said modified OXM is administered in a pharmaceutical composition comprising about 0.1-20 mg/ml modified OXM; and/or
iv. said modified OXM is administered in a pharmaceutical composition comprising between 1 nmol/kg and 30 nmol/kg of body weight of the patient.

68. (canceled)

69. (canceled)

70. (canceled)

71. The pharmaceutical composition according to claim 61, wherein said pharmaceutical composition comprises about 2-15 mM phosphate buffer, about 2-25 mg/ml propylene glycol, about 1-18 mg/ml phenol, and has a pH in the range of 7.0-9.0.

72. Use of a therapeutically effective amount of the modified OXM of claim 1, or the pharmaceutical composition of claim 61, in the manufacture of a medicament for treating a subject with cardiometabolic and associated diseases or for treating a subject in need thereof.

73. (canceled)
